(19) United States
(12) Patent Application Publication
(10) Pub. No.: US 2022/0073550 A1
(43) Pub. Date: Mar. 10, 2022

(54) ORGANOMETALLIC COMPOUND AND ORGANIC LIGHT-EMITTING DEVICE INCLUDING SAME

(71) Applicant: Samsung Display Co., Ltd., Yongin-si (KR)

(72) Inventors: Eunsoo Ahn, Yongin-si (KR); Soobyung Ko, Yongin-si (KR); Sungbum Kim, Yongin-si (KR); Eunyoung Lee, Yongin-si (KR); Jaesung Lee, Yongin-si (KR)

(21) Appl. No.: 17/447,275

(22) Filed: Sep. 9, 2021

(30) Foreign Application Priority Data
Sep. 10, 2020 (KR) 10-2020-0116096

(51) Int. Cl. C07F 15/00; H01L 51/00

(52) U.S. Cl. CPC C07F 15/0086; H01L 51/5016; H01L 51/0087

(57) ABSTRACT

Provided are an organometallic compound represented by Formula 1 and an organic light-emitting device including the organometallic compound. The organic light-emitting device includes; a first electrode; a second electrode facing the first electrode; an organic layer between the first electrode and the second electrode and comprising an emission layer; and at least one of the organometallic compound represented by Formula 1.

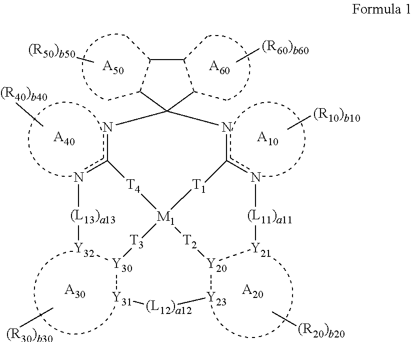

### CROSS-REFERENCE TO RELATED APPLICATION

This application claims priority to and the benefit of Korean Patent Application No. 10-2020-0116096, filed on Sep. 10, 2020, in the Korean Intellectual Property Office, the entire content of which is hereby incorporated by reference.

### BACKGROUND

### 1. Field

One or more embodiments of the present disclosure relate to an organometallic compound and an organic light-emitting device including the same.

### 2. Description of Related Art

Organic light-emitting devices (OLEDs) are self-emissive devices that, as compared with other devices of the related art, have wide viewing angles, high contrast ratios, short response times, and excellent characteristics in terms of brightness, driving voltage, and response speed, and produce full-color images.

OLEDs may include a first electrode on a substrate, and a hole transport region, an emission layer, an electron transport region, and a second electrode sequentially stacked on the first electrode. Holes provided from the first electrode may move toward the emission layer through the hole transport region, and electrons provided from the second electrode may move toward the emission layer through the electron transport region. Carriers, such as holes and electrons, recombine in the emission layer to produce excitons. These excitons transit (e.g., transition or relax) from an excited state to a ground state to thereby generate light.

### SUMMARY

One or more embodiments of the present disclosure include a novel organometallic compound and an organic light-emitting device including the same.

Additional aspects of embodiments will be set forth in part in the description which follows and, in part, will be apparent from the description, or may be learned by practice of the presented embodiments of the disclosure.

According to an aspect of an embodiment, an organometallic compound may be represented by Formula 1:

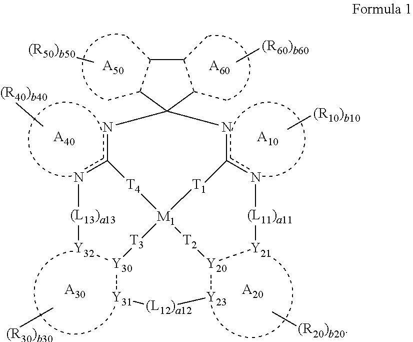

According to another aspect of an embodiment, an organic light-emitting device may include a first electrode; a second electrode; an organic layer between the first electrode and the second electrode and including an emission layer and at least one organometallic compound represented by Formula 1.

According to an aspect of another embodiment, an electronic apparatus may include the organic light-emitting device.

### BRIEF DESCRIPTION OF THE DRAWINGS

The above and other aspects and features of certain embodiments of the disclosure will be more apparent from the following description taken in conjunction with the accompanying drawings, in which:

FIG. 1 is a schematic cross-sectional view of a light-emitting device according to an embodiment;

FIG. 2 is a schematic cross-sectional view of an electronic apparatus according to an embodiment; and

FIG. 3 is a schematic cross-sectional view of an electronic apparatus according to another embodiment.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of embodiments of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

An organometallic compound may be represented by Formula 1:

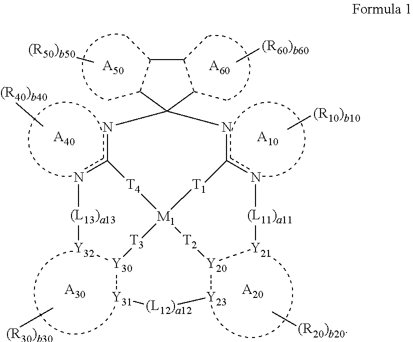

In Formula 1, M₁ may be selected from platinum (Pt), palladium (Pd), copper (Cu), silver (Ag), gold (Au), rhodium (Rh), iridium (Ir), ruthenium (Ru), osmium (Os), titanium (Ti), zirconium (Zr), hafnium (Hf), europium (Eu), terbium (Tb), and thulium (Tm).

In some embodiments, M₁ may be selected from Pt, Pd, Cu, Ag, Au, Rh, Ir, Ru, and Os. In some embodiments, M₁ may be Pt.

In Formula 1, A₁₀ and A₄₀ may each independently be a N-containing C₁-C₆₀ heterocyclic group.

In some embodiments, A₁₀ and A₄₀ may each independently be an imidazole group, a benzimidazole group, a naphthoimidazole group, a 4,5,6,7-tetrahydrobenzimidazole group, a 2,3-dihydrobenzimidazole group, an imidazopyridine group, a 2,3-dihydroimidazopyridine group, an imidazopyrimidine group, a 2,3-dihydroimidazopyrimidine group, an imidazopyrazine group, or a 2,3-dihydroimidazopyrazine group.

In Formula 1, A₂₀, A₃₀, A₅₀, and A₆₀ may each independently be a C₅-C₆₀ carbocyclic group or a C₁-C₆₀ heterocyclic group.

In some embodiments, A₂₀, A₃₀, A₅₀, and A₆₀ may each independently be selected from a benzene group, a naphthalene group, an anthracene group, a phenanthrene group, a triphenylene group, a pyrene group, a chrysene group, a cyclopentadiene group, a 1,2,3,4-tetrahydronaphthalene group, a furan group, a thiophene group, a silole group, an indene group, a fluorene group, an indole group, a carbazole group, a benzofuran group, a dibenzofuran group, a benzothiophene group, a dibenzothiophene group, a benzosilole group, a dibenzosilole group, an indenopyridine group, an indolopyridine group, a benzofuropyridine group, a benzothienopyridine group, a benzosilolopyridine group, an indenopyrimidine group, an indolopyrimidine group, a benzofuropyrimidine group, a benzothienopyrimidine group, a benzosilolopyrimidine group, a dihydropyridine group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a 2,3-dihydroimidazole group, a triazole group, a 2,3-dihydrotriazole group, an oxazole group, an iso-oxazole group, a thiazole group, an isothiazole group, an oxadiazole group, a thiadiazole group, a benzopyrazole group, a benzimidazole group, a 2,3-dihydrobenzimidazole group, an imidazopyridine group, a 2,3-dihydroimidazopyridine group, an imidazopyrimidine group, a 2,3-dihydroimidazopyrimidine group, an imidazopyrazine group, a 2,3-dihydroimidazopyrazine group, a benzoxazole group, a benzothiazole group, a benzoxadiazole group, a benzothiadiazole group, a 5,6,7,8-tetrahydroisoquinoline group, and a 5,6,7,8-tetrahydroquinoline group.

In some embodiments, A₂₀, A₃₀, A₅₀, and A₆₀ may each independently be a group represented by one selected from Formulae 2-1 to 2-43:

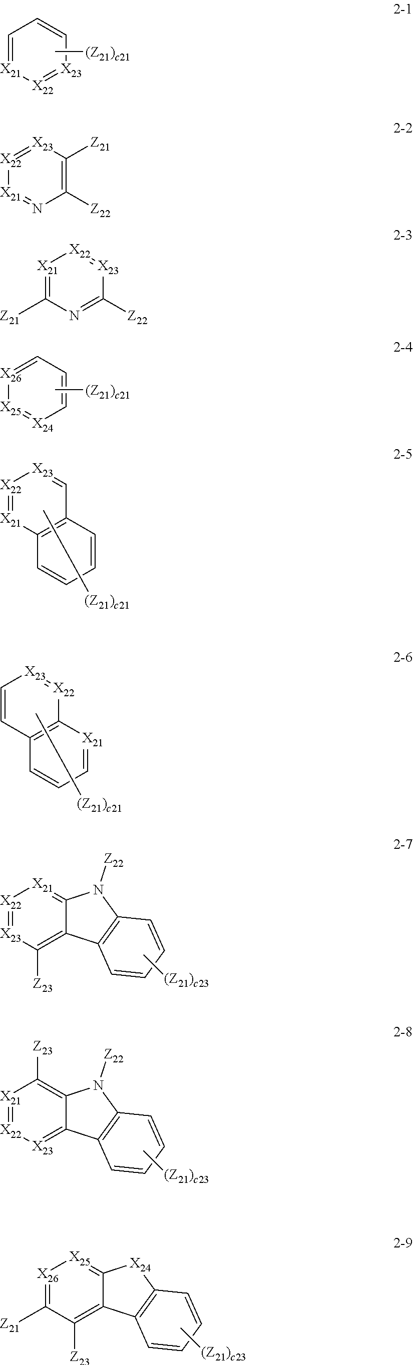

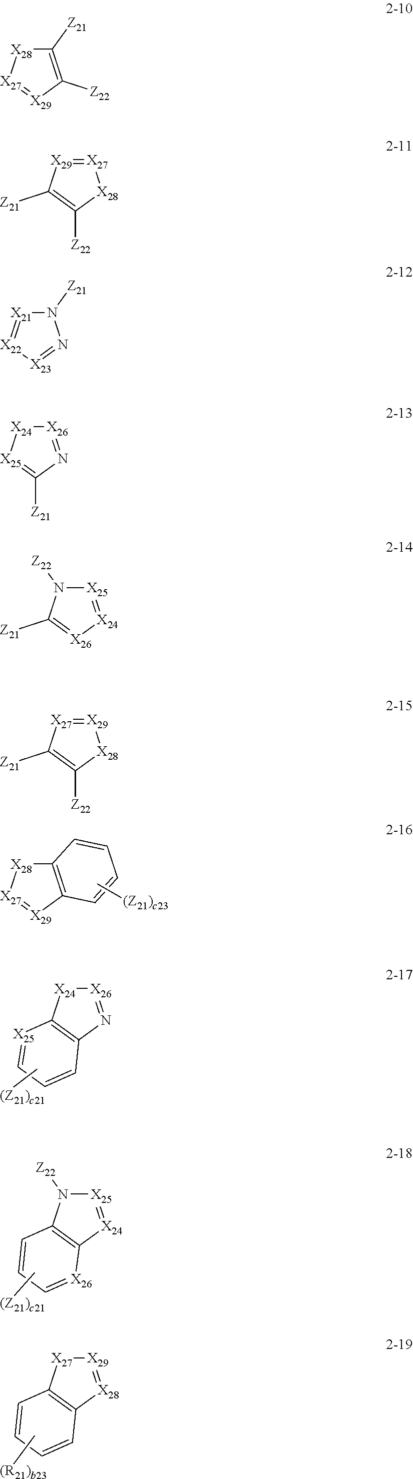

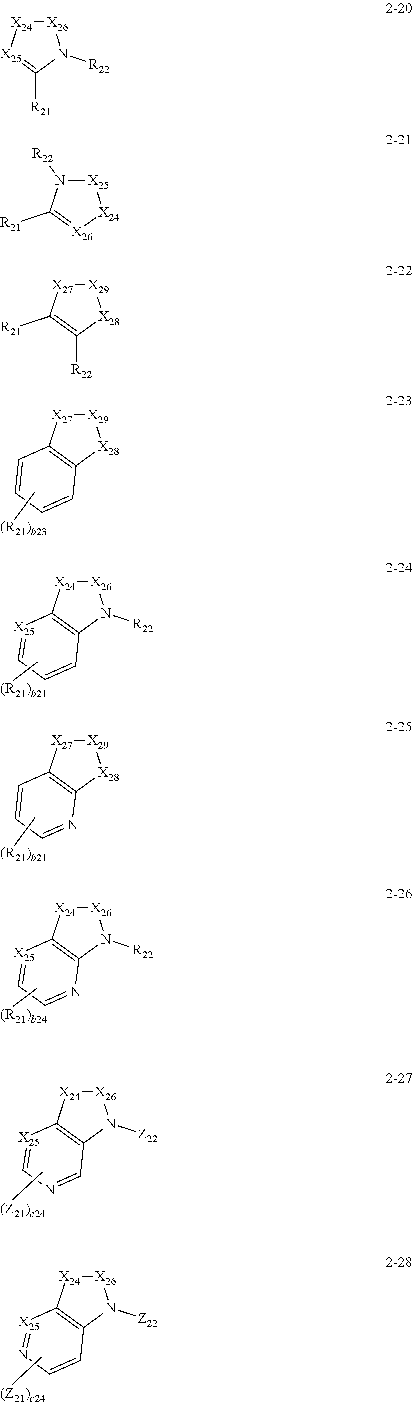

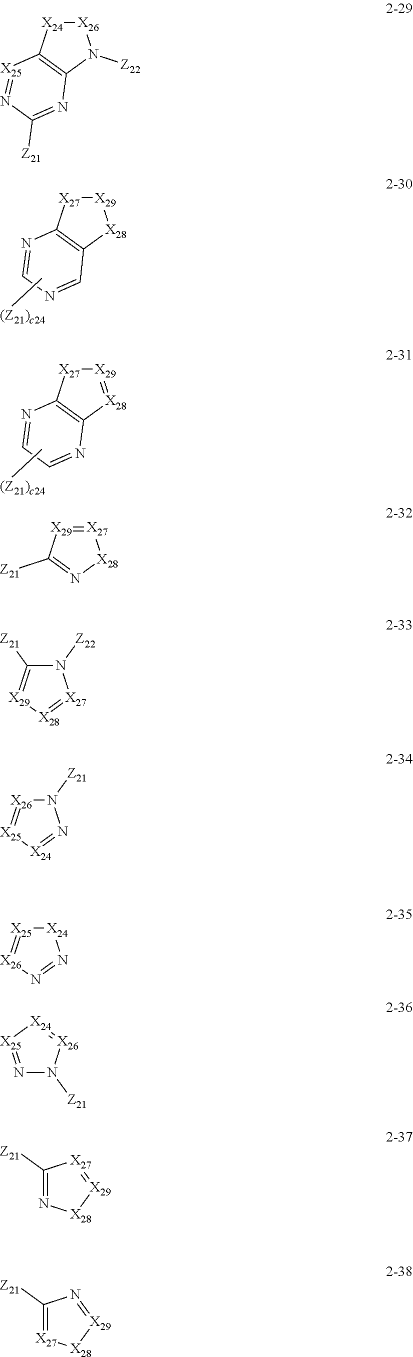

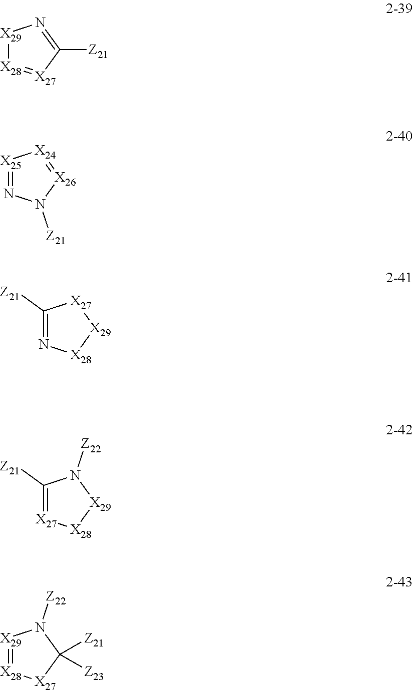

wherein, in Formulae 2-1 to 2-43,

X₂₁ to X₂₃ may each independently be C(Z₂₄) or C—*, and at least two selected from X₂₁ to X₂₃ may each be C—*,

X₂₄ may be N—*, X₂₅ and X₂₆ may each independently be C(Z₂₄) or C—*, and at least one selected from X₂₅ and X₂₆ may be C—*,

X₂₇ and X₂₈ may each independently be N, N(Z₂₅), or N—*, X₂₉ may be C(Z₂₄) or C—*, i) at least one selected from X₂₇ and X₂₈ may be N—*, and X₂₉ may be C—*, or ii) X₂₇ and X₂₈ may be N—*, and X₂₉ may be C(Z₂₄),

Z₂₁ to Z₂₅ may each independently be deuterium, —F, —Cl, —Br, —I, a hydroxyl group, a cyano group, a nitro group, an amidino group, a hydrazino group, a hydrazono group, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a naphthyl group, a fluorenyl group, a spiro-bifluorenyl group, a phenanthrenyl group, an anthracenyl group, a triphenylenyl group, a pyridinyl group, a pyrimidinyl group, a carbazolyl group, or a triazinyl group,

c21 may be 1, 2, or 3,

c22 may be 1, 2, 3, 4, or 5,

c23 may be 1, 2, 3, or 4,

c24 may be 1 or 2, and

* indicates a binding site to an adjacent atom.

In some embodiments, A₂₀, A₃₀, A₅₀, and A₆₀ may each independently be a benzene group, a naphthalene group, a fluorene group, a carbazole group, a dibenzofuran group, a dibenzothiophene group, a dibenzosilole group, a pyridine group, a pyrimidine group, a pyrazine group, a triazine group, a quinoline group, an isoquinoline group, a quinoxaline group, or a quinazoline group.

In some embodiments, A₂₀, A₃₀, A₅₀, and A₆₀ may each independently be a benzene group, a pyridine group, a pyrimidine group, or a triazine group.

In Formula 1, Y₂₀ and Y₃₀ may each independently be N or C.

In Formula 1, Y₂₁, Y₂₂, Y₃₁, and Y₃₂ may each independently be N or C.

In Formula 1, T₁ to T₄ may each indicate a chemical bond.

For example, T₁ to T₄ may each be a coordinate bond (e.g., a single coordinate covalent bond or dative bond) or a covalent bond (e.g., a single covalent bond).

In some embodiments, two selected from T₁ to T₄ may each be a coordinate bond, and the other two may each be a covalent bond. Accordingly, the organometallic compound represented by Formula 1 may not have a form of a salt including a cation and an anion and may be electrically neutral.

In some embodiments, T₁ and T₄ may each be a coordinate bond, and T₂ and T₃ may each be a covalent bond.

In Formula 1, L₁₁ to L₁₃ may each independently be selected from a single bond, *—O—*′, *—S—*′, *—C(R₁)(R₂)—*′, *—C(R₁)═*′, *═C(R₁)—*′, *—C(R₁)═C(R₂)—*′, *—C(═O)—*′, * C(═S)—*′, *—C≡C—*′, *—B(R₁)—*′, *—N(R₁)—*′, *—P(R₁)—*′, *—Si(R₁)(R₂)—*′, *—P(R₁)(R₂)—*′, and *—Ge(R₁)(R₂)*′.

In some embodiments, L₁₁ to L₁₃ may each independently be selected from a single bond (e.g., a single covalent bond), *—O—*′, *—S—*′, *—N(R₁)—*′, *—C(R₁)(R₂)—*′, *—Si(R₁)(R₂)—*′, and *—B(R₁)—*′.

In some embodiments, L₁₁ and L₁₃ may each be a single bond.

In some embodiments, L₁₂ may be *—O—*′, *—S—*′, *—N(R₁)*′, or *—C(R₁)(R₂)—*′.

In Formula 1, a11 to a13 may each independently be selected from 0, 1, 2, 3, 4, and 5.

In some embodiments, a11 and a12 may each be 1.

In some embodiments, a13 may be 0 or 1.

In Formula 1, R₁, R₂, R₁₀, R₂₀, R₃₀, R₄₀, R₅₀, and R₆₀ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),

at least two selected from R₁, R₂, R₁₀, R₂₀, R₃₀, R₄₀, R₅₀, and R₆₀ may optionally be bound to form a C₅-C₆₀ carbocyclic group unsubstituted or substituted with at least one R₁₀ₐ or a C₁-C₆₀ heterocyclic group unsubstituted or substituted with at least one R₁₀ₐ.

In some embodiments, R₁, R₂, R₁₀, R₂₀, R₃₀, R₄₀, R₅₀, and R₆₀ may each independently be: hydrogen, deuterium, —F, —Cl, —Br, —I, a cyano group, a C₁-C₂₀ alkyl group, or a C₁-C₂₀ alkoxy group;

a C₁-C₂₀ alkyl group, a C₁-C₂₀ alkoxy group, or a C₃-C₁₀ cycloalkyl group, each substituted with deuterium, —F, —Cl, —Br, —I, —CDH₂, —CD₂H, —CD₃, a cyano group, a phenyl group, a biphenyl group, or any combination thereof;

a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a benzoisoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphthosilolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indenocarbazolyl group, or an indolocarbazolyl group;

a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a benzoisoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphthosilolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indenocarbazolyl group, or an indolocarbazolyl group, each substituted with deuterium, —F, —Cl, —Br, —I, —CDH₂, —CD₂H, —CD₃, a cyano group, a C₁-C₂₀ alkyl group, a C₁-C₂₀ alkoxy group, a C₃-C₁₀ cycloalkyl group, a cyclopentyl group, a cyclohexyl group, a cycloheptyl group, a cyclopentenyl group, a cyclohexenyl group, a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a perylenyl group, a pentacenyl group, a pyrrolyl group, a thiophenyl group, a furanyl group, a silolyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an indolyl group, an isoindolyl group, an indazolyl group, a purinyl group, a quinolinyl group, an isoquinolinyl group, a benzoquinolinyl group, a benzoisoquinolinyl group, a phthalazinyl group, a naphthyridinyl group, a quinoxalinyl group, a benzoquinoxalinyl group, a quinazolinyl group, a benzoquinazolinyl group, a cinnolinyl group, a phenanthridinyl group, an acridinyl group, a phenanthrolinyl group, a phenazinyl group, a benzimidazolyl group, a benzofuranyl group, a benzothiophenyl group, a benzosilolyl group, a benzothiazolyl group, a benzoisothiazolyl group, a benzoxazolyl group, a benzoisoxazolyl group, a triazolyl group, a tetrazolyl group, a thiadiazolyl group, an oxadiazolyl group, a triazinyl group, a carbazolyl group, a dibenzofuranyl group, a dibenzothiophenyl group, a dibenzosilolyl group, a benzocarbazolyl group, a naphthobenzofuranyl group, a naphthobenzothiophenyl group, a naphthobenzosilolyl group, a dibenzocarbazolyl group, a dinaphthofuranyl group, a dinaphthothiophenyl group, a dinaphthosilolyl group, an imidazopyridinyl group, an imidazopyrimidinyl group, an oxazolopyridinyl group, a thiazolopyridinyl group, a benzonaphthyridinyl group, an azafluorenyl group, an azaspiro-bifluorenyl group, an azacarbazolyl group, an azadibenzofuranyl group, an azadibenzothiophenyl group, an azadibenzosilolyl group, an indenopyrrolyl group, an indolopyrrolyl group, an indenocarbazolyl group, an indolocarbazolyl group, —Si(Q₃₁)(Q₃₂)(Q₃₃), —N(Q₃₁)(Q₃₂), —B(Q₃₁)(Q₃₂), —C(═O)(Q₃₁), —S(═O)(Q₃₁), —S(═O)₂(Q₃₁), —P(═O)(Q₃₁)(Q₃₂), —P(═S)(Q₃₁)(Q₃₂), or any combination thereof; or

—Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂).

In some embodiments, R₁, R₂, R₁₀, R₂₀, R₃₀, R₄₀, R₅₀, and R₆₀ may each independently be: hydrogen, deuterium, —F, —Cl, —Br, —I, a cyano group, a C₁-C₂₀ alkyl group, or a C₁-C₂₀ alkoxy group;

a C₁-C₂₀ alkyl group or a C₁-C₂₀ alkoxy group, each substituted with deuterium, —F, —Cl, —Br, —I, a cyano group, a phenyl group, a biphenyl group, or any combination thereof; or

a group represented by one selected from Formulae 5-1 to 5-26 and Formulae 6-1 to 6-55:

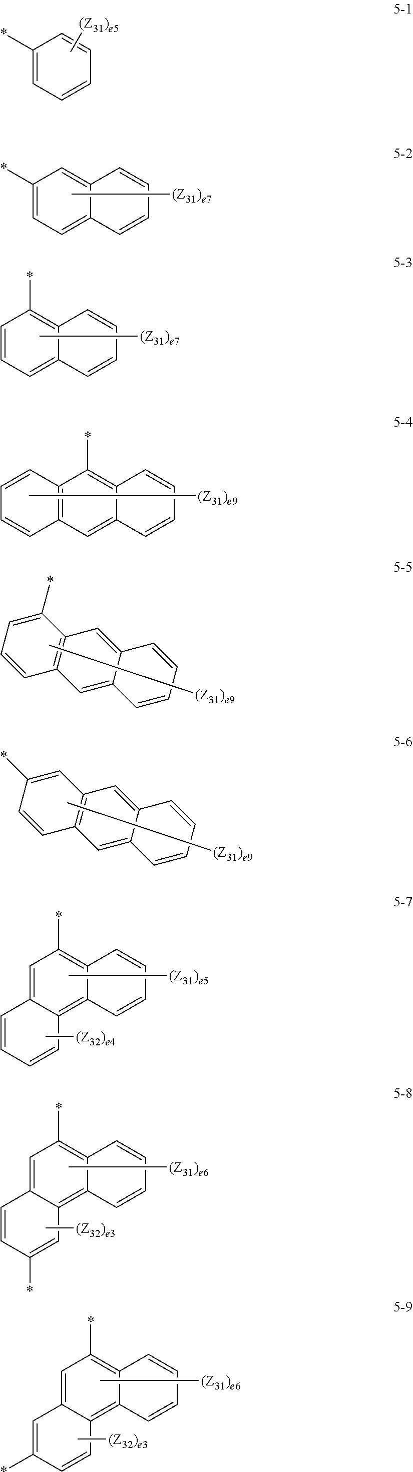

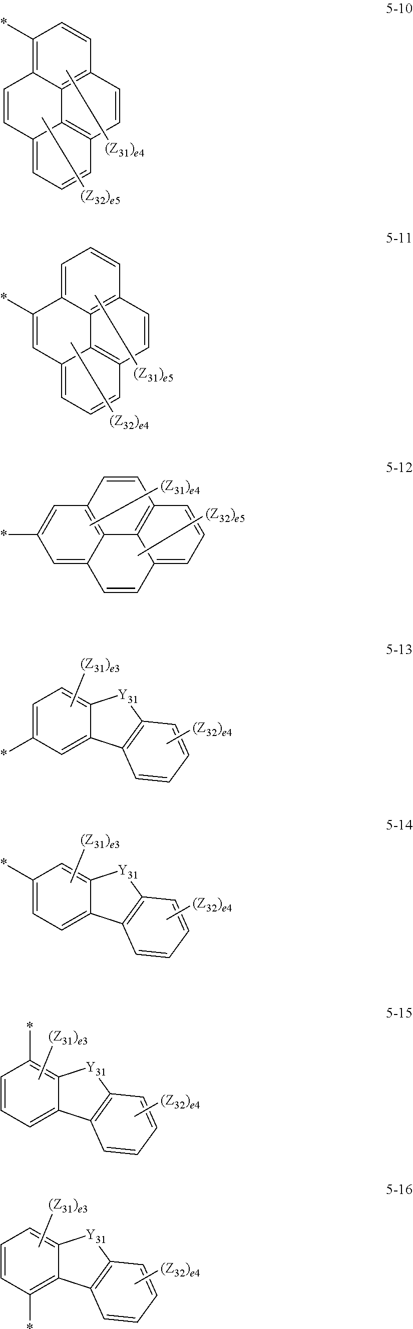

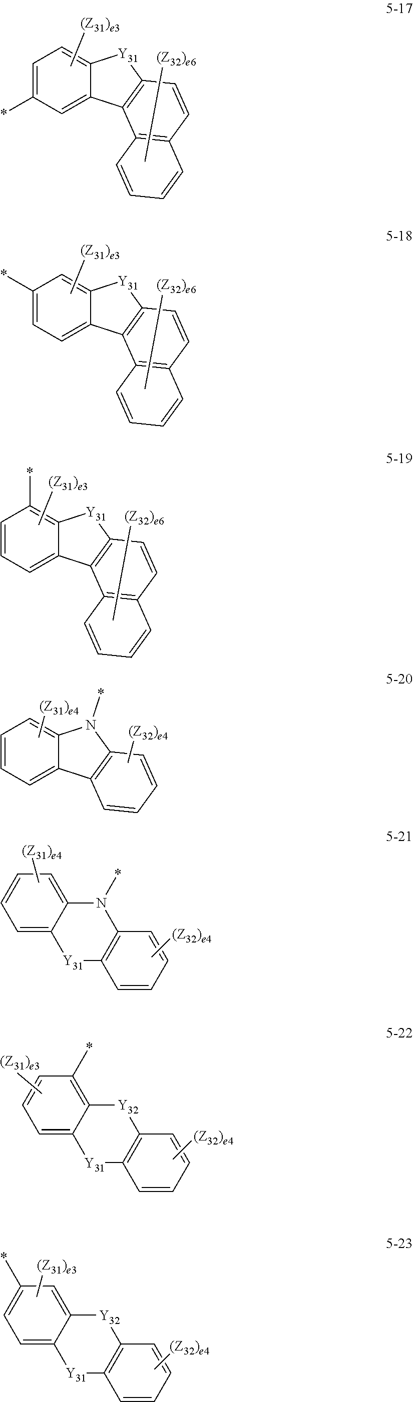

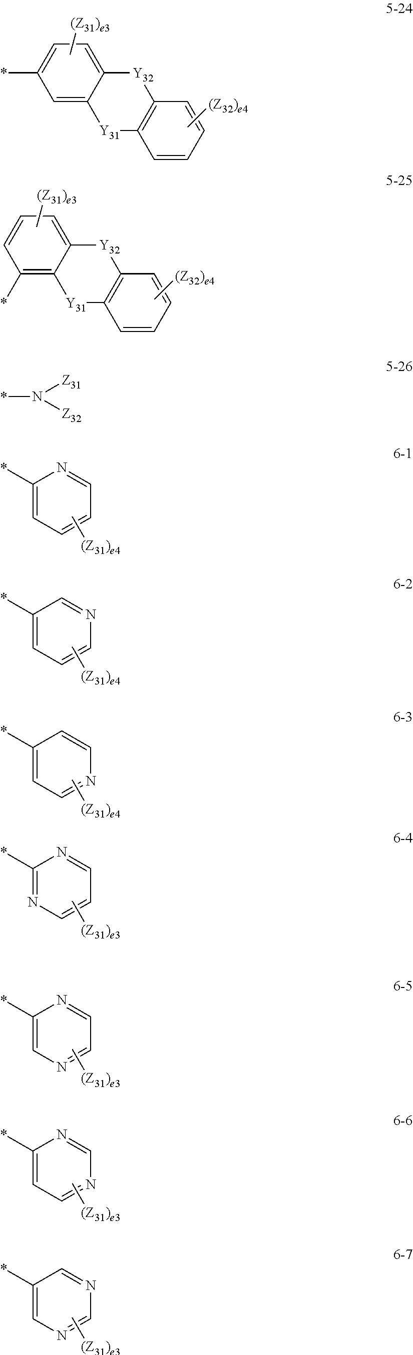

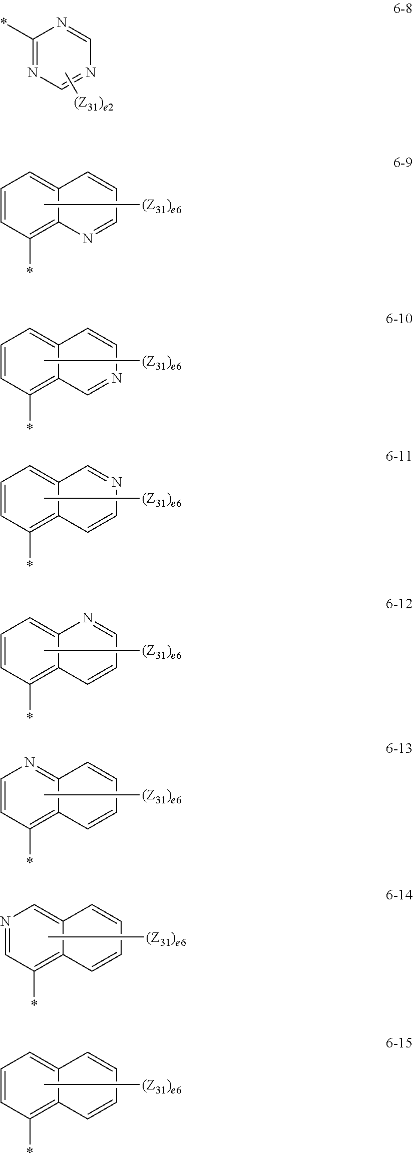

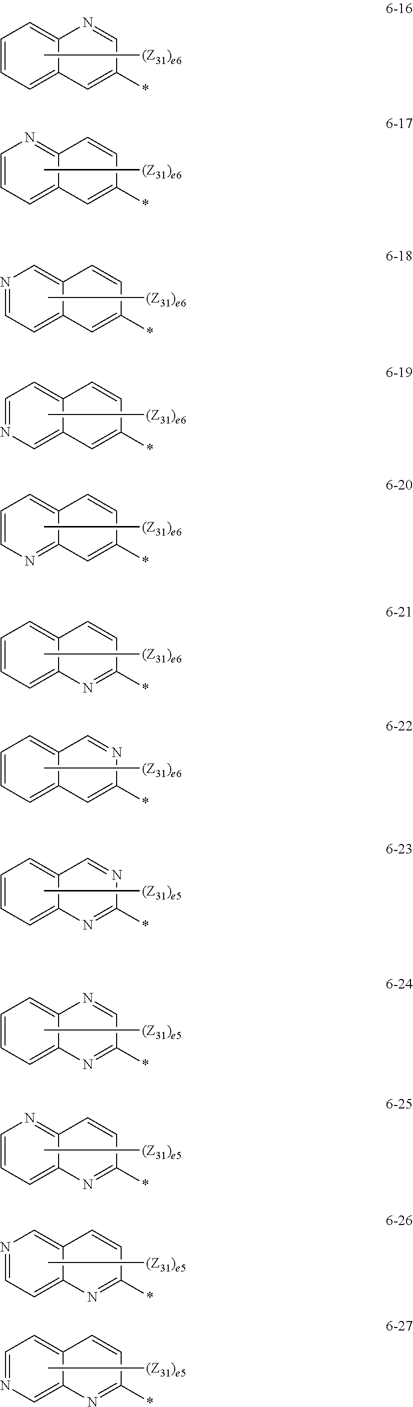

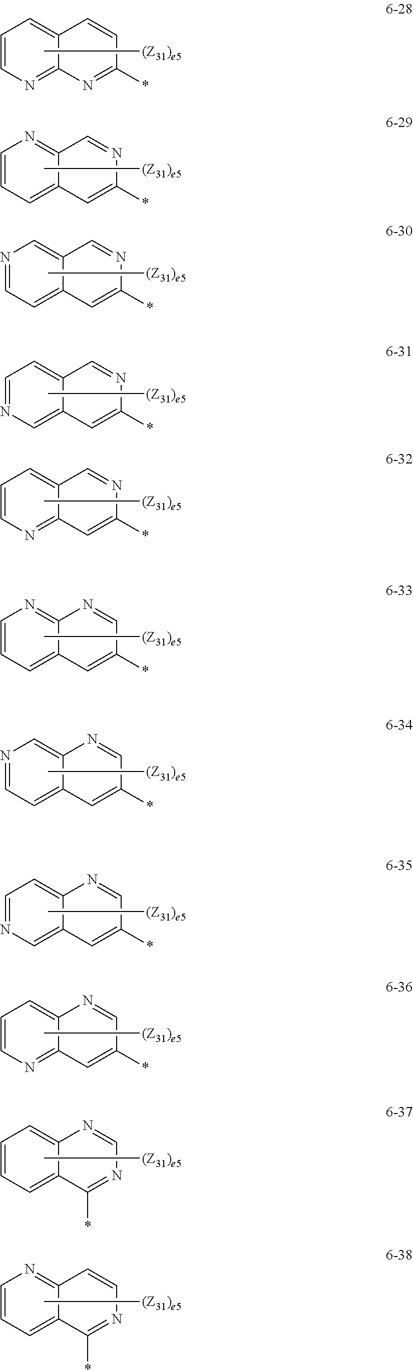

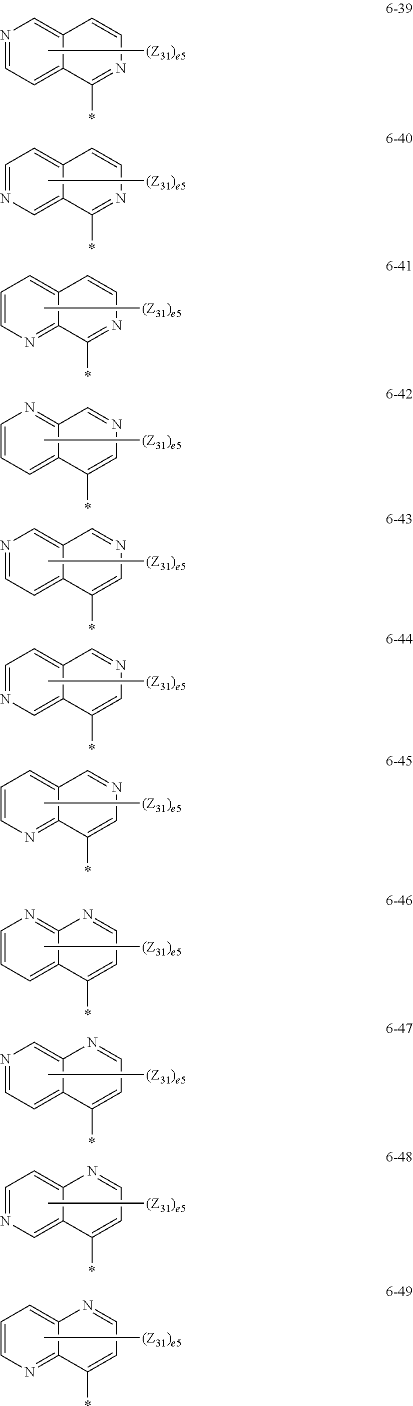

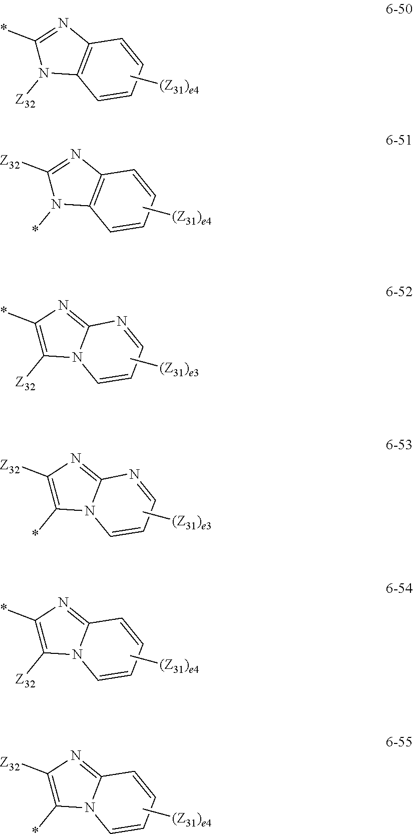

wherein, in Formulae 5-1 to 5-26 and 6-1 to 6-55,

Y₃₁ and Y₃₂ may each independently be O, S, C(Z₃₃)(Z₃₄), N(Z₃₃), or Si(Z₃₃)(Z₃₄),

Z₃₁ to Z₃₄ may each independently be selected from hydrogen, deuterium, —F, —Cl, —Br, —I, a hydroxyl group, a cyano group, a nitro group, an amidino group, a hydrazino group, a hydrazono group, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a naphthyl group, a fluorenyl group, a spiro-bifluorenyl group, a phenanthrenyl group, an anthracenyl group, a triphenylenyl group, a pyridinyl group, a pyrimidinyl group, a carbazolyl group, and a triazinyl group,

e2 may be 1 or 2,

e3 may be an integer from 1 to 3,

e4 may be an integer from 1 to 4,

e5 may be an integer from 1 to 5,

e6 may be an integer from 1 to 6,

e7 may be an integer from 1 to 7,

e9 may be an integer from 1 to 9, and

* indicates a binding site to an adjacent atom.

In some embodiments, at least two adjacent groups of R₁, R₂, R₁₀, R₂₀, R₃₀, R₄₀, R₅₀, and R₆₀ may optionally be bound to form:

a cyclopentane group, a cyclohexane group, a benzene group, a naphthalene group, a fluorene group, or a carbazole group; or

a cyclopentane group, a cyclohexane group, a benzene group, a naphthalene group, a fluorene group, or a carbazole group, each substituted with deuterium, —F, —Cl, —Br, —I, a cyano group, a phenyl group, a biphenyl group, or any combination thereof.

In some embodiments, R₁, R₂, R₁₀, R₂₀, R₃₀, R₄₀, R₅₀, and R₆₀ may each independently be a group represented by one selected from Formulae 5-1 to 5-26 and Formulae 6-1 to 6-55.

In some embodiments, R₁, R₂, R₁₀, R₂₀, R₃₀, R₄₀, R₅₀, and R₆₀ may each independently be selected from: hydrogen, deuterium, —F, —Cl, —Br, —I, a cyano group, a C₁-C₂₀ alkyl group, and a C₁-C₂₀ alkoxy group;

a C₁-C₂₀ alkyl group and a C₁-C₂₀ alkoxy group, each substituted with at least one selected from deuterium, —F, —Cl, —Br, —I, a cyano group, a phenyl group, and a biphenyl group;

a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a carbazolyl group, an acridinyl group, a dibenzofuranyl group, a dibenzothiophenyl group, a benzocarbazolyl group, and a dibenzocarbazolyl group; and

a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a carbazolyl group, an acridinyl group, a dibenzofuranyl group, a dibenzothiophenyl group, a benzocarbazolyl group, and a dibenzocarbazolyl group, each substituted with at least one selected from deuterium, —F, —Cl, —Br, —I, a cyano group, a C₁-C₂₀ alkyl group, a C₁-C₂₀ alkoxy group, a phenyl group, and a biphenyl group.

In Formula 1, at least two adjacent groups of R₁, R₂, R₁₀, R₂₀, R₃₀, R₄₀, R₅₀ and R₆₀ may optionally be bound to form a substituted or unsubstituted C₅-C₆₀ carbocyclic group or a substituted or unsubstituted C₁-C₆₀ heterocyclic group.

In some embodiments, at least two adjacent groups of R₁, R₂, R₁₀, R₂₀, R₃₀, R₄₀, R₅₀, and R₆₀ may optionally be bound to form: a cyclopentane group, a cyclohexane group, a benzene group, a naphthalene group, a fluorene group, or a carbazole group; or

a cyclopentane group, a cyclohexane group, a benzene group, a naphthalene group, a fluorene group, or a carbazole group, each substituted with deuterium, —F, —Cl, —Br, —I, a cyano group, a phenyl group, a biphenyl group, or any combination thereof.

In some embodiments, at least two adjacent groups of R₁, R₂, R₁₀, R₂₀, R₃₀, R₄₀, R₅₀, and R₆₀ may optionally be bound to form a cyclopentane group, a cyclohexane group, a benzene group, a naphthylene group, a fluorene group, or a carbazole group.

In some embodiments, at least two adjacent groups of R₁, R₂, R₁₀, R₂₀, R₃₀, R₄₀, R₅₀, and R₆₀ may optionally be bound to form a cyclopentane group, a cyclohexane group, a fluorene group, or a carbazole group.

In some embodiments, R₁ and R₂ may optionally be bound to form a cyclopentane group, a cyclohexane group, a fluorene group, or a carbazole group.

In Formula 1, b10, b20, b30, b40, b50, and b60 may each independently be 1, 2, 3, 4, 5, 6, 7, or 8.

In some embodiments, the organometallic compound represented by Formula 1 may be represented by Formula 11 or Formula 12:

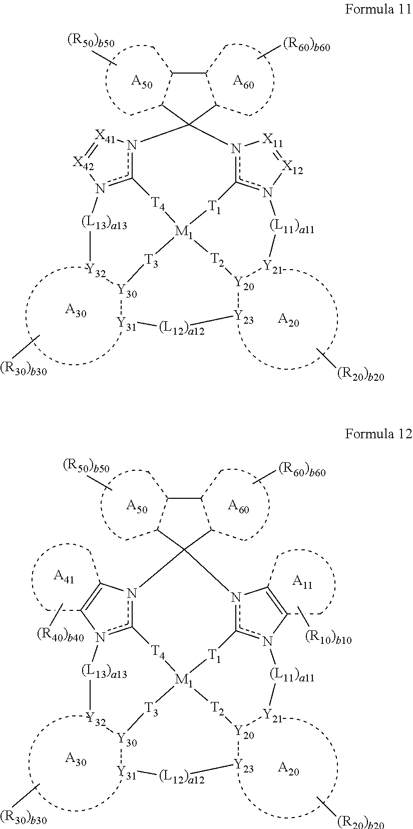

wherein, in Formulae 11 and 12,

M₁, A₂₀, A₃₀, A₅₀, A₆₀, T₁ to T₄, L₁₁ to L₁₃, a11 to a13, R₁₀, R₂₀, R₃₀, R₄₀, R₅₀, R₆₀, b10, b20, b30, b40, b50, and b60 may respectively be understood by referring to the descriptions of M₁, A₂₀, A₃₀, A₅₀, A₆₀, T₁ to T₄, L₁₁ to L₁₃, a11 to a13, R₁₀, R₂₀, R₃₀, R₄₀, R₅₀, R₆₀, b10, b20, b30, b40, b50, and b60 provided herein,

X₁₁ may be C(R₁₁) or N, and X₁₂ may be C(R₁₂) or N,

X₄₁ may be C(R₄₁) or N, and X₄₂ may be C(R₄₂) or N,

A₁₁ and A₄₁ may each independently be a cyclopentane group, a cyclohexane group, a benzene group, a naphthalene group, a fluorene group, an anthracene group, a phenanthrene group, a carbazole group, a dibenzofuran group, a dibenzothiophene group, a dibenzosilole group, a pyridine group, a pyrimidine group, a pyrazine group, a triazine group, a quinoline group, an isoquinoline group, a quinoxaline group, or a quinazoline group,

R₁₁ and R₁₂ may each independently be understood by referring to the description of R₁₀ provided herein, and

R₄₁ and R₄₂ may each independently be understood by referring to the description of R₄₀ provided herein.

In some embodiments, the organometallic compound represented by Formula 1 may be represented by one selected from Formulae 20-1 to 20-12:

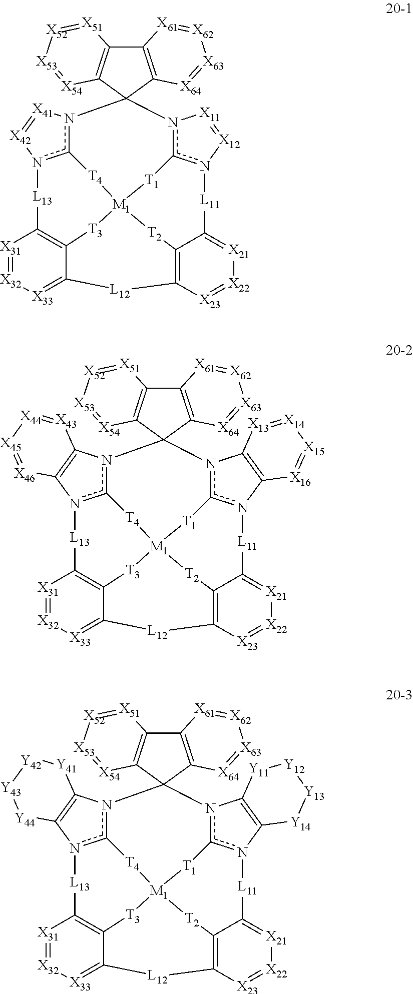

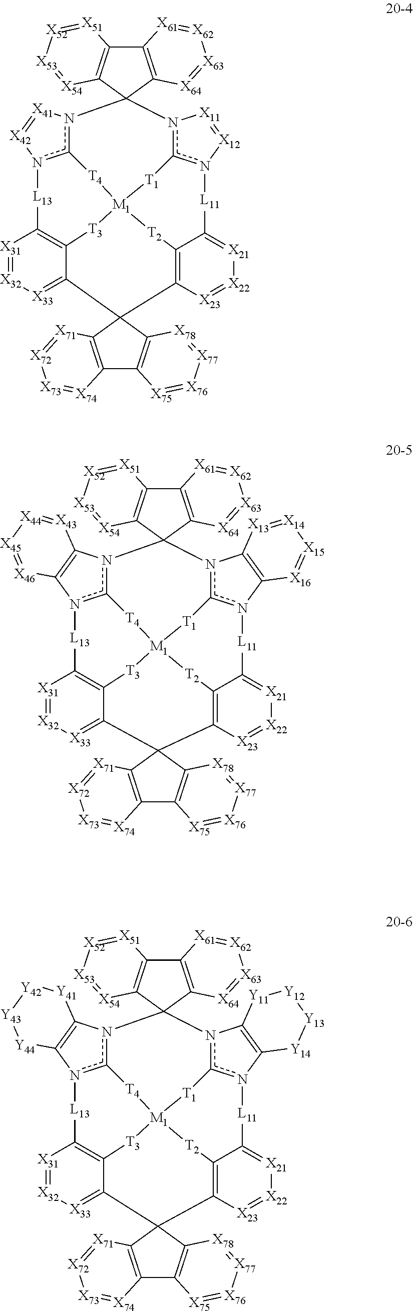

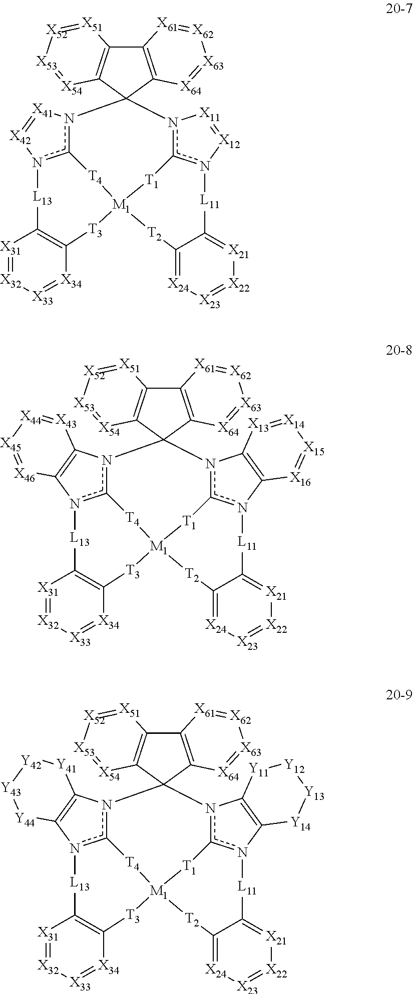

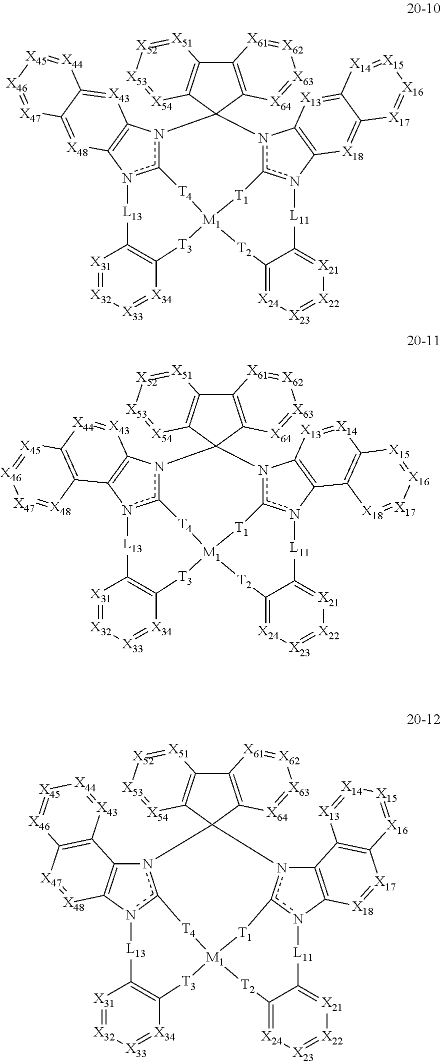

wherein, in Formulae 20-1 to 20-12,

M₁, T₁ to T₄, and L₁₁ to L₁₃ may respectively be understood by referring to the descriptions of M₁, T₁ to T₄, and L₁₁ to L₁₃ provided herein,

X₁₁ may be C(R₁₁) or N, X₁₂ may be C(R₁₂) or N, X₁₃ may be C(R₁₃) or N, X₁₄ may be C(R₁₄) or N, X₁₅ may be C(R₁₅) or N, X₁₆ may be C(R₁₆) or N, X₁₇ may be C(R₁₇) or N, and X₁₈ may be C(R₁₈) or N,

X₂₁ may be C(R₂₁) or N, X₂₂ may be C(R₂₂) or N, X₂₃ may be C(R₂₃) or N, and X₂₄ may be C(R₂₄) or N,

X₃₁ may be C(R₃₁) or N, X₃₂ may be C(R₃₂) or N, X₃₃ may be C(R₃₃) or N, and X₃₄ may be C(R₃₄) or N,

X₄₁ may be C(R₄₁) or N, X₄₂ may be C(R₄₂) or N, X₄₃ may be C(R₄₃) or N, X₄₄ may be C(R₄₄) or N, X₄₅ may be C(R₄₅) or N, X₄₆ may be C(R₄₆) or N, X₄₇ may be C(R₄₇) or N, and X₄₈ may be C(R₄₈) or N,

X₅₁ may be C(R₅₁) or N, X₅₂ may be C(R₅₂) or N, X₅₃ may be C(R₅₃) or N, and X₅₄ may be C(R₅₄) or N,

X₆₁ may be C(R₆₁) or N, X₆₂ may be C(R₆₂) or N, X₆₃ may be C(R₆₃) or N, and X₆₄ may be C(R₆₄) or N,

X₇₁ may be C(R₇₁) or N, X₇₂ may be C(R₇₂) or N, X₇₃ may be C(R₇₃) or N, X₇₄ may be C(R₇₄) or N, X₇₅ may be C(R₇₅) or N, X₇₆ may be C(R₇₆) or N, X₇₇ may be C(R₇₇) or N, and X₇₈ may be C(R₇₈) or N,

Y₁₁ may be C(R₁₁)(R₁₂), Y₁₂ may be C(R₁₃)(R₁₄), Y₁₃ may be C(R₁₅)(R₁₆), and Y₁₄ may be C(R₁₇)(R₁₈),

Y₄₁ may be C(R₄₁)(R₄₂), Y₄₂ may be C(R₄₃)(R₄₄), Y₄₃ may be C(R₄₅)(R₄₆), and Y₄₄ may be C(R₄₇)(R₄₈),

R₁₁ to R₁₈ may each independently be understood by referring to the description of R₁₀ provided herein,

R₂₁ to R₂₄ may each independently be understood by referring to the description of R₂₀ provided herein,

R₃₁ to R₃₄ may each independently be understood by referring to the description of R₃₀ provided herein,

R₄₁ to R₄₈ may each independently be understood by referring to the description of R₄₀ provided herein,

R₅₁ to R₅₄ may each independently be understood by referring to the description of R₅₀ provided herein,

R₆₁ to R₆₄ may each independently be understood by referring to the description of R₆₀ provided herein, and

R₇₁ to R₇₈ may each independently be: hydrogen, deuterium, —F, —Cl, —Br, —I, a cyano group, a C₁-C₂₀ alkyl group, or a C₁-C₂₀ alkoxy group,

a C₁-C₂₀ alkyl group or a C₁-C₂₀ alkoxy group, each substituted with deuterium, —F, —Cl, —Br, —I, a cyano group, a phenyl group, a biphenyl group, or any combination thereof;

a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a carbazolyl group, an acridinyl group, a dibenzofuranyl group, a dibenzothiophenyl group, a benzocarbazolyl group, or a dibenzocarbazolyl group; or

a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a carbazolyl group, an acridinyl group, a dibenzofuranyl group, a dibenzothiophenyl group, a benzocarbazolyl group, or a dibenzocarbazolyl group, each substituted with deuterium, —F, —Cl, —Br, —I, a cyano group, a C₁-C₂₀ alkyl group, a C₁-C₂₀ alkoxy group, a phenyl group, a biphenyl group, or any combination thereof.

In some embodiments, the organometallic compound represented by Formula 1 may be represented by one selected from Formulae 30-1 to 30-54:

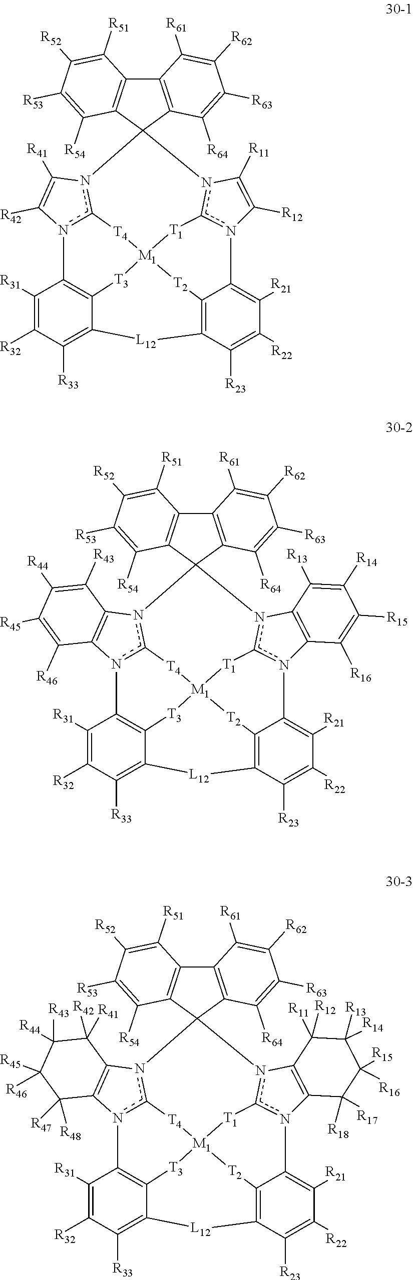

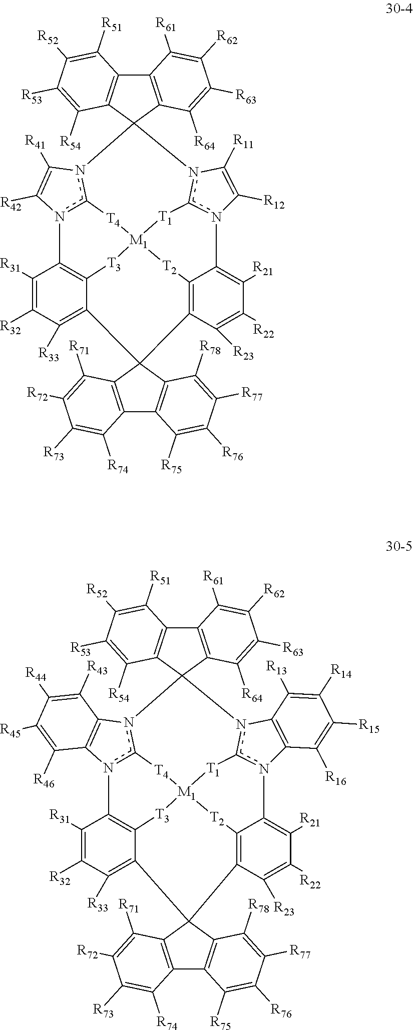

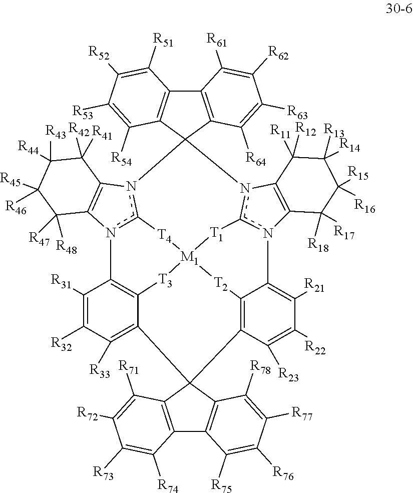

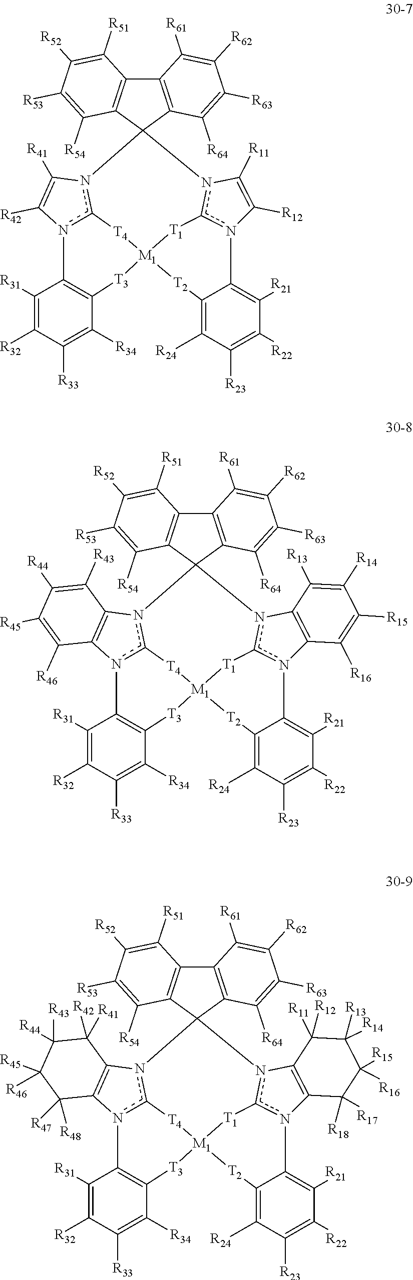

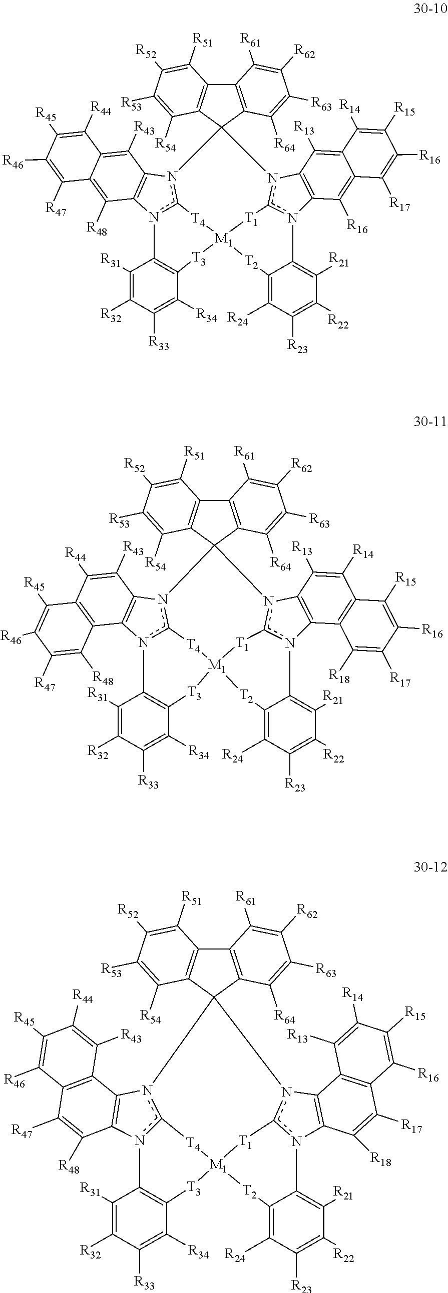

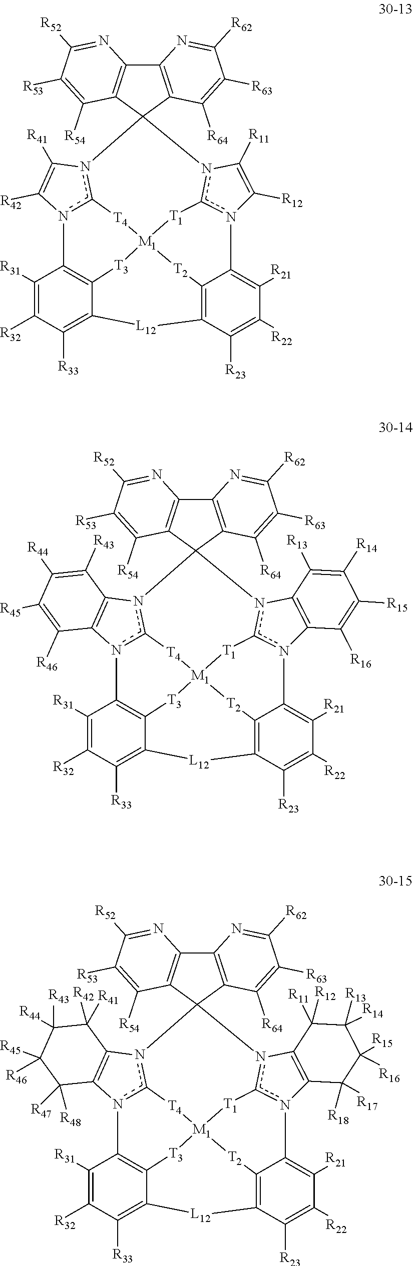

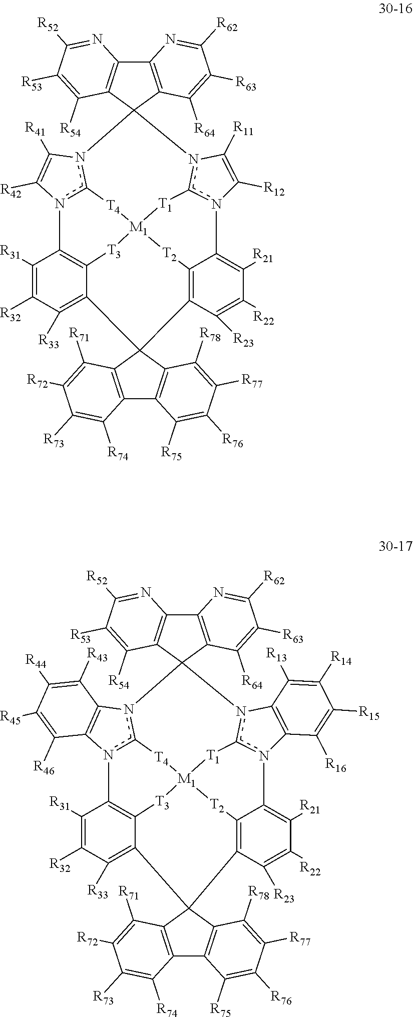

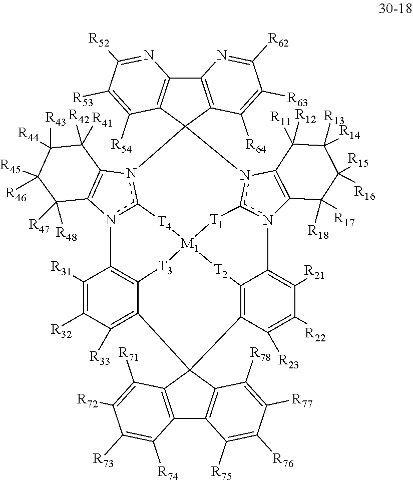

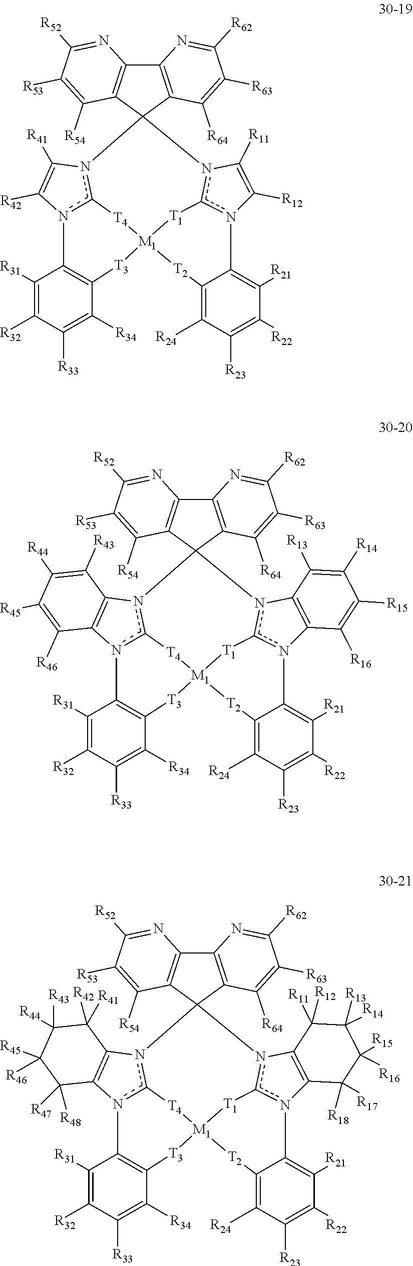

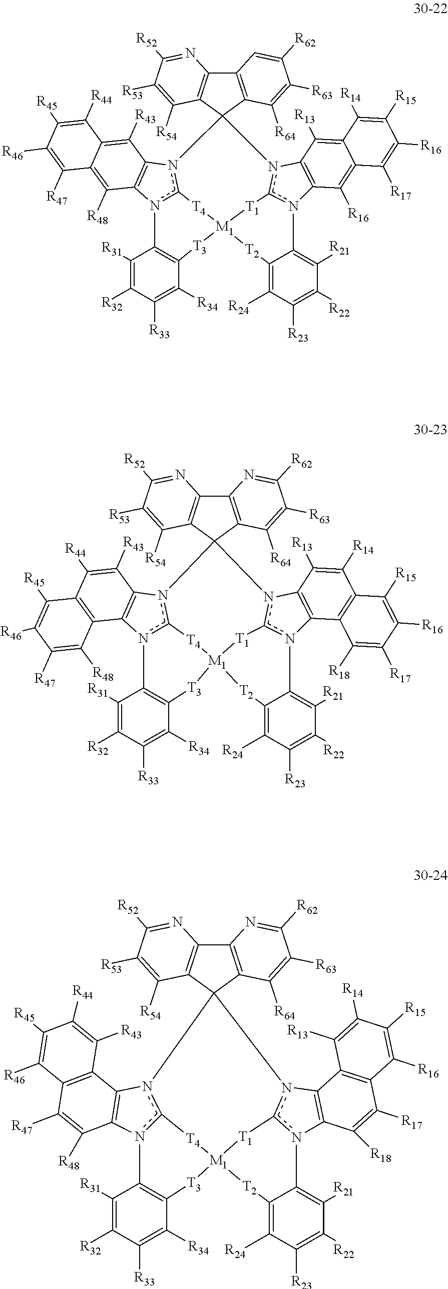

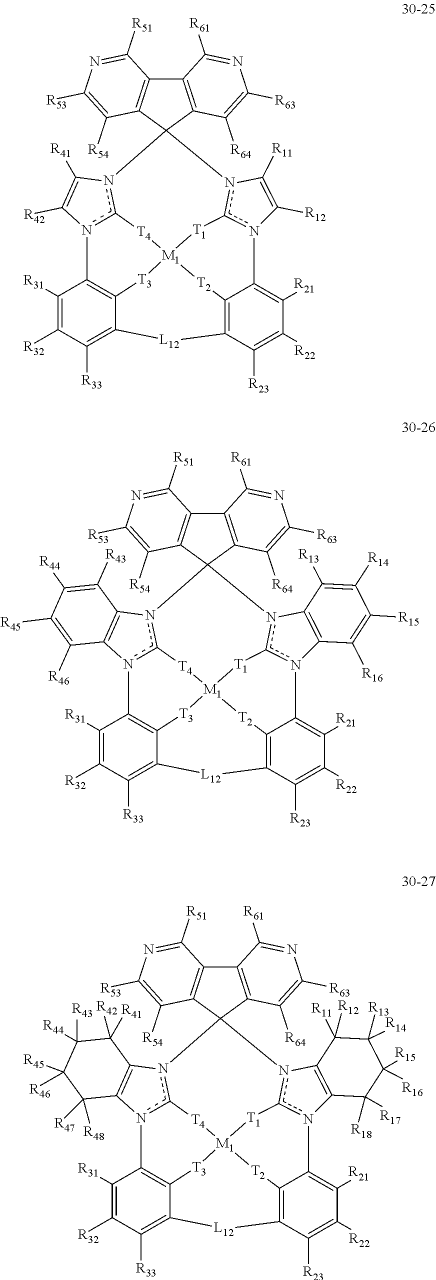

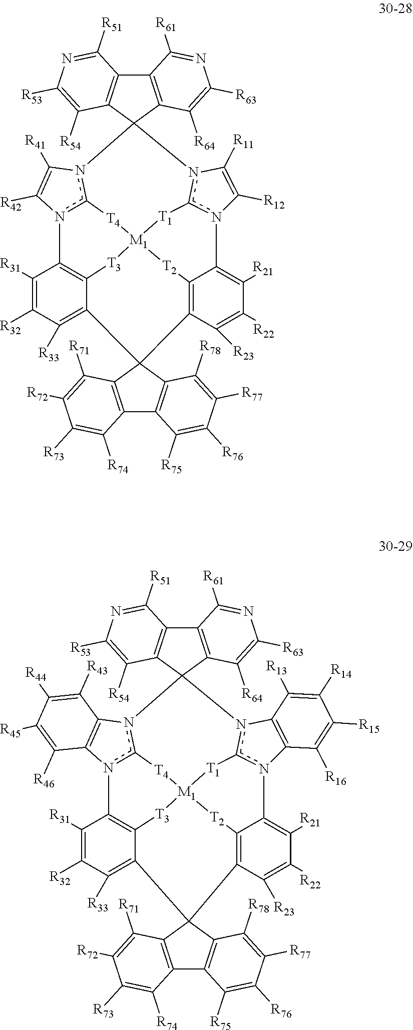

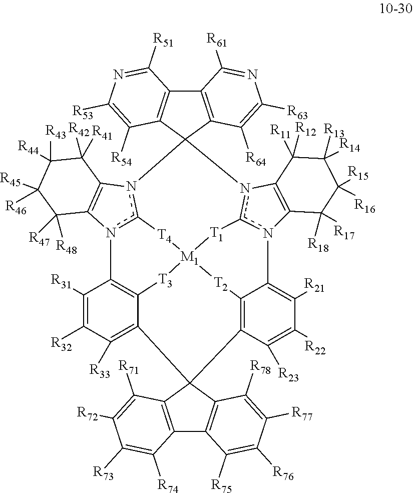

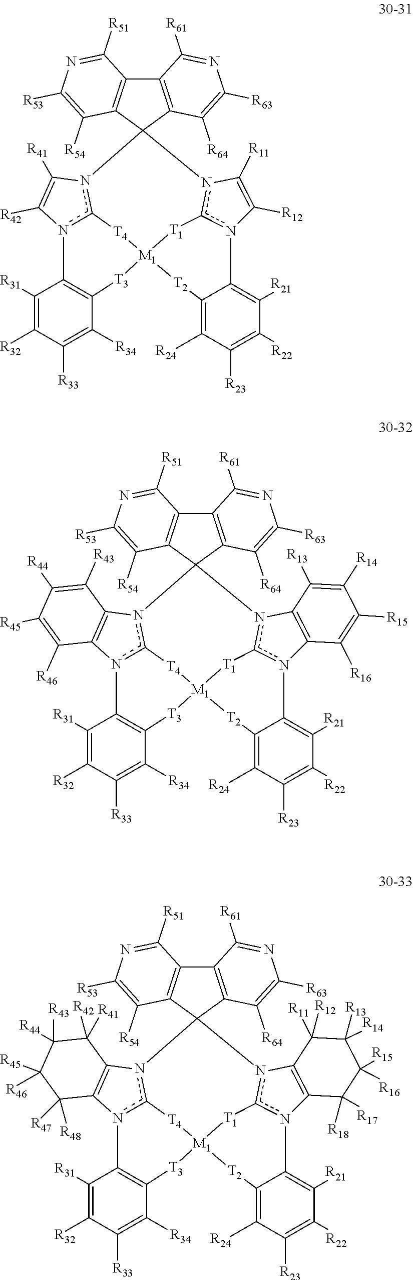

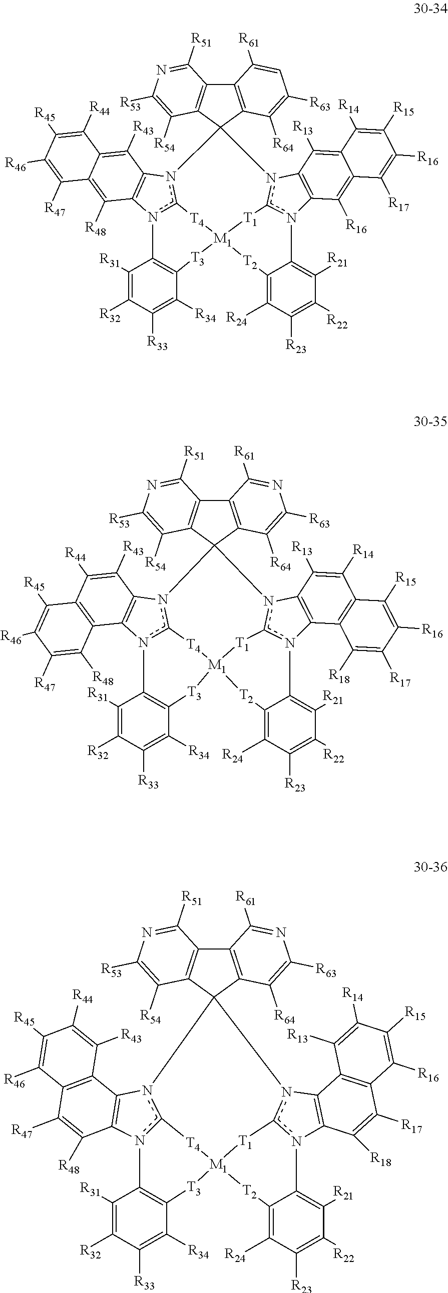

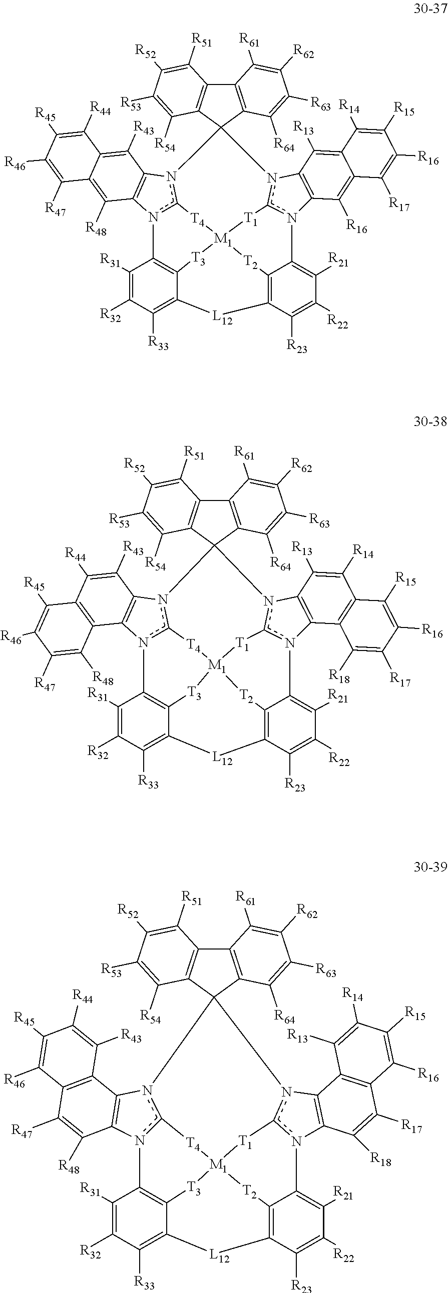

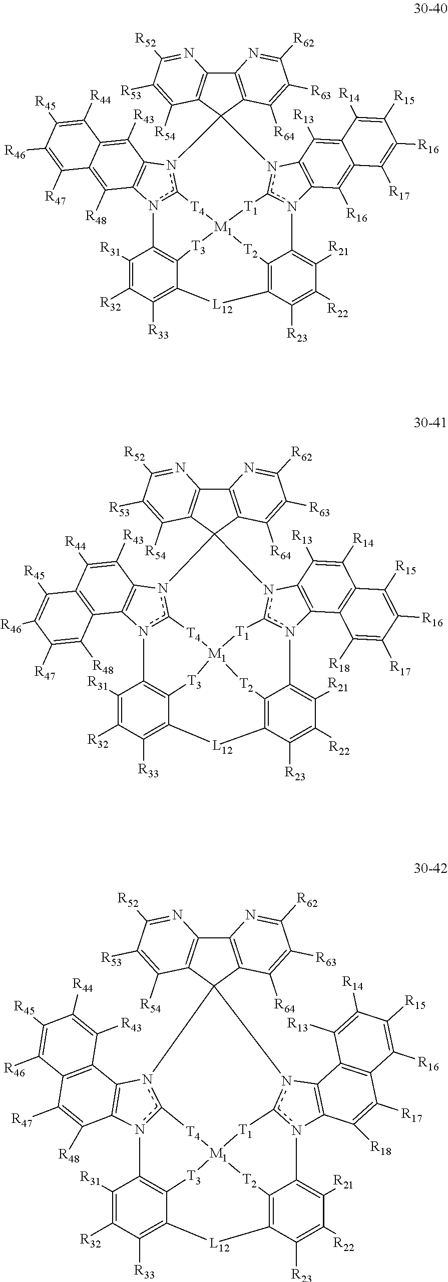

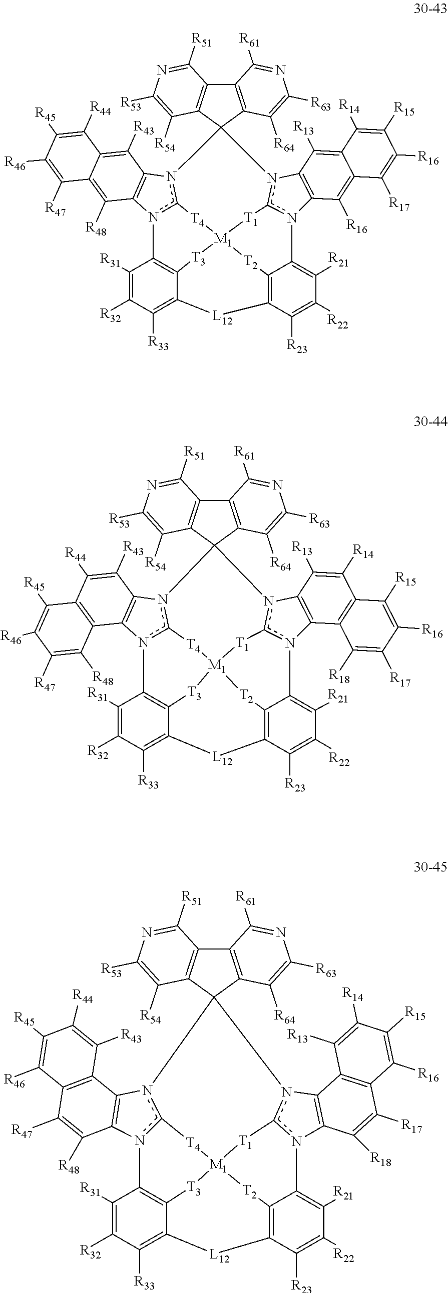

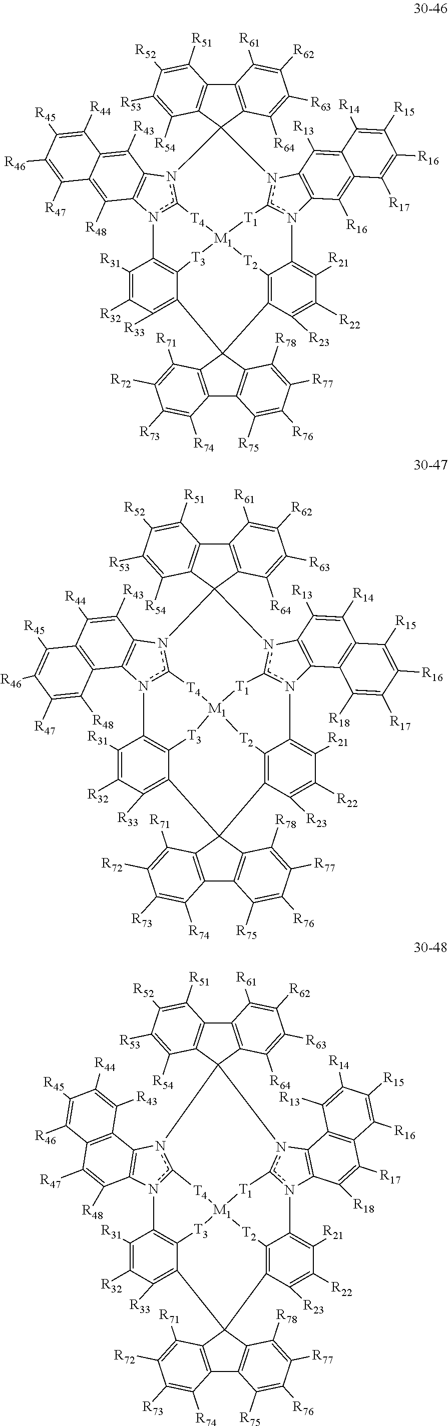

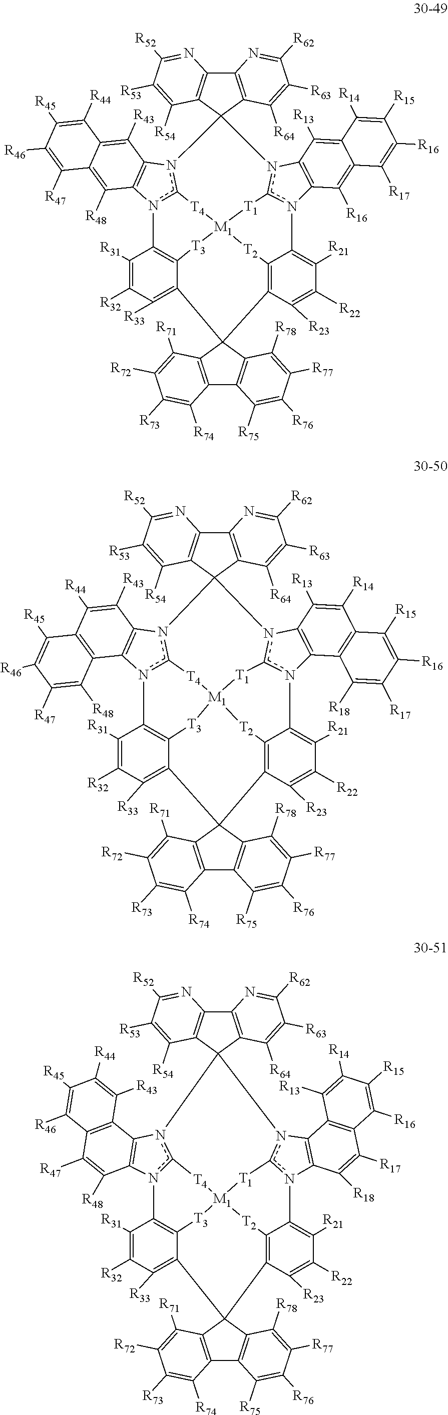

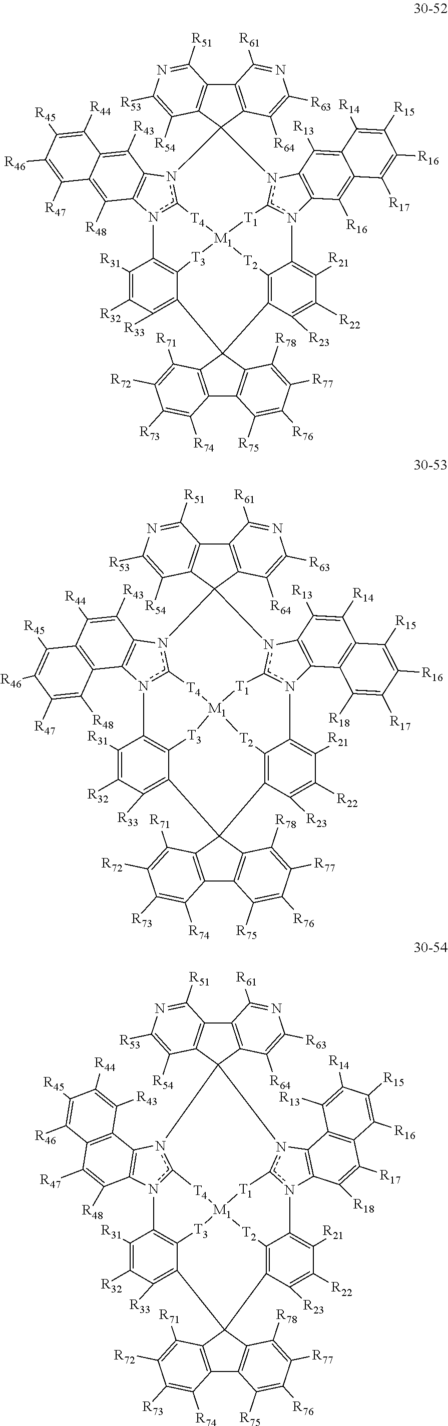

wherein, in Formulae 30-1 to 30-54,

M₁, T₁ to T₄ and L₁₂ may respectively be understood by referring to the descriptions of M₁, T₁ to T₄ and L₁₂ provided herein,

R₁₁ to R₁₈, R₂₁ to R₂₄, R₃₁ to R₃₄, R₄₁ to R₄₈, R₅₁ to R₅₄, R₆₁ to R₆₄, and R₇₁ to R₇₈ may respectively be understood by referring to the descriptions of R₁₁ to R₁₈, R₂₁ to R₂₄, R₃₁ to R₃₄, R₄₁ to R₄₈, R₅₁ to R₅₄, R₆₁ to R₆₄, and R₇₁ to R₇₈ provided herein.

In some embodiments, the organometallic compound represented by Formula 1 may be selected from Compounds BD1 to BD66, but embodiments are not limited thereto:

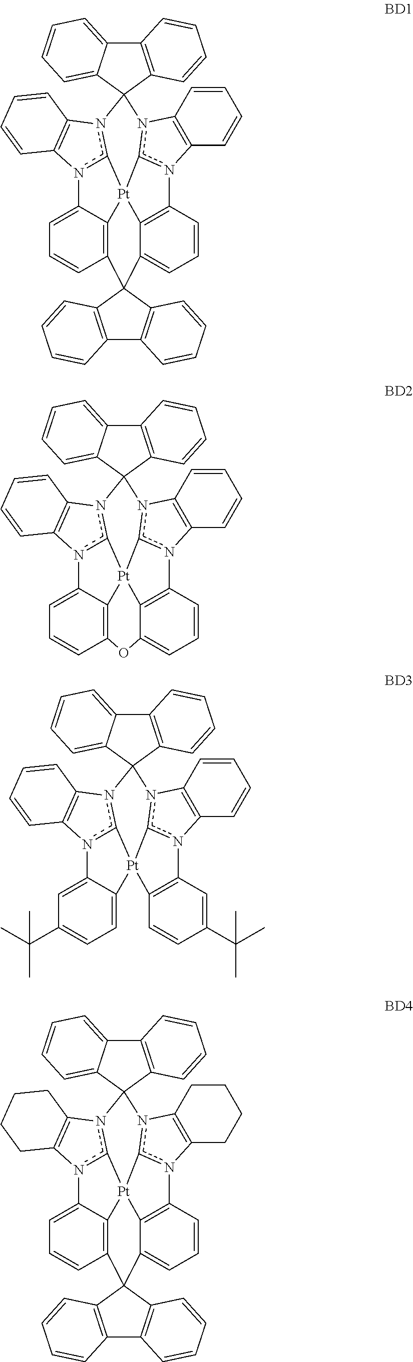

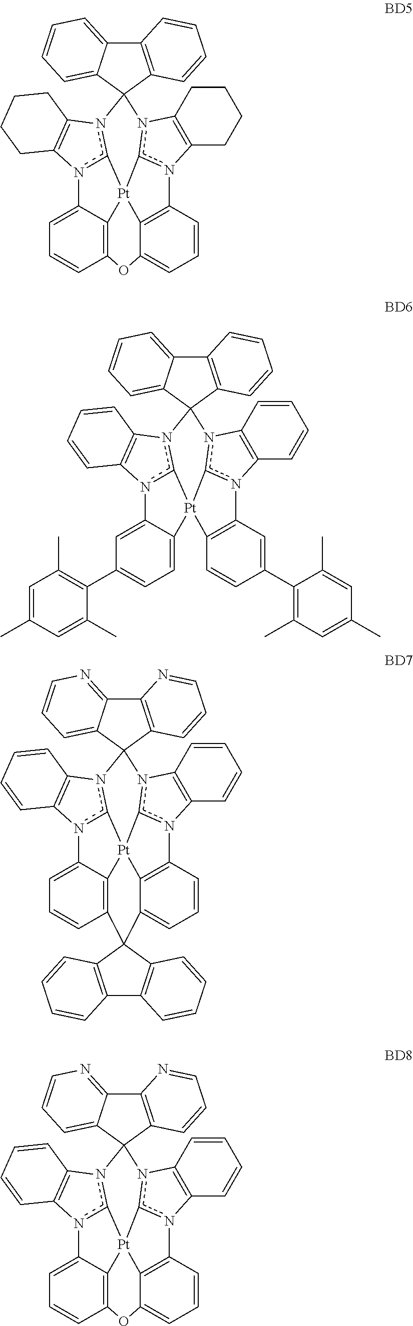

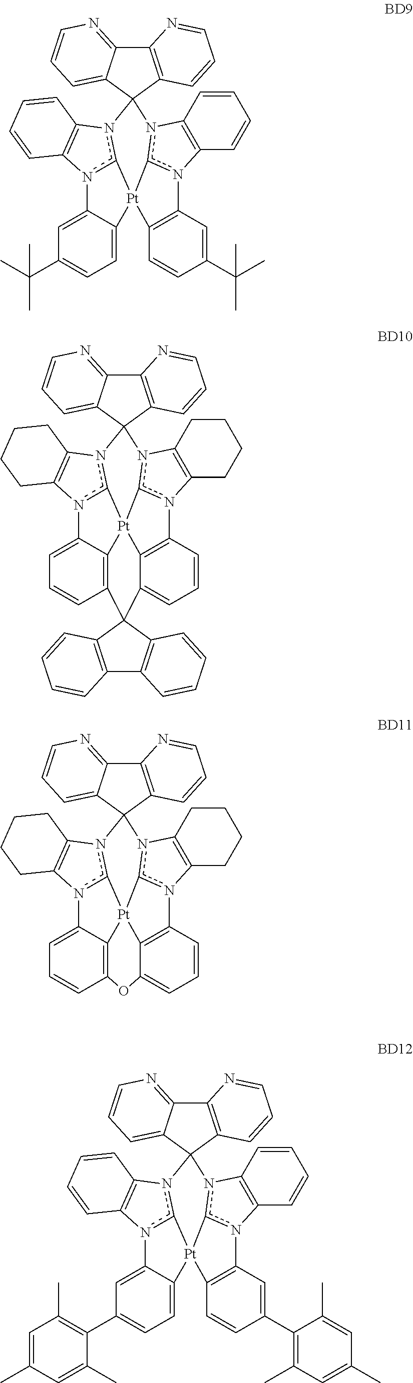

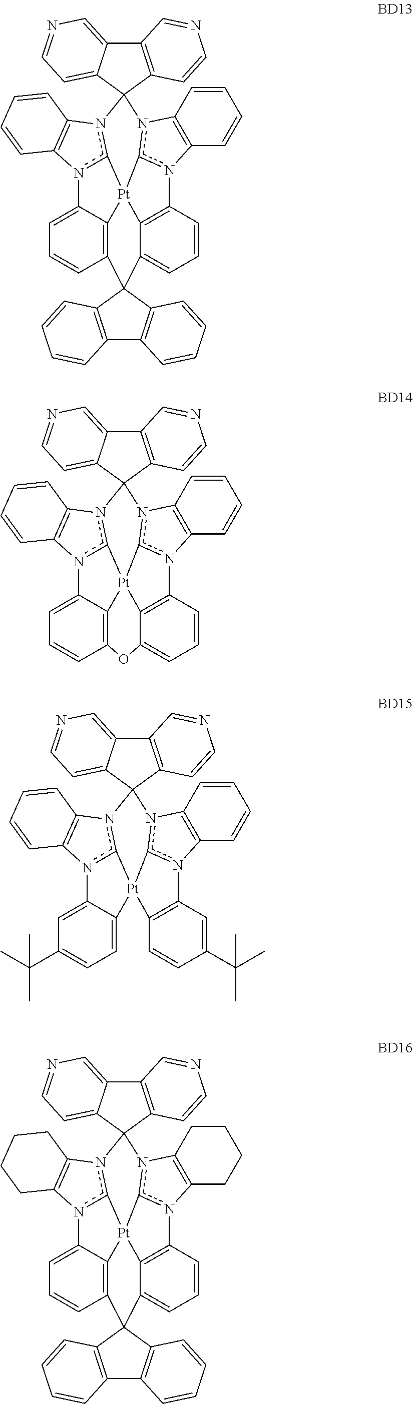

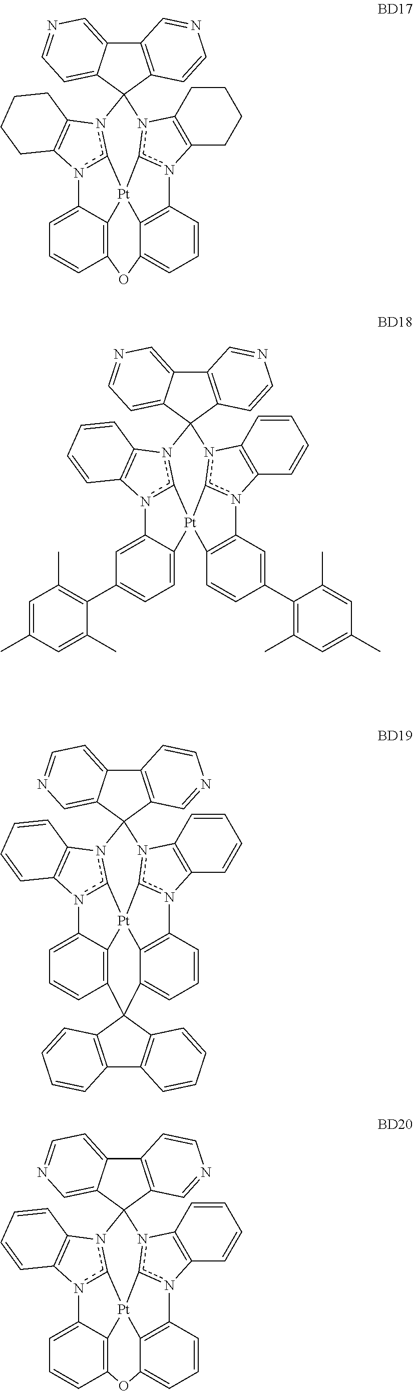

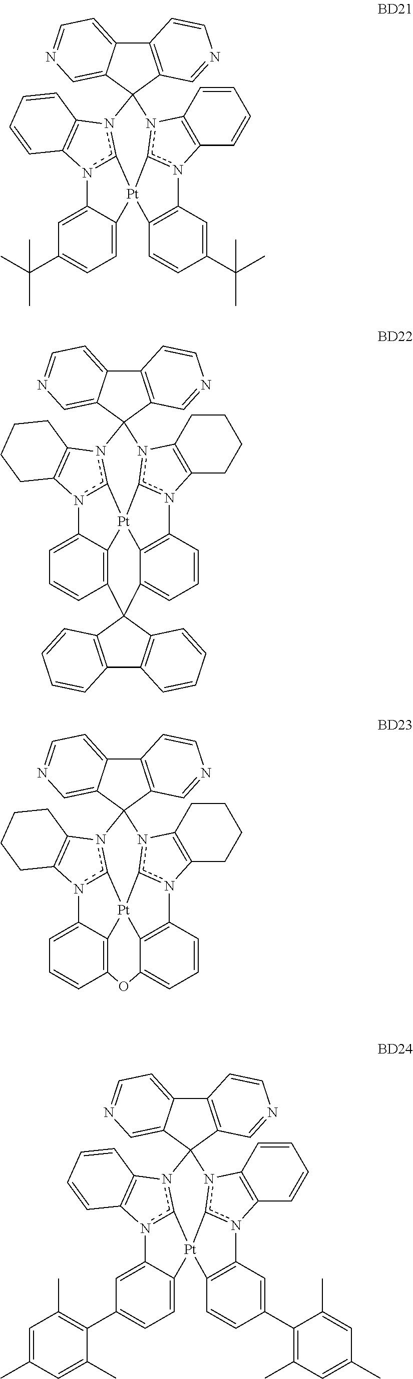

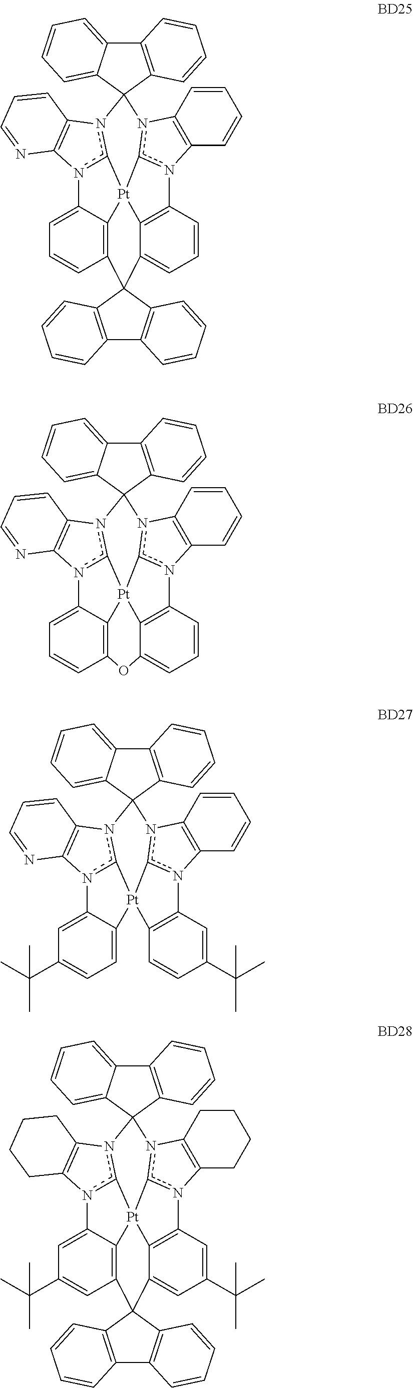

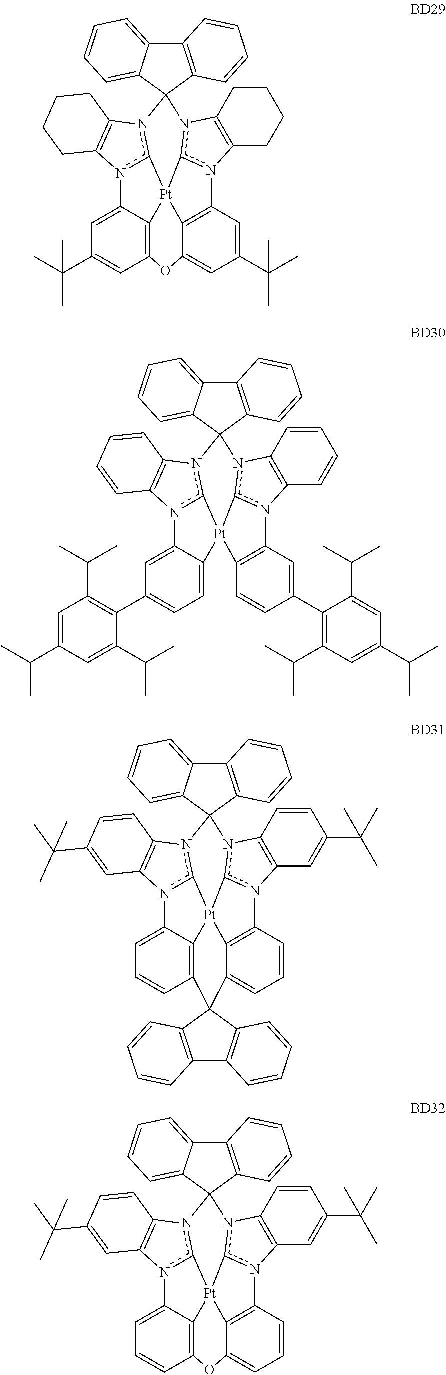

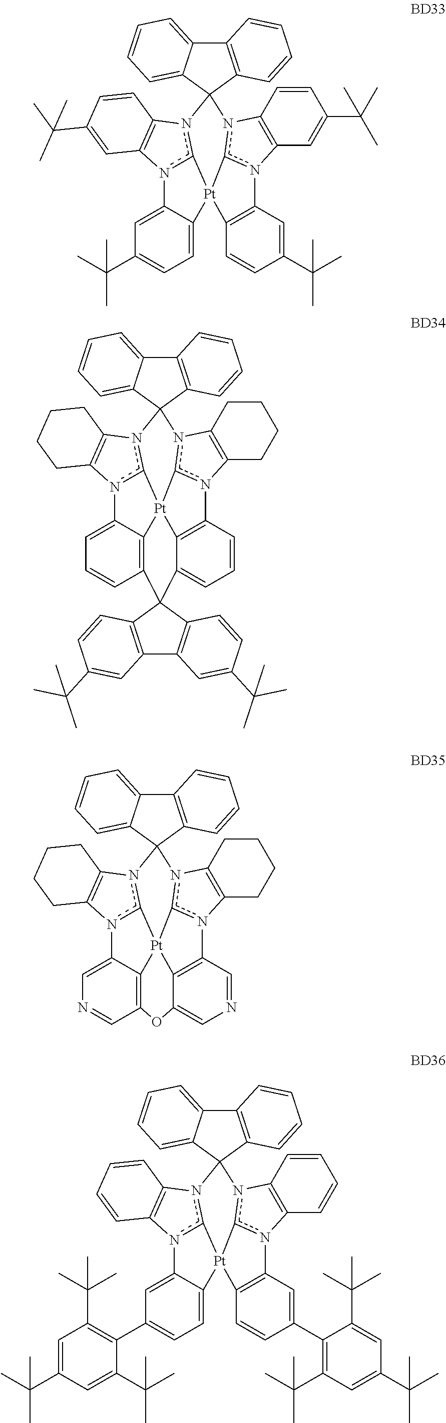

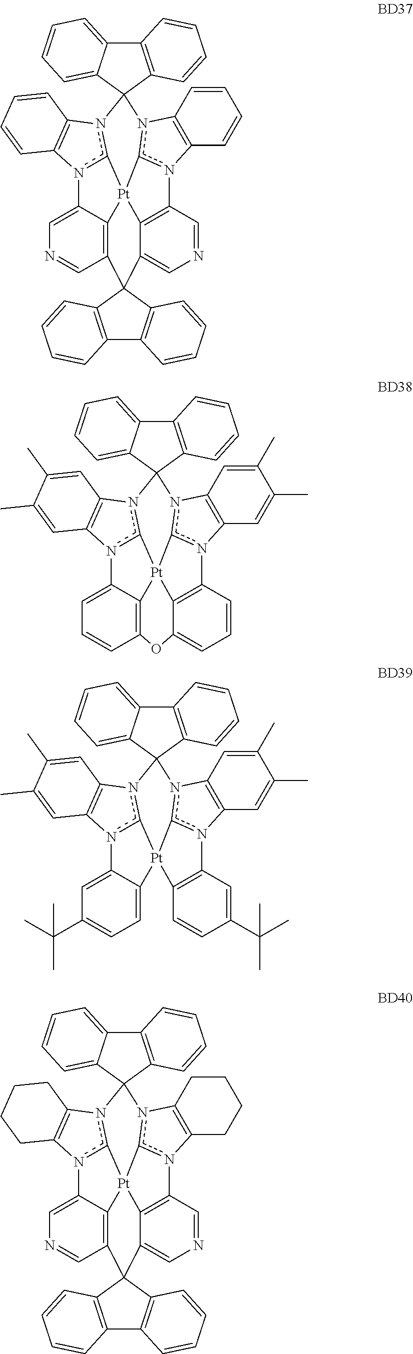

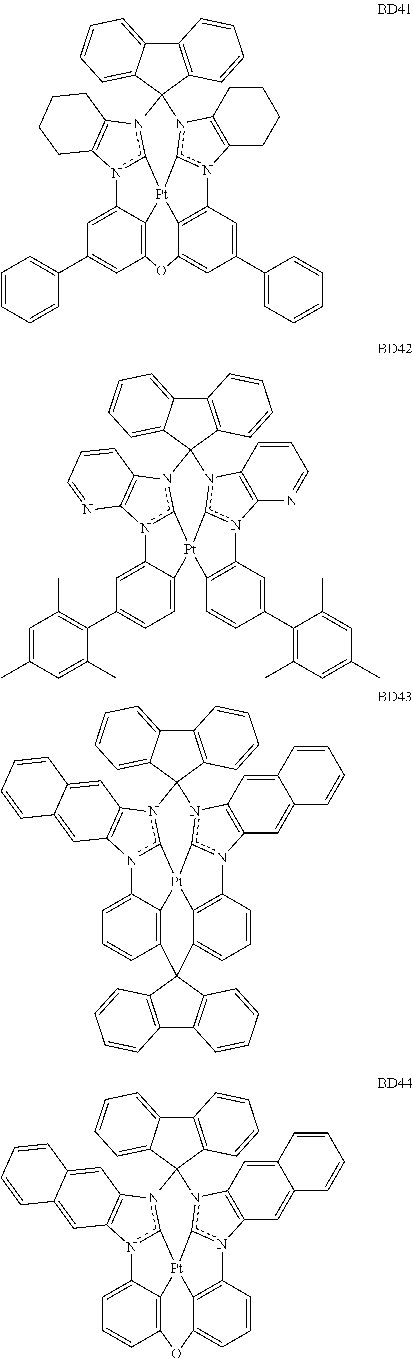

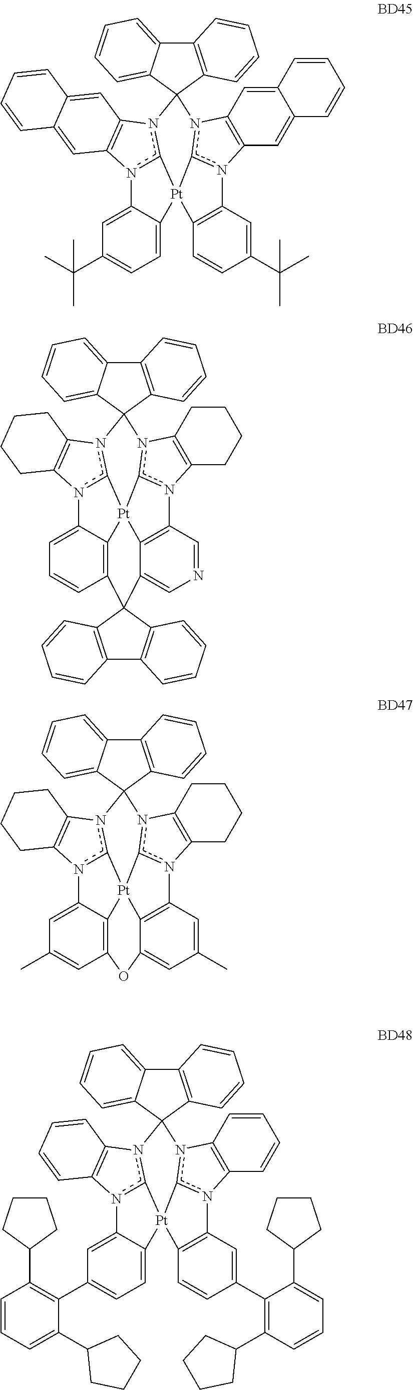

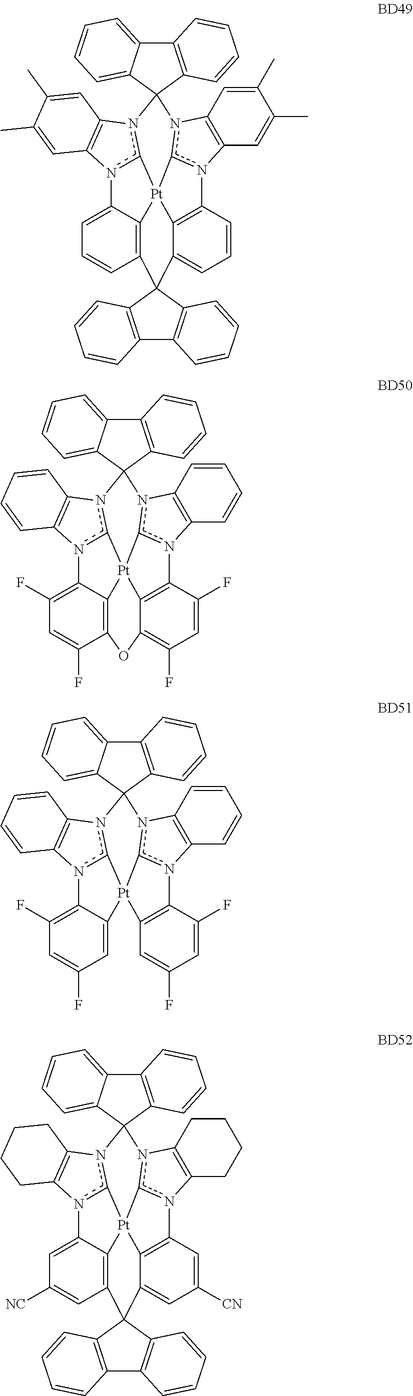

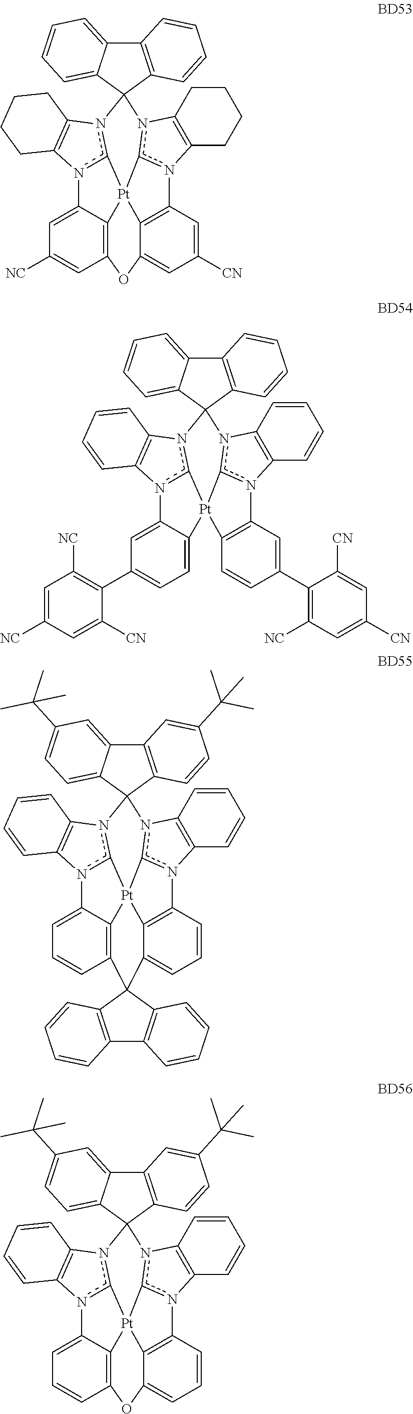

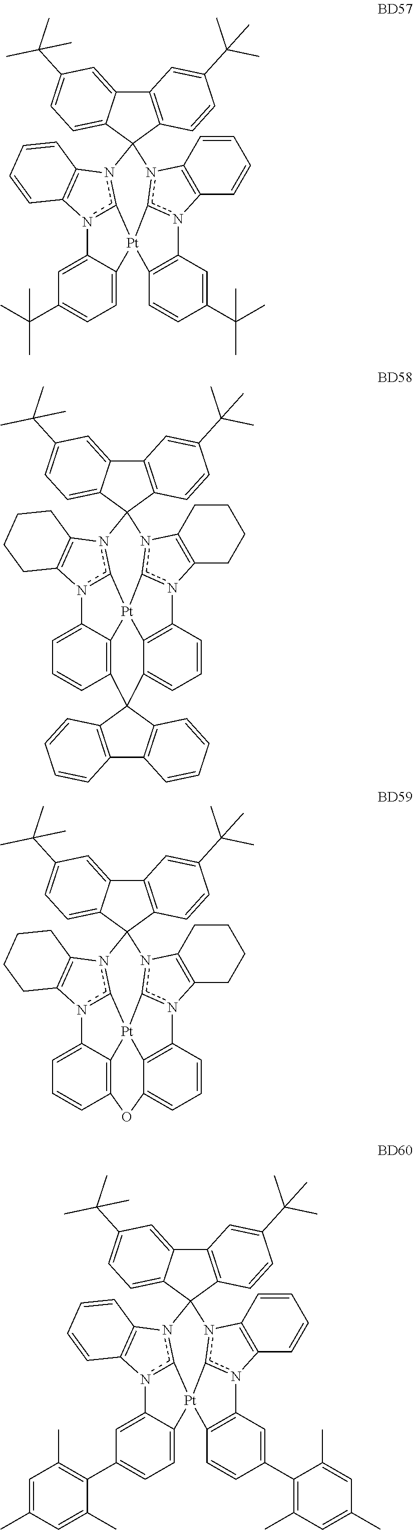

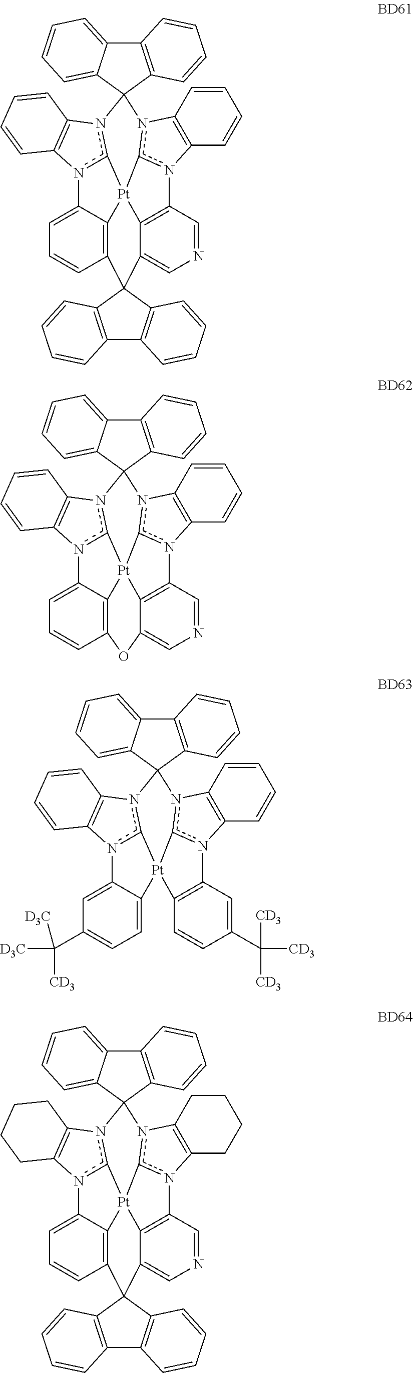

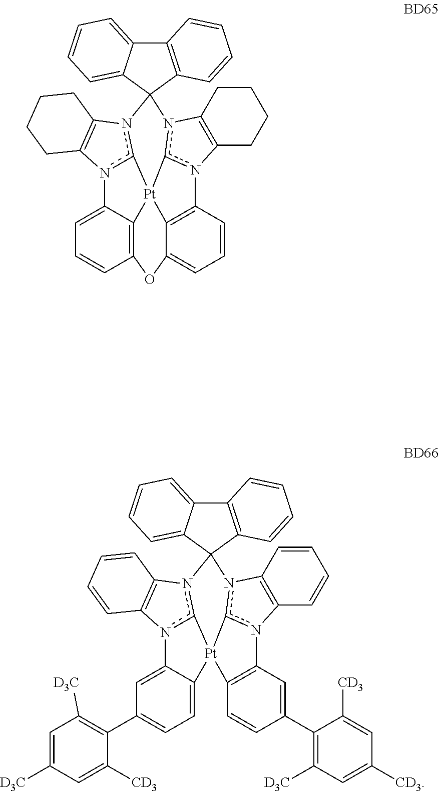

The organometallic compound represented by Formula 1 may include a structure in which ring A₁₀ and ring A₄₀ may each include a N—C—N bond, and, as a result, may have excellent stability between a ligand-metal bond. In addition, in Formula 1, ring A₁₀ and ring A₄₀ may be bound to each other via a

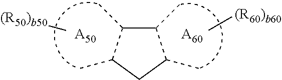

moiety, and, as a result, may have excellent structural stability. Accordingly, when the organometallic compound represented by Formula 1 is applied to an organic light-emitting device, energy transfer may be facilitated, luminescence efficiency may be excellent, and efficiency and lifespan characteristics may be improved due to suppression or reduction of exciplex formation.

The organometallic compound may emit blue light. In some embodiments, the organometallic compound may emit blue light having a maximum emission wavelength in a range of about 400 nanometers (nm) to about 500 nm, e.g., about 410 nm to about 490 nm (bottom emission CIE_{x,y} color-coordinate X=0.15, Y=0.05 to 0.15), but embodiments of the present disclosure are not limited thereto. Accordingly, the organometallic compound represented by Formula 1 may be used effectively in the manufacture of an organic light-emitting device that emits blue light.

Methods of synthesizing the organometallic compound represented by Formula 1 should be readily apparent to those of ordinary skill in the art by referring to the Examples described herein.

In some embodiments,

the first electrode of the light-emitting device may be an anode,

the second electrode of the light-emitting device may be a cathode,

the interlayer may further include a hole transport region located between the first electrode and the emission layer and an electron transport region located between the emission layer and the second electrode,

the hole transport region may include a hole injection layer, a hole transport layer, an emission auxiliary layer, an electron blocking layer, or any combination thereof, and

the electron transport region may include a buffer layer, a hole blocking layer, an electron control layer, an electron transport layer, an electron injection layer, or a combination thereof.

In one or more embodiments, the emission layer may include the organometallic compound represented by Formula 1. In some embodiments, the emission layer may emit blue light having a maximum emission wavelength in a range of about 410 nm to about 500 nm.

In one or more embodiments, the emission layer in the light-emitting device may include a dopant and a host, and the organometallic compound represented by Formula 1 may be included in the dopant. For example, the organometallic compound may serve as a dopant. The emission layer may emit, for example, blue light. The blue light may have a maximum emission wavelength in a range of about 410 nm to about 450 nm.

In one or more embodiments, the electron transport region in the light-emitting device may include a hole blocking layer, and the hole blocking layer may include a phosphine oxide-containing compound, a silicone-containing compound, or any combination thereof. In some embodiments, the hole blocking layer may be in direct contact (e.g., physical contact) with the emission layer.

In one or more embodiments, the light-emitting device may further include at least one selected from a first capping layer located outside a first electrode and a second capping layer located outside a second electrode, and at least one selected from the first capping layer and the second capping layer may include the organometallic compound represented by Formula 1. The first capping layer and the second capping layer may respectively be understood by referring to the descriptions of the first capping layer and the second capping layer provided herein.

In some embodiments, the light-emitting device may include:

a first capping layer located outside the first electrode and including the organometallic compound represented by Formula 1;

a second capping layer located outside the second electrode and including the organometallic compound represented by Formula 1; or

the first capping layer and the second capping layer.

The expression that an “(interlayer and/or a capping layer) includes an organometallic compound,” as used herein, may be construed as meaning that the “(interlayer and/or the capping layer) may include one organometallic compound represented by Formula 1 or two different organometallic compounds represented by Formula 1”.

For example, the interlayer and/or the capping layer may include Compound 1 only as the organometallic compound. In this embodiment, Compound 1 may be included in the emission layer of the light-emitting device. In some embodiments, Compounds 1 and 2 may be included in the interlayer as organometallic compounds. In this embodiment, Compounds 1 and 2 may be included in the same layer (for example, both Compounds 1 and 2 may be included in an emission layer) or in different layers (for example, Compound 1 may be included in an emission layer, and Compound 2 may be included in an electron transport region).

The term “interlayer,” as used herein, refers to a single layer and/or a plurality of all layers located between a first electrode and a second electrode in a light-emitting device.

According to one or more embodiments, an electronic apparatus may include the light-emitting device. The electronic apparatus may further include a thin-film transistor. In some embodiments, the electronic apparatus may further include a thin-film transistor including a source electrode and drain electrode, and a first electrode of the light-emitting device may be electrically coupled to the source electrode or the drain electrode. The electronic apparatus may further include a color filter, a color-conversion layer, a touchscreen layer, a polarization layer, or any combination thereof. The electronic apparatus may be understood by referring to the description of the electronic apparatus provided herein.

According to one or more embodiments, an organometallic compound may be represented by Formula 1: wherein Formula 1 may be understood by referring to the description of Formula 1 provided herein.

Therefore, a light-emitting device (e.g., an organic light-emitting device) including the organometallic compound represented by Formula 1 may have high color purity, high luminescence efficiency, low driving voltage, and long lifespan characteristics.

In one or more embodiments, the organometallic compound represented by Formula 1 may emit blue light. In some embodiments, the organometallic compound represented by Formula 1 may emit blue light having a maximum emission wavelength in a range of about 390 nm to about 500 nm, about 410 nm to about 500 nm, about 410 nm to about 490 nm, about 430 nm to about 480 nm, about 440 nm to about 475 nm, or about 455 nm to about 470 nm.

In one or more embodiments, the organometallic compound represented by Formula 1 may have a color purity of a bottom emission CIEx coordinate in a range of about 0.12 to about 0.15 or about 0.13 to about 0.14 and a bottom emission CIEy coordinate in a range of about 0.06 to about 0.25, about 0.10 to about 0.20, or about 0.13 to about 0.20.

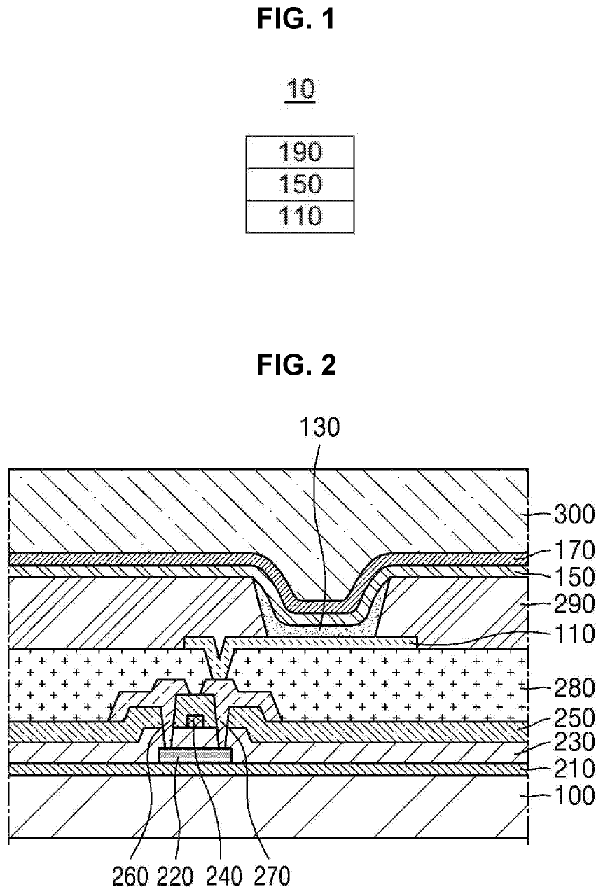

### Description of FIG. 1

FIG. 1 is a schematic view of a light-emitting device 10 according to an embodiment. The light-emitting device 10 may include a first electrode 110, an interlayer 130, and a second electrode 150.

Hereinafter, the structure of the light-emitting device 10 according to an embodiment and a method of manufacturing the light-emitting device 10 according to an embodiment will be described in connection with FIG. 1.

### First Electrode 110

In FIG. 1, a substrate may be additionally located under the first electrode 110 and/or above the second electrode 150. The substrate may be a glass substrate and/or a plastic substrate. The substrate may be a flexible substrate including plastic having excellent heat resistance and durability, for example, polyimide, polyethylene terephthalate (PET), polycarbonate, polyethylene naphthalate, polyarylate (PAR), polyetherimide, or any combination thereof.

The first electrode 110 may be formed by depositing or sputtering, onto the substrate, a material for forming the first electrode 110. When the first electrode 110 is an anode, a high work function material that may easily inject holes may be used as a material for a first electrode 110.

The first electrode 110 may be a reflective electrode, a semi-transmissive electrode, or a transmissive electrode. When the first electrode 110 is a transmissive electrode, a material for forming the first electrode 110 may be indium tin oxide (ITO), indium zinc oxide (IZO), tin oxide (SnO₂), zinc oxide (ZnO), or any combinations thereof. In some embodiments, when the first electrode 110 is a semi-transmissive electrode or a reflective electrode, magnesium (Mg), silver (Ag), aluminum (AI), aluminum-lithium (Al—Li), calcium (Ca), magnesium-indium (Mg—In), magnesium-silver (Mg—Ag), or any combination thereof may be used as a material for forming the first electrode 110.

The first electrode 110 may have a single-layered structure including (e.g., consisting of) a single layer or a multi-layered structure including two or more layers. In some embodiments, the first electrode 110 may have a triple-layered structure of ITO/Ag/ITO.

### Interlayer 130

The interlayer 130 may be on the first electrode 110. The interlayer 130 may include an emission layer.

The interlayer 130 may further include a hole transport region between the first electrode 110 and the emission layer and an electron transport region between the emission layer and the second electrode 150.

The interlayer 130 may further include metal-containing compounds such as organometallic compounds, inorganic materials such as quantum dots, and/or the like, in addition to various suitable organic materials.

The interlayer 130 may include: i) at least two emitting units sequentially stacked between the first electrode 110 and the second electrode 150; and ii) a charge-generation layer located between the at least two emitting units. When the interlayer 130 includes the at least two emitting units and the charge-generation layer, the light-emitting device 10 may be a tandem light-emitting device.

### Hole Transport Region in Interlayer 130

The hole transport region may have i) a single-layered structure including (e.g., consisting of) a single layer including (e.g., consisting of) a single material, ii) a single-layered structure including (e.g., consisting of) a single layer including a plurality of different materials, or iii) a multi-layered structure having a plurality of layers including a plurality of different materials.

the hole transport region may include a hole injection layer (HIL), a hole transport layer (HTL), an emission auxiliary layer, an electron blocking layer (EBL), or a combination thereof.

For example, the hole transport region may have a multi-layered structure, e.g., a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein layers of each structure are sequentially stacked on the first electrode 110 in each stated order.

The hole transport region may include the compound represented by Formula 201, the compound represented by Formula 202, or any combination thereof:

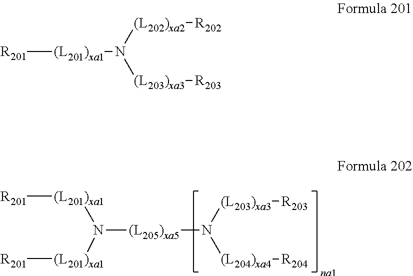

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

R₂₀₁ and R₂₀₂ may optionally be bound to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (e.g., a carbazole group and/or the like) unsubstituted or substituted with at least one R₁₀ₐ (e.g., Compound HT16 described herein),

R₂₀₃ and R₂₀₄ may optionally be bound to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and

na1 may be an integer from 1 to 4.

In some embodiments, Formulae 201 and 202 may each include at least one selected from groups represented by Formulae CY201 to CY217:

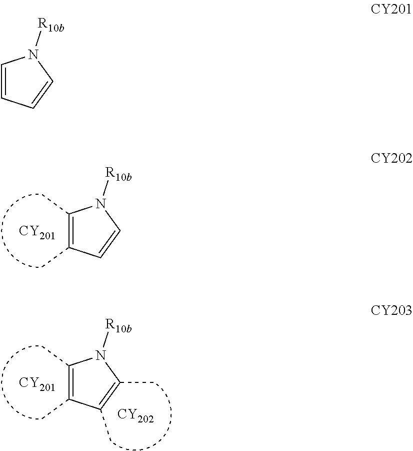

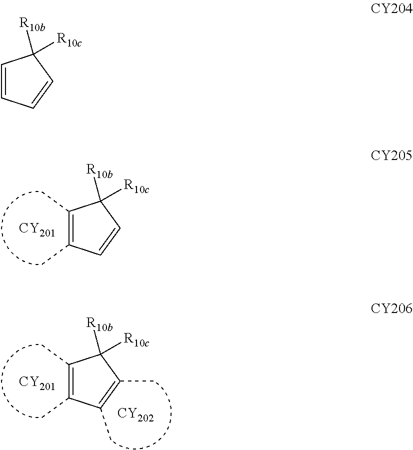

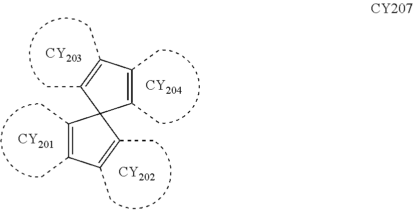

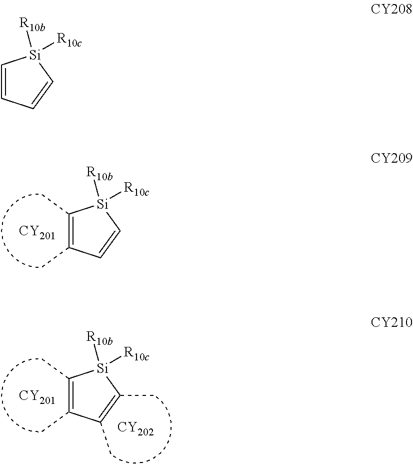

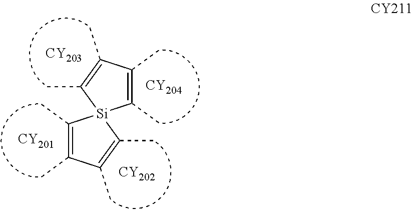

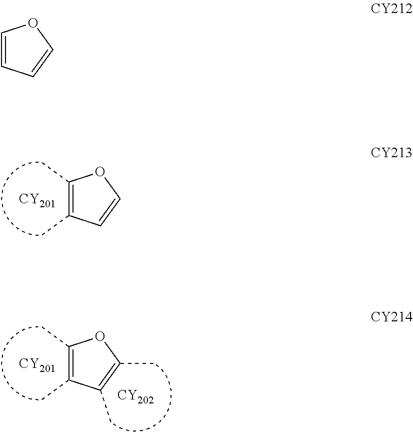

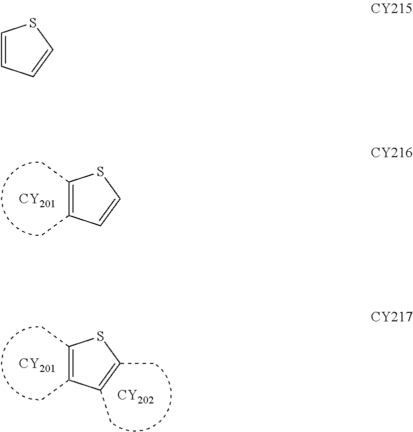

wherein, in Formulae CY201 to CY217, R_{10b} and R_{10c} may each be understood by referring to the descriptions of R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ.

In some embodiments, in Formulae CY201 to CY217, ring CY₂₀₁ to ring CY₂₀₄ may each independently be a benzene group, a naphthalene group, a phenanthrene group, or an anthracene group.

In one or more embodiments, Formulae 201 and 202 may each include at least one selected from groups represented by Formula CY201 to CY203.

In one or more embodiments, Formula 201 may include at least one selected from groups represented by Formulae CY201 to CY203 and at least one selected from groups represented by Formulae CY204 to CY217.

In one or more embodiments, in Formula 201, xa1 may be 1, R₂₀₁ may be a group represented by any one selected from Formulae CY201 to CY203, xa2 may be 0, and R₂₀₂ may be a group represented any one selected from Formulae CY204 to CY207.

In one or more embodiments, Formula 201 and 202 may each not include groups represented by Formulae CY201 to CY203.

In one or more embodiments, Formula 201 and 202 may each not include groups represented by Formulae CY201 to CY203, and include at least one selected from groups represented by Formulae CY204 to CY217.

In one or more embodiments, Formula 201 and 202 may each not include groups represented by Formulae CY201 to CY217.

In some embodiments, the hole transport region may include one selected from Compounds HT1 to HT44, m-MTDATA, TDATA, 2-TNATA, NPB (NPD), β-NPB, TPD, spiro-TPD, spiro-NPB, methylated-NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphorsulfonic acid (PANI/CSA), polyaniline/poly(4-styrenesulfonate (PANI/PSS), or any combination thereof:

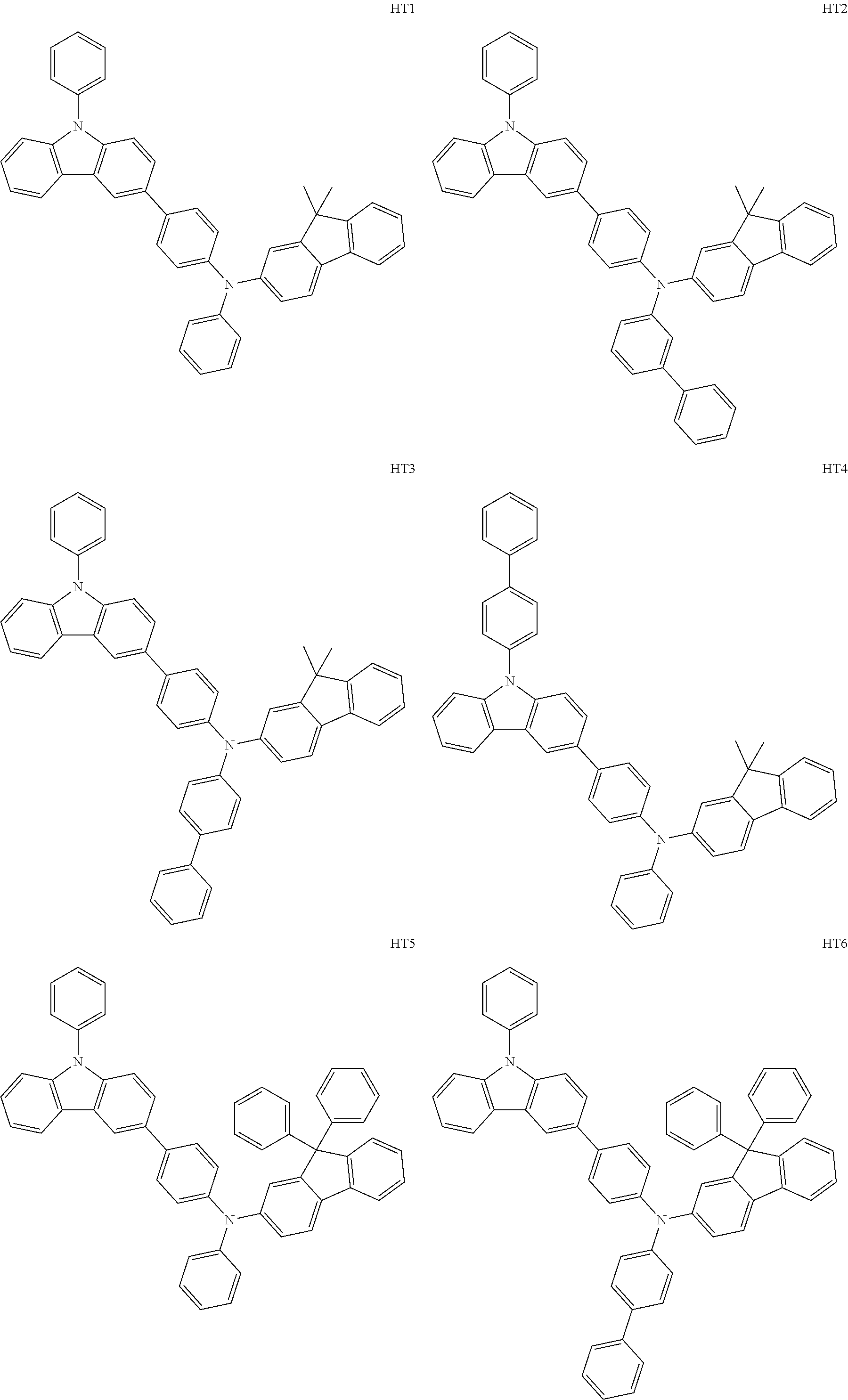

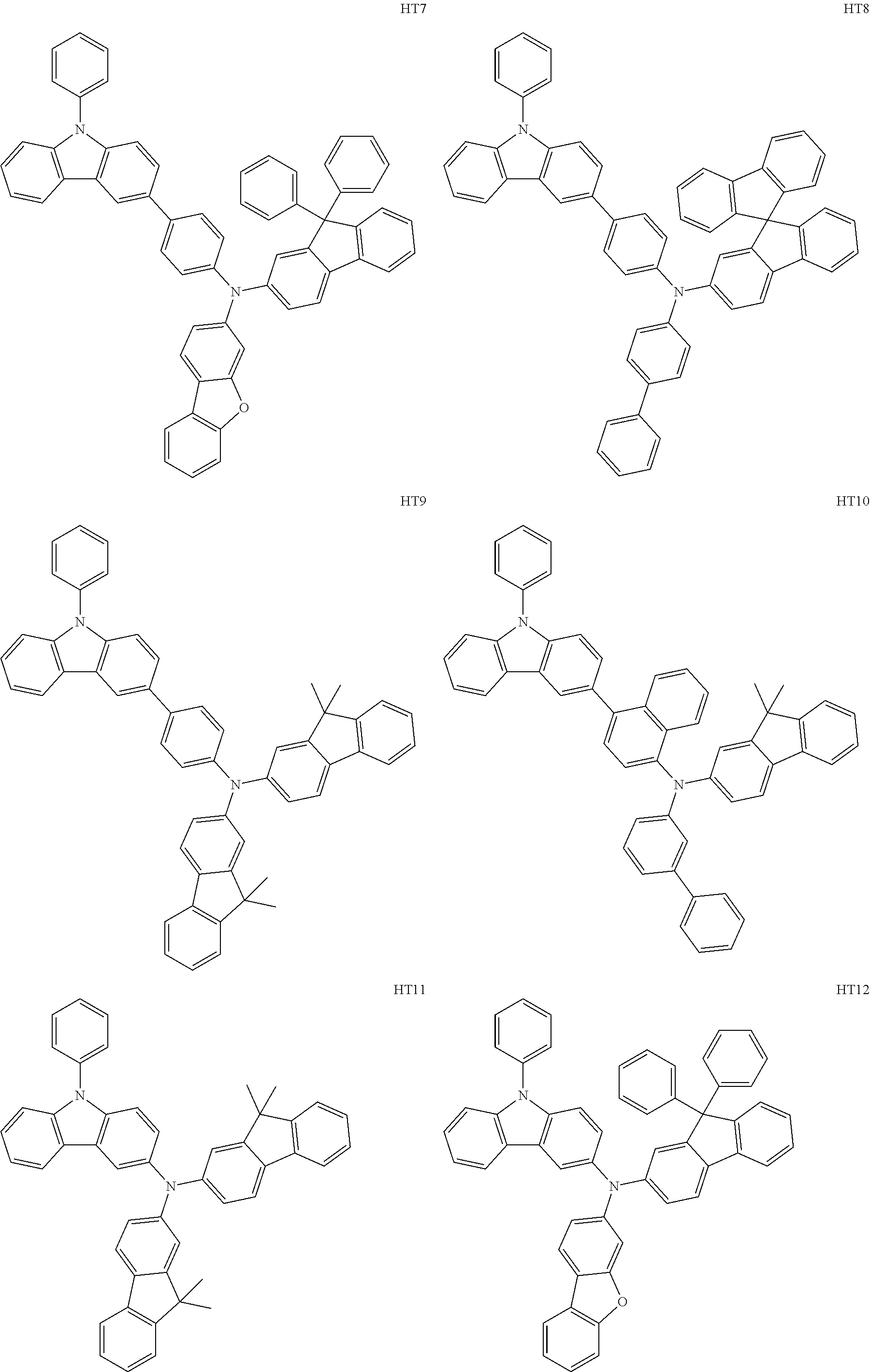

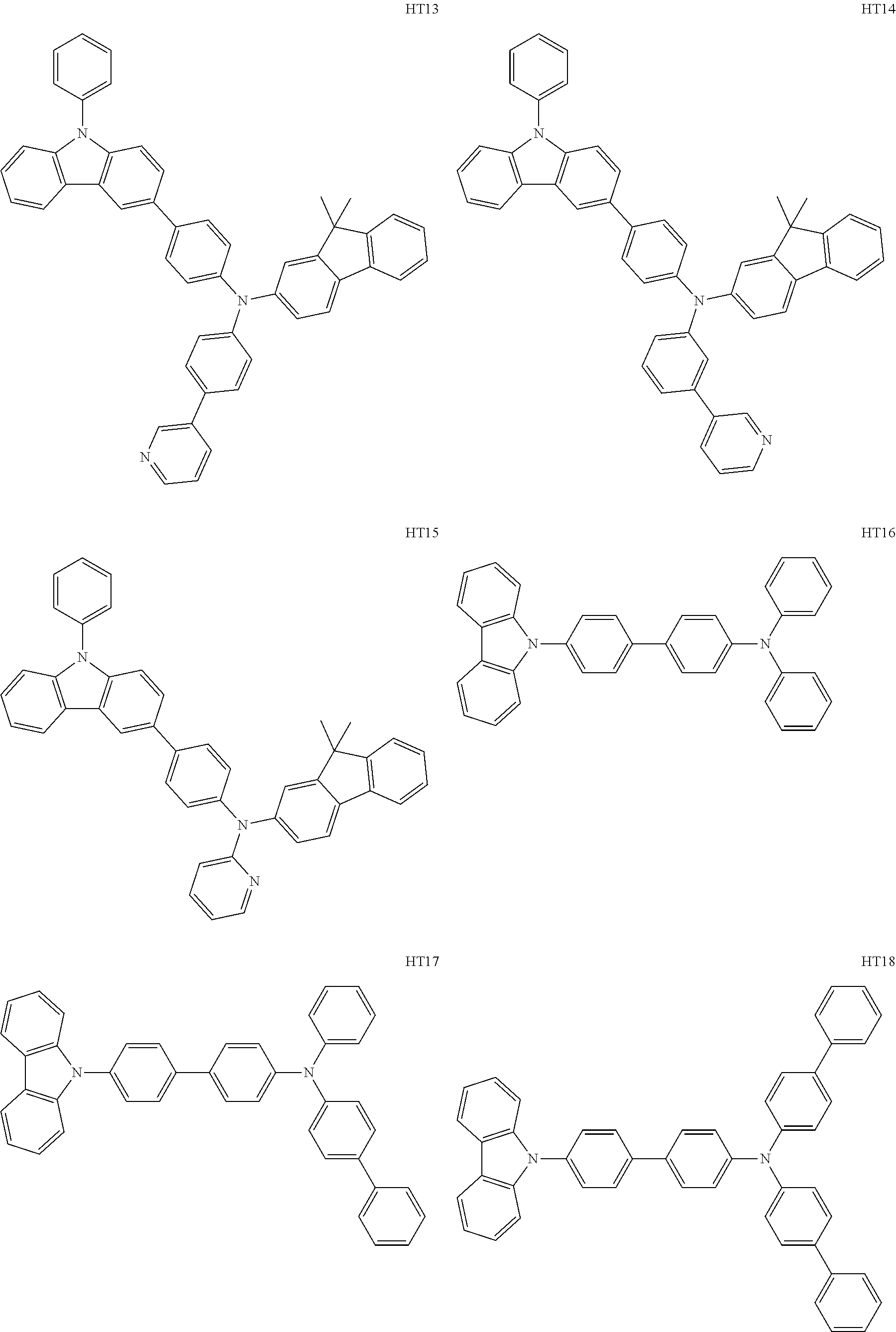

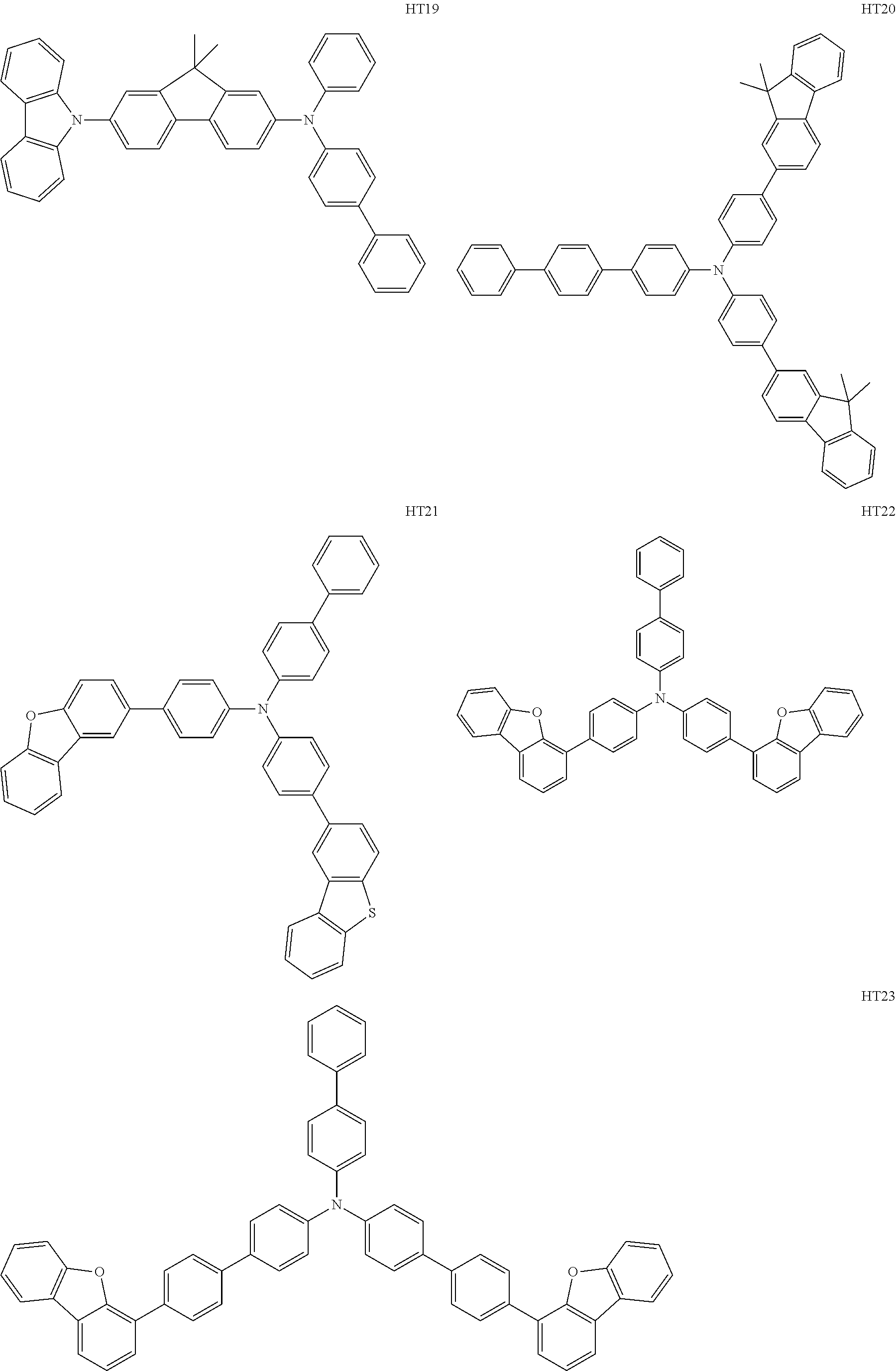

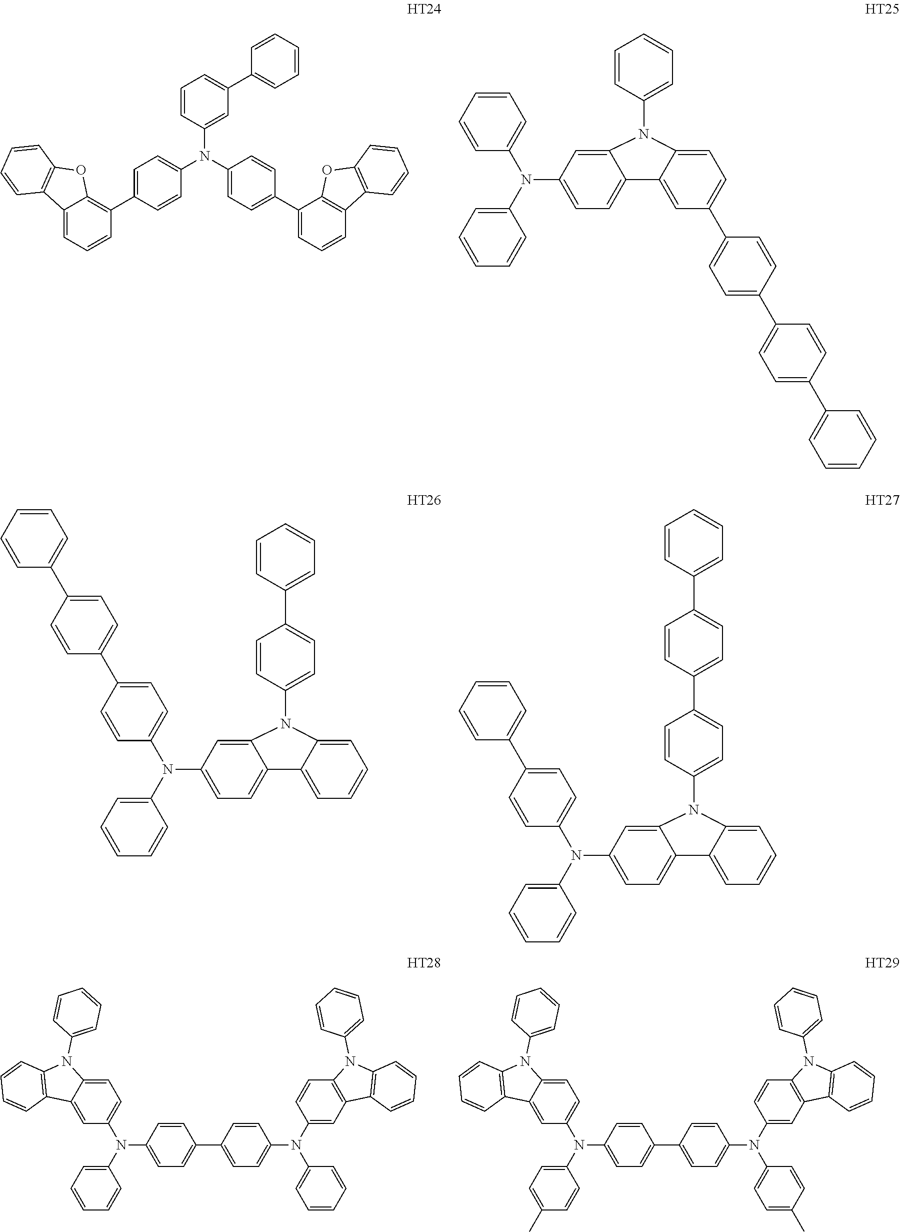

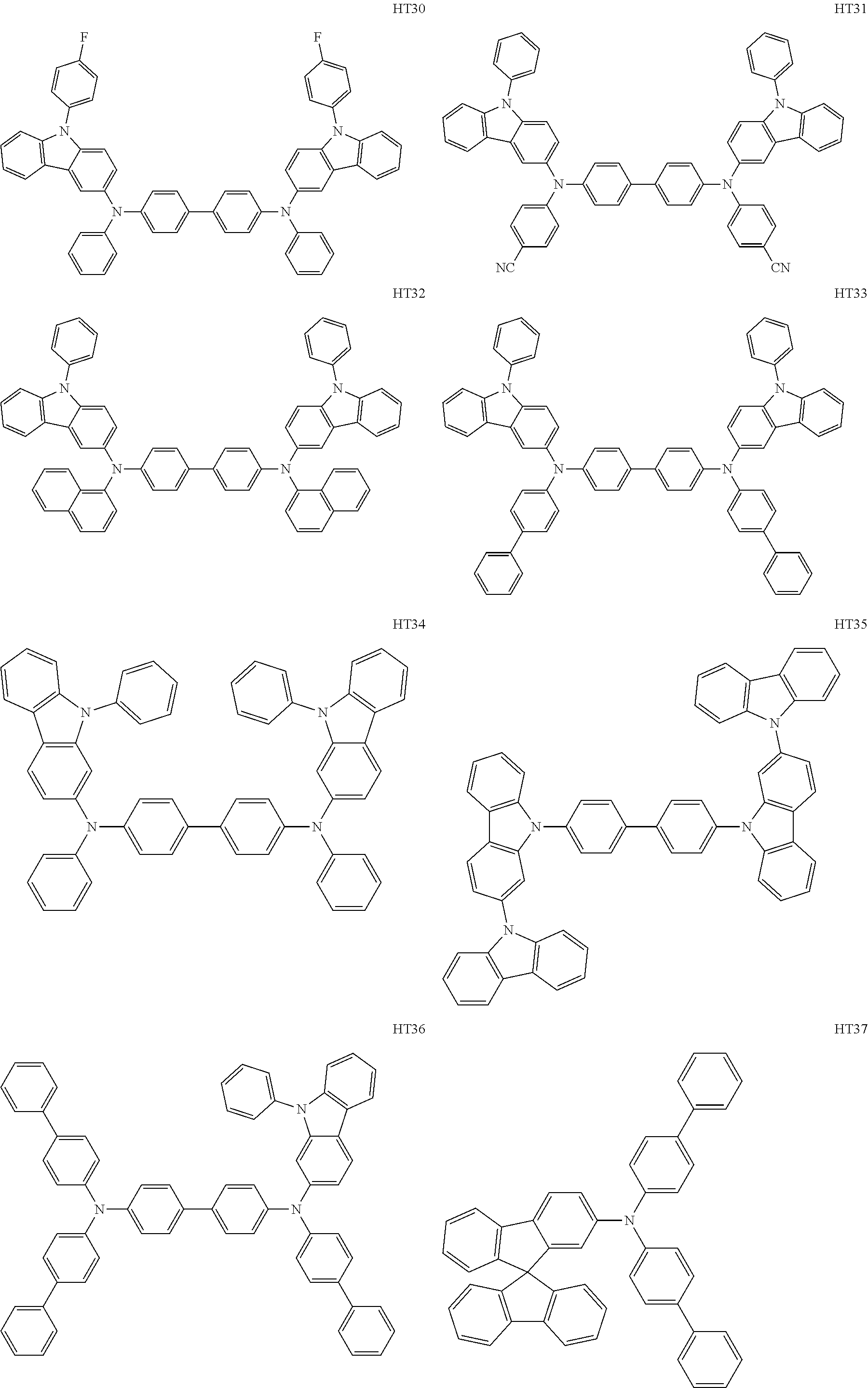

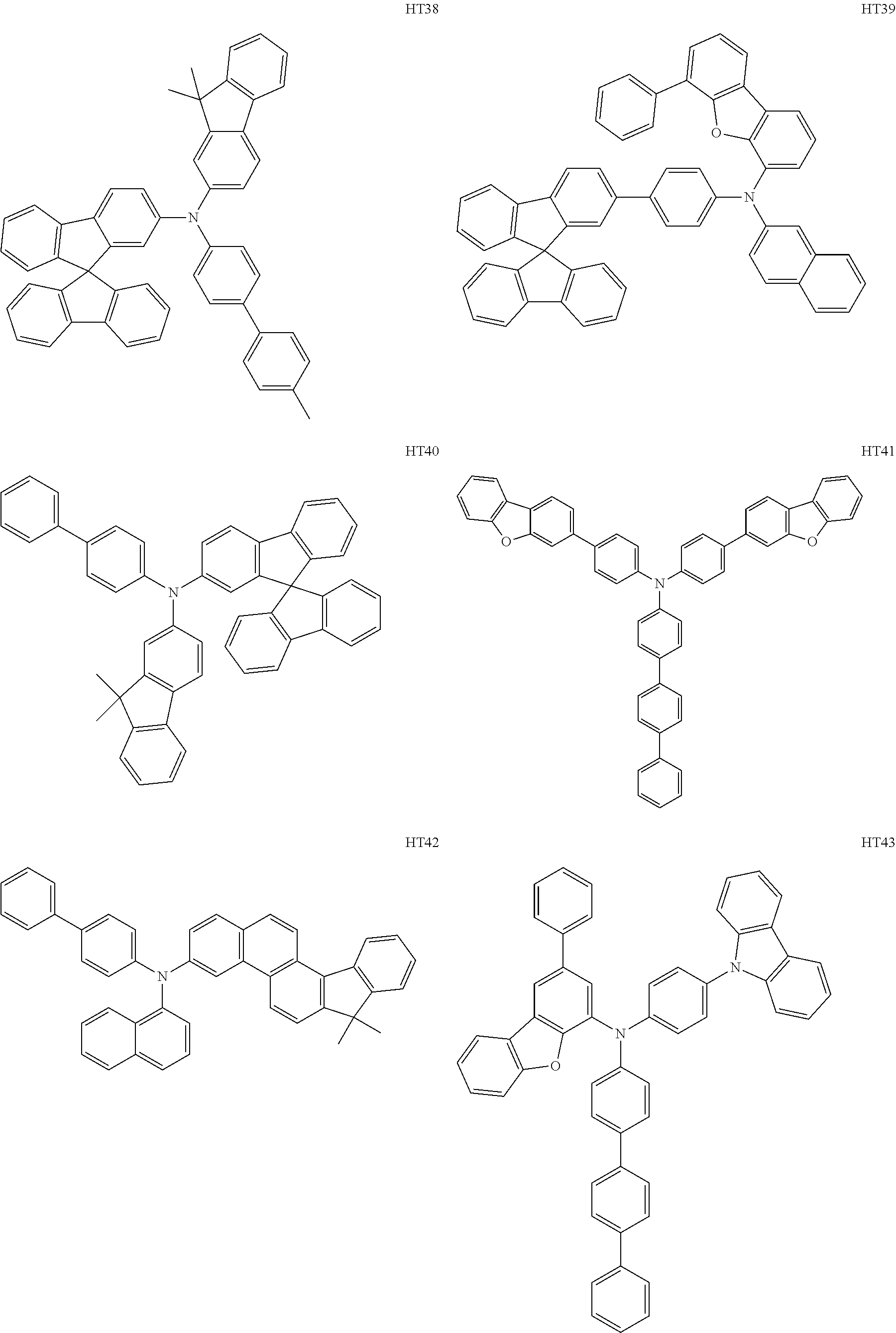

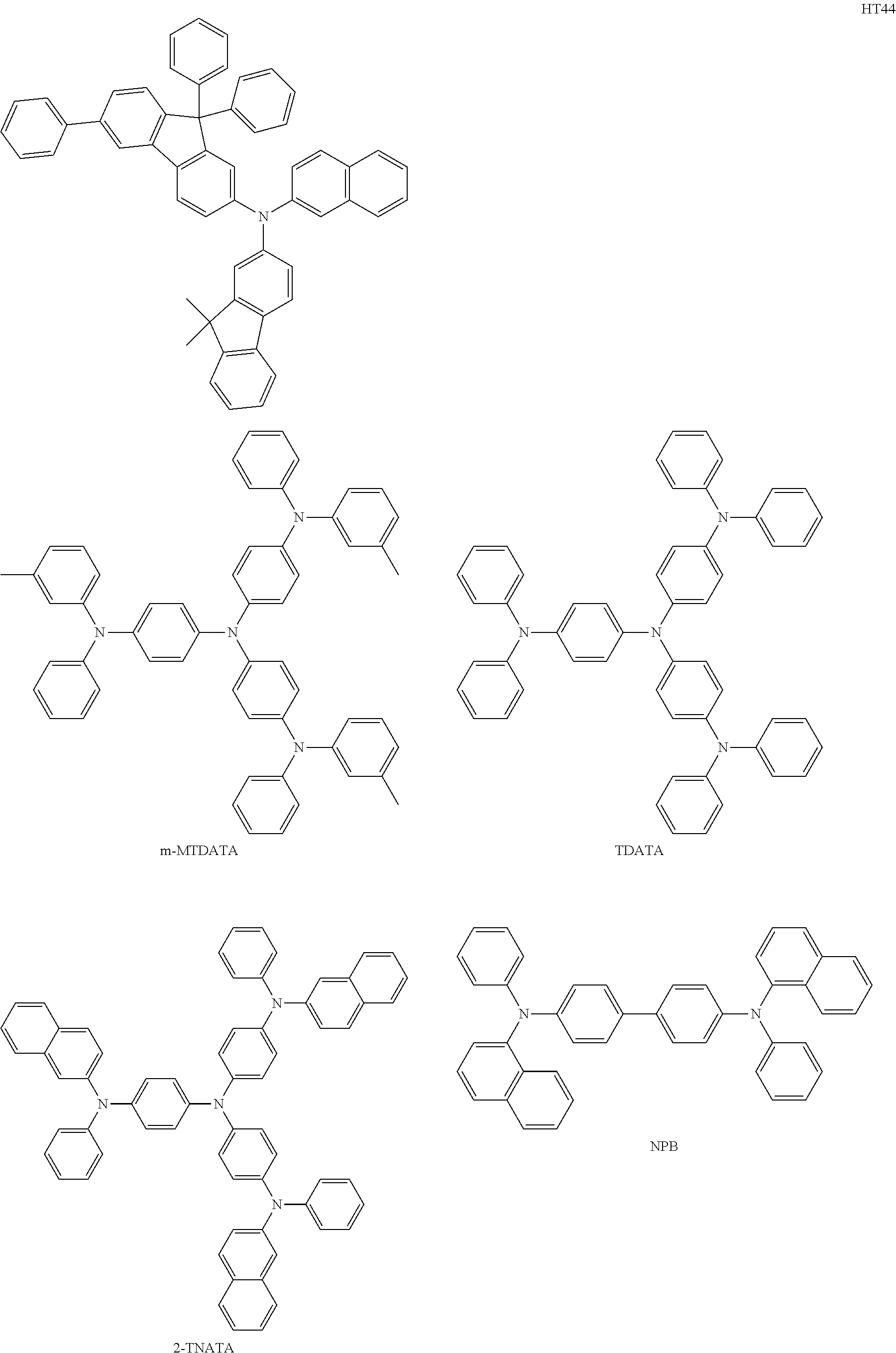

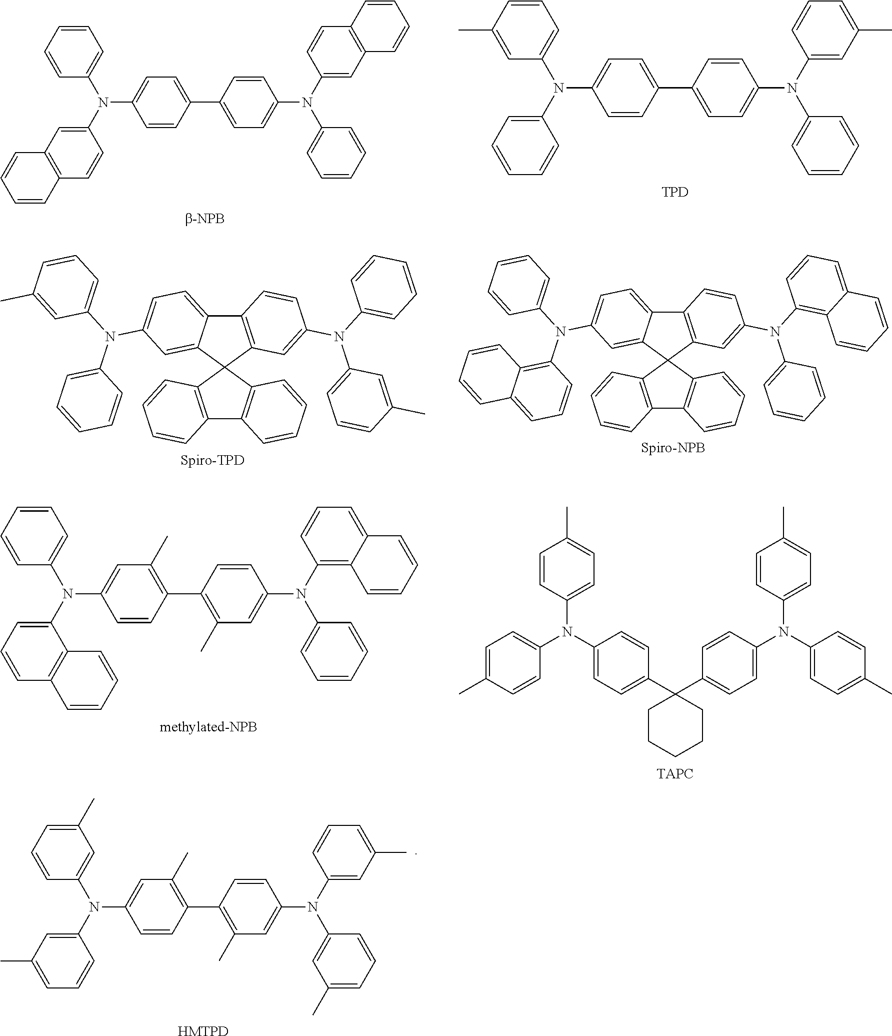

The thickness of the hole transport region may be in a range of about 50 Angstroms (Å) to about 10,000 Å, and in some embodiments, about 100 Å to about 4,000 Å. When the hole transport region includes a hole injection layer, a hole transport layer, or any combination thereof, the thickness of the hole injection layer may be in a range of about 100 Å to about 9,000 Å, and in some embodiments, about 100 Å to about 1,000 Å, and the thickness of the hole transport layer may be in a range of about 50 Å to about 2,000 Å, and in some embodiments, about 100 Å to about 1,500 Å. When the thicknesses of the hole transport region, the hole injection layer, and the hole transport layer are within any of these ranges, excellent hole transport characteristics may be obtained without a substantial increase in driving voltage.

The emission auxiliary layer may increase light emission efficiency by compensating for an optical resonance distance according to the wavelength of light emitted by an emission layer. The electron blocking layer may reduce or eliminate the flow of electrons from an electron transport region. The emission auxiliary layer and the electron blocking layer may include the aforementioned materials.

p-Dopant

The hole transport region may include a charge generating material as well as the aforementioned materials to improve conductive properties of the hole transport region. The charge generating material may be substantially homogeneously or non-homogeneously dispersed (for example, as a single layer including (e.g., consisting of) charge generating material) in the hole transport region.

The charge generating material may include, for example, a p-dopant.

In some embodiments, a lowest unoccupied molecular orbital (LUMO) energy level of the p-dopant may be −3.5 eV or less.

In some embodiments, the p-dopant may include a quinone derivative, a cyano group-containing compound, elements EL1 and EL2-containing compound, or any combination thereof.

Examples of the quinone derivative may include TCNQ, F4-TCNQ, and the like.

Examples of the cyano group-containing compound include HAT-CN, a compound represented by Formula 221, and the like:

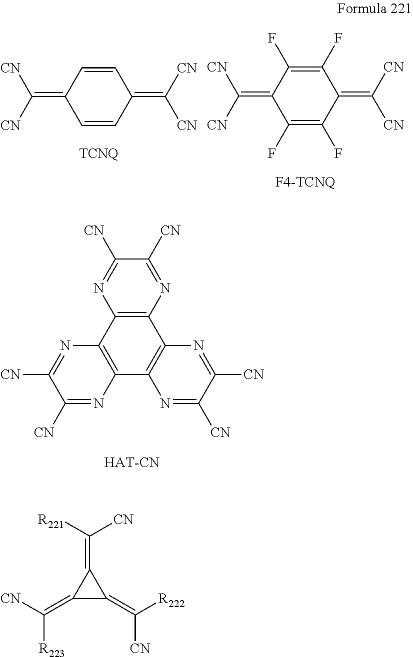

wherein, in Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one selected from R₂₂₁ to R₂₂₃ may each independently be: a C₃-C₆₀ carbocyclic group or a C₁-C₆₀ heterocyclic group, substituted with a cyano group; —F; —Cl; —Br; —I; a C₁-C₂₀ alkyl group substituted with a cyano group, —F, —Cl, —Br, —I, or any combination thereof; or any combination thereof.

In the elements EL1 and EL2-containing compound, element EL1 may be a metal, a metalloid, or a combination thereof, and element EL2 may be a non-metal, a metalloid, or a combination thereof.

Examples of the metal may include: an alkali metal (e.g., lithium (Li), sodium (Na), potassium (K), rubidium (Rb), cesium (Cs), and/or the like); an alkaline earth metal (e.g., beryllium (Be), magnesium (Mg), calcium (Ca), strontium (Sr), barium (Ba), and/or the like); a transition metal (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); post-transition metal (e.g., zinc (Zn), indium (In), tin (Sn), or the like); a lanthanide metal (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like); and the like.

Examples of the metalloid may include silicon (Si), antimony (Sb), tellurium (Te), and the like.

Examples of the non-metal may include oxygen (O), halogen (e.g., F, Cl, Br, I, and the like), and the like.

For example, examples of the elements EL1 and EL2-containing compound may include a metal oxide, a metal halide (e.g., metal fluoride, metal chloride, metal bromide, metal iodide, and the like), a metalloid halide (e.g., a metalloid fluoride, a metalloid chloride, a metalloid bromide, a metalloid iodide, and the like), a metal telluride, or any combination thereof.

Examples of the metal oxide may include tungsten oxide (e.g., WO, W₂O₃, WO₂, WO₃, W₂O₅, and the like), vanadium oxide (e.g., VO, V₂O₃, VO₂, V₂O₅, and the like), molybdenum oxide (e.g., MoO, Mo₂O₃, MoO₂, MoO₃, Mo₂O₅, and the like), rhenium oxide (e.g., ReO₃, and the like), and the like.

Examples of the metal halide may include alkali metal halide, alkaline earth metal halide, transition metal halide, post-transition metal halide, lanthanide metal halide, and the like.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, CsI, and the like.

Examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, BaI₂, and the like.

Examples of the transition metal halide may include titanium halide (e.g., TiF₄, TiCl₄, TiBr₄, TiI₄, and the like), zirconium halide (e.g., ZrF₄, ZrCl₄, ZrBr₄, ZrI₄, and the like), hafnium halide (e.g., HfF₄, HfCl₄, HfBr₄, HfI₄, and the like), vanadium halide (e.g., VF₃, VCl₃, VBr₃, VI₃, and the like), niobium halide (e.g., NbF₃, NbCl₃, NbBr₃, NbI₃, and the like), tantalum halide (e.g., TaF₃, TaCl₃, TaBr₃, TaI₃, and the like), chromium halide (e.g., CrF₃, CrCl₃, CrBr₃, CrI₃, and the like), molybdenum halide (e.g., MoF₃, MoCl₃, MoBr₃, MoI₃, and the like), tungsten halide (e.g., WF₃, WCl₃, WBr₃, WI₃, and the like), manganese halide (e.g., MnF₂, MnCl₂, MnBr₂, MnI₂, and the like), technetium halide (e.g., TcF₂, TcCl₂, TcBr₂, TcI₂, and the like), rhenium halide (e.g., ReF₂, ReCl₂, ReBr₂, ReI₂, and the like), iron halide (e.g., FeF₂, FeCl₂, FeBr₂, FeI₂, and the like), ruthenium halide (e.g., RuF₂, RuCl₂, RuBr₂, RuI₂, and the like), osmium halide (e.g., OsF₂, OsCl₂, OsBr₂, OsI₂, and the like), cobalt halide (e.g., CoF₂, CoCl₂, CoBr₂, CoI₂, and the like), rhodium halide (e.g., RhF₂, RhCl₂, RhBr₂, RhI₂, and the like), iridium halide (e.g., IrF₂, IrCl₂, IrBr₂, IrI₂, and the like), nickel halide (e.g., NiF₂, NiCl₂, NiBr₂, NiI₂, and the like), palladium halide (e.g., PdF₂, PdCl₂, PdBr₂, PdI₂, and the like), platinum halide (e.g., PtF₂, PtCl₂, PtBr₂, PtI₂, and the like), copper halide (e.g., CuF, CuCl, CuBr, CuI, and the like), silver halide (e.g., AgF, AgCl, AgBr, AgI, and the like), gold halide (e.g., AuF, AuCl, AuBr, AuI, and the like), and the like.

Examples of the post-transition metal halide may include zinc halide (e.g., ZnF₂, ZnCl₂, ZnBr₂, ZnI₂, and the like), indium halide (e.g., InI₃ and the like), tin halide (e.g., SnI₂ and the like), and the like.

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and the like.

Examples of the metalloid halide may include antimony halide (e.g., SbCl₅ and the like) and the like.

Examples of the metal telluride may include alkali metal telluride (e.g., Li₂Te, Na₂Te, K₂Te, Rb₂Te, Cs₂Te, and the like), alkaline earth metal telluride (e.g., BeTe, MgTe, CaTe, SrTe, BaTe, and the like), transition metal telluride (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and the like), post-transition metal telluride (e.g., ZnTe and the like), lanthanide metal telluride (e.g., LaTe, CeTe, PrTe, NdTe, PmTe, EuTe, GdTe, TbTe, DyTe, HoTe, ErTe, TmTe, YbTe, LuTe, and the like), and the like.

### Emission Layer in Interlayer 130

When the light-emitting device 10 is a full color light-emitting device, the emission layer may be patterned into a red emission layer, a green emission layer, and/or a blue emission layer, according to a sub-pixel. In one or more embodiments, the emission layer may have a stacked structure. The stacked structure may include two or more layers selected from a red emission layer, a green emission layer, and a blue emission layer. The two or more layers may be in direct contact (e.g., physical contact) with each other. In some embodiments, the two or more layers may be separated from each other. In one or more embodiments, the emission layer may include two or more materials. The two or more materials may include a red light-emitting material, a green light-emitting material, or a blue light-emitting material. The two or more materials may be mixed together with each other in a single layer. The two or more materials mixed together with each other in the single layer may emit white light.

The emission layer may include a host and a dopant. The dopant may be a phosphorescent dopant, a fluorescent dopant, or any combination thereof.

In some embodiments, the dopant may include the organometallic compound represented by Formula 1 described herein.

The amount of the dopant in the emission layer may be in a range of about 0.01 parts to about 15 parts by weight based on 100 parts by weight of the host.

In some embodiments, the emission layer may include a quantum dot.

The emission layer may include a delayed fluorescence material. The delayed fluorescence material may serve as a host or a dopant in the emission layer.

The thickness of the emission layer may be in a range of about 100 Å to about 1,000 Å, and in some embodiments, about 200 Å to about 600 Å. When the thickness of the emission layer is within any of these ranges, improved luminescence characteristics may be obtained without a substantial increase in driving voltage.

### Host

The host in the emission layer may include any suitable host.

In some embodiments, the host may further include a compound represented by Formula 301:

[Ar₃₀₁]_{xb11}-[(L₃₀₁)_{xb1}-R₃₀₁]_{xb21} Formula 301

wherein, in Formula 301,

Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 1 to 5, and

Q₃₀₁ to Q₃₀₃ may each be understood by referring to the description of Q₁ provided herein.

In some embodiments, when xb11 in Formula 301 is 2 or greater, at least two Ar₃₀₁(s) may be bound via a single bond.

In some embodiments, the host may include a compound represented by Formula 301-1, a compound represented by Formula 301-2, or any combination thereof:

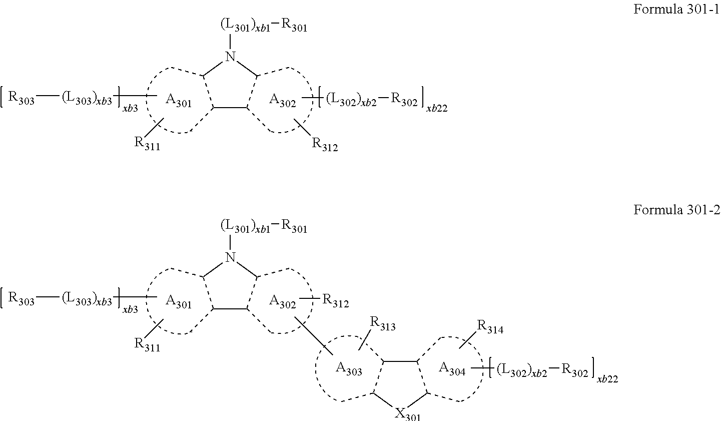

wherein, in Formulae 301-1 and 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ may respectively be understood by referring to the descriptions of L₃₀₁, xb1, and R₃₀₁ provided herein,

L₃₀₂ to L₃₀₄ may each be understood by referring to the description of L₃₀₁ provided herein,

xb2 to xb4 may each be understood by referring to the descriptions of xb1 provided herein, and

R₃₀₂ to R₃₀₅ and R₃₁₁ to R₃₁₄ may each be understood by referring to the descriptions of R₃₀₁ provided herein.

In some embodiments, the host may include an alkaline earth metal complex. For example, the host may include a Be complex (e.g., Compound H55), a Mg complex, a Zn complex, or any combination thereof.

In some embodiments, the host may include one selected from Compounds H1 to H124, 9,10-di(2-naphthyl)anthracene (ADN), 2-methyl-9,10-bis(naphthalen-2-yl)anthracene (MADN), 9,10-di(2-naphthyl)-2-t-butyl-anthracene (TBADN), 4,4′-bis(N-carbazolyl)-1,1′-biphenyl (CBP), 1,3-di(carbazol-9-yl)benzene (mCP), 1,3,5-tri(carbazol-9-yl)benzene (TCP), 3,3-di(9H-carbazol-9-yl)biphenyl (mCBP), or any combination thereof:

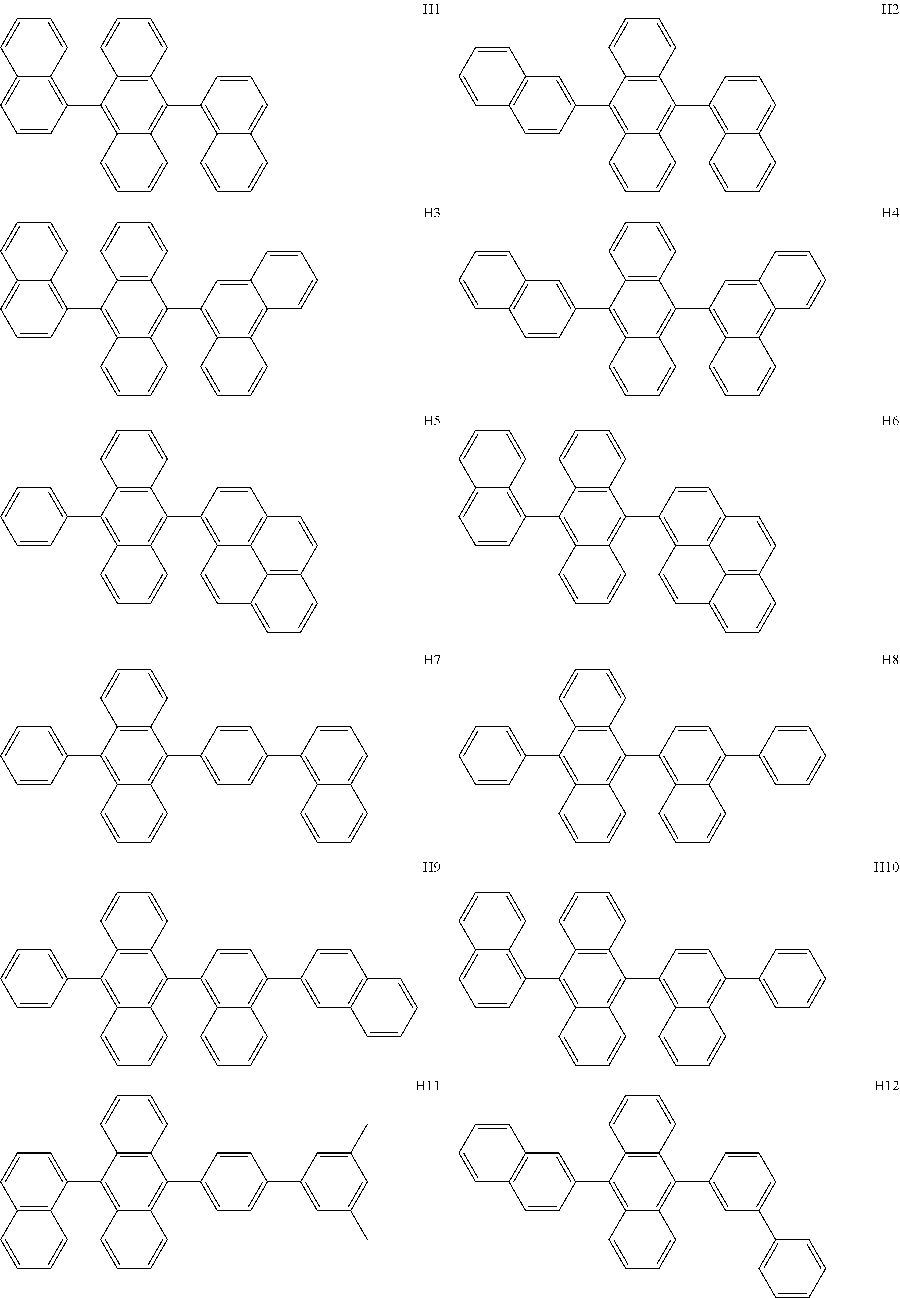

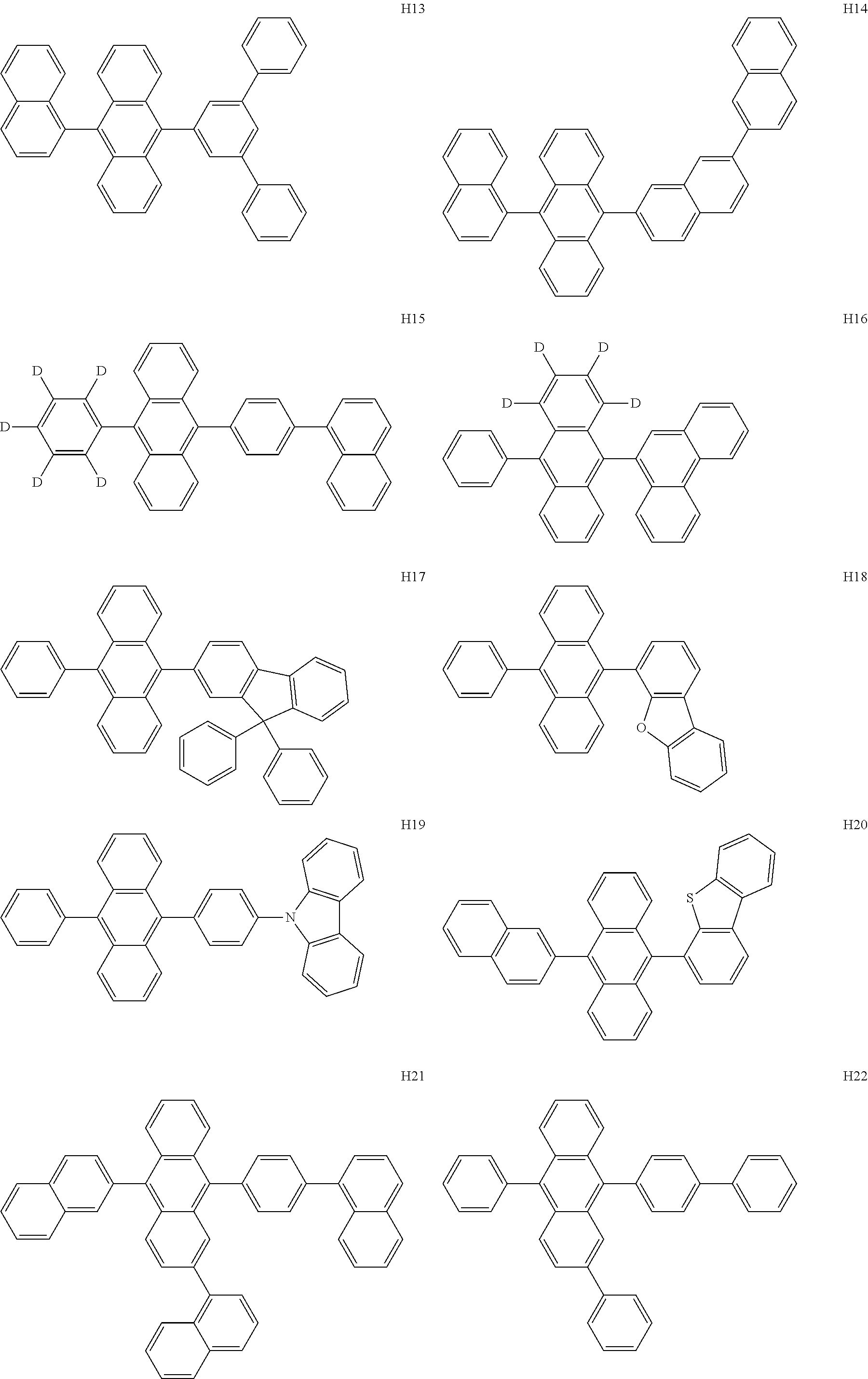

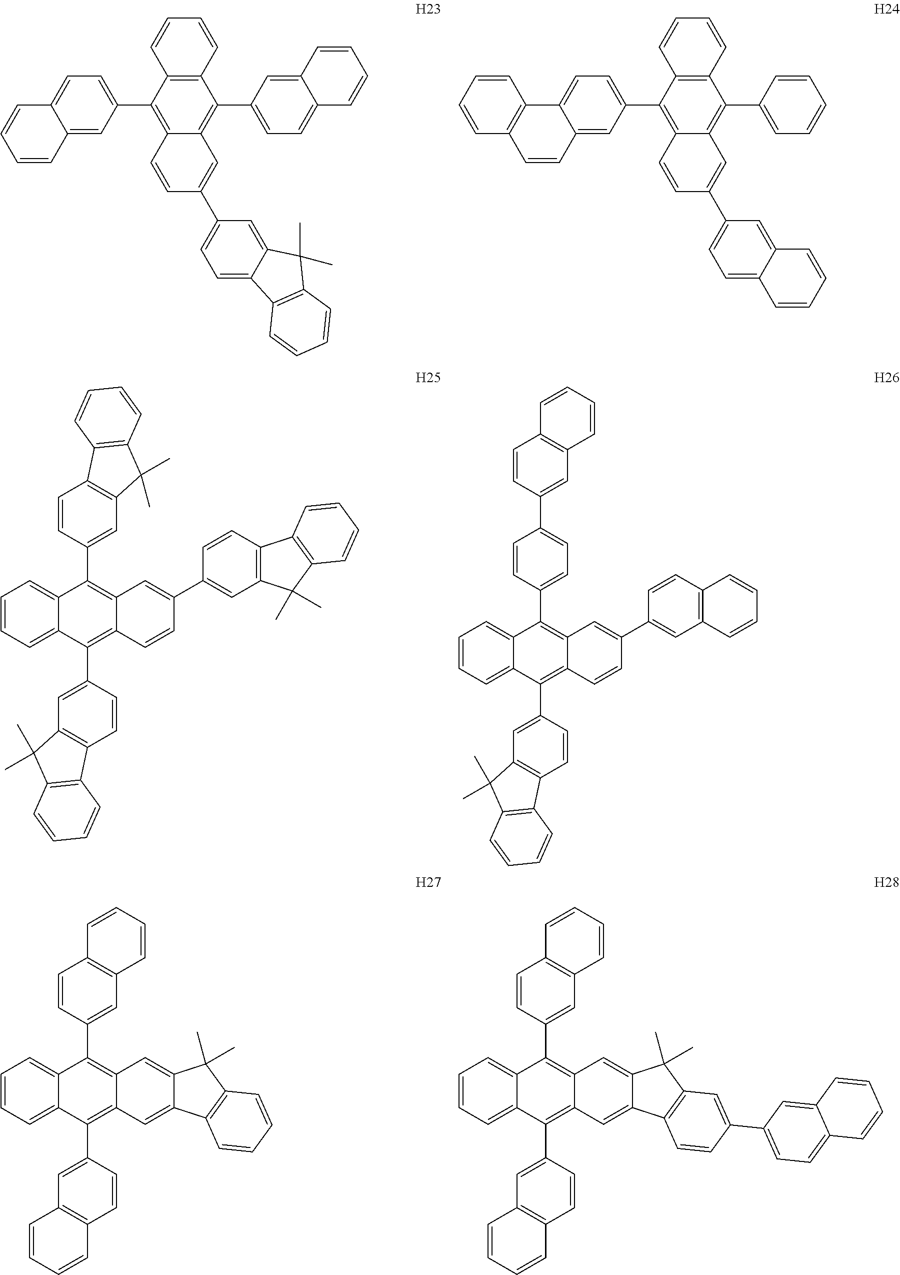

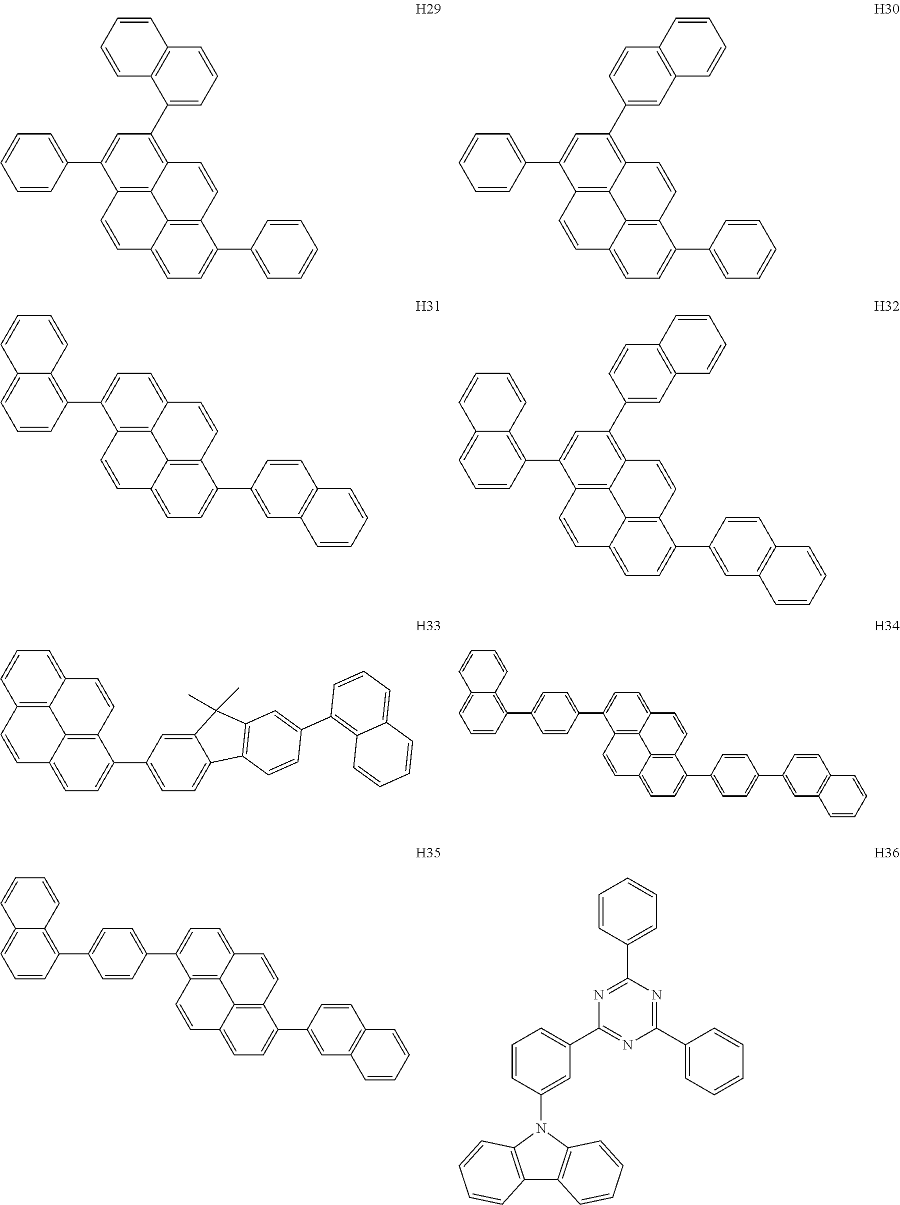

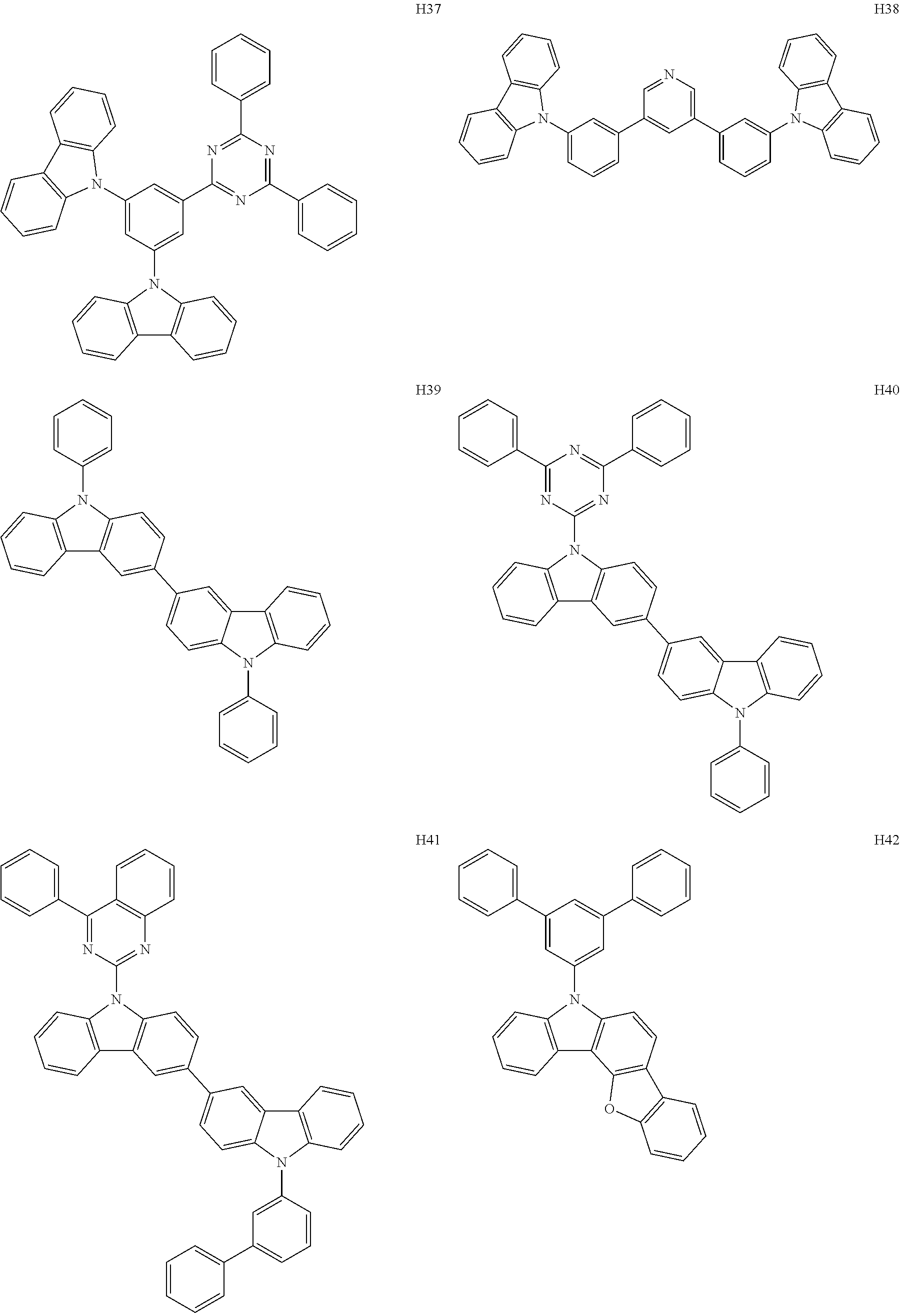

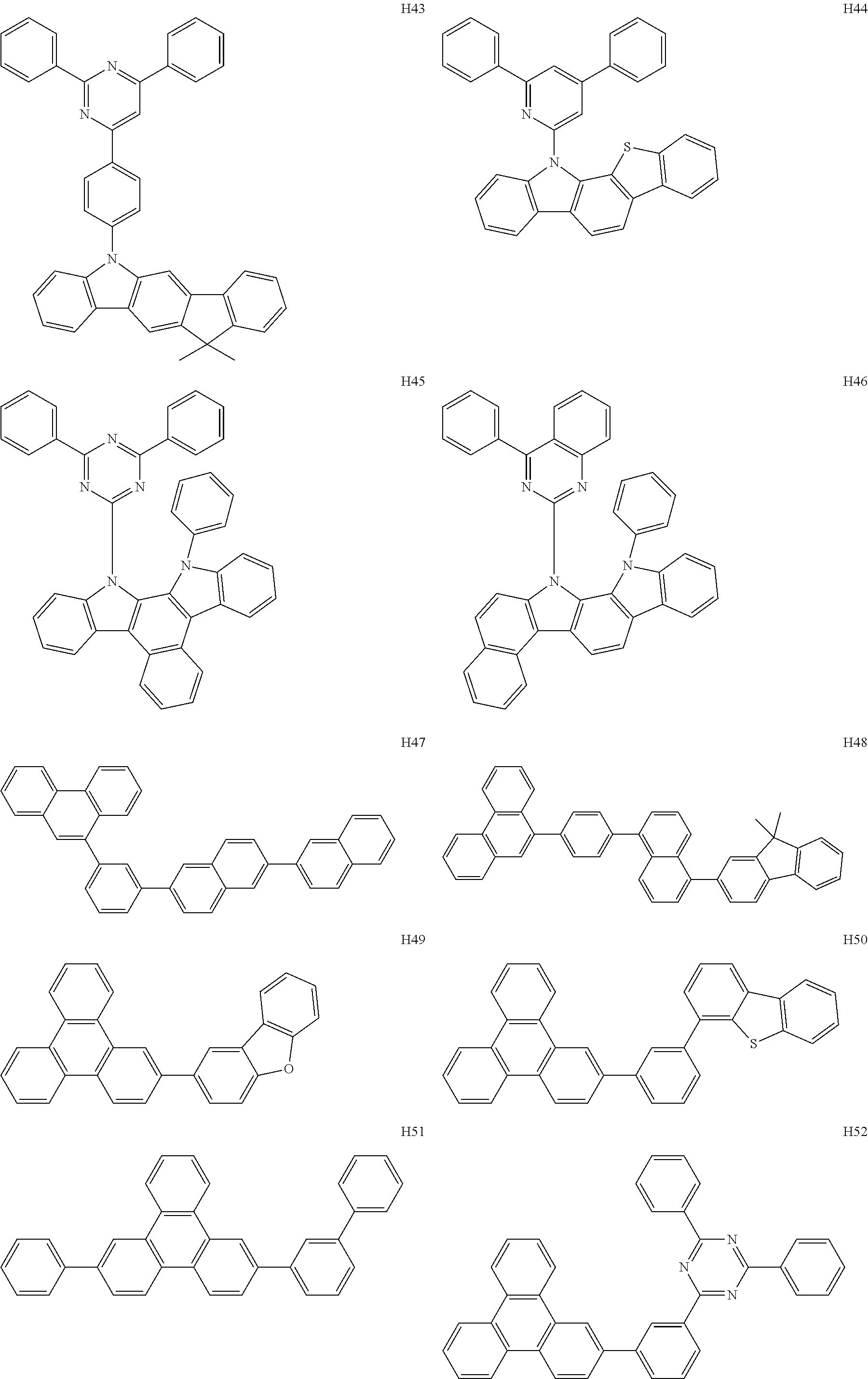

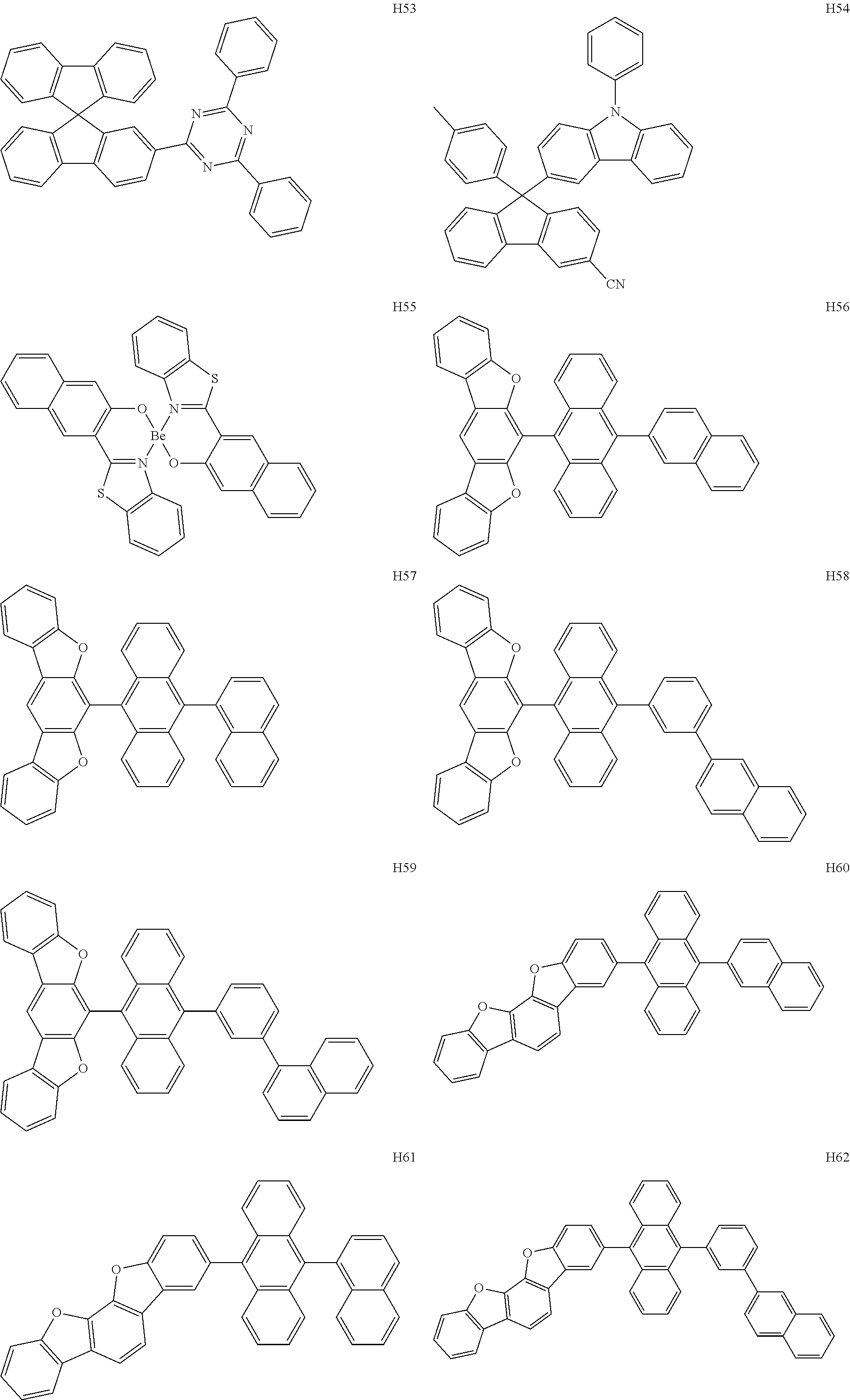

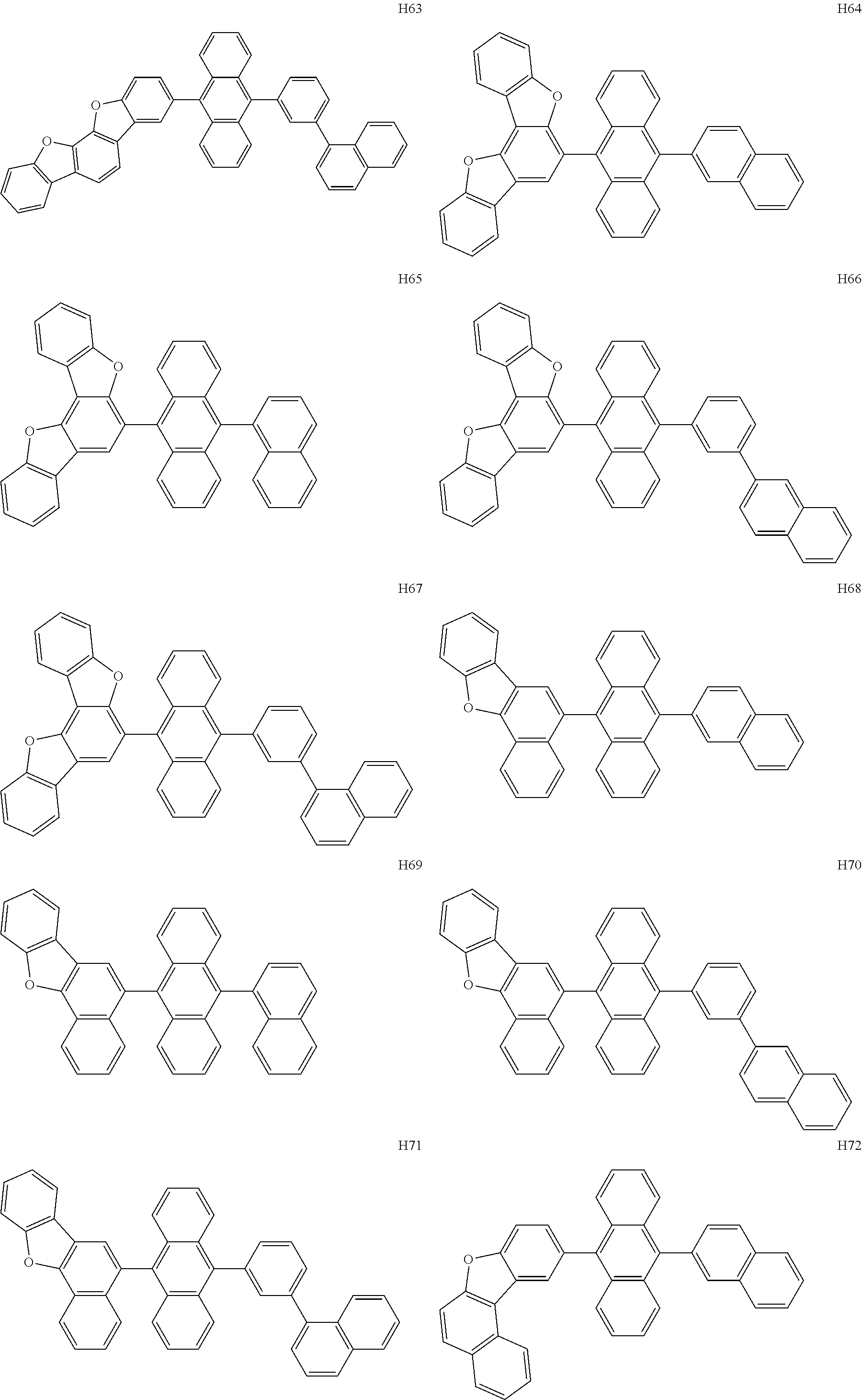

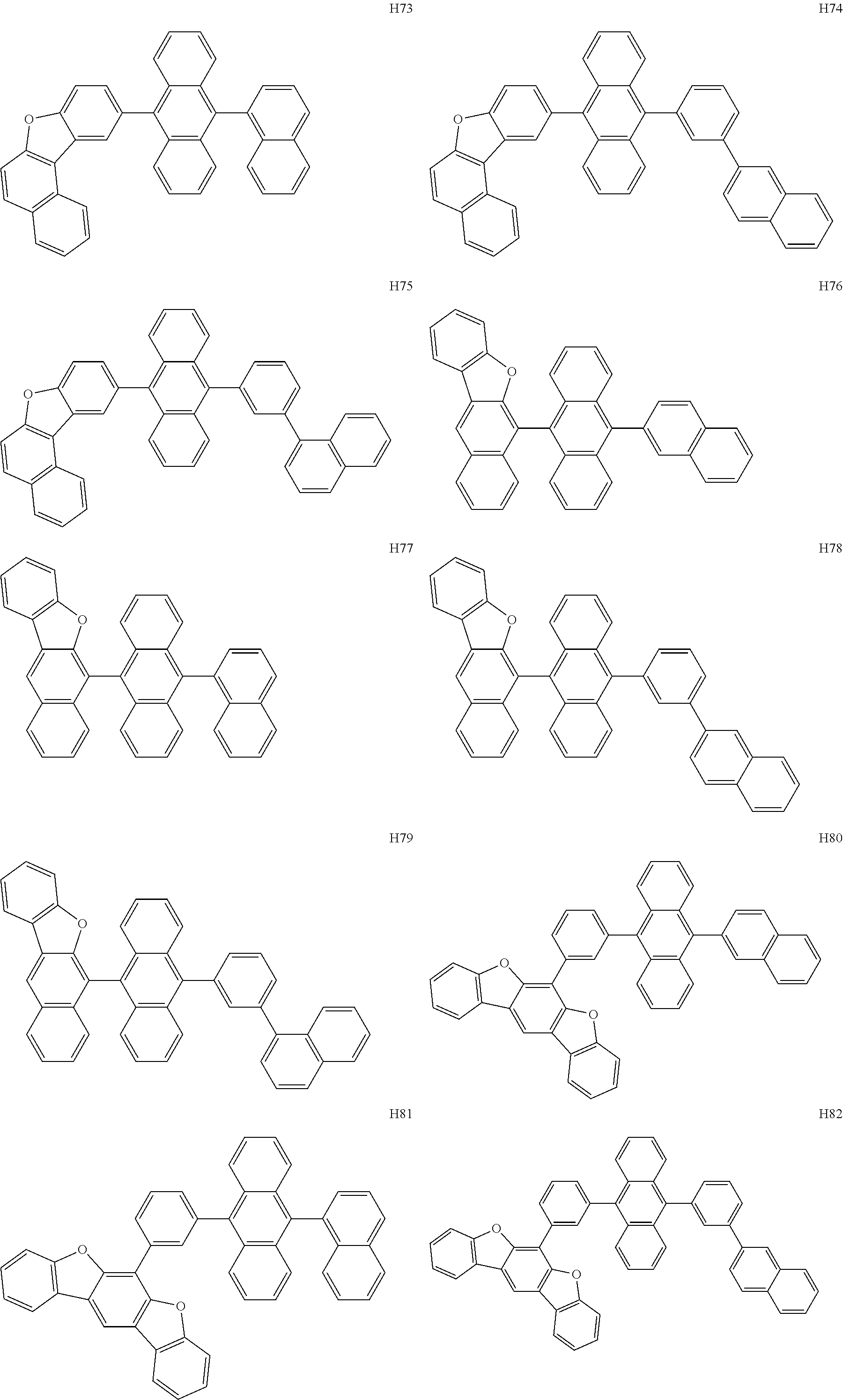

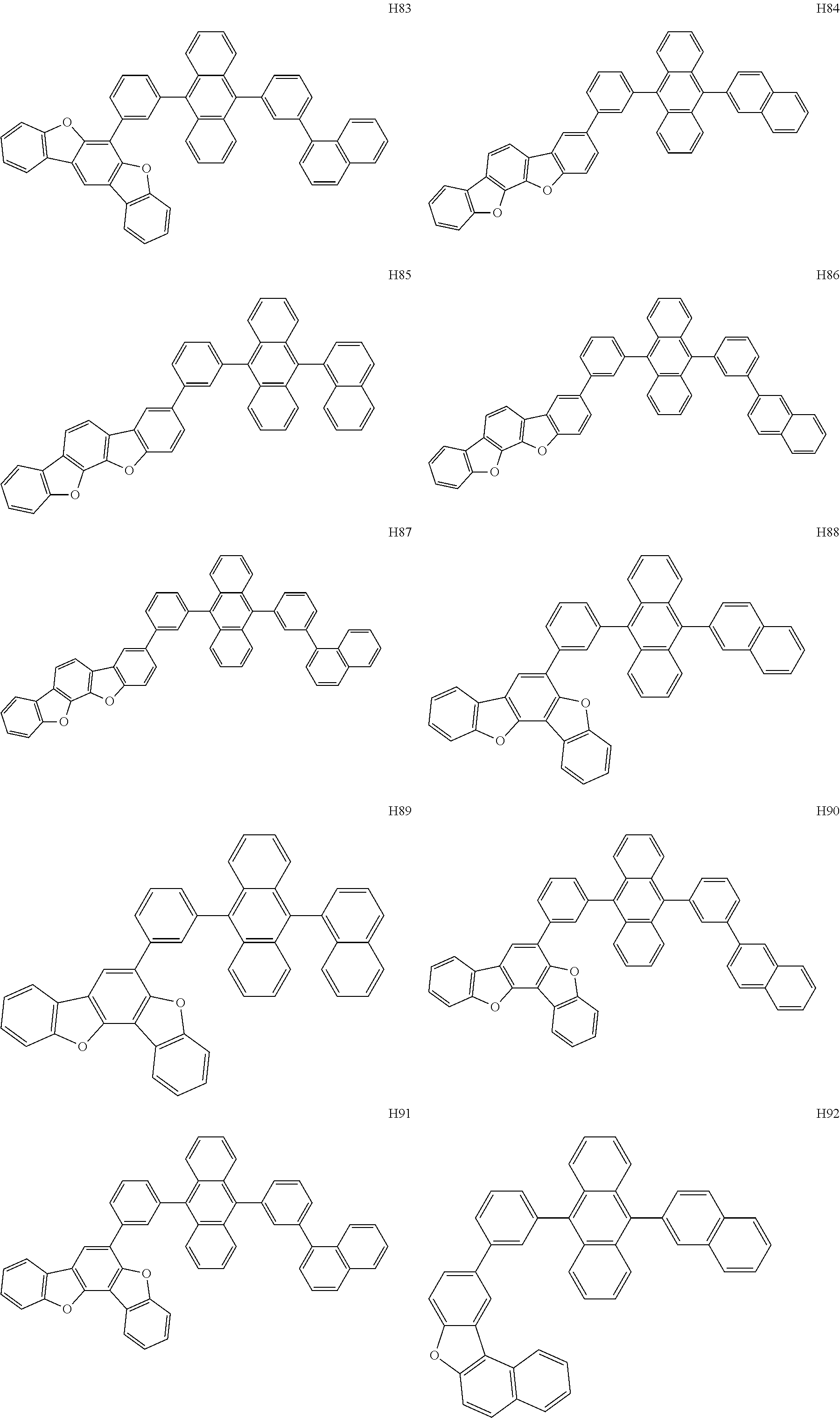

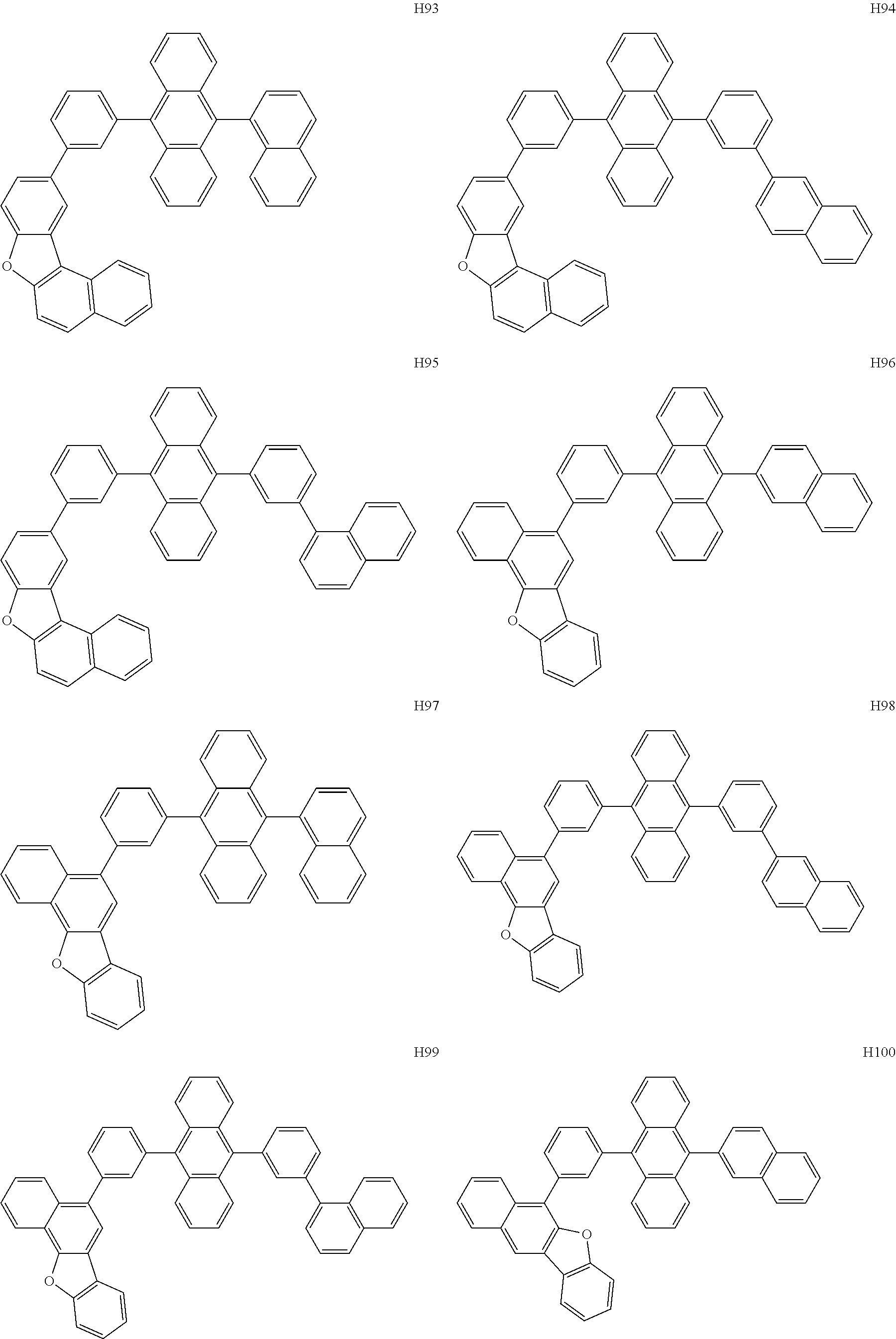

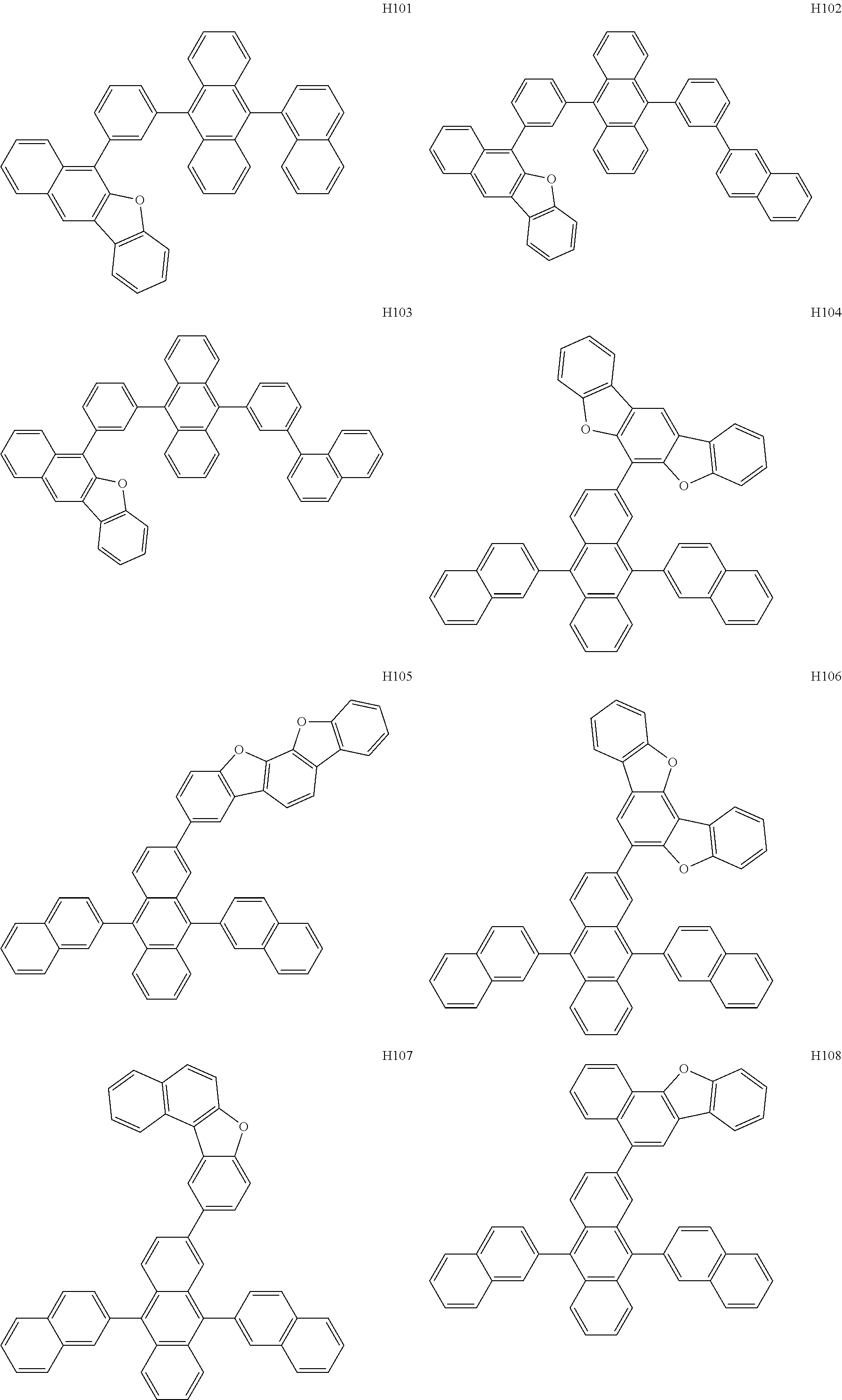

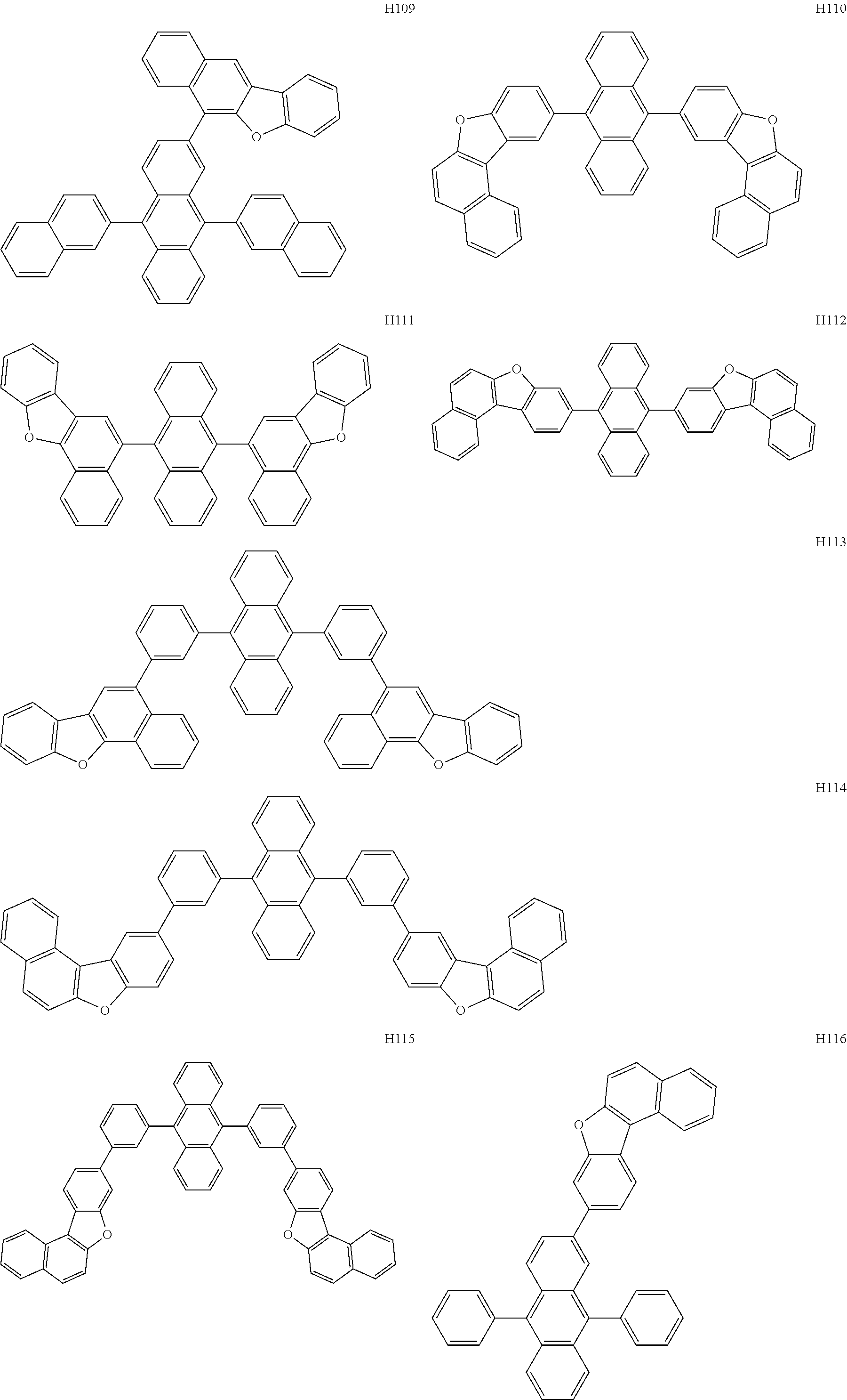

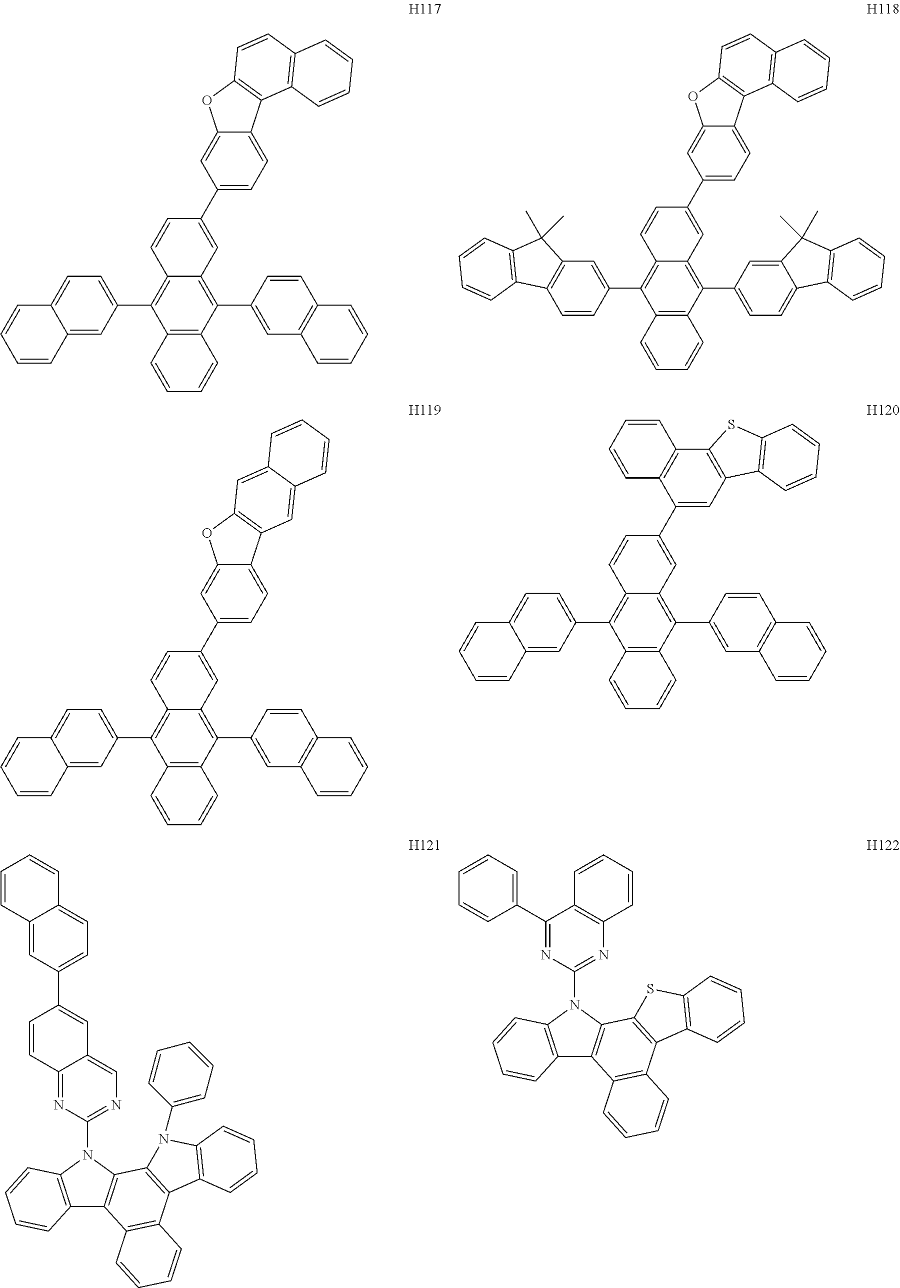

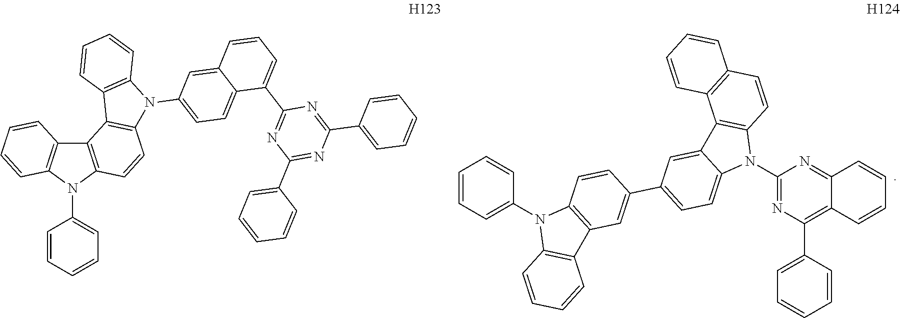 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

In some embodiments, the host may include a silicone-containing compound (e.g., BCPDS and/or the like), a phosphine oxide-containing compound (e.g., POPCPA and/or the like), or any combination thereof.

The host may include one type (or kind) of compounds only or two or more different types (or kinds) of compounds (for example, the constituent hosts may be BCPDS and POPCPA). As such, embodiments may be modified in various suitable ways.

### Phosphorescent Dopant

The emission layer may include the organometallic compound represented by Formula 1 described herein as a phosphorescent dopant.

### Delayed Fluorescence Material

The emission layer may include a delayed fluorescence material.

The delayed fluorescence material described herein may be any suitable compound that may emit delayed fluorescence according to a delayed fluorescence emission mechanism.

The delayed fluorescence material included in the emission layer may serve as a host or a dopant, depending on types (or kinds) of other materials included in the emission layer.

In some embodiments, a difference between a triplet energy level (eV) of the delayed fluorescence material and a singlet energy level (eV) of the delayed fluorescence material may be about 0 eV or greater and about 0.5 eV or smaller. When the difference between a triplet energy level (eV) of the delayed fluorescence material and a singlet energy level (eV) of the delayed fluorescence material is within this range, up-conversion from a triplet state to a singlet state in the delayed fluorescence material may be effectively occurred or achieved, thereby improving luminescence efficiency and/or the like of the light-emitting device 10.

In some embodiments, the delayed fluorescence material may include: i) a material including at least one electron donor (e.g., a π electron-rich C₃-C₆₀ cyclic group such as a carbazole group and/or the like) and at least one electron acceptor (e.g., a sulfoxide group, a cyano group, a π electron-deficient nitrogen-containing C₁-C₆₀ cyclic group, and/or the like), ii) a material including a C₈-C₆₀ polycyclic group including at least two cyclic groups condensed to each other (e.g., combined together with each other) and sharing boron (B), and/or the like.

Examples of the delayed fluorescence material may include at least one selected from Compounds DF1 to DF9:

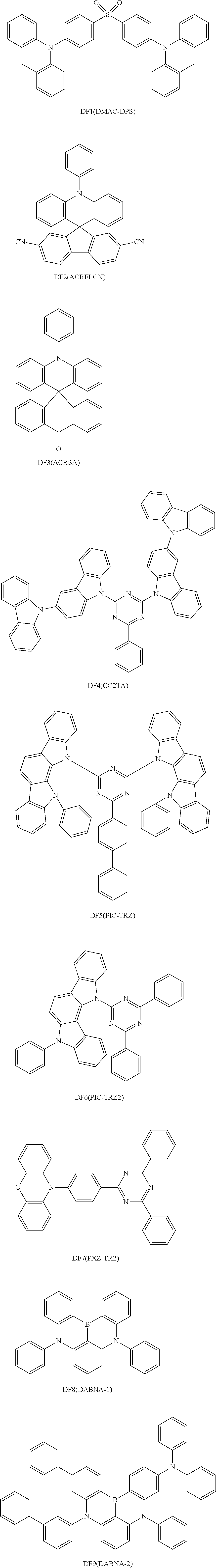

### Quantum Dot

The emission layer may include quantum dots.

The term “quantum dot,” as used herein, refers to a crystal of a semiconductor compound and may include any suitable material capable of emitting emission wavelengths of various suitable lengths according to the size of the crystal.

The diameter of the quantum dot may be, for example, in a range of about 1 nm to about 10 nm.

Quantum dots may be synthesized by a wet chemical process, a metal organic chemical vapor deposition process, a molecular beam epitaxy process, and/or any similar process.

The wet chemical process is a method of growing a quantum dot particle crystal by mixing a precursor material with an organic solvent. When the crystal grows, the organic solvent may naturally serve as a dispersant coordinated on the surface of the quantum dot crystal and control the growth of the crystal. Thus, the wet chemical method may be easier than the vapor deposition process such as the metal organic chemical vapor deposition (MOCVD) or the molecular beam epitaxy (MBE) process. Further, the growth of quantum dot particles may be controlled with a lower manufacturing cost.

The quantum dot may include a Group III-VI semiconductor compound; a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group III-VI semiconductor compound may include a binary compound such as In₂S₃; a ternary compound such as AgInS, AgInS₂, CuInS, and/or CuInS₂; or any combination thereof.

Examples of the Group II-VI semiconductor compound may include a binary compound such as CdS, CdSe, CdTe, ZnS, ZnSe, ZnTe, ZnO, HgS, HgSe, HgTe, MgSe, and/or MgS; a ternary compound such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Examples of the Group III-V semiconductor compound may include a binary compound such as GaN, GaP, GaAs, GaSb, AlN, AlP, AlAs, AlSb, InN, InP, InAs, and/or InSb; a ternary compound such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and/or InAlPSb; or any combination thereof. In some embodiments, the Group III-V semiconductor compound may further include a Group II element. Examples of the Group III-V semiconductor compound further including the Group II element may include InZnP, InGaZnP, InAlZnP, and the like.

Examples of the Group III-VI semiconductor compound may include a binary compound such as GaS, GaSe, Ga₂Se₃, GaTe, InS, In₂S₃, InSe, In₂Se₃, InTe, and the like; a ternary compound such as InGaS₃, InGaSe₃, and the like; or any combination thereof.

Examples of the Group I-III-VI semiconductor compound may include a ternary compound such as AgInS, AgInS₂, CuInS, CuInS₂, CuGaO₂, AgGaO₂, AgAlO₂, or any combination thereof.

Examples of the Group IV-VI semiconductor compound may include a binary compound such as SnS, SnSe, SnTe, PbS, PbSe, and/or PbTe; a ternary compound such as SnSeS, SnSeTe, SnSTe, PbSeS, PbSeTe, PbSTe, SnPbS, SnPbSe, and/or SnPbTe; a quaternary compound such as SnPbSSe, SnPbSeTe, and/or SnPbSTe; or any combination thereof.

The Group IV element or compound may be a single element such as Si or Ge; a binary compound such as SiC and/or SiGe; or any combination thereof.

Individual elements included in the multi-element compound, such as a binary compound, a ternary compound, and a quaternary compound, may be present in a particle thereof at a uniform or non-uniform concentration.

The quantum dot may have a single structure in which the concentration of each element included in the quantum dot is uniform (e.g., substantially uniform) or a core-shell double structure. In some embodiments, materials included in the core may be different from materials included in the shell.

The shell of the quantum dot may serve as a protective layer for preventing or reducing chemical denaturation of the core to maintain semiconductor characteristics and/or as a charging layer for imparting electrophoretic characteristics to the quantum dot. The shell may be a monolayer or a multilayer. An interface between a core and a shell may have a concentration gradient where a concentration of elements present in the shell decreases along a direction toward the core.

Examples of the shell of the quantum dot include metal or nonmetal oxide, a semiconductor compound, or a combination thereof. Examples of the metal oxide or the nonmetal oxide may include: a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO; a ternary compound such as MgAl₂O₄, CoFe₂O₄, NiFe₂O₄, and/or CoMn₂O₄; and any combination thereof. Example of the semiconductor compound may include a Group III-VI semiconductor compound; a Group II-VI semiconductor compound; a Group III-V semiconductor compound; a Group III-VI semiconductor compound; a Group I-III-VI semiconductor compound; a Group IV-VI semiconductor compound; or any combination thereof. In some embodiments, the semiconductor compound may be CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AIAs, AlP, AISb, or any combination thereof.

The quantum dot may have a full width of half maximum (FWHM) of a spectrum of an emission wavelength of about 45 nm or less, about 40 nm or less, or about 30 nm or less. When the FWHM of the quantum dot is within this range, color purity or color reproducibility may be improved. In addition, because light emitted through the quantum dot is emitted in all directions (or substantially all directions), an optical viewing angle may be improved.

In addition, the quantum dot may be, for example, a spherical, pyramidal, multi-arm, or cubic nanoparticle, nanotube, nanowire, nanofiber, and/or nanoplate particle.

By adjusting the size of the quantum dot, the energy band gap may also be adjusted, thereby obtaining light of various suitable wavelengths in the quantum dot emission layer. By using quantum dots of various suitable sizes, a light-emitting device that may emit light of various suitable wavelengths may be realized. In some embodiments, the size of the quantum dot may be selected such that the quantum dot may emit red, green, and/or blue light. In addition, the size of the quantum dot may be selected such that the quantum dot may emit white light by combining light having various suitable colors.

### Electron Transport Region in Interlayer 130

The electron transport region may have i) a single-layered structure including (e.g., consisting of) a single layer including (e.g., consisting of) a single material, ii) a single-layered structure including (e.g., consisting of) a single layer including a plurality of different materials, or iii) a multi-layered structure having a plurality of layers including a plurality of different materials.

The electron transport region may include a buffer layer, a hole blocking layer, an electron control layer, an electron transport layer, and/or an electron injection layer.

In some embodiments, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein layers of each structure are sequentially stacked on the emission layer in the respectively stated order.

The electron transport region (e.g., a buffer layer, a hole blocking layer, an electron control layer, and/or an electron transport layer in the electron transport region) may include a metal-free compound including at least one π electron-deficient nitrogen-containing C₁-C₆₀ cyclic group.

In some embodiments, the electron transport region may include a compound represented by Formula 601:

[Ar₆₀₁]ₓₑ₁₁-[(L₆₀₁)ₓₑ₁-R₆₀₁]ₓₑ₂₁ Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),

Q₆₀₁ to Q₆₀₃ may each be understood by referring to the description of Q₁ provided herein,

xe21 may be 1, 2, 3, 4, or 5, and

at least one selected from Ar₆₀₁, L₆₀₁, and R₆₀₁ may independently be a π electron-deficient nitrogen-containing C₁-C₆₀ cyclic group unsubstituted or substituted with at least one R₁₀ₐ.

In some embodiments, when xe11 in Formula 601 is 2 or greater, at least two Ar₆₀₁(s) may be bound via a single bond.

In some embodiments, in Formula 601, Ar₆₀₁ may be a substituted or unsubstituted anthracene group.

In some embodiments, the electron transport region may include a compound represented by Formula 601-1:

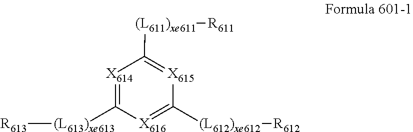

wherein, in Formula 601-1,

X₆₁₄ may be N or C(R₆₁₄), X₆₁₅ may be N or C(R₆₁₅), X₆₁₆ may be N or C(R₆₁₆), at least one selected from X₆₁₄ to X₆₁₆ may be N,

L₆₁₁ to L₆₁₃ may each be understood by referring to the description of L₆₀₁ provided herein,

xe611 to xe613 may each be understood by referring to the description of xe1 provided herein,

R₆₁₁ to R₆₁₃ may each be understood by referring to the description of R₆₀₁ provided herein, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, in Formulae 601 and 601-1, xe1 and xe611 to xe613 may each independently be 0, 1, or 2.

The electron transport region may include one selected from Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, diphenyl(4-(triphenylsilyl)phenyl)-phosphine oxide (TSPO1), or any combination thereof:

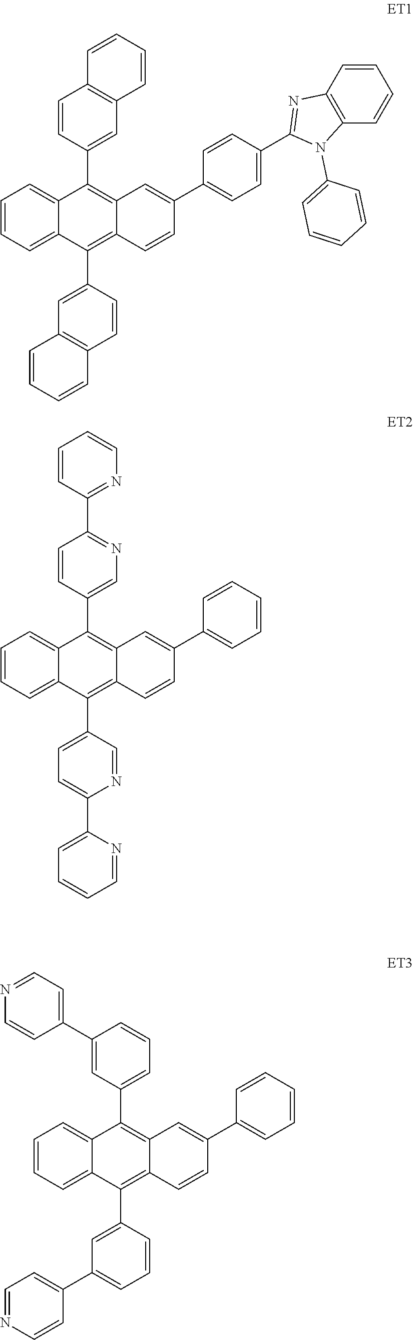

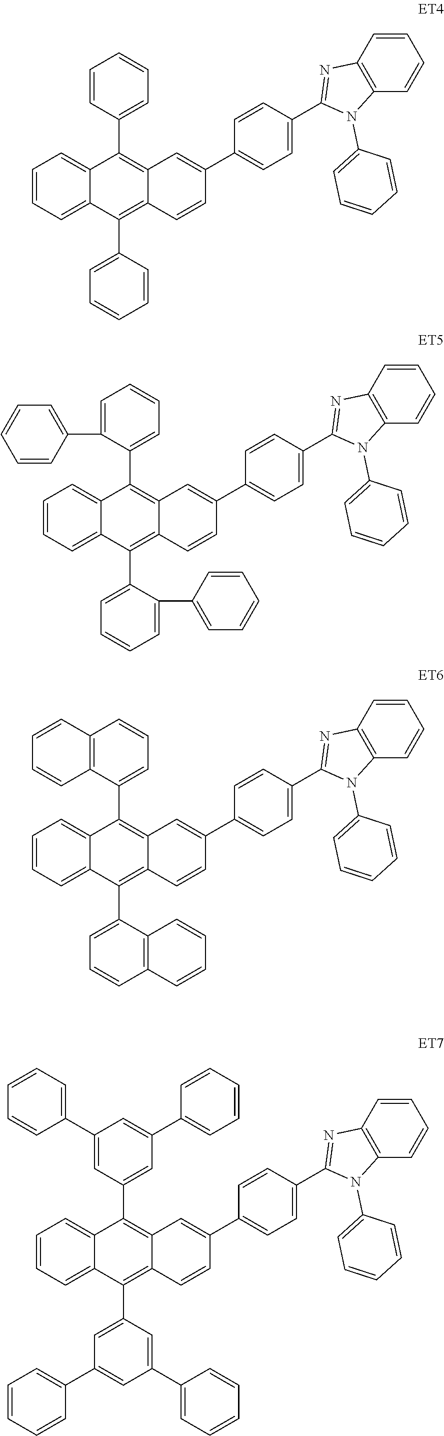 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

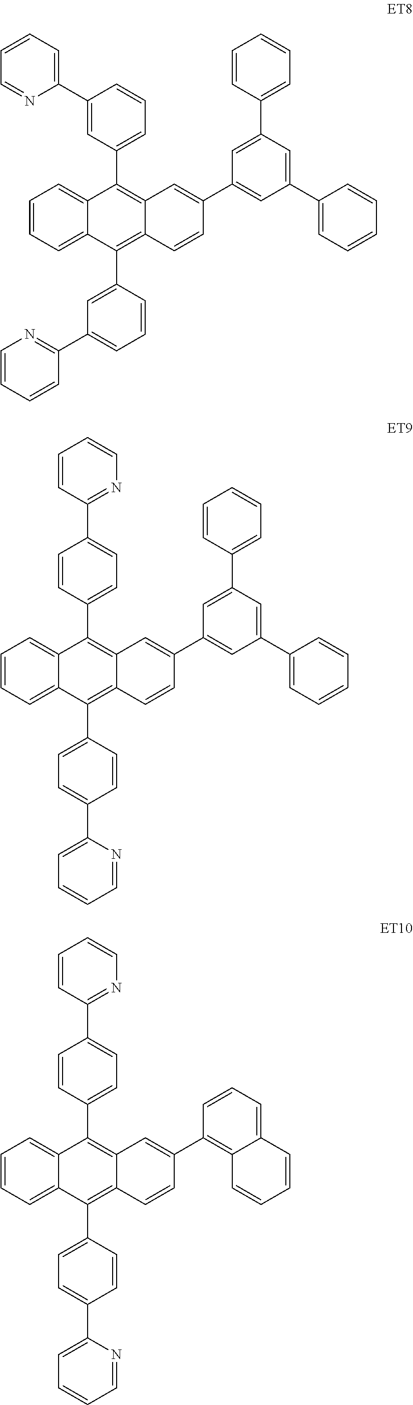

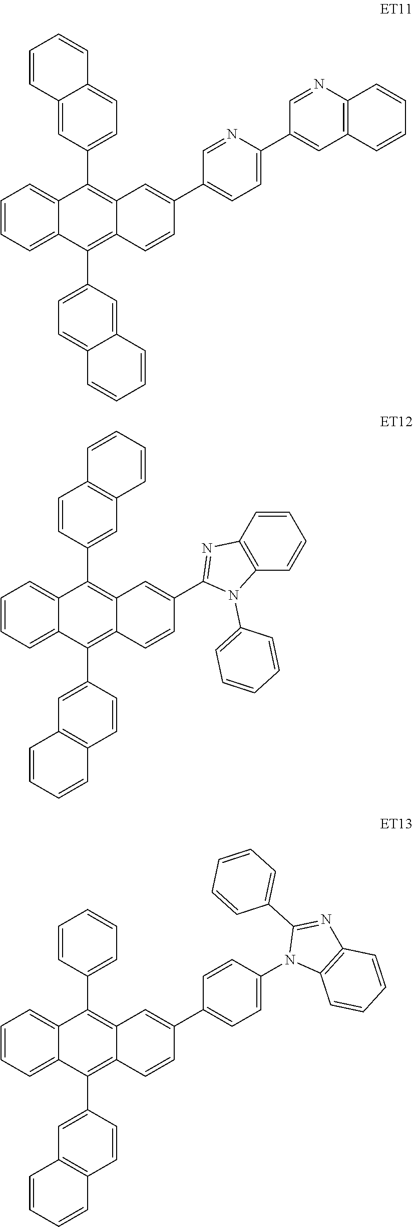

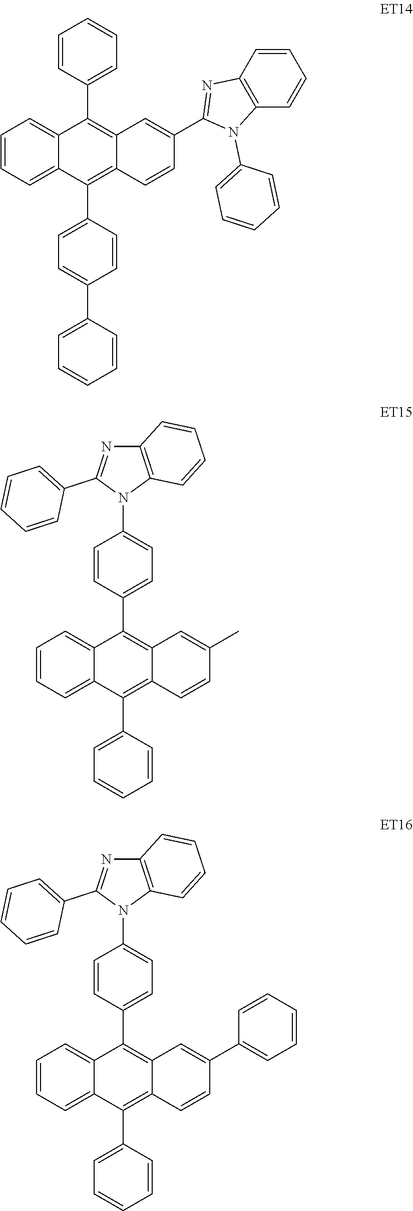 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

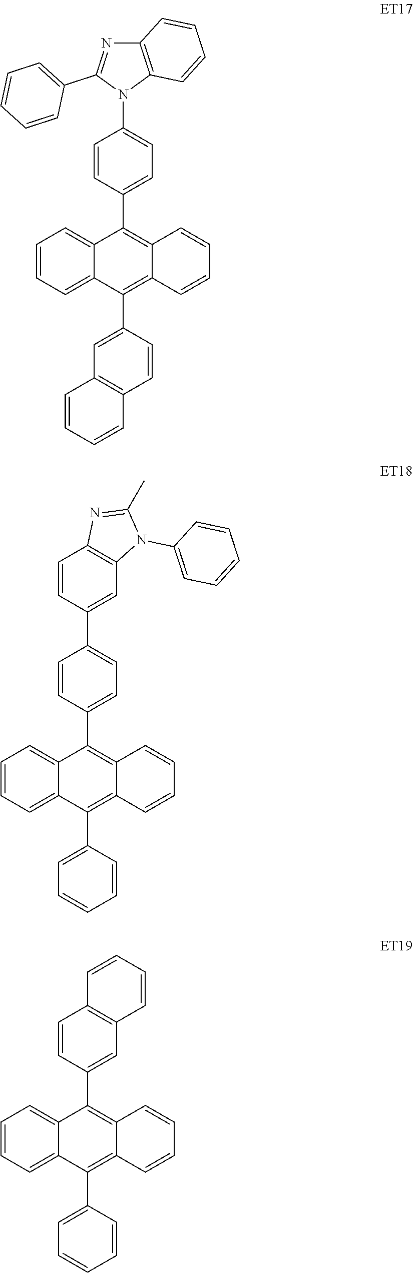

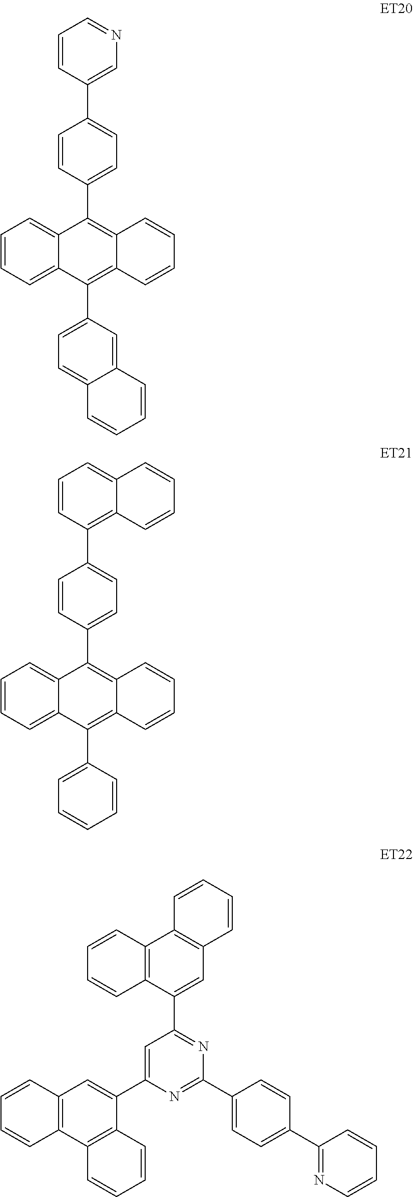

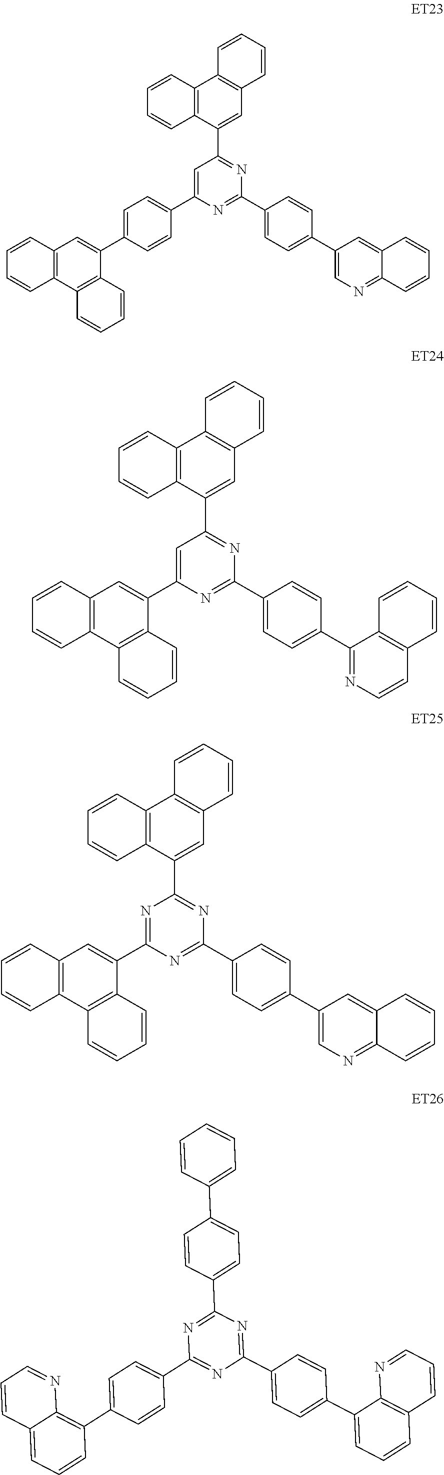 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

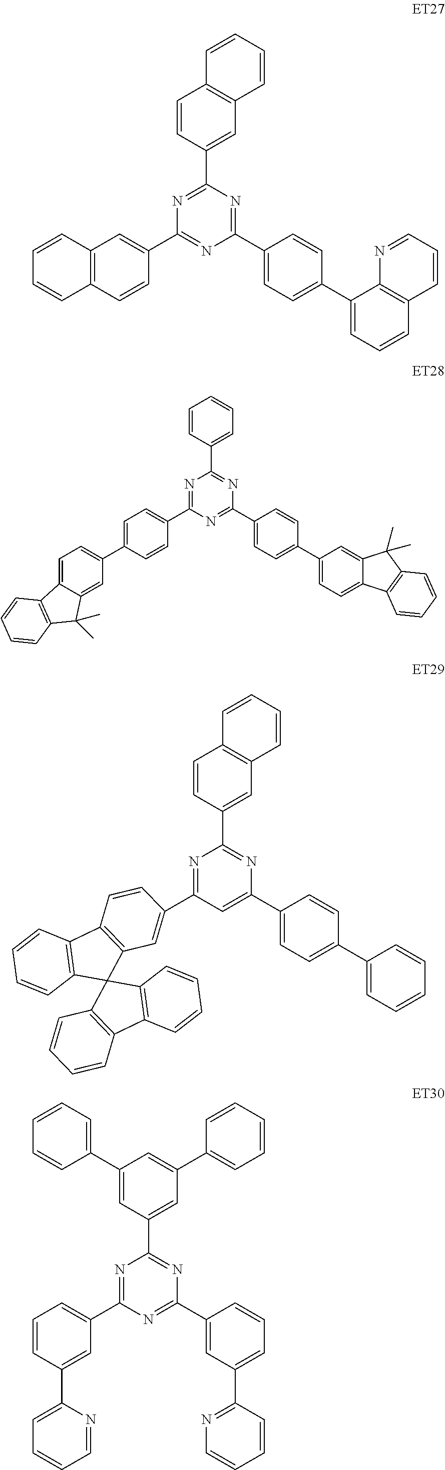

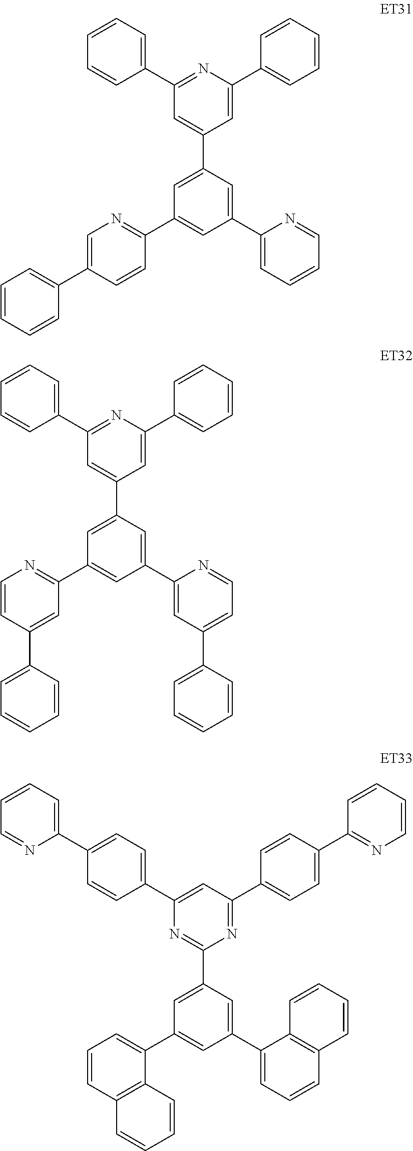

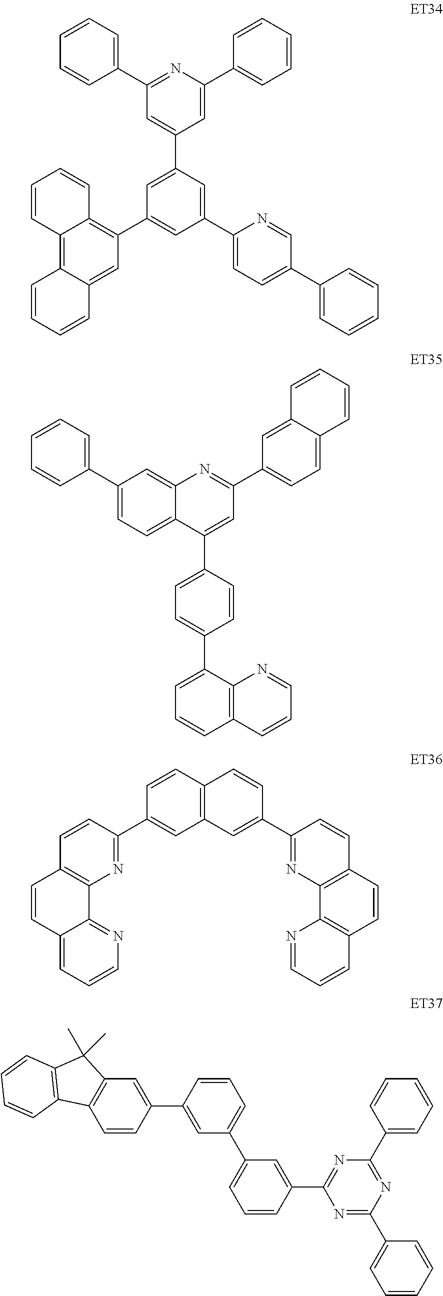

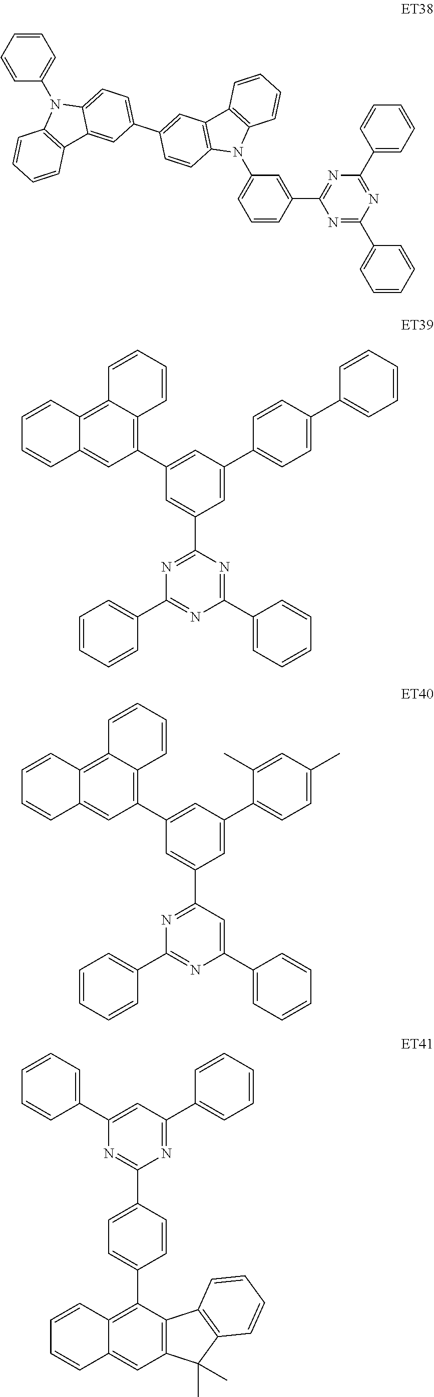

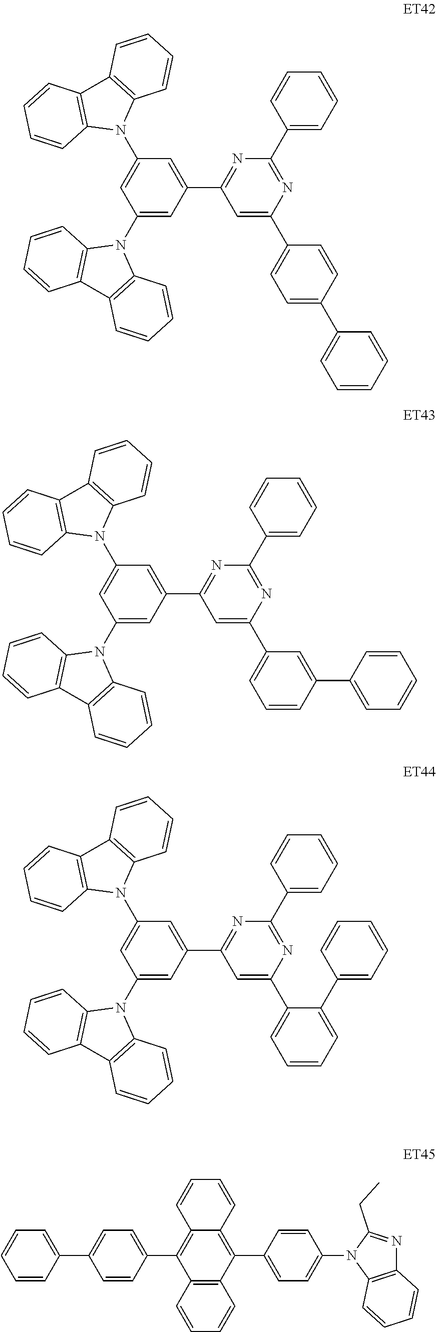 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

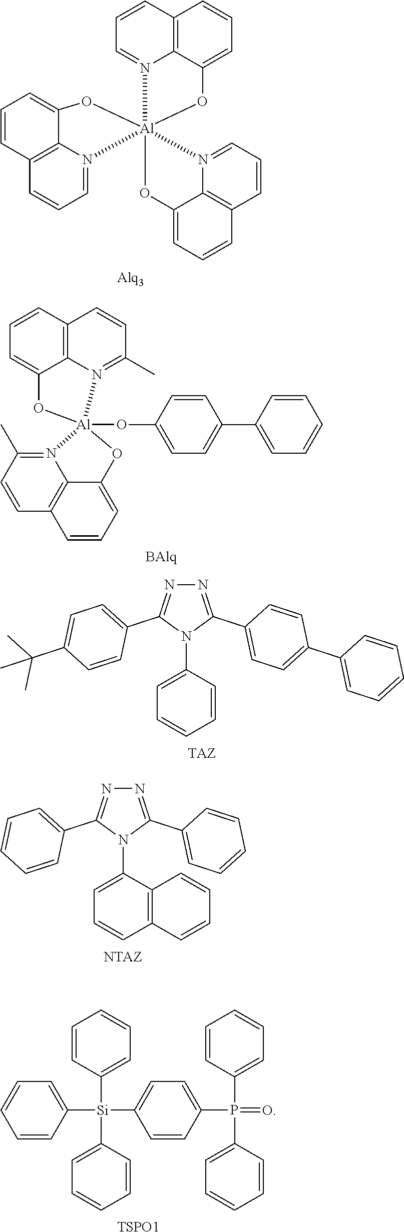

The thickness of the electron transport region may be in a range of about 100 Å to about 5,000 Å, and in some embodiments, about 160 Å to about 4,000 Å. When the electron transport region includes a buffer layer, a hole blocking layer, an electron control layer, an electron transport layer, or any combination thereof, the thicknesses of the buffer layer, the hole blocking layer, or the electron control layer may each independently be in a range of about 20 Å to about 1,000 Å, for example, about 30 Å to about 300 Å, and the thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron injection layer are each within these ranges, excellent electron transport characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a lithium (Li) ion, a sodium (Na) ion, a potassium (K) ion, a rubidium (Rb) ion, or a cesium (Cs) ion. A metal ion of the alkaline earth metal complex may be a beryllium (Be) ion, a magnesium (Mg) ion, a calcium (Ca) ion, a strontium (Sr) ion, or a barium (Ba) ion. Each ligand coordinated with the metal ion of the alkali metal complex and the alkaline earth metal complex may independently be hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, the metal-containing material may include a Li complex. The Li complex may include, e.g., Compound ET-D1 (LiQ) or Compound ET-D2:

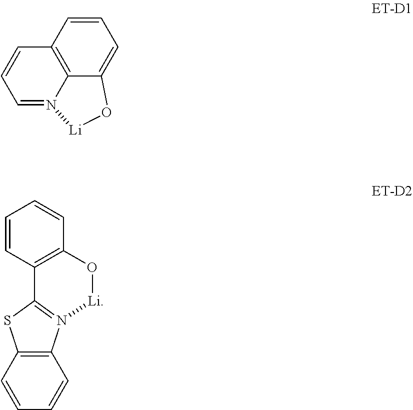

The electron transport region may include an electron injection layer that facilitates injection of electrons from the second electrode 150. The electron injection layer may be in direct contact (e.g., physical contact) with the second electrode 150.

The electron injection layer may have i) a single-layered structure including (e.g., consisting of) a single layer including (e.g., consisting of) a single material, ii) a single-layered structure including (e.g., consisting of) a single layer including a plurality of different materials, or iii) a multi-layered structure having a plurality of layers including a plurality of different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may be Li, Na, K, Rb, Cs or any combination thereof. The alkaline earth metal may be Mg, Ca, Sr, Ba, or any combination thereof. The rare earth metal may be Sc, Y, Ce, Tb, Yb, Gd, or any combination thereof.

The alkali metal-containing compound, the alkaline earth metal-containing compound, and the rare earth metal-containing compound may respectively be oxides, halides (e.g., fluorides, chlorides, bromides, or iodines), tellurides, or any combination thereof of each of the alkali metal, the alkaline earth metal, and the rare earth metal.

The alkali metal-containing compound may be alkali metal oxides such as Li₂O, Cs₂O, and/or K₂O, alkali metal halides such as LiF, NaF, CsF, KF, LiI, NaI, CsI, and/or KI, or any combination thereof. The alkaline earth-metal-containing compound may include alkaline earth-metal oxides, such as BaO, SrO, CaO, BaₓSr₁₋ₓO (wherein x is a real number that satisfies 0<x<1), and/or BaₓCa₁₋ₓO (wherein x is a real number that satisfies 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In some embodiments, the rare earth metal-containing compound may include a lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and the like.

The alkali metal complex, the alkaline earth metal complex, and the rare earth metal complex may include: i) one selected from ions of the alkali metal, alkaline earth metal, and rare earth metal described above, and ii) a ligand bound to the metal ion, e.g., hydroxyquinoline, hydroxyisoquinoline, hydroxybenzoquinoline, hydroxyacridine, hydroxyphenanthridine, hydroxyphenyloxazole, hydroxyphenylthiazole, hydroxydiaphenyloxadiazole, hydroxydiphenylthiadiazole, hydroxyphenylpyridine, hydroxyphenylbenzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In some embodiments, the electron injection layer may further include an organic material (e.g., a compound represented by Formula 601).

In some embodiments, the electron injection layer may include (e.g., consist of) i) an alkali metal-containing compound (e.g., alkali metal halide), or ii) a) an alkali metal-containing compound (e.g., alkali metal halide); and b) an alkali metal, an alkaline earth metal, a rare earth metal, or any combination thereof. In some embodiments, the electron injection layer may be a KI:Yb co-deposition layer, a RbI:Yb co-deposition layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth metal complex, the rare earth metal complex, or any combination thereof may be homogeneously or non-homogeneously dispersed in a matrix including the organic material.

The thickness of the electron injection layer may be in a range of about 1 Å to about 100 Å, and in some embodiments, about 3 Å to about 90 Å. When the thickness of the electron injection layer is within any of these ranges, excellent electron injection characteristics may be obtained without a substantial increase in driving voltage.

### Second Electrode 150

The second electrode 150 may be on the interlayer 130. In an embodiment, the second electrode 150 may be a cathode that is an electron injection electrode. In this embodiment, a material for forming the second electrode 150 may be a material having a low work function, for example, a metal, an alloy, an electrically conductive compound, or any combination thereof.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure, or a multi-layered structure including two or more layers.

### Capping Layer

A first capping layer may be located outside the first electrode 110, and/or a second capping layer may be located outside the second electrode 150. In some embodiments, the light-emitting device 10 may have a structure in which the first capping layer, the first electrode 110, the interlayer 130, and the second electrode 150 are sequentially stacked in this stated order, a structure in which the first electrode 110, the interlayer 130, the second electrode 150, and the second capping layer are sequentially stacked in this stated order, or a structure in which the first capping layer, the first electrode 110, the interlayer 130, the second electrode 150, and the second capping layer are sequentially stacked in this stated order.

In the light-emitting device 10, light emitted from the emission layer in the interlayer 130 may pass through the first electrode 110 (which may be a semi-transmissive electrode or a transmissive electrode) and through the first capping layer to the outside. In the light-emitting device 10, light emitted from the emission layer in the interlayer 130 may pass through the second electrode 150 (which may be a semi-transmissive electrode or a transmissive electrode) and through the second capping layer to the outside.

The first capping layer and the second capping layer may improve the external luminescence efficiency based on the principle of constructive interference. Accordingly, the optical extraction efficiency of the light-emitting device 10 may be increased, thereby improving luminescence efficiency of the light-emitting device 10.

The first capping layer and the second capping layer may each include a material having a refractive index of 1.6 or higher (at 589 nm).

The first capping layer and the second capping layer may each independently be an organic capping layer including an organic material, an inorganic capping layer including an inorganic material, or a composite capping layer including an organic material and an inorganic material.

At least one selected from the first capping layer and the second capping layer may each independently include carbocyclic compounds, heterocyclic compounds, amine group-containing compounds, porphine derivatives, phthalocyanine derivatives, naphthalocyanine derivatives, alkali metal complexes, alkaline earth metal complexes, or any combination thereof. The carbocyclic compound, the heterocyclic compound, and the amine group-containing compound may optionally be substituted with a substituent of O, N, S, Se, Si, F, Cl, Br, I, or any combination thereof.

In some embodiments, at least one selected from the first capping layer and the second capping layer may each independently include an amine group-containing compound.

In some embodiments, at least one selected from the first capping layer and the second capping layer may each independently include the compound represented by Formula 201, the compound represented by Formula 202, or any combination thereof.

In one or more embodiments, at least one selected from the first capping layer and the second capping layer may each independently include one selected from of Compounds HT28 to HT33, one selected from Compounds CP1 to CP6, p-NPB, or any combination thereof:

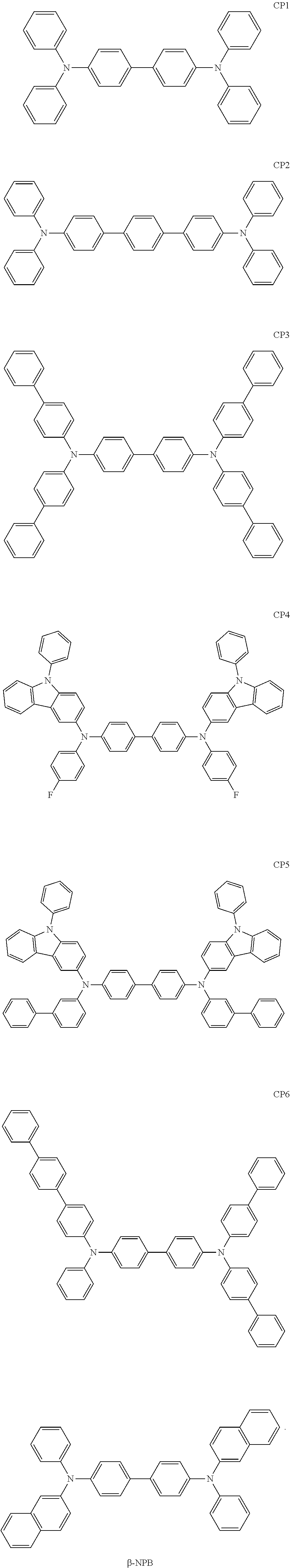

### Electronic Apparatus

The light-emitting device may be included in various suitable electronic apparatuses. In some embodiments, an electronic apparatus including the light-emitting device may be an emission apparatus and/or an authentication apparatus.

The electronic apparatus (e.g., an emission apparatus) may further include, in addition to the light-emitting device, i) a color filter, ii) a color-conversion layer, or iii) a color filter and a color-conversion layer. The color filter and/or the color-conversion layer may be on at least one traveling direction of light emitted from the light-emitting device. For example, light emitted from the light-emitting device may be blue light or white light. The light-emitting device may be understood by referring to the descriptions provided herein. In some embodiments, the color-conversion layer may include quantum dots.

The electronic apparatus may include a first substrate. The first substrate may include a plurality of sub-pixel areas, the color filter may include a plurality of color filter areas respectively corresponding to the plurality of sub-pixel areas, and the color-conversion layer may include a plurality of color-conversion areas respectively corresponding to the plurality of sub-pixel areas.

A pixel defining film may be located between the plurality of sub-pixel areas to define each sub-pixel area.

The color filter may further include a plurality of color filter areas and light-blocking patterns between the plurality of color filter areas, and the color-conversion layer may further include a plurality of color-conversion areas and light-blocking patterns between the plurality of color-conversion areas.

The plurality of color filter areas (or a plurality of color-conversion areas) may include: a first area emitting first color light; a second area emitting second color light; and/or a third area emitting third color light, and the first color light, the second color light, and/or the third color light may have different maximum emission wavelengths. In some embodiments, the first color light may be red light, the second color light may be green light, and the third color light may be blue light. In some embodiments, the plurality of color filter areas (or the plurality of color-conversion areas) may each include quantum dots. In some embodiments, the first area may include red quantum dots, the second area may include green quantum dots, and the third area may not include a quantum dot. The quantum dot may be understood by referring to the description of the quantum dot provided herein. The first area, the second area, and/or the third area may each further include an emitter.

In some embodiments, the light-emitting device may emit first light, the first area may absorb the first light to emit a 1-1 color light (e.g., a first first color light), the second area may absorb the first light to emit a 2-1 color light (e.g., a second first color light), and the third area may absorb the first light to emit a 3-1 color light (e.g., a third first color light). In this embodiment, the 1-1 color light, the 2-1 color light, and the 3-1 color light may each have a different maximum emission wavelength. In some embodiments, the first light may be blue light, the 1-1 color light may be red light, the 2-1 color light may be green light, and the 3-1 color light may be blue light.

The electronic apparatus may further include a thin-film transistor, in addition to the light-emitting device. The thin-film transistor may include a source electrode, a drain electrode, and an active layer, wherein one selected from the source electrode and the drain electrode may be electrically coupled to one selected from the first electrode and the second electrode of the light-emitting device.

The thin-film transistor may further include a gate electrode, a gate insulating film, and/or the like.

The active layer may include a crystalline silicon, an amorphous silicon, an organic semiconductor, and/or an oxide semiconductor.

The electronic apparatus may further include an encapsulation unit for sealing the light-emitting device. The encapsulation unit may be located between the color filter and/or the color-conversion layer and the light-emitting device. The encapsulation unit may allow light to pass to the outside from the light-emitting device and prevent or reduce permeation of air and/or moisture to the light-emitting device at the same time. The encapsulation unit may be a sealing substrate including a transparent glass and/or a plastic substrate. The encapsulation unit may be a thin-film encapsulating layer including at least one of an organic layer and/or an inorganic layer. When the encapsulation unit is a thin film encapsulating layer, the electronic apparatus may be flexible.

In addition to the color filter and/or the color-conversion layer, various suitable functional layers may be on the encapsulation unit depending on the use of an electronic apparatus. Examples of the functional layer may include a touch screen layer, a polarization layer, and/or the like. The touch screen layer may be a resistive touch screen layer, a capacitive touch screen layer, and/or an infrared beam touch screen layer. The authentication apparatus may be, for example, a biometric authentication apparatus that identifies an individual according biometric information (e.g., a fingertip, a pupil, and/or the like).

The authentication apparatus may further include a biometric information collecting unit, in addition to the light-emitting device described above.

The electronic apparatus may be applicable to various suitable displays, an optical source, lighting, a personal computer (e.g., a mobile personal computer), a cellphone, a digital camera, an electronic note, an electronic dictionary, an electronic game console, a medical device (e.g., an electronic thermometer, a blood pressure meter, a glucometer, a pulse measuring device, a pulse wave measuring device, an electrocardiograph recorder, an ultrasonic diagnosis device, and/or an endoscope display device), a fish finder, various suitable measurement devices, gauges (e.g., gauges of an automobile, an airplane, and/or a ship), and/or a projector.

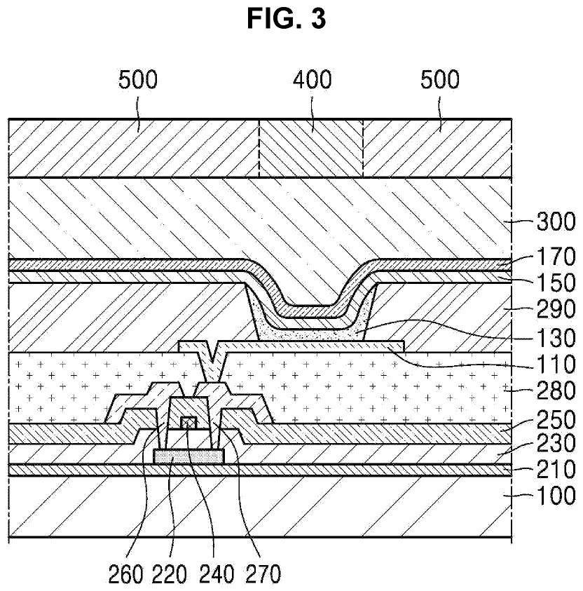

### Descriptions of FIGS. 2 and 3

FIG. 2 is a schematic cross-sectional view of a light-emitting apparatus according to an embodiment.

An emission apparatus in FIG. 2 may include a substrate 100, a thin-film transistor, a light-emitting device, and an encapsulation unit 300 sealing the light-emitting device.

The substrate 100 may be a flexible substrate, a glass substrate, and/or a metal substrate. A buffer layer 210 may be on the substrate 100. The buffer layer 210 may prevent or reduce penetration of impurities through the substrate 100 and provide a flat surface on the substrate 100.

A thin-film transistor may be on the buffer layer 210. The thin-film transistor may include an active layer 220, a gate electrode 240, a source electrode 260, and a drain electrode 270.

The active layer 220 may include an inorganic semiconductor such as silicon and/or polysilicon, an organic semiconductor, and/or an oxide semiconductor and include a source area, a drain area, and a channel area.

A gate insulating film 230 for insulating the active layer 220 and the gate electrode 240 may be on the active layer 220, and the gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270 to provide insulation therebetween.

The source electrode 260 and the drain electrode 270 may be on the interlayer insulating film 250. The interlayer insulating film 250 and the gate insulating film 230 may be formed to expose the source area and the drain area of the active layer 220, and the source electrode 260 and the drain electrode 270 may be adjacent to the exposed source area and the exposed drain area of the active layer 220.

Such a thin-film transistor may be electrically coupled to a light-emitting device to drive the light-emitting device and may be protected by a passivation layer 280. The passivation layer 280 may include an inorganic insulating film, an organic insulating film, or a combination thereof. A light-emitting device may be on the passivation layer 280. The light-emitting device may include a first electrode 110, an interlayer 130, and a second electrode 150.

The first electrode 110 may be on the passivation layer 280. The passivation layer 280 may not fully cover the drain electrode 270 and expose a set or specific area of the drain electrode 270, and the first electrode 110 may be coupled to the exposed drain electrode 270.

A pixel-defining film 290 may be on the first electrode 110. The pixel-defining film 290 may expose a set or specific area of the first electrode 110, and the interlayer 130 may be formed in the exposed area. The pixel-defining film 290 may be a polyimide or polyacryl organic film. In some embodiments, some higher layers of the interlayer 130 may extend to the upper portion of the pixel-defining film 290 and may be in the form of a common layer.

The second electrode 150 may be on the interlayer 130, and a capping layer 170 may be additionally formed on the second electrode 150. The capping layer 170 may be formed to cover the second electrode 150.

The encapsulation unit 300 may be on the capping layer 170. The encapsulation unit 300 may be on the light-emitting device to protect a light-emitting device from moisture or oxygen. The encapsulation unit 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxy methylene, poly aryllate, hexamethyl disiloxane, an acrylic resin (e.g., polymethyl methacrylate, polyacrylic acid, and the like), an epoxy resin (e.g., aliphatic glycidyl ether (AGE) and the like), or any combination thereof; or a combination of the inorganic film and the organic film.

FIG. 3 is a schematic cross-sectional view of another light-emitting apparatus according to an embodiment.

The emission apparatus shown in FIG. 3 may be substantially identical to the emission apparatus shown in FIG. 2, except that a light-shielding pattern 500 and a functional area 400 are additionally located on the encapsulation unit 300. The functional area 400 may be i) a color filter area, ii) a color-conversion area, or iii) a combination of a color filter area and a color-conversion area. In some embodiments, the light-emitting device shown in FIG. 3 included in the emission apparatus may be a tandem light-emitting device.

### Manufacturing Method

The layers constituting the hole transport region, the emission layer, and the layers constituting the electron transport region may be formed in a set or specific region by using one or more suitable methods such as vacuum deposition, spin coating, casting, Langmuir-Blodgett (LB) deposition, ink-jet printing, laser printing, and/or laser-induced thermal imaging.

When the layers constituting the hole transport region, the emission layer, and the layers constituting the electron transport region are each formed by vacuum deposition, the vacuum deposition may be performed at a deposition temperature in a range of about 100° C. to about 500° C. at a vacuum degree in a range of about 10⁻⁸ torr to about 10⁻³ torr, and at a deposition rate in a range of about 0.01 Angstroms per second (Å/sec) to about 100 Å/sec, depending on the material to be included in each layer and the structure of each layer to be formed.

### General Definitions of Terms

The term “C₃-C₆₀ carbocyclic group,” as used herein, refers to a cyclic group including (e.g., consisting of) carbon atoms only and having 3 to 60 carbon atoms. The term “C₁-C₆₀ heterocyclic group,” as used herein, refers to a cyclic group having 1 to 60 carbon atoms in addition to a heteroatom other than carbon atoms. The C₃-C₆₀ carbocyclic group and the C₁-C₆₀ heterocyclic group may each be a monocyclic group including (e.g., consisting of) one ring or a polycyclic group in which at least two rings are condensed (e.g., combined together). For example, the number of ring-forming atoms in the C₁-C₆₀ heterocyclic group may be in a range of 3 to 61.

The term “cyclic group,” as used herein, may include the C₃-C₆₀ carbocyclic group and the C₁-C₆₀ heterocyclic group.

As used herein, the term “π electron-rich C₃-C₆₀ cyclic group” refers to a cyclic group having 3 to 60 carbon atoms and not including *—N═*′ as a ring-forming moiety. The term “π electron-deficient nitrogen-containing C₁-C₆₀ cyclic group,” as used herein, refers to a heterocyclic group having 1 to 60 carbon atoms and *—N═*′ as a ring-forming moiety.

In some embodiments,

the C₃-C₆₀ carbocyclic group may be i) a T1 group or ii) a group in which at least two T1 groups are condensed (e.g., combined together, for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) a T2 group, ii) a group in which at least two T2 groups are condensed (e.g., combined together), or iii) a group in which at least one T2 group is condensed with at least one T1 group (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonapthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like),

the π electron-rich C₃-C₆₀ cyclic group may be i) a T1 group, ii) a condensed group in which at least two T1 groups are condensed (e.g., combined together), iii) a T3 group, iv) a condensed group in which at least two T3 groups are condensed (e.g., combined together), or v) a condensed group in which at least one T3 group is condensed (e.g., combined together) with at least one T1 group (for example, a C₃-C₆₀ carbocyclic group, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonapthothiophene group, a benzonaphthosilole group, a benzofurodibenzofuran group, a benzofurodibenzothiophene group, a benzothienodibenzothiophene group, and the like), and

the π electron-deficient nitrogen-containing C₁-C₆₀ cyclic group may be i) a T4 group, ii) a group in which at least twos T4 groups are condensed (e.g., combined together), iii) a group in which at least one T4 group is condensed (e.g., combined together) with at least one T1 group, iv) a group in which at least one T4 group is condensed (e.g., combined together) with at least one T3 group, or v) a group in which at least one T4 group, at least one T1 group, and at least one T3 group are condensed (e.g., combined together, for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and the like),

wherein the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group,

the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group,

the T3 group may be a furan group, a thiophene group, a 1H-pyrrole group, a silole group, or a borole group, and

the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The term “cyclic group,” “C₃-C₆₀ carbocyclic group,” “C₁-C₆ heterocyclic group,” “π electron-rich C₃-C₆₀ cyclic group,” or “π electron-deficient nitrogen-containing C₁-C₆₀ cyclic group,” as used herein, may be a group condensed (e.g., combined together) with any suitable cyclic group, a monovalent group, or a polyvalent group (e.g., a divalent group, a trivalent group, a quadvalent group, or the like), depending on the structure of the formula to which the term is applied. For example, a “benzene group” may be a benzo group, a phenyl group, a phenylene group, or the like, and this may be understood by one of ordinary skill in the art, depending on the structure of the formula including the “benzene group.”

Examples of the monovalent C₃-C₆₀ carbocyclic group and the monovalent C₁-C₆ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the monovalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group,” as used herein, refers to a linear or branched aliphatic hydrocarbon monovalent group having 1 to 60 carbon atoms, and examples thereof include a methyl group, an ethyl group, an n-propyl group, an iso-propyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an iso-hexyl group, a sec-hexyl group, a tert-hexyl group, an n-heptyl group, an iso-heptyl group, a sec-heptyl group, a tert-heptyl group, an n-octyl group, an iso-octyl group, a sec-octyl group, a tert-octyl group, an n-nonyl group, an iso-nonyl group, a sec-nonyl group, a tert-nonyl group, an n-decyl group, an iso-decyl group, a sec-decyl group, and a tert-decyl group. The term “C₁-C₆₀ alkylene group,” as used herein, refers to a divalent group having substantially the same structure as the C₁-C₆₀ alkyl group.

The term “C₂-C₆₀ alkenyl group,” as used herein, refers to a hydrocarbon group having at least one carbon-carbon double bond at a main chain (e.g., in the middle) or at a terminal end (e.g., the terminus) of the C₂-C₆₀ alkyl group. Examples thereof include an ethenyl group, a propenyl group, and a butenyl group. The term “C₂-C₆₀ alkenylene group,” as used herein, refers to a divalent group having substantially the same structure as the C₂-C₆₀ alkenyl group.

The term “C₂-C₆₀ alkynyl group,” as used herein, refers to a monovalent hydrocarbon group having at least one carbon-carbon triple bond at a main chain (e.g., in the middle) or at a terminal end (e.g., the terminus of the C₂-C₆₀ alkyl group. Examples thereof include an ethynyl group and a propynyl group. The term “C₂-C₆₀ alkynylene group,” as used herein, refers to a divalent group having substantially the same structure as the C₂-C₆₀ alkynyl group.

The term “C₁-C₆₀ alkoxy group,” as used herein, refers to a monovalent group represented by —OA₁₀₁ (wherein A₁₀₁ is a C₁-C₁ alkyl group). Examples thereof include a methoxy group, an ethoxy group, and an isopropyloxy group.

The term “C₃-C₁₀ cycloalkyl group,” as used herein, refers to a monovalent saturated hydrocarbon monocyclic group including 3 to 10 carbon atoms. Examples of the C₃-C₁₀ cycloalkyl group as used herein include a cyclopropyl group, a cyclobutyl group, a cyclopentyl group, a cyclohexyl group, a cycloheptyl group, a cyclooctyl group, an adamantanyl group, a norbornanyl (bicyclo[2.2.1]heptyl) group, a bicyclo[1.1.1]pentyl group, a bicyclo[2.1.1]hexyl group, or a bicyclo[2.2.2]octyl group. The term “C₃-C₁₀ cycloalkylene group,” as used herein, refers to a divalent group having substantially the same structure as the C₃-C₁₀ cycloalkyl group.

The term “C₁-C₁₀ heterocycloalkyl group,” as used herein, refers to a monovalent cyclic group including at least one heteroatom other than carbon atoms as a ring-forming atom and having 1 to 10 carbon atoms. Examples thereof include a 1,2,3,4-oxatriazolidinyl group, a tetrahydrofuranyl group, and a tetrahydrothiophenyl group. The term “C₁-C₁₀ heterocycloalkylene group” as used herein refers to a divalent group having substantially the same structure as the C₁-C₁₀ heterocycloalkyl group.

The term “C₃-C₁₀ cycloalkenyl group” as used herein refers to a monovalent cyclic group that has 3 to 10 carbon atoms and at least one carbon-carbon double bond in its ring, and is not aromatic. Examples thereof include a cyclopentenyl group, a cyclohexenyl group, and a cycloheptenyl group. The term “C₃-C₁₀ cycloalkenylene group,” as used herein, refers to a divalent group having substantially the same structure as the C₃-C₁₀ cycloalkenyl group.

The term “C₁-C₁₀ heterocycloalkenyl group,” as used herein, refers to a monovalent cyclic group including at least one heteroatom other than carbon atoms as a ring-forming atom, 1 to 10 carbon atoms, and at least one double bond in its ring. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkylene group,” as used herein, refers to a divalent group having substantially the same structure as the C₁-C₁₀ heterocycloalkyl group.

The term “C₆-C₆₀ aryl group,” as used herein, refers to a monovalent group having a carbocyclic aromatic system having 6 to 60 carbon atoms. The term “C₆-C₆₀ arylene group,” as used herein, refers to a divalent group having a carbocyclic aromatic system having 6 to 60 carbon atoms. Examples of the C₆-C₆₀ aryl group include a fluorenyl group, a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each independently include two or more rings, the respective rings may be fused (e.g., combined together).

The term “C₁-C₆₀ heteroaryl group,” as used herein, refers to a monovalent group having a heterocyclic aromatic system further including at least one heteroatom other than carbon atoms as a ring-forming atom and 1 to 60 carbon atoms. The term “C₁-C₆₀ heteroarylene group,” as used herein, refers to a divalent group having a heterocyclic aromatic system further including at least one heteroatom other than carbon atoms as a ring-forming atom and 1 to 60 carbon atoms. Examples of the C₁-C₆₀ heteroaryl group include a carbazolyl group, a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each independently include two or more rings, the respective rings may be fused (e.g., combined together).

The term “monovalent non-aromatic condensed polycyclic group,” as used herein, refers to a monovalent group that has two or more rings condensed (e.g., combined together) and only carbon atoms as ring forming atoms (e.g., 8 to 60 carbon atoms), wherein the entire molecular structure is non-aromatic (e.g., is not aromatic when considered as a whole). Examples of the monovalent non-aromatic condensed polycyclic group include an indenyl group, a fluorenyl group, a spiro-bifluorenyl group, a benzofluorenyl group, an indenophenanthrenyl group, and an indenoanthracenyl group. The term “divalent non-aromatic condensed polycyclic group,” as used herein, refers to a divalent group having substantially the same structure as the monovalent non-aromatic condensed polycyclic group.

The term “monovalent non-aromatic condensed heteropolycyclic group,” as used herein, refers to a monovalent group that has two or more condensed rings and at least one heteroatom other than carbon atoms (e.g., 1 to 60 carbon atoms), as a ring-forming atom, wherein the entire molecular structure is non-aromatic (e.g., is not aromatic when considered as a whole). Examples of the monovalent non-aromatic condensed heteropolycyclic group include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzooxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group,” as used herein, refers to a divalent group having substantially the same structure as the monovalent non-aromatic condensed heteropolycyclic group.

The term “C₆-C₆₀ aryloxy group,” as used herein, is represented by —OA₁₀₂ (wherein A₁₀₂ is the C₆-C₆₀ aryl group). The term “C₆-C₆₀ arylthio group,” as used herein, is represented by —SA₁₀₃ (wherein A₁₀₃ is the C₆-C₆₀ aryl group).

The term “R₁₀ₐ,” as used herein, may be:

deuterium (-D), —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ may each independently be hydrogen; deuterium; —F; —Cl; —Br; —I; a hydroxyl group; a cyano group; a nitro group; a C₁-C₆ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom,” as used herein, refers to any atom other than a carbon atom. Examples of the heteroatom may include O, S, N, P, Si, B, Ge, Se, or any combination thereof.

The term “Ph,” as used herein, refers to a phenyl group. The term “Me,” as used herein, refers to a methyl group. The term “Et,” as used herein, refers to an ethyl group. The term “ter-Bu” or “Bu^{t},” as used herein, refers to a tert-butyl group. The term “OMe,” as used herein, refers to a methoxy group.

The term “biphenyl group,” as used herein, refers to a phenyl group substituted with a phenyl group. The “biphenyl group” may be a substituted phenyl group having a C₆-C₆₀ aryl group as a substituent.

The term “terphenyl group,” as used herein, refers to a phenyl group substituted with a biphenyl group. The “terphenyl group” may be a substituted phenyl group having a C₆-C₆₀ aryl group substituted with a C₆-C₆₀ aryl group as a substituent.

The symbols * and *′, as used herein, unless defined otherwise, refer to a binding site to an adjacent atom in a corresponding formula.

Hereinafter, compounds and a light-emitting device according to one or more embodiments will be described in more detail with reference to Synthesis Examples and Examples. The wording “B was used instead of A” used in describing Synthesis Examples refers to that an identical number of molar equivalents of B was used in place of A.

### SYNTHESIS EXAMPLES

### Synthesis Example 1: Synthesis of Compound BD3

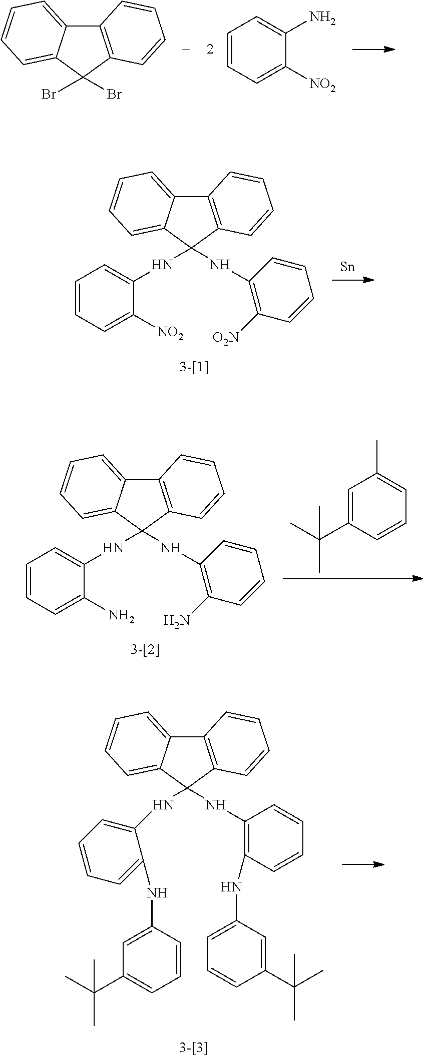

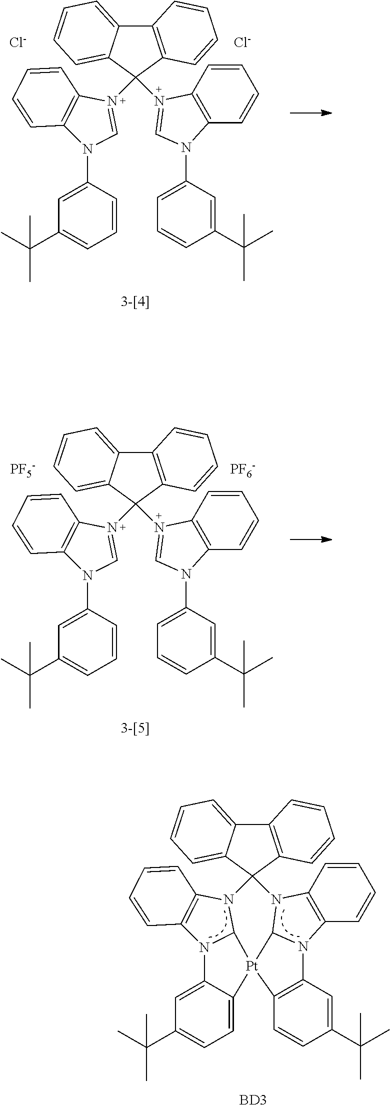

### (1) Synthesis of Intermediate Compound 3-[1]

9,9-dibromo-9H-fluorene (1.0 eq), 2-nitroaniline (2.5 eq), CuI (0.01 eq), K₂CO₃ (2.0 eq), and L-proline (0.02 eq) were dissolved in 0.1 molar (M) dimethyl sulfonate, and the resultant mixture was stirred at a temperature of 160° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 3-[1] was synthesized (yield: 67%).

### (2) Synthesis of Intermediate Compound 3-[2]

Intermediate Compound 3-[1] (1 eq), Sn (2.5 eq), and HCl (2.0 eq) were dissolved in EtOH, followed by stirring at a temperature of 80° C. for 12 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 3-[2] was synthesized (yield: 90%).

### (3) Synthesis of Intermediate Compound 3-[3]

Intermediate Compound 3-[2] (1.0 eq), 1-(tert-butyl)-3-iodobenzene (2.5 eq), CuI (0.01 eq), K₂CO₃ (2.0 eq), and L-proline (0.02 eq) were dissolved in 0.1 M dimethyl sulfonate, and the resultant mixture was stirred at a temperature of 160° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 3-[3] was synthesized (yield: 50%).

### (4) Synthesis of Intermediate Compound 3-[4]

Intermediate Compound 3-[3] (1.0 eq), triethylorthoformate (50 eq), and HCl (25 eq) were dissolved, followed by stirring at a temperature of 80° C. for 12 hours. The resultant reaction mixture was cooled to room temperature, triethylorthoformate was removed therefrom, and then an extraction process was performed thereon three times using ethyl acetate and water to thereby obtain an organic layer. The obtained organic layer was dried using magnesium sulfate, and then concentrated. By using column chromatography, Intermediate Compound 3-[4] was synthesized (yield: 90%).

### (5) Synthesis of Intermediate Compound 3-[5]

Intermediate Compound 3-[4] was dissolved in methanol (0.1 M), and distilled water (0.025 M) was slowly added thereto, followed by addition of NH₄PF₆ (1.2 eq) and stirring at room temperature for 12 hours. The formed solid was filtered, and the resulting product was washed three times using diethyl ether, followed by drying, thereby obtaining Intermediate Compound 3-[5] (yield: 95%).

### (6) Synthesis of Compound BD3

Intermediate Compound 3-[5] (1.0 eq), dichloro(1,2-dicyclooctadiene)platinum (Pt(COD)Cl₂) (1.1 eq), and sodium acetate (2.0 eq) were dissolved in dioxane (0.1 M), followed by stirring at a temperature of 120° C. for 72 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Compound BD3 was obtained (yield: 31%).

### Synthesis Example 2: Synthesis of Compound BD6

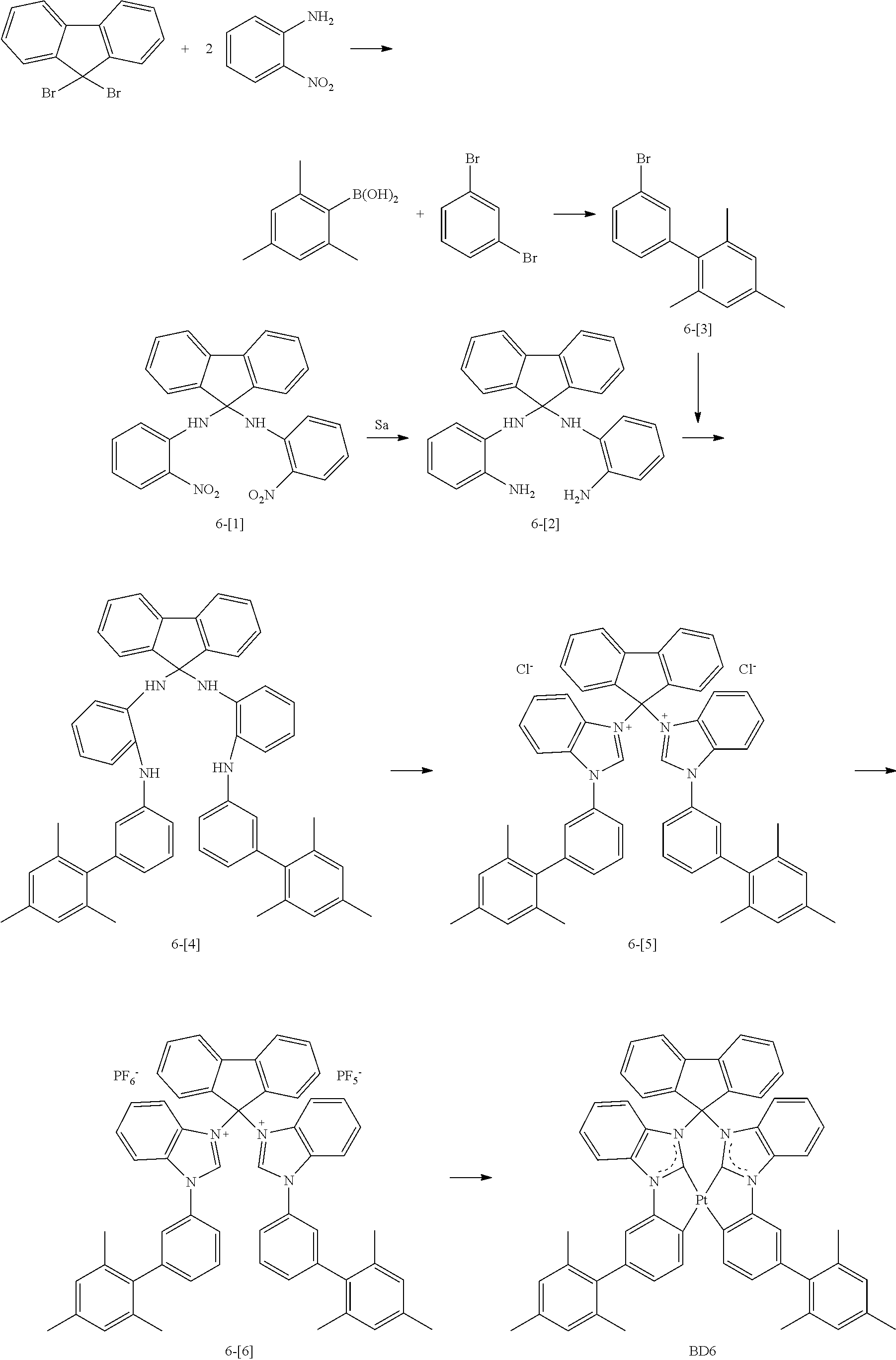

### (1) Synthesis of Intermediate Compound 6-[1]

9,9-dibromo-9H-fluorene (1.0 eq), 2-nitroaniline (2.5 eq), CuI (0.01 eq), K₂CO₃ (2.0 eq), and L-proline (0.02 eq) were dissolved in 0.1 molar (M) dimethyl sulfonate, and the resultant mixture was stirred at a temperature of 160° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 6-[1] was synthesized (yield: 67%).

### (2) Synthesis of Intermediate Compound 6-[2]

Intermediate Compound 6-[1] (1 eq), Sn (2.5 eq), and HCl (2.0 eq) were dissolved in EtOH, followed by stirring at a temperature of 80° C. for 12 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 6-[2] was synthesized (yield: 90%).

### (3) Synthesis of Intermediate Compound 6-[3]

1,3-dibromobenzene (1.2 eq), mesitylboronic acid (1 eq), Ph(PPh₃)₄ (0.02 eq), and K₂CO₃ (2.0 eq) were dissolved in toluene, followed by stirring at a temperature of 120° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 6-[3] was synthesized (yield: 80%).

### (4) Synthesis of Intermediate Compound 6-[4]

Intermediate Compound 6-[2] (1 eq), Intermediate Compound 6-[3] (2.5 eq), CuI (0.01 eq), K₂CO₃ (2.0 eq), and L-proline (0.02 eq) were dissolved in 0.1 M dimethyl sulfonate, and the resultant mixture was stirred at a temperature of 160° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 6-[4] was synthesized (yield: 56%).

### (5) Synthesis of Intermediate Compound 6-[5]

Intermediate Compound 6-[4] (1.0 eq), triethylorthoformate (50 eq), and HCl (25 eq) were dissolved, followed by stirring at a temperature of 80° C. for 12 hours. The resultant reaction mixture was cooled to room temperature, triethylorthoformate was removed therefrom, and then an extraction process was performed thereon three times using ethyl acetate and water to thereby obtain an organic layer. The obtained organic layer was dried using magnesium sulfate, and then concentrated. By using column chromatography, Intermediate Compound 6-[5] was synthesized (yield: 92%).

### (6) Synthesis of Intermediate Compound 6-[6]

Intermediate Compound 6-[5] was dissolved in methanol (0.1 M), and distilled water (0.025 M) was slowly added thereto, followed by addition of NH₄PF₆ (1.2 eq) and stirring at room temperature for 12 hours. The formed solid was filtered, and the resulting product was washed three times using diethyl ether, followed by drying, thereby obtaining Intermediate Compound 6-[6] (yield: 94%).

### (7) Synthesis of Compound BD6

Intermediate Compound 6-[6] (1.0 eq), dichloro(1,2-dicyclooctadiene)platinum (Pt(COD)Cl₂) (1.1 eq), and sodium acetate (2.0 eq) were dissolved in dioxane (0.1 M), followed by stirring at a temperature of 120° C. for 72 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Compound BD6 was obtained (yield: 28%).

### Synthesis Example 3: Synthesis of Compound BD26

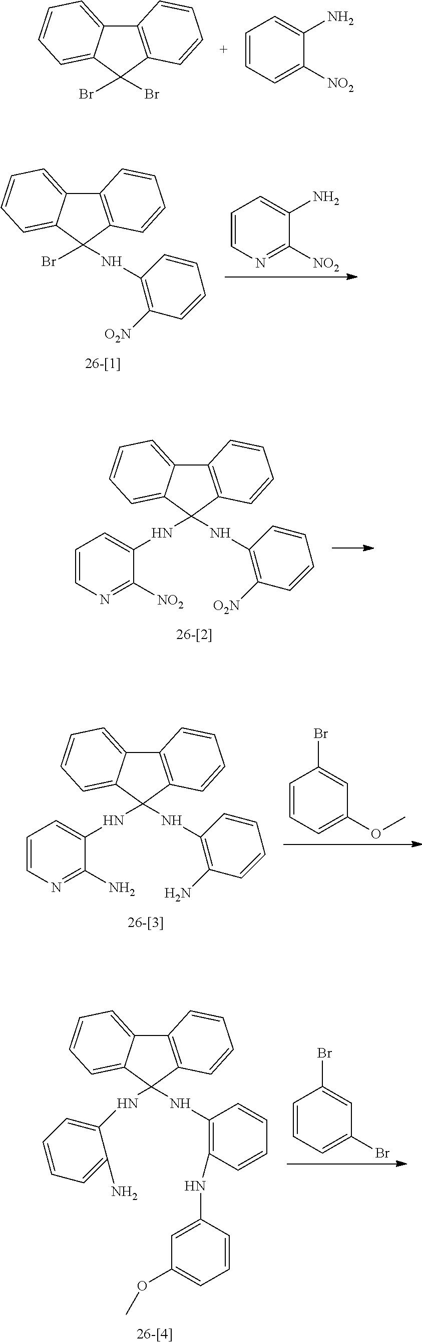

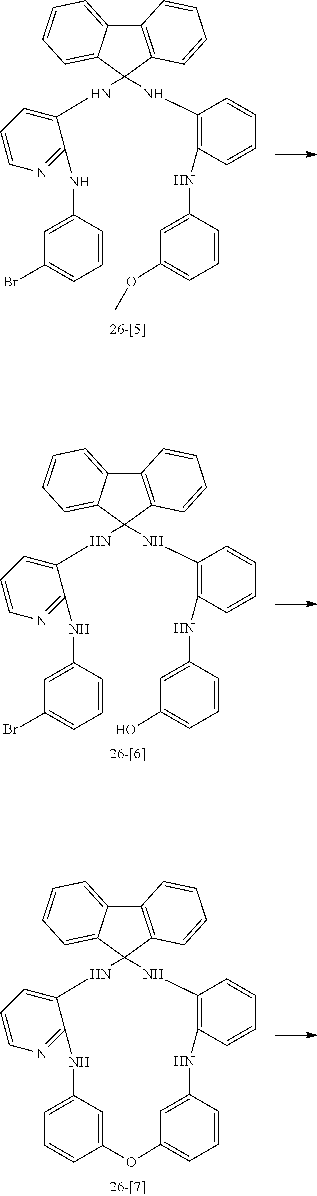 [SMILES: COc1cccc(Nc2ccccc2NC2(Nc3cccnc3Nc3cccc(Br)c3)c3ccccc3-c3ccccc32)c1 ; Oc1cccc(Nc2ccccc2NC2(Nc3cccnc3Nc3cccc(Br)c3)c3ccccc3-c3ccccc32)c1 ; c1cc2cc(c1)Oc1cccc(c1)Nc1ncccc1NC1(Nc3ccccc3N2)c2ccccc2-c2ccccc21]

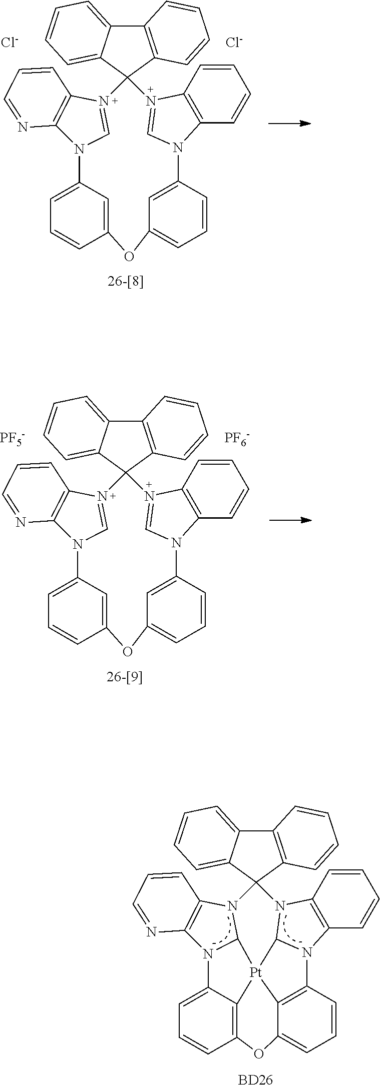

### (1) Synthesis of Intermediate Compound 26-[1]

9,9-dibromo-9H-fluorene (1.0 eq), 2-nitroaniline (1.0 eq), CuI (0.01 eq), K₂CO₃ (2.0 eq), and L-proline (0.02 eq) were dissolved in 0.1 molar (M) dimethyl sulfonate, and the resultant mixture was stirred at a temperature of 160° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 26-[1] was synthesized (yield: 52%).

### (2) Synthesis of Intermediate Compound 26-[2]

Intermediate Compound 26-[1] (1.0 eq), 2-nitropyridin-3-amine (1.0 eq), CuI (0.01 eq), K₂CO₃ (2.0 eq), and L-proline (0.02 eq) were dissolved in 0.1 M dimethyl sulfonate, and the resultant mixture was stirred at a temperature of 160° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 26-[2] was synthesized (yield: 38%).

### (3) Synthesis of Intermediate Compound 26-[3]

Intermediate Compound 26-[2] (1 eq), Sn (2.5 eq), and HCl (2.0 eq) were dissolved in EtOH, followed by stirring at a temperature of 80° C. for 12 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 26-[3] was synthesized (yield: 94%).

### (4) Synthesis of Intermediate Compound 26-[4]

Intermediate Compound 26-[3] (1.0 eq), 1-bromo-3-methoxybenzene (1.0 eq), CuI (0.01 eq), K₂CO₃ (2.0 eq), and L-proline (0.02 eq) were dissolved in 0.1 M dimethyl sulfonate, and the resultant mixture was stirred at a temperature of 160° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 26-[4] was synthesized (yield: 29%).

### (5) Synthesis of Intermediate Compound 26-[5]

Intermediate Compound 26-[4] (1.0 eq), 1,3-dibromobenzene (1.5 eq), CuI (0.01 eq), K₂CO₃ (2.0 eq), and L-proline (0.02 eq) were dissolved in 0.1 M dimethyl sulfonate, and the resultant mixture was stirred at a temperature of 160° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 26-[5] was synthesized (yield: 88%).

### (6) Synthesis of Intermediate Compound 26-[6]

Intermediate Compound 26-[5] (1.0 eq) was dissolved in a mixture of hydrobromic acid aqueous solution and acetic acid (at 3:7, 1 M), followed by stirring at a temperature of 120° C. for 12 hours. Once the resultant reaction mixture was cooled to room temperature, the reaction mixture was neutralized with sodium hydroxide aqueous solution (5 M). The reaction mixture underwent an extraction process three times using dichloromethane and water to thereby obtain an organic layer. The obtained organic layer was dried using magnesium sulfate, and then concentrated. By using column chromatography, Intermediate Compound 26-[6] was synthesized (yield: 77%).

### (7) Synthesis of Intermediate Compound 26-[7]

Intermediate Compound 26-[6] (1.0 eq), CuI (0.01 eq), K₂CO₃ (2.0 eq), and L-proline (0.02 eq) were dissolved in 0.1 M dimethyl sulfonate, and the resultant mixture was stirred at a temperature of 160° C. for 48 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 26-[7] was synthesized (yield: 21%).

### (8) Synthesis of Intermediate Compound 26-[8]

Intermediate Compound 26-[7] (1.0 eq), triethylorthoformate (50 eq), and HCl (25 eq) were dissolved, followed by stirring at a temperature of 80° C. for 12 hours. The resultant reaction mixture was cooled to room temperature, triethylorthoformate was removed therefrom, and then an extraction process was performed thereon three times using ethyl acetate and water to thereby obtain an organic layer. The obtained organic layer was dried using magnesium sulfate, and then concentrated. By using column chromatography, Intermediate Compound 26-[8] was synthesized (yield: 87%).

### (9) Synthesis of Intermediate Compound 26-[9]

Intermediate Compound 26-[8] was dissolved in methanol (0.1 M), and distilled water (0.025 M) was slowly added thereto, followed by addition of NH₄PF₆ (1.2 eq) and stirring at room temperature for 12 hours. The formed solid was filtered, and the resulting product was washed three times using diethyl ether, followed by drying, thereby obtaining Intermediate Compound 26-[9] (yield: 76%).

### (10) Synthesis of Compound BD26

Intermediate Compound 26-[9] (1.0 eq), dichloro(1,2-dicyclooctadiene)platinum (Pt(COD)Cl₂) (1.1 eq), and sodium acetate (2.0 eq) were dissolved in dioxane (0.1 M), followed by stirring at a temperature of 120° C. for 72 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Compound BD26 was synthesized (yield: 35%).

### Synthesis Example 4: Synthesis of Compound BD43

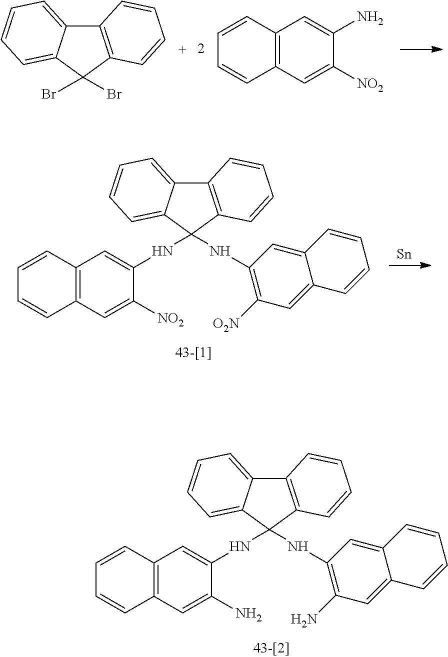

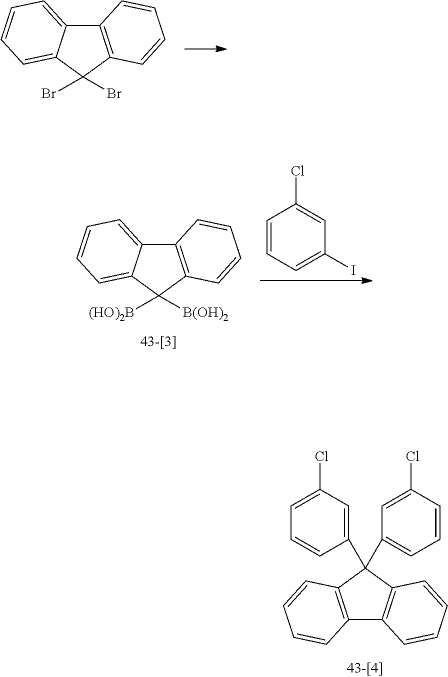

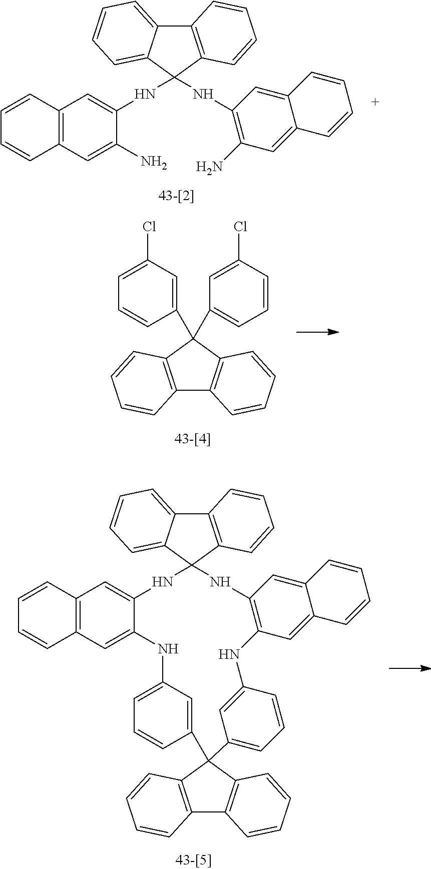 [SMILES: Nc1cc2ccccc2cc1NC1(Nc2cc3ccccc3cc2N)c2ccccc2-c2ccccc21 ; Clc1cccc(C2(c3cccc(Cl)c3)c3ccccc3-c3ccccc32)c1 ; c1cc2cc(c1)C1(c3cccc(c3)Nc3cc4ccccc4cc3NC3(Nc4cc5ccccc5cc4N2)c2ccccc2-c2ccccc23)c2ccccc2-c2ccccc21]

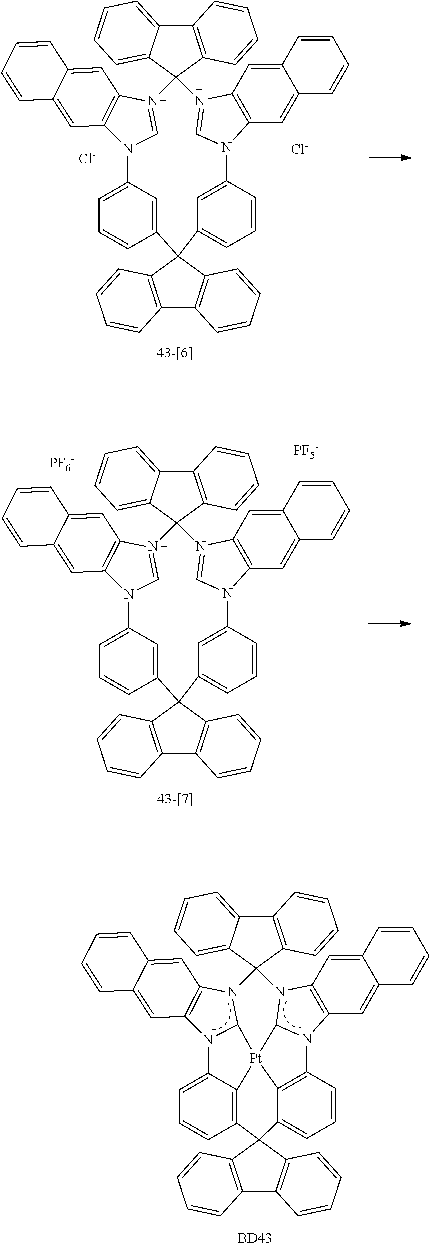

### (1) Synthesis of Intermediate Compound 43-[1]

9,9-dibromo-9H-fluorene (1.0 eq), 3-nitronaphthalen-2-amine (2.5 eq), CuI (0.01 eq), K₂CO₃ (2.0 eq), and L-proline (0.02 eq) were dissolved in 0.1 molar (M) dimethyl sulfonate, and the resultant mixture was stirred at a temperature of 160° C. for 36 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 43-[1] was synthesized (yield: 57%).

### (2) Synthesis of Intermediate Compound 43-[2]

Intermediate Compound 43-[1] (1 eq), Sn (2.5 eq), and HCl (2.0 eq) were dissolved in EtOH, followed by stirring at a temperature of 80° C. for 12 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 43-[2] was synthesized (yield: 98%).

### (3) Synthesis of Intermediate Compound 43-[3]

9,9-dibromo-9H-fluorene (1.0 eq) was dissolved in tetrahydrofuran (THF, 0.1 M), and n-butyl lithium (2.5 M, 2.5 eq) was added thereto at a temperature of −78° C., followed by stirring for 1 hour. Subsequently, trimethylborate (2.5 eq) was added dropwise thereto, followed by stirring at room temperature for 12 hours. 2 M HCl (2.5 eq) was added to the resultant reaction mixture, followed by stirring for 1 hour. Then, an extraction process was performed three times by using dichloromethane and water to thereby obtain an organic layer. The obtained organic layer was dried using magnesium sulfate, and then recrystallized by using hexane. Thus, Intermediate Compound 43-[3] was synthesized (yield: 59%).

### (4) Synthesis of Intermediate Compound 43-[4]

Intermediate Compound 43-[3] (1 eq), 1-chloro-3-iodobenzene (3 eq), Ph(PPh₃)₄ (0.02 eq), and K₂CO₃ (2.0 eq) were dissolved in toluene, followed by stirring at a temperature of 120° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 43-[4] was synthesized (yield: 61%).

### (5) Synthesis of Intermediate Compound 43-[5]

Intermediate Compound 43-[2] (1.0 eq), Intermediate Compound 43-[4] (1.0 eq), CuI (0.01 eq), K₂CO₃ (2.0 eq), and L-proline (0.02 eq) were dissolved in 0.1 M dimethyl sulfonate, and the resultant mixture was stirred at a temperature of 160° C. for 48 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 43-[5] was synthesized (yield: 32%).

### (6) Synthesis of Intermediate Compound 43-[6]

Intermediate Compound 43-[5] (1.0 eq), triethylorthoformate (50 eq), and HCl (25 eq) were dissolved, followed by stirring at a temperature of 80° C. for 12 hours. The resultant reaction mixture was cooled to room temperature, triethylorthoformate was removed therefrom, and then an extraction process was performed thereon three times using ethyl acetate and water to thereby obtain an organic layer. The obtained organic layer was dried using magnesium sulfate, and then concentrated. By using column chromatography, Intermediate Compound 43-[6] was synthesized (yield: 89%).

### (7) Synthesis of Intermediate Compound 43-[7]

Intermediate Compound 43-[6] was dissolved in methanol (0.1 M), and distilled water (0.025 M) was slowly added thereto, followed by addition of NH₄PF₆ (1.2 eq) and stirring at room temperature for 12 hours. The formed solid was filtered, and the resulting product was washed three times using diethyl ether, followed by drying, thereby obtaining Intermediate Compound 43-[7] (yield: 91%).

### (8) Synthesis of Compound BD43

Intermediate Compound 43-[7] (1.0 eq), dichloro(1,2-dicyclooctadiene)platinum (Pt(COD)Cl₂) (1.1 eq), and sodium acetate (2.0 eq) were dissolved in dioxane (0.1 M), followed by stirring at a temperature of 120° C. for 72 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Compound BD43 was synthesized (yield: 28%).

### Synthesis Example 5: Synthesis of Compound BD49

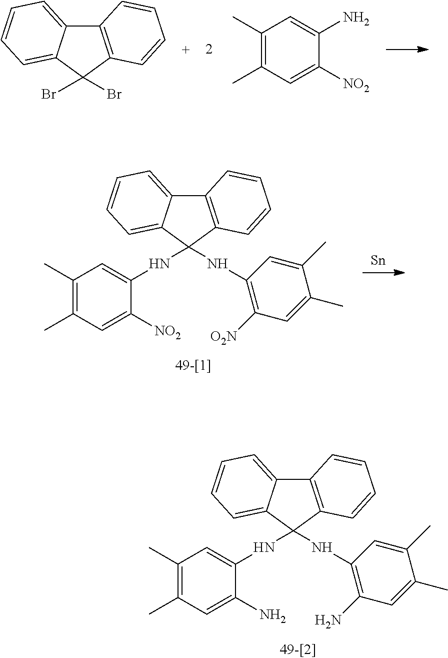

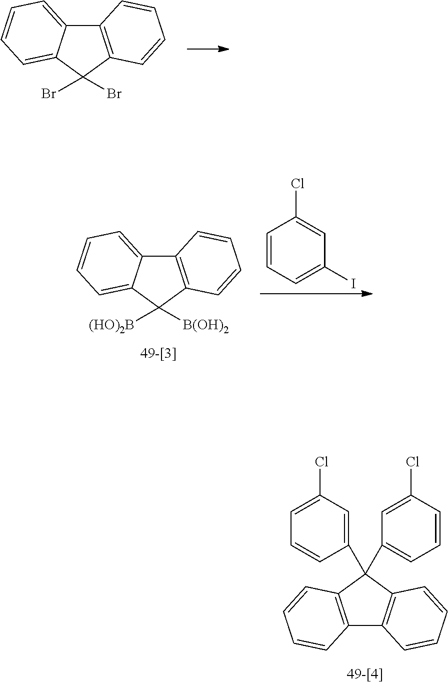

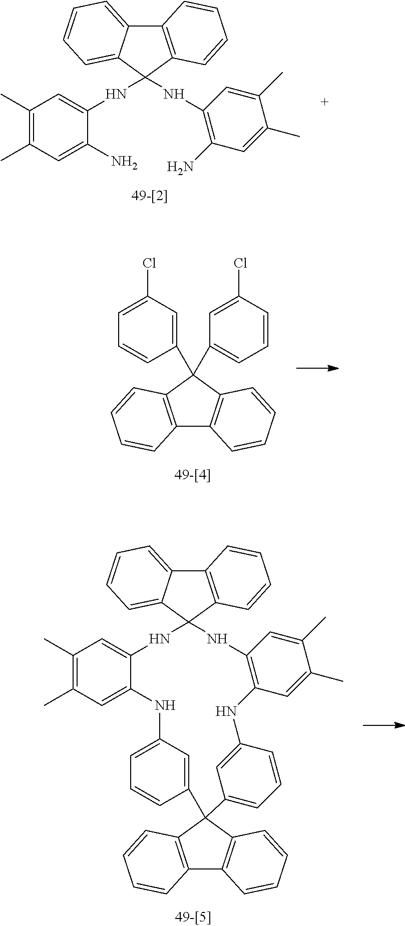

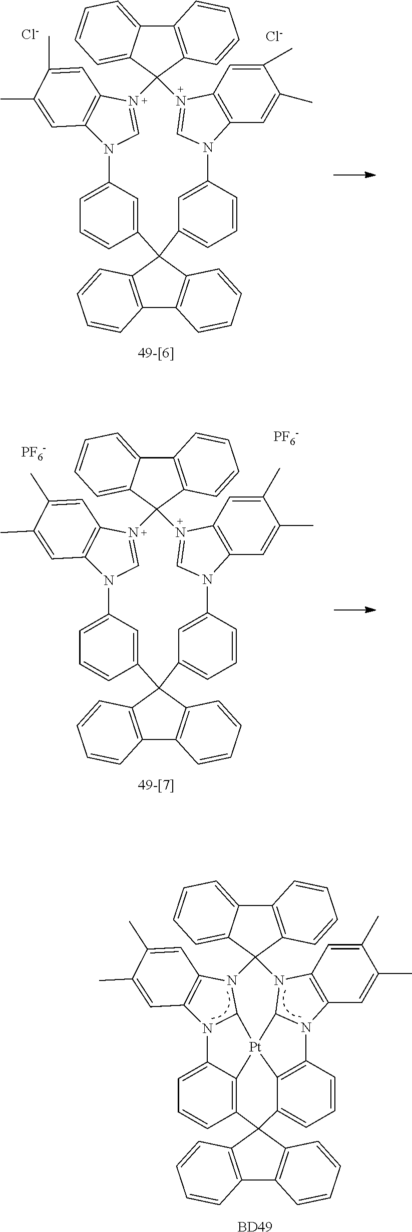

### (1) Synthesis of Intermediate Compound 49-[1]

9,9-dibromo-9H-fluorene (1.0 eq), 4,5-dimethyl-2-nitroaniline (2.5 eq), CuI (0.01 eq), K₂CO₃ (2.0 eq), and L-proline (0.02 eq) were dissolved in 0.1 M dimethyl sulfonate, and the resultant mixture was stirred at a temperature of 160° C. for 36 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 49-[1] was synthesized (yield: 64%).

### (2) Synthesis of Intermediate Compound 49-[2]

Intermediate Compound 49-[1] (1 eq), Sn (2.5 eq), and HCl (2.0 eq) were dissolved in EtOH, followed by stirring at a temperature of 80° C. for 12 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 49-[2] was synthesized (yield: 91%).

### (3) Synthesis of Intermediate Compound 49-[3]

9,9-dibromo-9H-fluorene (1.0 eq) was dissolved in tetrahydrofuran (THF, 0.1 M), and n-butyl lithium (2.5 M, 2.5 eq) was added thereto at a temperature of −78° C., followed by stirring for 1 hour. Subsequently, trimethylborate (2.5 eq) was added dropwise thereto, followed by stirring at room temperature for 12 hours. 2 M HCl (2.5 eq) was added to the resultant reaction mixture, followed by stirring for 1 hour. Then, an extraction process was performed three times by using dichloromethane and water to thereby obtain an organic layer. The obtained organic layer was dried using magnesium sulfate, and then recrystallized by using hexane. Thus, Intermediate Compound 49-[3] was synthesized (yield: 50%).

### (4) Synthesis of Intermediate Compound 49-[4]

Intermediate Compound 49-[3] (1 eq), 1-chloro-3-iodobenzene (3 eq), Ph(PPh₃)₄ (0.02 eq), and K₂CO₃ (2.0 eq) were dissolved in toluene, followed by stirring at a temperature of 120° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 49-[4] was synthesized (yield: 67%).

### (5) Synthesis of Intermediate Compound 49-[5]

Intermediate Compound 49-[2] (1.0 eq), Intermediate Compound 49-[4] (1.0 eq), CuI (0.01 eq), K₂CO₃ (2.0 eq), and L-proline (0.02 eq) were dissolved in 0.1 M dimethyl sulfonate, and the resultant mixture was stirred at a temperature of 160° C. for 48 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 49-[5] was synthesized (yield: 37%).

### (6) Synthesis of Intermediate Compound 49-[6]

Intermediate Compound 49-[5] (1.0 eq), triethylorthoformate (50 eq), and HCl (25 eq) were dissolved, followed by stirring at a temperature of 80° C. for 12 hours. The resultant reaction mixture was cooled to room temperature, triethylorthoformate was removed therefrom, and then an extraction process was performed thereon three times using ethyl acetate and water to thereby obtain an organic layer. The obtained organic layer was dried using magnesium sulfate, and then concentrated. By using column chromatography, Intermediate Compound 49-[6] was synthesized (yield: 80%).

### (7) Synthesis of Intermediate Compound 49-[7]

Intermediate Compound 49-[6] was dissolved in methanol (0.1 M), and distilled water (0.025 M) was slowly added thereto, followed by addition of NH₄PF₆ (1.2 eq) and stirring at room temperature for 12 hours. The formed solid was filtered, and the resulting product was washed three times using diethyl ether, followed by drying, thereby obtaining Intermediate Compound 49-[7] (yield: 90%).

### (8) Synthesis of Compound BD49

Intermediate Compound 49-[7] (1.0 eq), dichloro(1,2-dicyclooctadiene)platinum (Pt(COD)Cl₂) (1.1 eq), and sodium acetate (2.0 eq) were dissolved in dioxane (0.1 M), followed by stirring at a temperature of 120° C. for 72 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Compound BD49 was synthesized (yield: 18%).

### Synthesis Example 6: Synthesis of Compound BD33

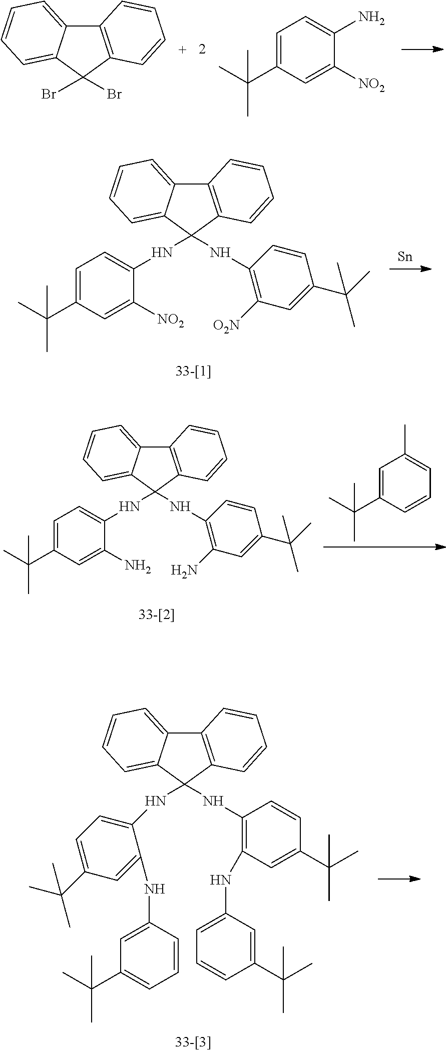

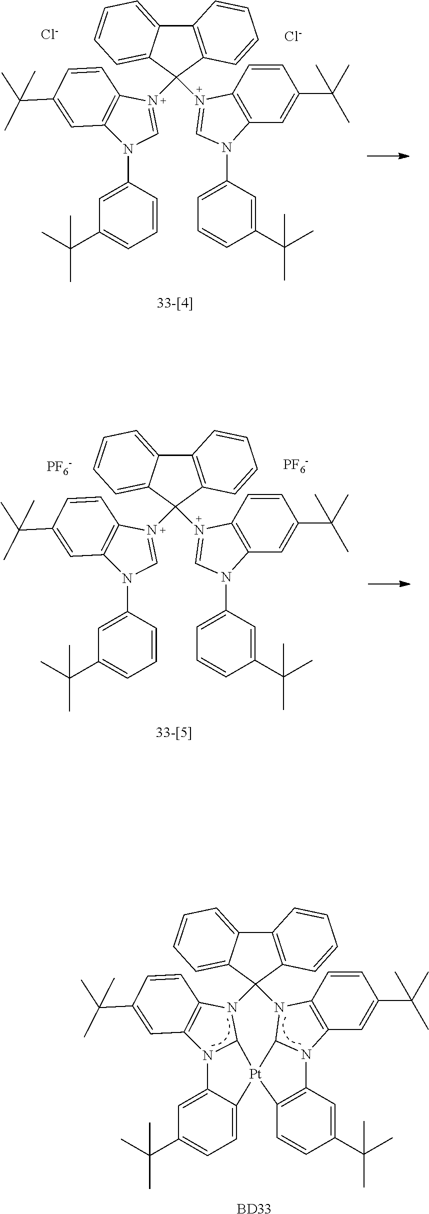

### (1) Synthesis of Intermediate Compound 33-[1]

9,9-dibromo-9H-fluorene (1.0 eq), 4-(tert-butyl)-2-nitroaniline (2.5 eq), CuI (0.01 eq), K₂CO₃ (2.0 eq), and L-proline (0.02 eq) were dissolved in 0.1 molar (M) dimethyl sulfonate, and the resultant mixture was stirred at a temperature of 160° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 33-[1] was synthesized (yield: 68%).

### (2) Synthesis of Intermediate Compound 33-[2]

Intermediate Compound 33-[1] (1 eq), Sn (2.5 eq), and HCl (2.0 eq) were dissolved in EtOH, followed by stirring at a temperature of 80° C. for 12 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 33-[2] was synthesized (yield: 92%).

### (3) Synthesis of Intermediate Compound 33-[3]

Intermediate Compound 33-[2] (1.0 eq), 1-(tert-butyl)-3-iodobenzene (2.5 eq), CuI (0.01 eq), K₂CO₃ (2.0 eq), and L-proline (0.02 eq) were dissolved in 0.1 M dimethyl sulfonate, and the resultant mixture was stirred at a temperature of 160° C. for 24 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Intermediate Compound 33-[3] was synthesized (yield: 54%).

### (4) Synthesis of Intermediate Compound 33-[4]

Intermediate Compound 33-[2] (1.0 eq), triethylorthoformate (50 eq), and HCl (25 eq) were dissolved, followed by stirring at a temperature of 80° C. for 12 hours. The resultant reaction mixture was cooled to room temperature, triethylorthoformate was removed therefrom, and then an extraction process was performed thereon three times using ethyl acetate and water to thereby obtain an organic layer. The obtained organic layer was dried using magnesium sulfate, and then concentrated. By using column chromatography, Intermediate Compound 33-[4] was synthesized (yield: 91%).

### (5) Synthesis of Intermediate Compound 33-[5]

Intermediate Compound 33-[4] was dissolved in methanol (0.1 M), and distilled water (0.025 M) was slowly added thereto, followed by addition of NH₄PF₆ (1.2 eq) and stirring at room temperature for 12 hours. The formed solid was filtered, and the resulting product was washed three times using diethyl ether, followed by drying, thereby obtaining Intermediate Compound 33-[5] (yield: 97%).

### (6) Synthesis of Compound BD33

Intermediate Compound 33-[5] (1.0 eq), dichloro(1,2-dicyclooctadiene)platinum (Pt(COD)Cl₂) (1.1 eq), and sodium acetate (2.0 eq) were dissolved in dioxane (0.1 M), followed by stirring at a temperature of 120° C. for 72 hours. The resultant reaction mixture was cooled to room temperature, and then an extraction process was performed thereon three times using dichloromethane and water to thereby obtain an organic layer. The obtained organic layer was dried using magnesium sulfate, and then concentrated. By using column chromatography, Compound BD33 was synthesized (yield: 33%).

Compounds synthesized in the Synthesis Examples were identified by ¹H nuclear magnetic resonance (NMR) and mass spectroscopy/fast atom bombardment (MS/FAB). The results thereof are shown in Table 1.

TABLE 1

| Compound | | MS/FAB | |
|---|---|---|---|
| No. | ¹H NMR (CDCl₃, 400 MHz) | found | calc. |
| BD3 | δ = 7.9 (d, 2H), 7.55 (d, 2H), 7.45 (d, | 855.26 | 855.95 |
| | 2H), 7.45-7.38 (m, 6H), 7.28 (t, 2H), | | |
| | 7.21 (d, 2H), 7.08 (d, 2H), 6.97 (d, 2H), | | |
| | 6.71 (d, 2H), 1, 25 (s, 18H) | | |
| BD6 | δ = 7.92 (d, 2H), 7.53 (d, 2H), 7.41 (d, | 979.32 | 980.09 |
| | 2H), 7.45-7.35 (m, 6H), 7.30 (t, 2H), | | |
| | 7.23 (d, 2H), 7.00 (d, 2H), 6.90(d, 2H), | | |
| | 6.72 (d, 2H), 2.92 (s, 12H), 1, 23 (s, 6H) | | |
| BD26 | δ = 7.90 (d, 2H), 7.68 (d, 1H), 7.57 (d, | 758.14 | 758.70 |
| | 2H), 7.43 (d, 2H), 7.28-7.20 (m, 4H), | | |
| | 7.17 (t, 2H), 7.08 (d, 2H), 6.90 (d, 2H), | | |
| | 6.79(d, 2H), 6.66 (d, 2H) | | |
| BD43 | δ = 7.95 (d, 2H), 7.75 (d, 2H), 7.67 (d, | 1005.24 | 1006.04 |
| | 2H), 7.60 (d, 2H), 7.50-7.41 (m, 2H) | | |
| | 7.28-7.20 (m, 4H), 7.11 (t, 2H), 7.09 (d, | | |
| | 2H), 6.87 (d, 2H), 6.77(d, 2H), 6.60 (d, | | |
| | 2H) | | |
| BD49 | δ = 7.95 (d, 2H), 7.75 (d, 2H), 7.67 (d, | 961.27 | 962.03 |
| | 2H), 7.60 (d, 2H), 7.50-7.41 (m, 2H), | | |
| | 7.11 (t, 2H), 7.09 (d, 2H), 6.87 (d, 2H), | | |
| | 6.77(d, 2H), 6.60 (d, 2H), 2.47 (s, 6H), | | |
| | 2.38 (s, 6H) | | |
| BD33 | δ = 7.92 (d, 2H), 7.52 (d, 2H), 7.48-7.38 | 967.42 | 968.17 |
| | (m, 6H), 7.21 (t, 2H), 7.19 (d, 2H), 7.04 | | |
| | (d, 2H), 6.88 (d, 2H), 6.71 (d, 2H), 2.77 | | |
| | (s, 18H), 1, 25 (s, 18H) | | |

Methods of synthesizing compounds other than compounds shown in Table 1 may be easily understood to those skilled in the art by referring to the synthesis schemes and raw materials described above.

### EXAMPLES

### Example 1

As an anode, a 15 Ohms per square centimeter (Q/cm²) (1,200 Å) glass substrate (available from Corning Co., Ltd), on which an ITO electrode was formed, was cut to a size of 50 millimeters (mm)×50 mm×0.7 mm, sonicated in isopropyl alcohol and pure water for 5 minutes in each solvent, cleaned with ultraviolet rays for 30 minutes, and then ozone, and was mounted on a vacuum deposition apparatus.

2-TNATA was vacuum-deposited on the ITO electrode (anode) to form a hole injection layer having a thickness of 600 Å, and 4,4′-bis[N-(1-naphthyl)-N-phenylamino]biphenyl (hereinafter, referred as “NPB”) was vacuum-deposited on the hole injection layer to form a hole transport layer having a thickness of 300 Å.

A dopant (Compound BD3) and a host (3,3-di(9H-carbazol-9-yl)biphenyl, mCBP) were co-deposited on the hole transport layer to a weight ratio of 90:10 to form an emission layer having a thickness of 300 Å.

Diphenyl(4-(triphenylsilyl)phenyl)-phosphine oxide (TSPO1) was deposited on the emission layer to form a hole blocking layer having a thickness of 50 Å, Alq₃ was vacuum-deposited on the hole blocking layer to form an electron transport layer having a thickness of 300 Å, LiF was vacuum-deposited on the electron transport layer to form an electron injection layer having a thickness of 10 Å, and Al was vacuum-deposited on the electron injection layer having a thickness of 3,000 Å, thereby completing the manufacture of an organic light-emitting device.

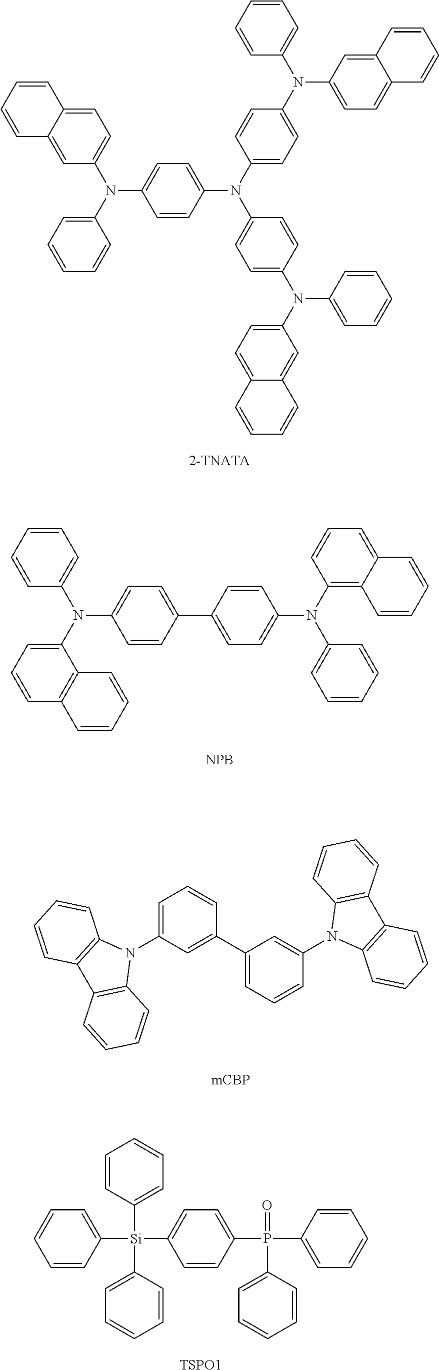

### Examples 2 to 6 and Comparative Examples 1 to 4

Organic light-emitting devices were manufactured in substantially the same manner as in Example 1, except that the compounds listed in Table 2 were respectively used in Examples 2 to 6 and Comparative Examples 1 to 4 instead of Compound 1 as a dopant in the formation of the emission layer.

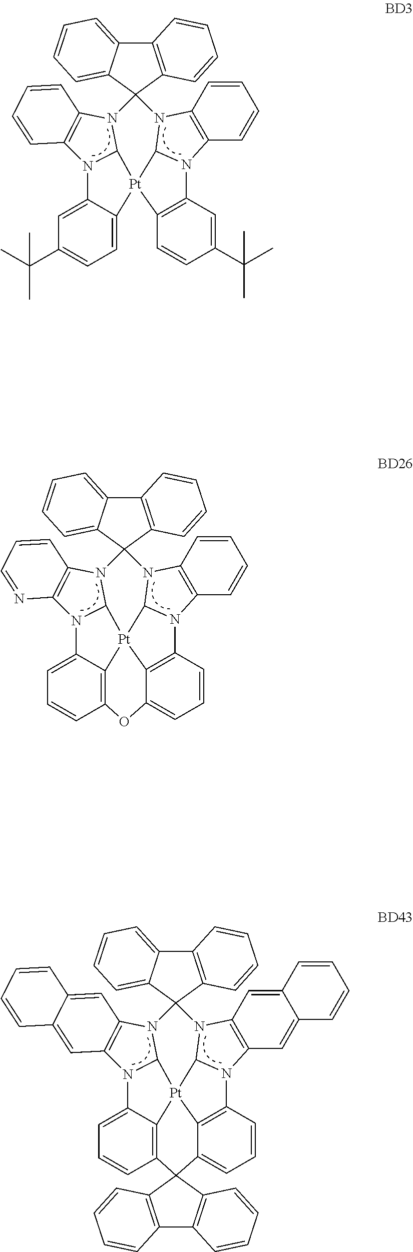

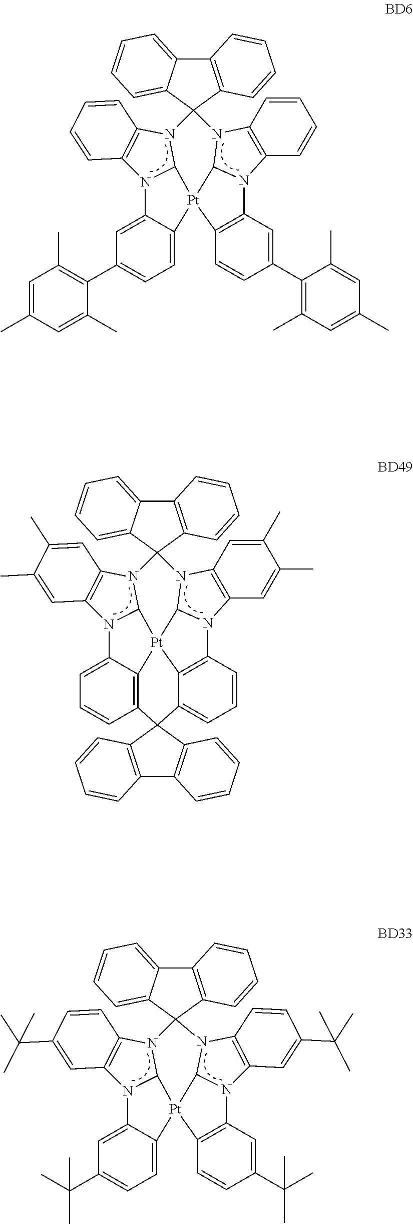

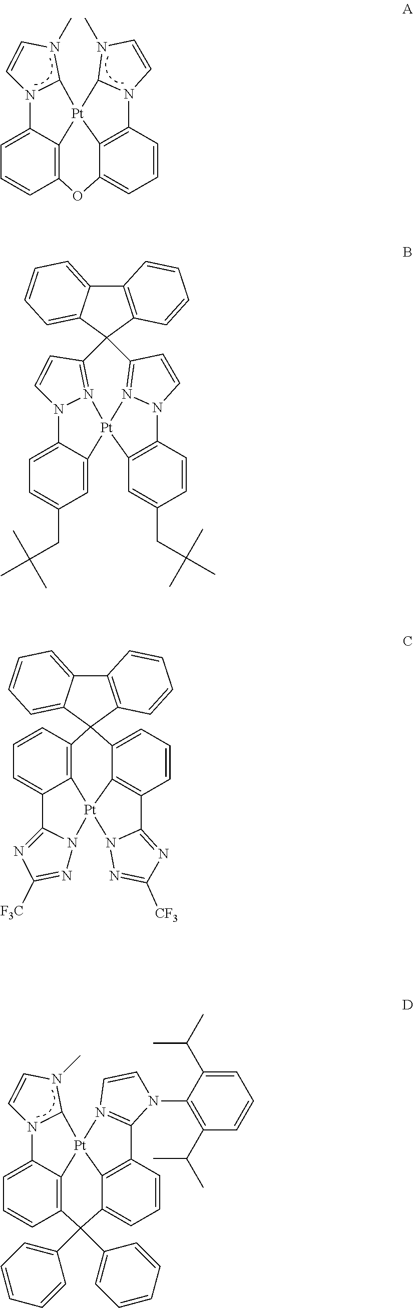

### Evaluation Example 1

The driving voltage, current density, luminance, luminescence efficiency, emission color, and maximum emission wavelength of the organic light-emitting devices manufactured according to Examples 1 to 6 and Comparative Examples 1 to 4 were measured by using a Keithley SMU 236 and a luminance meter PR650. The results thereof are shown in Table 2.

TABLE 2

| | | | | | | | Maximum |
|---|---|---|---|---|---|---|---|
| | | Driving | Current | | | | emission |
| | Emission layer | voltage | density | Luminance | Efficiency | Emission | wavelength |
| | Dopant | (V) | (mA/cm²) | (cd/m²) | (cd/A) | color | (nm) |
| Example 1 | BD3 | 3.3 | 50 | 15 | 100 | Blue | 450 |
| Example 2 | BD6 | 3.4 | 50 | 15 | 95 | Blue | 459 |
| Example 3 | BD26 | 3.2 | 50 | 15 | 96 | Blue | 461 |
| Example 4 | BD43 | 3.3 | 50 | 15 | 45 | Blue | 463 |
| Example 5 | BD49 | 3.5 | 50 | 15 | 35 | Blue | 457 |
| Example 6 | BD33 | 3.3 | 50 | 15 | 110 | Blue | 450 |
| Comparative | Compound A | 4.0 | 50 | 15 | 10 | Green | 482 |
| Example 1 | | | | | | | |
| Comparative | Compound B | 3.9 | 50 | 15 | 25 | Blue | 463 |
| Example 2 | | | | | | | |
| Comparative | Compound C | 3.8 | 50 | 15 | 9.5 | Green | 477 |
| Example 3 | | | | | | | |
| Comparative | Compound D | 3.8 | 50 | 15 | 32 | Blue | 465 |
| Example 4 | | | | | | | |

Referring to the results of Table 2, each of the organic light-emitting devices of Examples 1 to 6 were found to have a low driving voltage and excellent luminescence efficiency and to be suitable for blue light emission, as compared with the organic light-emitting devices of Comparative Examples 1 to 4.

Accordingly, an organic light-emitting device including the organometallic compound may have improved efficiency.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims, and equivalents thereof.

What is claimed is:

1. An organometallic compound represented by Formula 1: 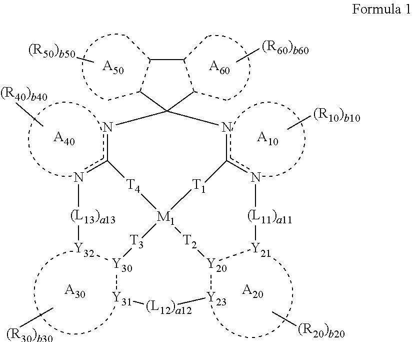
wherein, in Formula 1,
M₁ is platinum (Pt), palladium (Pd), copper (Cu), silver (Ag), gold (Au), rhodium (Rh), iridium (Ir), ruthenium (Ru), osmium (Os), titanium (Ti), zirconium (Zr), hafnium (Hf), europium (Eu), terbium (Tb), or thulium (Tm),
A₁₀ and A₄₀ are each independently a N-containing C₁-C₆₀ heterocyclic group,
A₂₀, A₃₀, A₅₀, and A₆₀ are each independently a C₅-C₆₀ carbocyclic group or a C₁-C₆₀ heterocyclic group,
Y₂₀ and Y₃₀ are each independently N or C,
Y₂₁, Y₂₂, Y₃₁, and Y₃₂ are each independently N or C,
T₁ to T₄ each indicate a chemical bond,
L₁₁ to L₁₃ are each independently a single bond, *—O—*′, *—S—*′, *—C(R₁)(R₂)—*′, *—C(R₁)═*′, *═C(R₁)—*′, *—C(R₁)═C(R₂)—*′, *—C(═O)—*′, *—C(═S)—*′, *—C≡C—*′, *—B(R₁)—*′, *—N(R₁)—*′, *—P(R₁)*′, *—Si(R₁)(R₂)—*′, *—P(═O)(R₁)*′, or *—Ge(R₁)(R₂)—*′,
a11, a12, and a13 are each independently 0, 1, 2, 3, 4, or 5,
R₁, R₂, R₁₀, R₂₀, R₃₀, R₄₀, R₅₀, and R₆₀ are each independently hydrogen, deuterium, —F, —Cl, —Br, —I, a hydroxyl group, a cyano group, a nitro group, an amidino group, a hydrazino group, a hydrazono group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₁₀ cycloalkyl group unsubstituted or substituted with at least one R₁₀ₐ, a C₁-C₁₀ heterocycloalkyl group unsubstituted or substituted with at least one R₁₀ₐ, a C₃-C₁₀ cycloalkenyl group unsubstituted or substituted with at least one R₁₀ₐ, a C₁-C₁₀ heterocycloalkenyl group unsubstituted or substituted with at least one R₁₀ₐ, a C₆-C₆₀ aryl group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₁-C₆₀ heteroaryl group unsubstituted or substituted with at least one R₁₀ₐ, a C₁-C₆₀ heteroaryloxy group unsubstituted or substituted with at least one R₁₀ₐ, a C₁-C₆₀ heteroarylthio group unsubstituted or substituted with at least one R₁₀ₐ, a monovalent non-aromatic condensed polycyclic group unsubstituted or substituted with at least one R₁₀ₐ, a monovalent non-aromatic condensed heteropolycyclic group unsubstituted or substituted with at least one R₁₀ₐ, —Si(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂),
at least two selected from R₁, R₂, R₁₀, R₂₀, R₃₀, R₄₀, R₅₀, and R₆₀ are optionally bound to form a C₅-C₆₀ carbocyclic group unsubstituted or substituted with at least one R₁₀ₐ or a C₁-C₆₀ heterocyclic group unsubstituted or substituted with at least one R₁₀ₐ,
b10, b20, b30, b40, b50, and b60 are each independently 1, 2, 3, 4, 5, 6, 7, or 8,
* and *′ each indicate a binding site to an adjacent atom, and
R₁₀ₐ is:
deuterium (-D), —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with at deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
wherein Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

2. The organometallic compound of claim 1, wherein M₁ is Pt, Pd, Cu, Ag, or Au.

3. The organometallic compound of claim 1, wherein A₁₀ and A₄₀ are each independently an imidazole group, a benzimidazole group, a naphthoimidazole group, a 4,5,6,7-tetrahydrobenzimidazole group, a 2,3-dihydrobenzimidazole group, an imidazopyridine group, a 2,3-dihydroimidazopyridine group, an imidazopyrimidine group, a 2,3-dihydroimidazopyrimidine group, an imidazopyrazine group, or a 2,3-dihydroimidazopyrazine group.

4. The organometallic compound of claim 1, wherein A₂₀, A₃₀, A₅₀, and A₆₀ are each independently a benzene group, a naphthalene group, a fluorene group, a carbazole group, a dibenzofuran group, a dibenzothiophene group, a dibenzosilole group, a pyridine group, a pyrimidine group, a pyrazine group, a triazine group, a quinoline group, an isoquinoline group, a quinoxaline group, or a quinazoline group.

5. The organometallic compound of claim 1, wherein any two selected from T₁ to T₄ are each a coordinate bond, and the other two selected from T₁ to T₄ are each a covalent bond.

6. The organometallic compound of claim 1, wherein L₁₁ to L₁₃ are each independently selected from a single bond, *—O—*′, *—S—*′, *—N(R₁)—*′, *—C(R₁)(R₂)—*′, *—Si(R₁)(R₂)—*′, and *—B(R₁)—*′.

7. The organometallic compound of claim 1, wherein R₁, R₂, R₁₀, R₂₀, R₃₀, R₄₀, R₅₀, and R₆₀ are each independently: hydrogen, deuterium, —F, —Cl, —Br, —I, a cyano group, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, a cyano group, a phenyl group, a biphenyl group, or any combination thereof; or
a group represented by one selected from Formulae 5-1 to 5-26 and Formulae 6-1 to 6-55,
wherein at least two adjacent groups of R₁, R₂, R₁₀, R₂₀, R₃₀, R₄₀, R₅₀, and R₆₀ are optionally bound to form:
a cyclopentane group, a cyclohexane group, a benzene group, a naphthalene group, a fluorene group, or a carbazole group; or
a cyclopentane group, a cyclohexane group, a benzene group, a naphthalene group, a fluorene group, or a carbazole group, each substituted with deuterium, —F, —Cl, —Br, —I, a cyano group, a phenyl group, a biphenyl group, or any combination thereof: 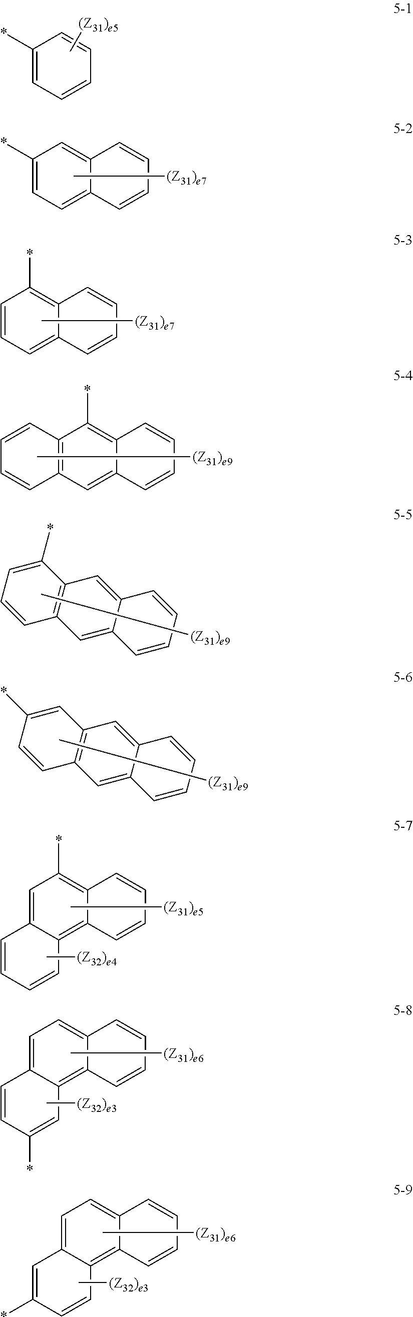 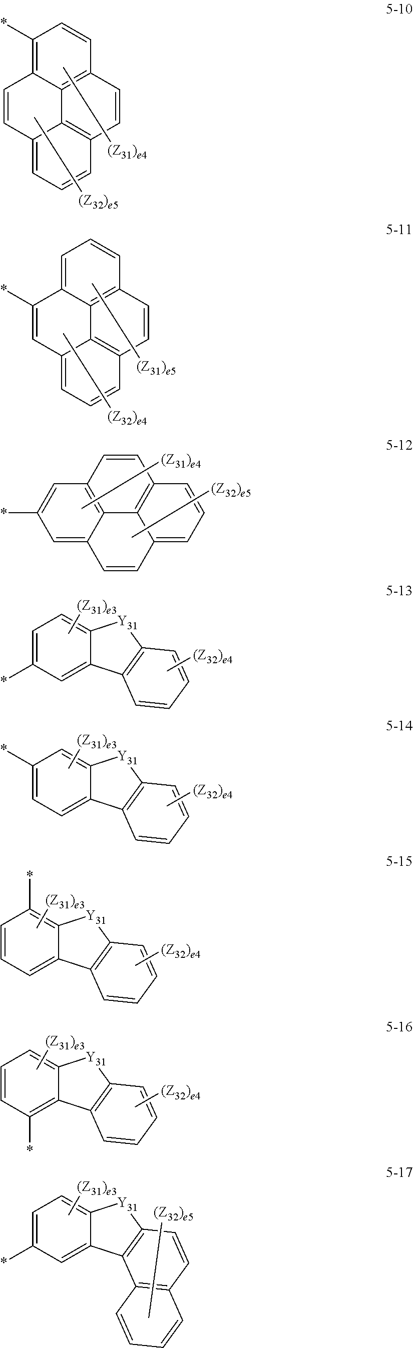 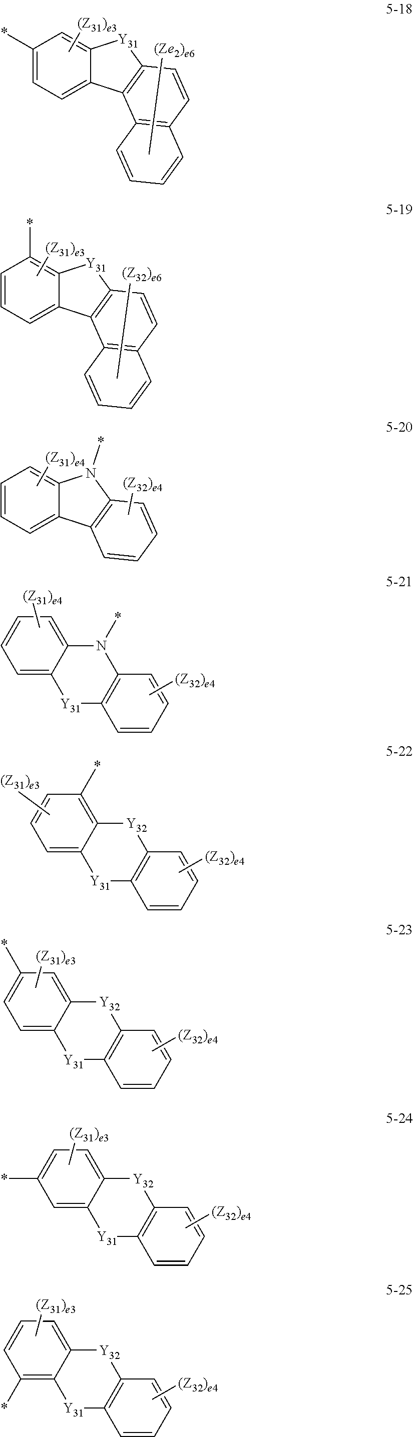 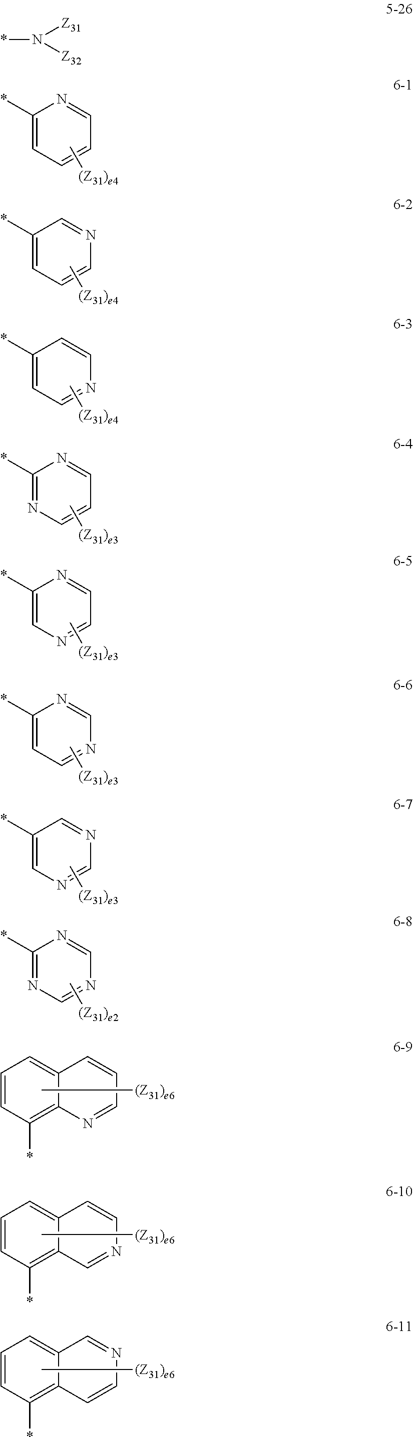 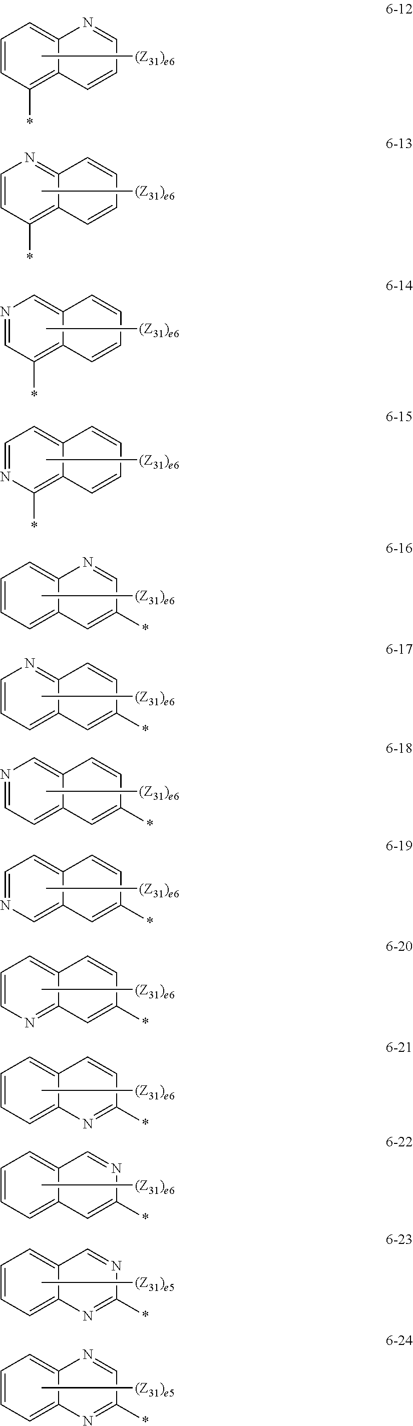 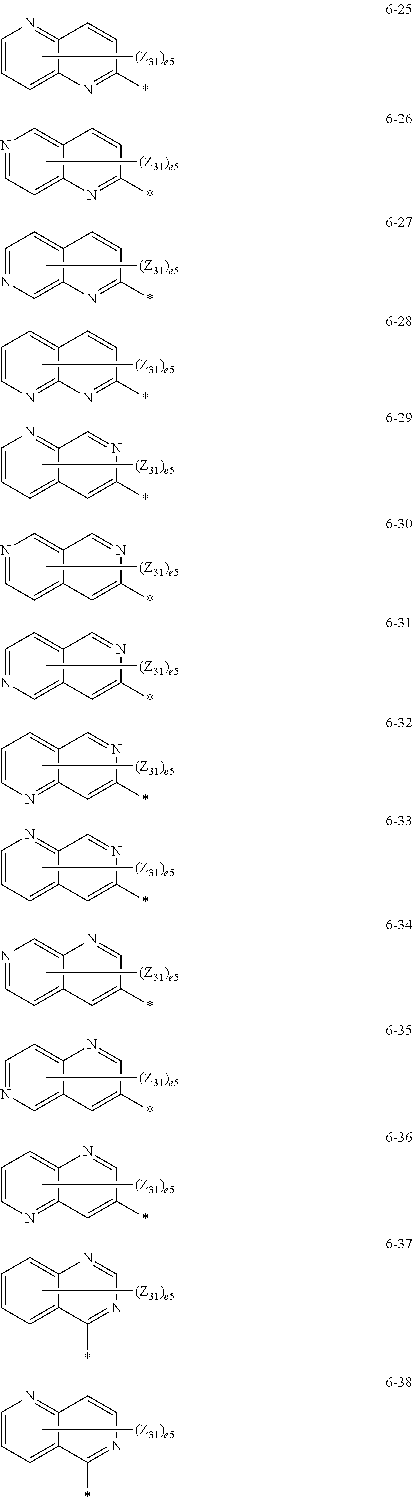 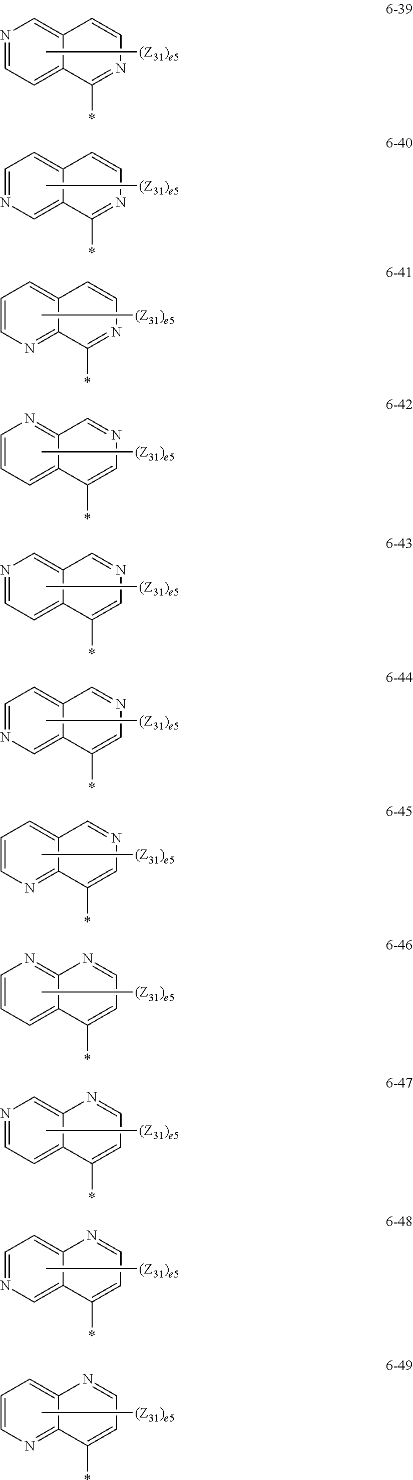 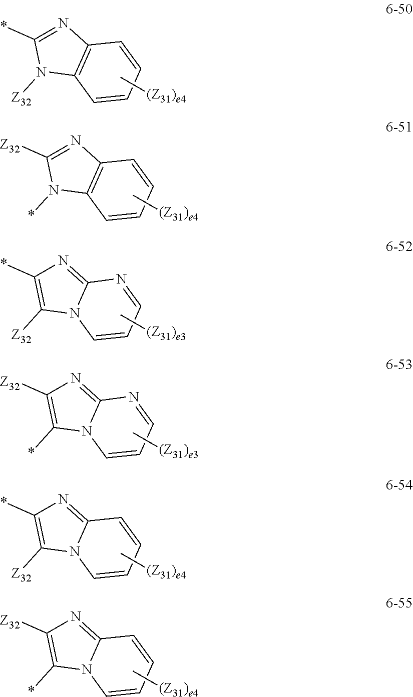
wherein, in Formulae 5-1 to 5-26 and 6-1 to 6-55,
Y₃₁ and Y₃₂ are each independently O, S, C(Z₃₃)(Z₃₄), N(Z₃₃), or Si(Z₃₃)(Z₃₄),
Z₃₁ to Z₃₄ are each independently selected from hydrogen, deuterium, —F, —Cl, —Br, —I, a hydroxyl group, a cyano group, a nitro group, an amidino group, a hydrazino group, a hydrazono group, a C₁-C₂₀ alkyl group, a C₂-C₂₀ alkenyl group, a C₂-C₂₀ alkynyl group, a C₁-C₂₀ alkoxy group, a phenyl group, a biphenyl group, a terphenyl group, a naphthyl group, a fluorenyl group, a spiro-bifluorenyl group, a phenanthrenyl group, an anthracenyl group, a triphenylenyl group, a pyridinyl group, a pyrimidinyl group, a carbazolyl group, and a triazinyl group,
e2 is 1 or 2,
e3 is an integer from 1 to 3,
e4 is an integer from 1 to 4,
e5 is an integer from 1 to 5,
e6 is an integer from 1 to 6,
e7 is an integer from 1 to 7,
e9 is an integer from 1 to 9, and
* indicates a binding site to an adjacent atom.

8. The organometallic compound of claim 1, wherein R₁, R₂, R₁₀, R₂₀, R₃₀, R₄₀, R₅₀, and R₆₀ are each independently: hydrogen, deuterium, —F, —Cl, —Br, —I, a cyano group, a C₁-C₂₀ alkyl group, or a C₁-C₂₀ alkoxy group;
a C₁-C₂₀ alkyl group, a C₁-C₂₀ alkoxy group, or a C₃-C₁₀ cycloalkyl group, each substituted with deuterium, —F, —Cl, —Br, —I, —CDH₂, —CD₂H, —CD₃, a cyano group, a phenyl group, a biphenyl group, or any combination thereof;
a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a carbazolyl group, an acridinyl group, a dibenzofuranyl group, a dibenzothiophenyl group, a benzocarbazolyl group, or a dibenzocarbazolyl group; or
a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a carbazolyl group, an acridinyl group, a dibenzofuranyl group, a dibenzothiophenyl group, a benzocarbazolyl group, or a dibenzocarbazolyl group, each substituted with deuterium, —F, —Cl, —Br, —I, —CDH₂, —CD₂H, —CD₃, a cyano group, a C₁-C₂₀ alkyl group, a C₁-C₂₀ alkoxy group, a C₃-C₁₀ cycloalkyl group, a phenyl group, a biphenyl group, or any combination thereof,
wherein at least two adjacent groups of R₁, R₂, R₁₀, R₂₀, R₃₀, R₄₀, R₅₀, and R₆₀ are optionally bound to form:
a cyclopentane group, a cyclohexane group, a fluorene group, or a carbazole group; or
a cyclopentane group, a cyclohexane group, a fluorene group, or a carbazole group, each substituted with deuterium, —F, —Cl, —Br, —I, a cyano group, a phenyl group, a biphenyl group, or any combination thereof.

9. The organometallic compound of claim 1, wherein the organometallic compound represented by Formula 1 is a compound represented by Formula 11 or Formula 12: 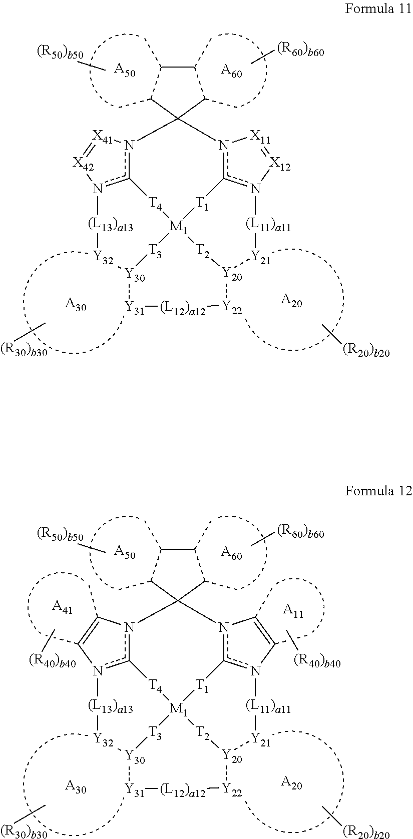
wherein, in Formulae 11 and 12,
M₁, A₂₀, A₃₀, A₅₀, A₆₀, T₁ to T₄, L₁₁ to L₁₃, a11 to a13, R₁₀, R₂₀, R₃₀, R₄₀, R₅₀, R₆₀, b10, b20, b30, b40, b50, and b60 are respectively understood by referring to the descriptions of M₁, A₂₀, A₃₀, A₅₀, A₆₀, T₁ to T₄, L₁₁ to L₁₃, a11 to a13, R₁₀, R₂₀, R₃₀, R₄₀, R₅₀, R₆₀, b10, b20, b30, b40, b50, and b60 in claim 1,
X₁₁ is C(R₁₁) or N, and X₁₂ is C(R₁₂) or N,
X₄₁ is C(R₄₁) or N, and X₄₂ is C(R₄₂) or N, and
A₁₁ and A₄₁ are each independently a cyclopentane group, a cyclohexane group, a benzene group, a naphthalene group, a fluorene group, an anthracene group, a phenanthrene group, a carbazole group, a dibenzofuran group, a dibenzothiophene group, a dibenzosilole group, a pyridine group, a pyrimidine group, a pyrazine group, a triazine group, a quinoline group, an isoquinoline group, a quinoxaline group, or a quinazoline group,
R₁₁, R₁₂, R₄₁ and R₄₂ are each independently understood by referring to the description of R₁₀ in claim 1.

10. The organometallic compound of claim 1, wherein the organometallic compound represented by Formula 1 is represented by any one selected from Formulae 20-1 to 20-12: 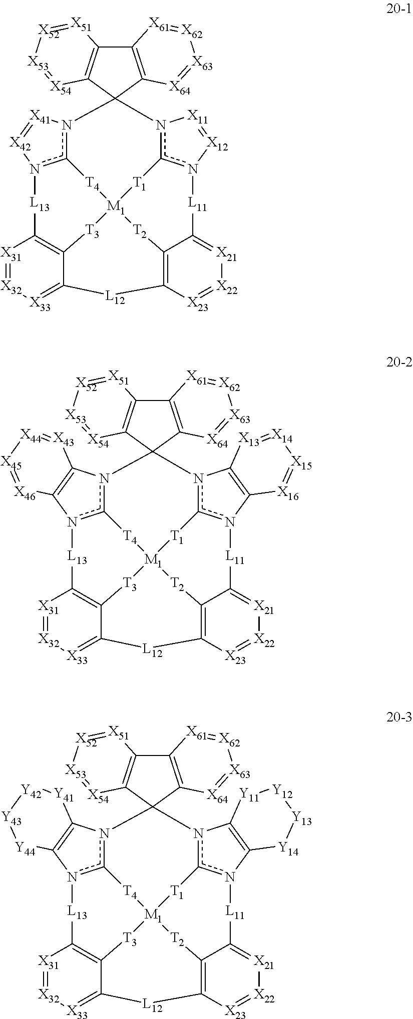 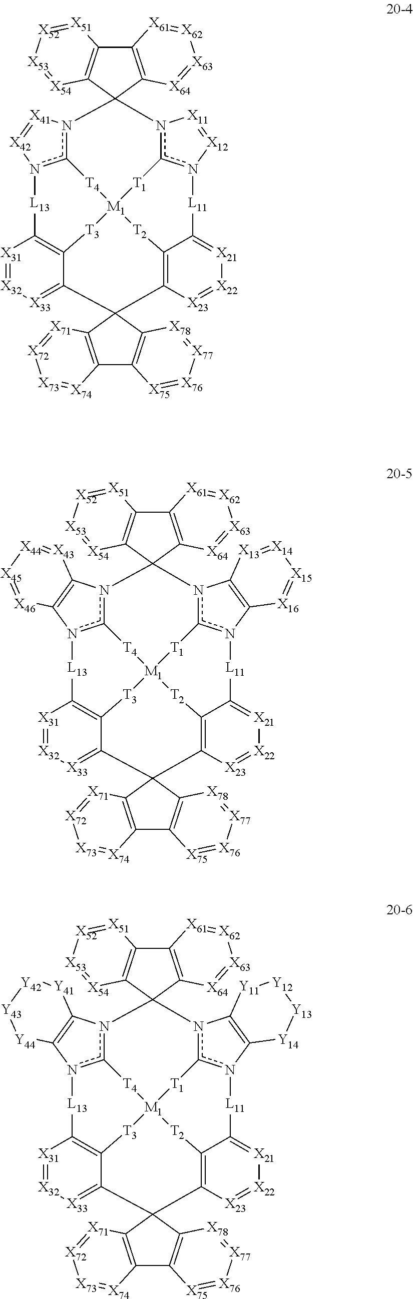 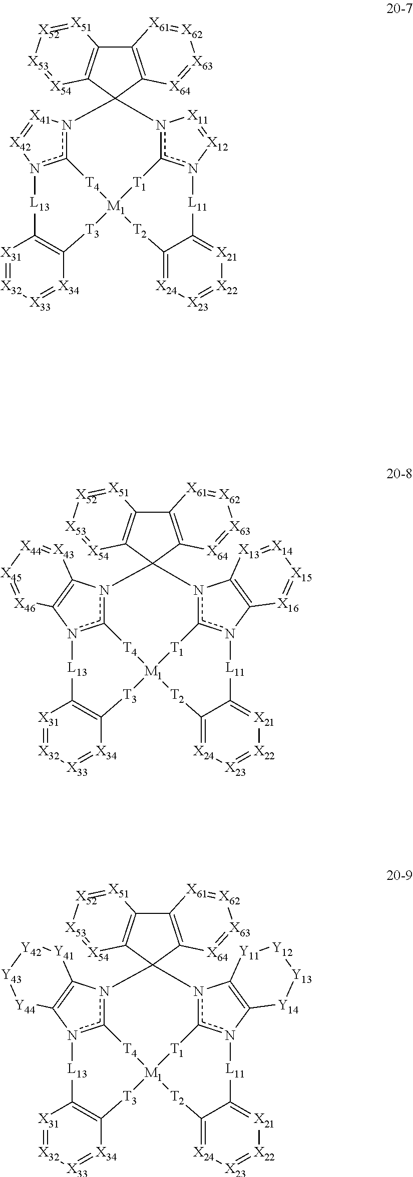 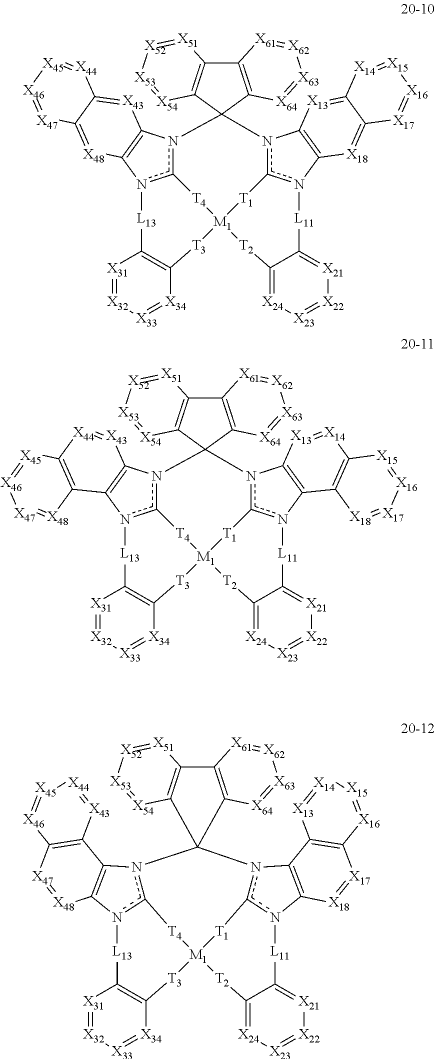
wherein, in Formulae 20-1 to 20-12,
M₁, T₁ to T₄, and L₁₁ to L₁₃ are respectively understood by referring to the descriptions of M₁, T₁ to T₄, and L₁₁ to L₁₃ in claim 1,
X₁₁ is C(R₁₁) or N, X₁₂ is C(R₁₂) or N, X₁₃ is C(R₁₃) or N, X₁₄ is C(R₁₄) or N, X₁₅ is C(R₁₅) or N, X₁₆ is C(R₁₆) or N, X₁₇ is C(R₁₇) or N, and X₁₈ is C(R₁₈) or N,
X₂₁ is C(R₂₁) or N, X₂₂ is C(R₂₂) or N, X₂₃ is C(R₂₃) or N, and X₂₄ is C(R₂₄) or N,
X₃₁ is C(R₃₁) or N, X₃₂ is C(R₃₂) or N, X₃₃ is C(R₃₃) or N, and X₃₄ is C(R₃₄) or N,
X₄₁ is C(R₄₁) or N, X₄₂ is C(R₄₂) or N, X₄₃ is C(R₄₃) or N, X₄₄ is C(R₄₄) or N, X₄₅ is C(R₄₅) or N, X₄₆ is C(R₄₆) or N, X₄₇ is C(R₄₇) or N, and X₄₈ is C(R₄₈) or N,
X₅₁ is C(R₅₁) or N, X₅₂ is C(R₅₂) or N, X₅₃ is C(R₅₃) or N, and X₅₄ is C(R₅₄) or N,
X₆₁ is C(R₆₁) or N, X₆₂ is C(R₆₂) or N, X₆₃ is C(R₆₃) or N, and X₆₄ is C(R₆₄) or N,
X₇₁ is C(R₇₁) or N, X₇₂ is C(R₇₂) or N, X₇₃ is C(R₇₃) or N, X₇₄ is C(R₇₄) or N, X₇₅ is C(R₇₅) or N, X₇₆ is C(R₇₆) or N, X₇₇ is C(R₇₇) or N, and X₇₈ is C(R₇₈) or N,
Y₁₁ is C(R₁₁)(R₁₂), Y₁₂ is C(R₁₃)(R₁₄), Y₁₃ is C(R₁₅)(R₁₆), and Y₁₄ is C(R₁₇)(R₁₈),
Y₄₁ is C(R₄₁)(R₄₂), Y₄₂ is C(R₄₃)(R₄₄), Y₄₃ is C(R₄₅)(R₄₆), and Y₄₄ is C(R₄₇)(R₄₈),
R₁₁ to R₁₈ are each independently understood by referring to the description of R₁₀ in claim 1,
R₂₁ to R₂₄ are each independently understood by referring to the description of R₂₀ in claim 1,
R₃₁ to R₃₄ are each independently understood by referring to the description of R₃₀ in claim 1,
R₄₁ to R₄₈ are each independently understood by referring to the description of R₄₀ in claim 1,
R₅₁ to R₅₄ are each independently understood by referring to the description of R₅₀ in claim 1,
R₆₁ to R₆₄ are each independently understood by referring to the description of R₆₀ in claim 1,
R₇₁ to R₇₈ are each independently: hydrogen, deuterium, —F, —Cl, —Br, —I, a cyano group, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, a cyano group, a phenyl group, a biphenyl group, or any combination thereof;
a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a carbazolyl group, an acridinyl group, a dibenzofuranyl group, a dibenzothiophenyl group, a benzocarbazolyl group, or a dibenzocarbazolyl group; or
a phenyl group, a biphenyl group, a terphenyl group, a pentalenyl group, an indenyl group, a naphthyl group, an azulenyl group, an indacenyl group, an acenaphthyl group, a fluorenyl group, a spiro-bifluorenyl group, a benzofluorenyl group, a dibenzofluorenyl group, a carbazolyl group, an acridinyl group, a dibenzofuranyl group, a dibenzothiophenyl group, a benzocarbazolyl group, or a dibenzocarbazolyl group, each substituted with deuterium, —F, —Cl, —Br, —I, a cyano group, a C₁-C₂₀ alkyl group, a C₁-C₂₀ alkoxy group, a phenyl group, a biphenyl group, or any combination thereof.

11. The organometallic compound of claim 10, wherein the organometallic compound represented by Formula 1 is represented by any one selected from Formulae 30-1 to 30-54: 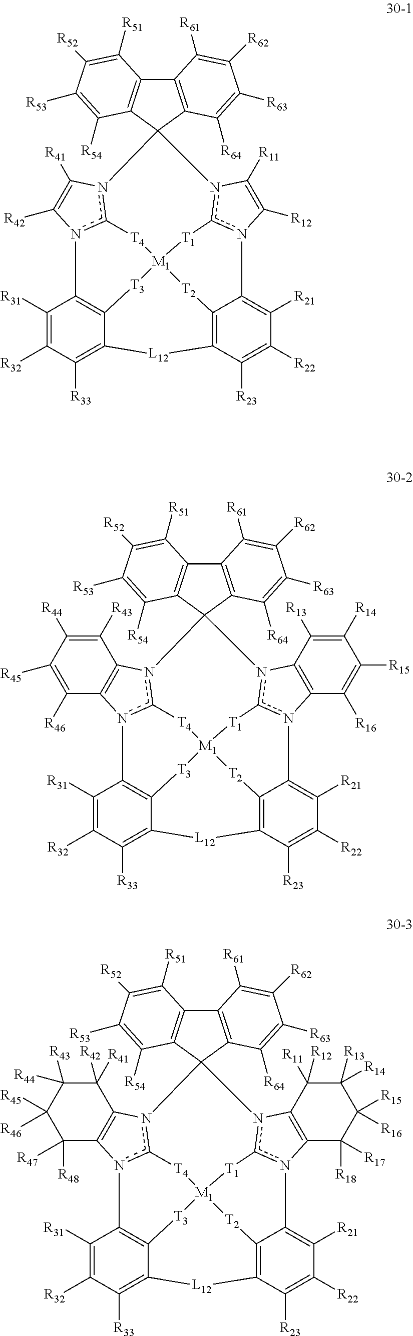 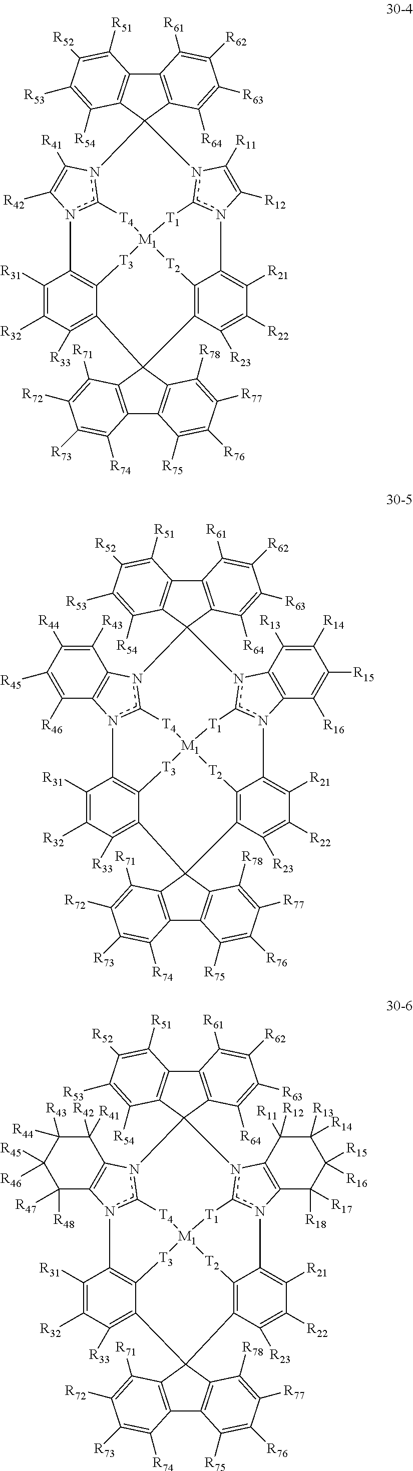 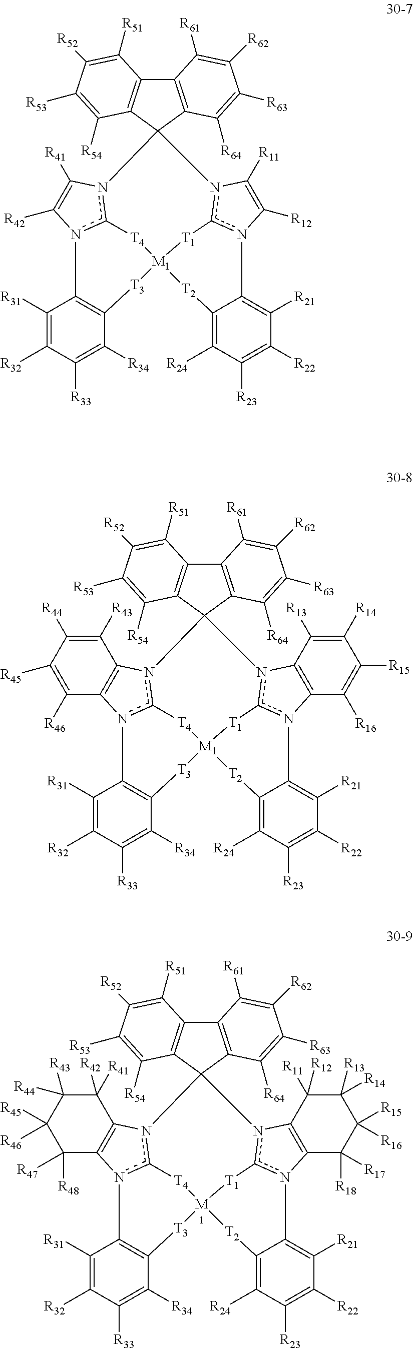 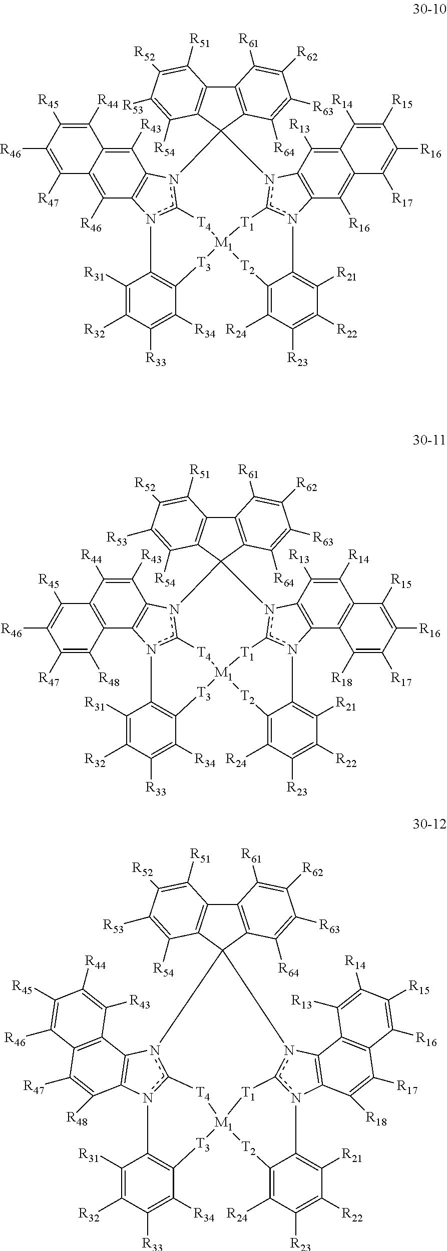 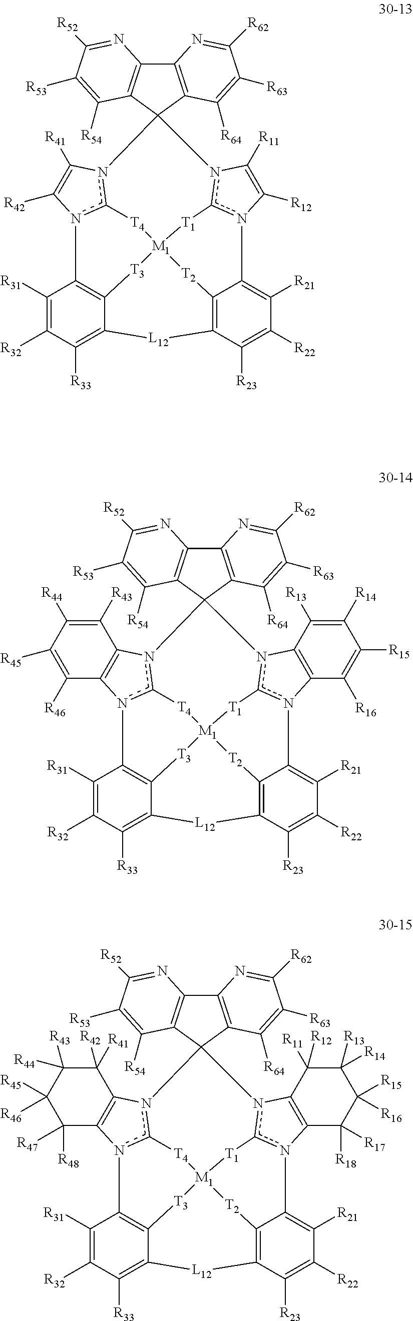 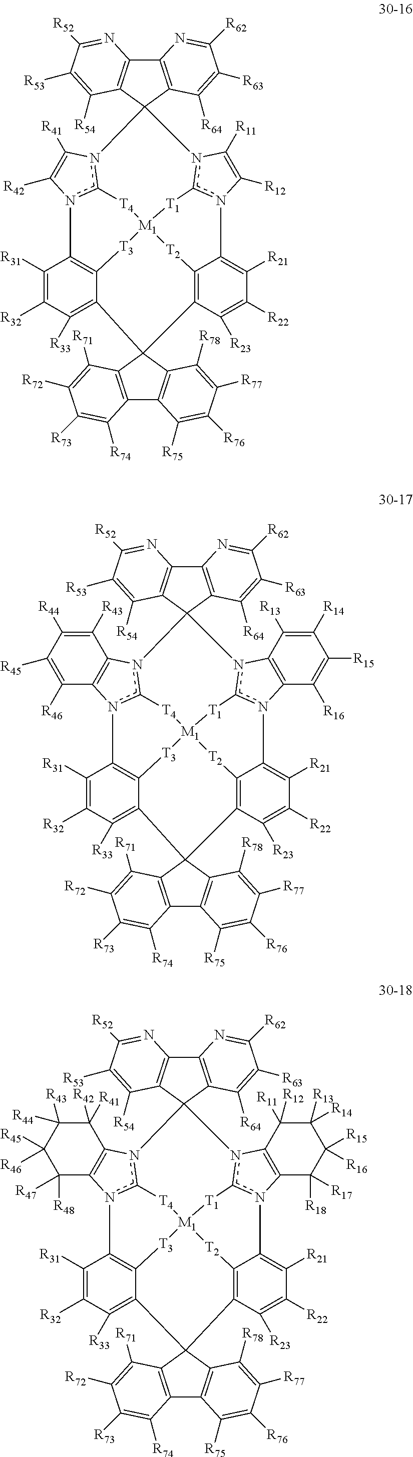 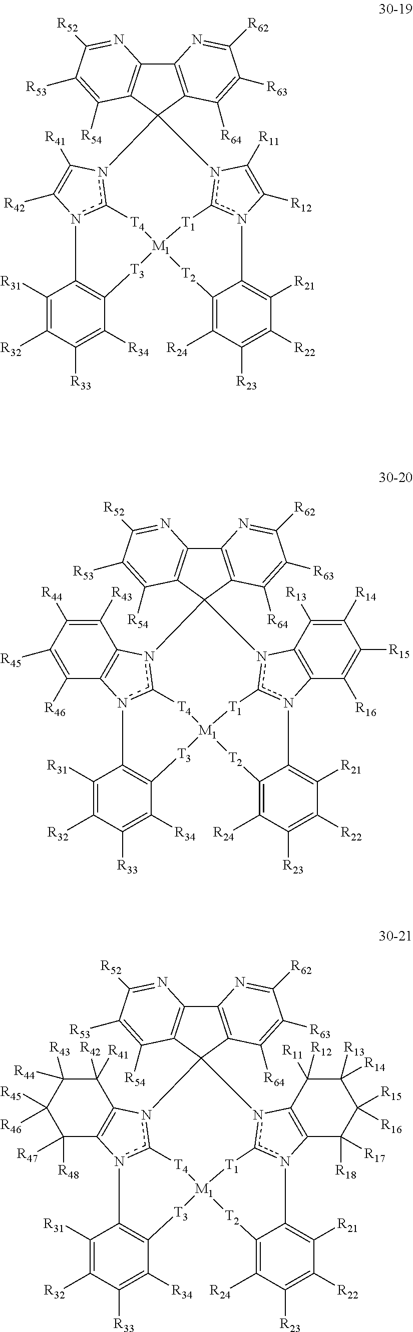 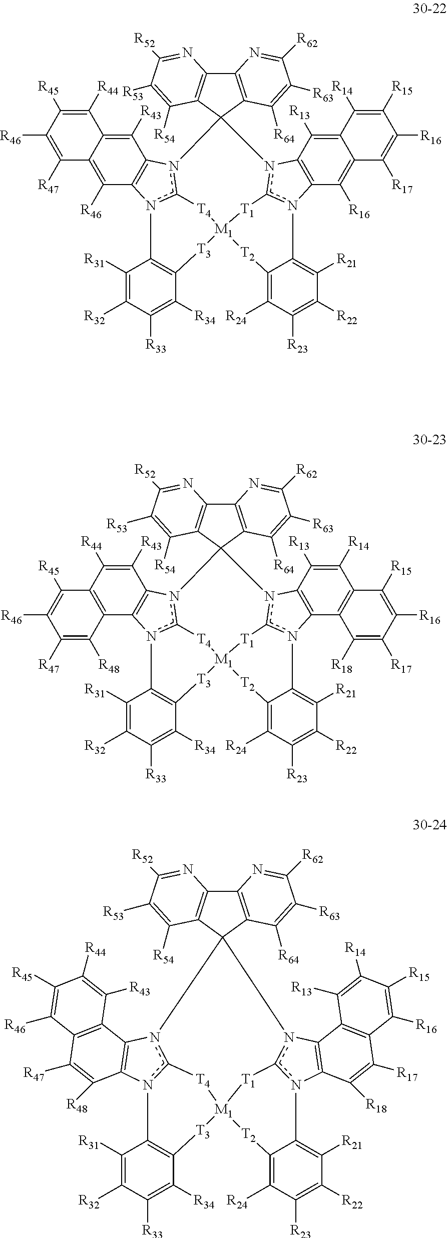 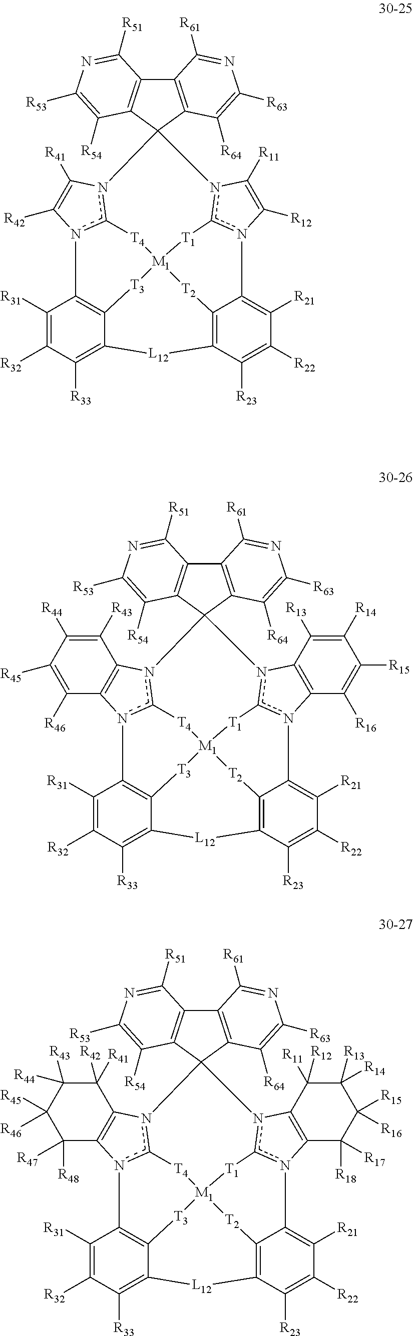 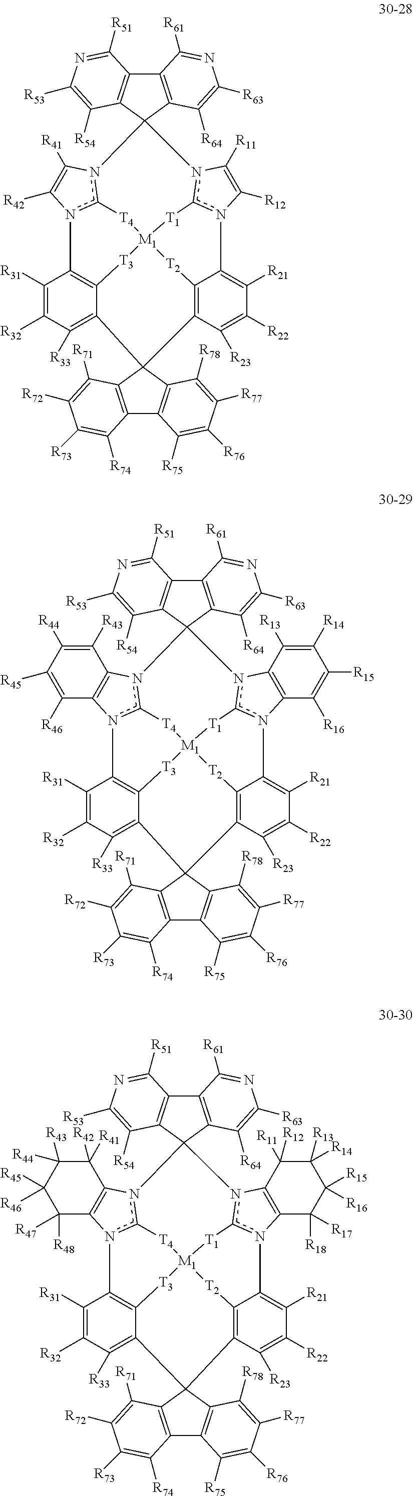 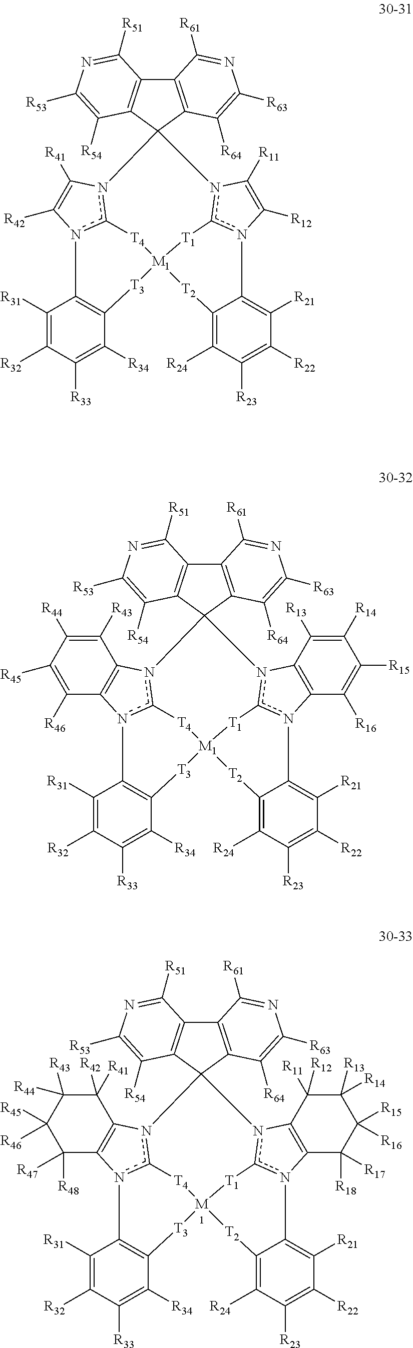 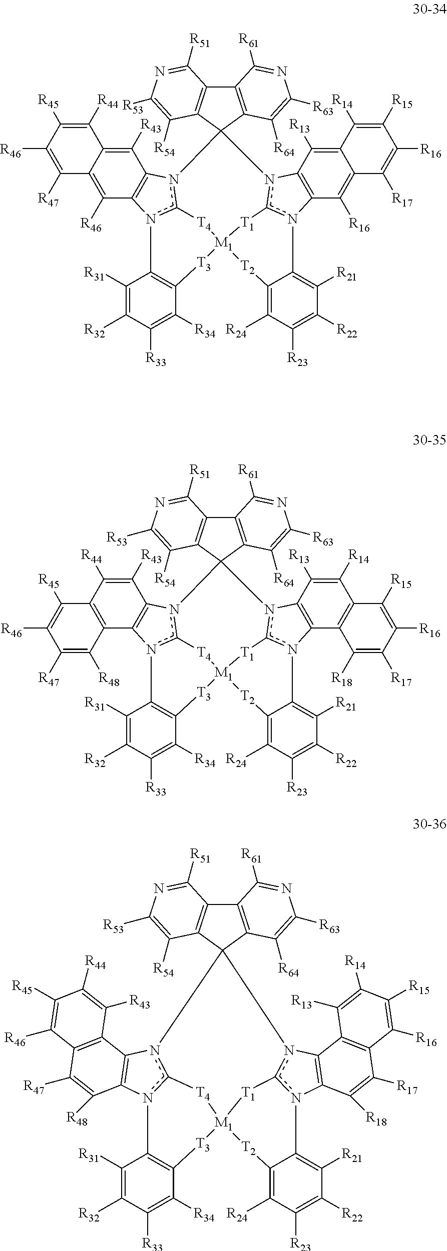 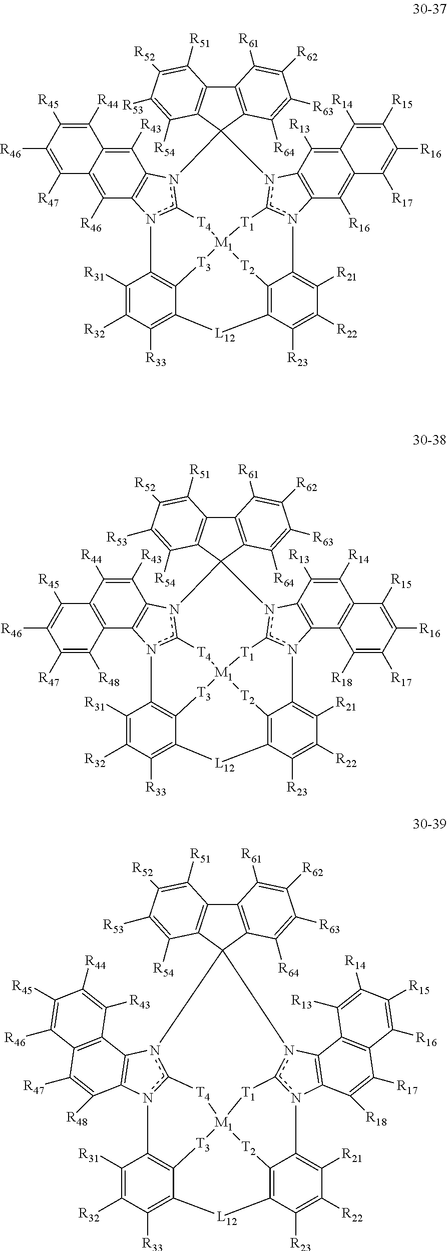 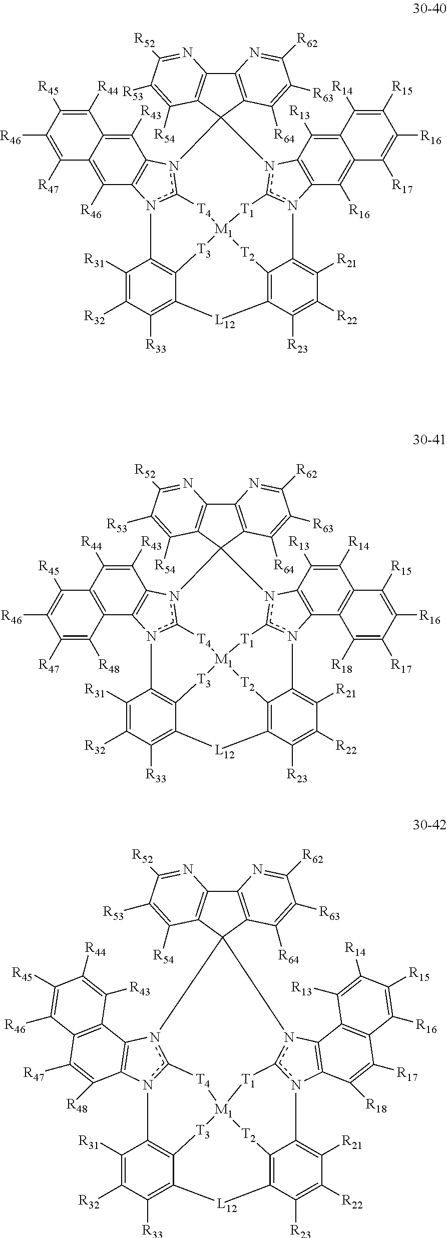 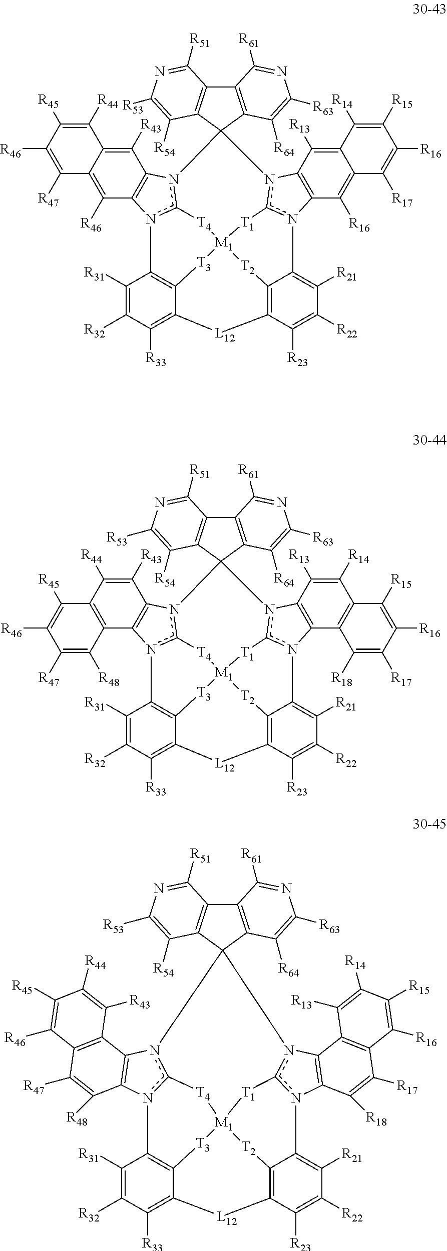 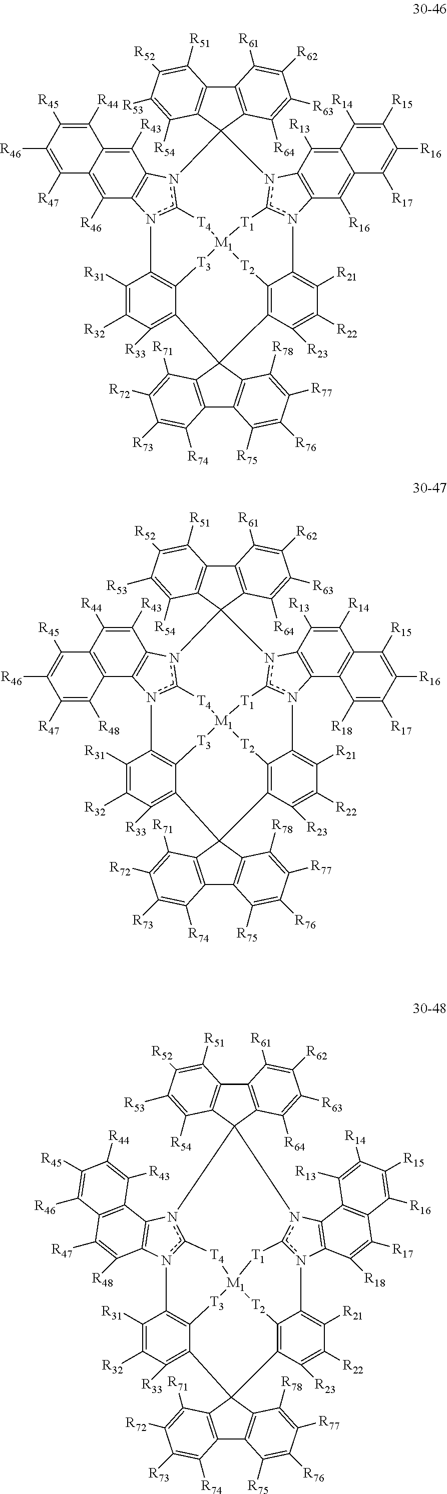 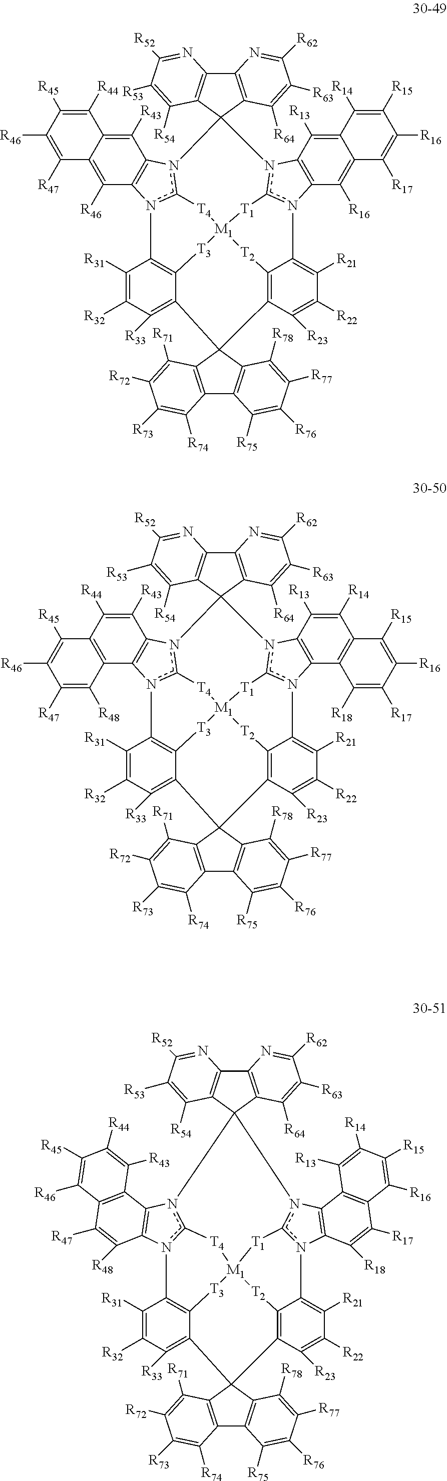 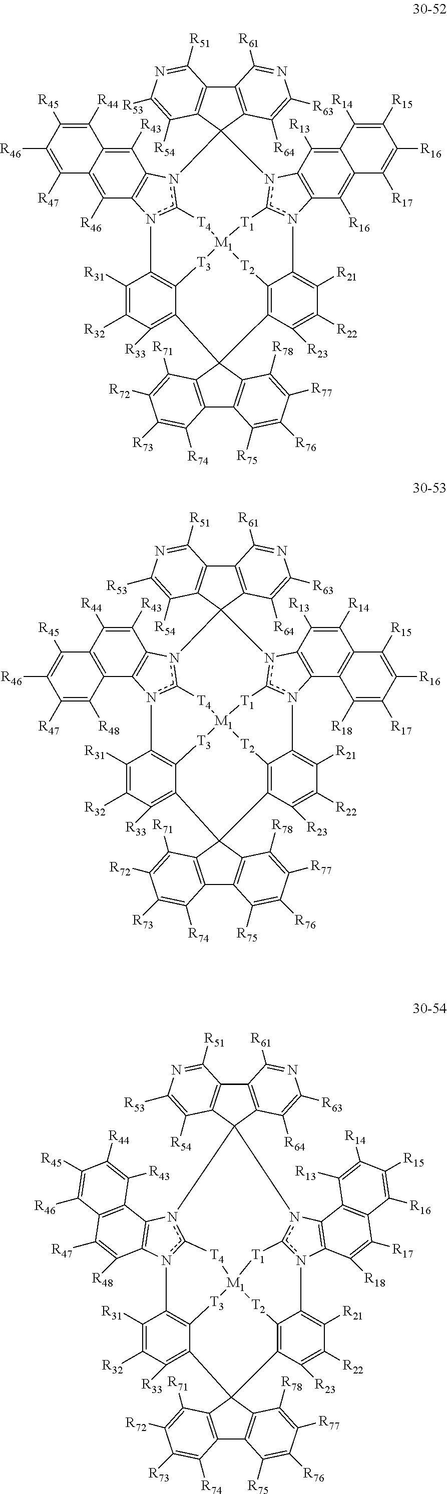
wherein, in Formulae 30-1 to 30-54,
M₁, T₁ to T₄, and L₁₂ are respectively understood by referring to the descriptions of M₁, T₁ to T₄, and L₁₂ in connection with Formula 1, and
R₁₁ to R₁₈, R₂₁ to R₂₄, R₃₁ to R₃₄, R₄₁ to R₄₈, R₅₁ to R₅₄, R₆₁ to R₆₄, and R₇₁ to R₇₈ are respectively understood by referring to the descriptions of R₁₁ to R₁₈, R₂₁ to R₂₄, R₃₁ to R₃₄, R₄₁ to R₄₈, R₅₁ to R₅₄, R₆₁ to R₆₄, and R₇₁ to R₇₈ in connection with Formulae 20-1 to 20-12.

12. The organometallic compound of claim 1, wherein the organometallic compound is electrically neutral.

13. The organometallic compound of claim 1, wherein the organometallic compound represented by Formula 1 is selected from Compounds BD1 to BD66:

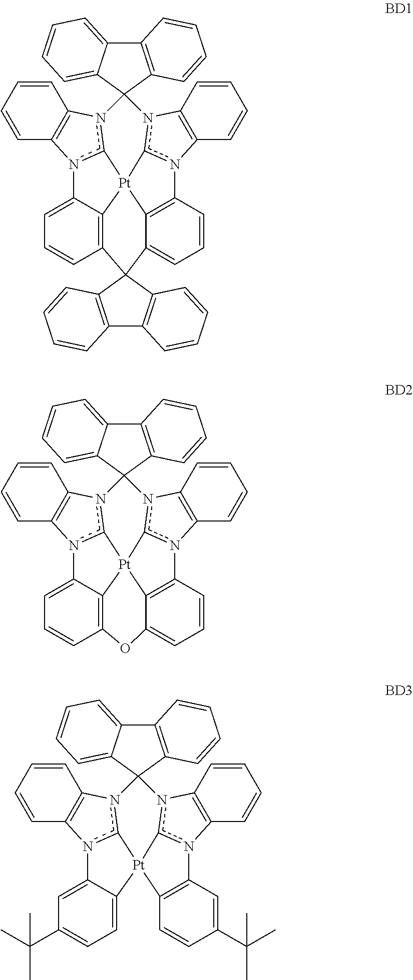

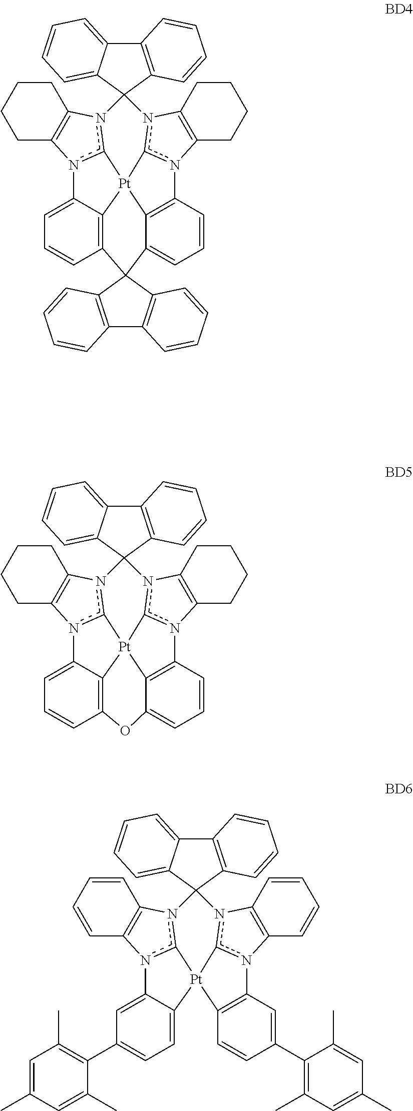

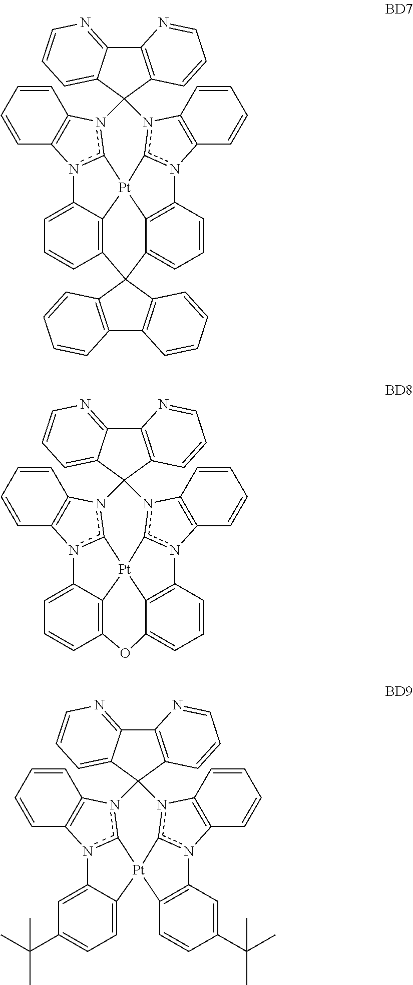

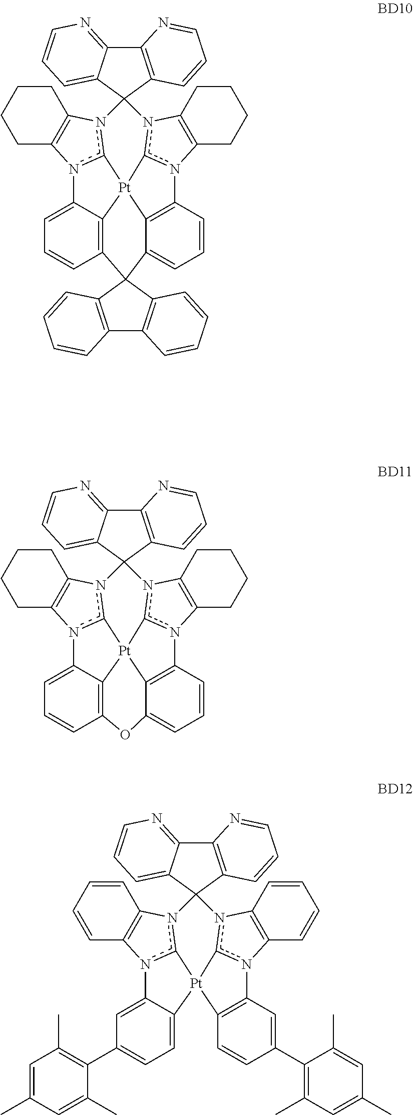

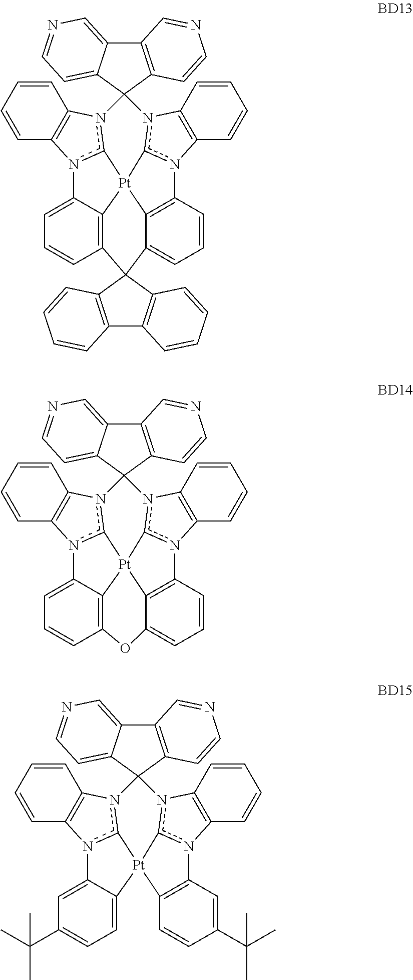

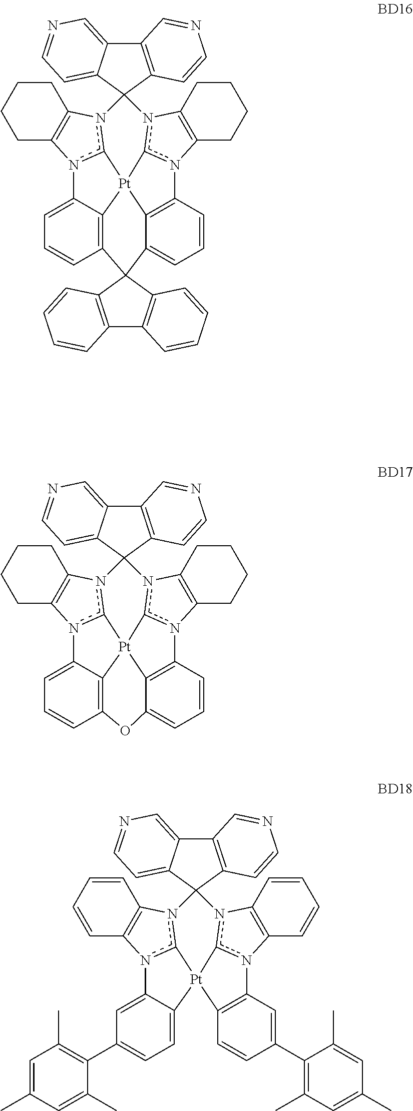

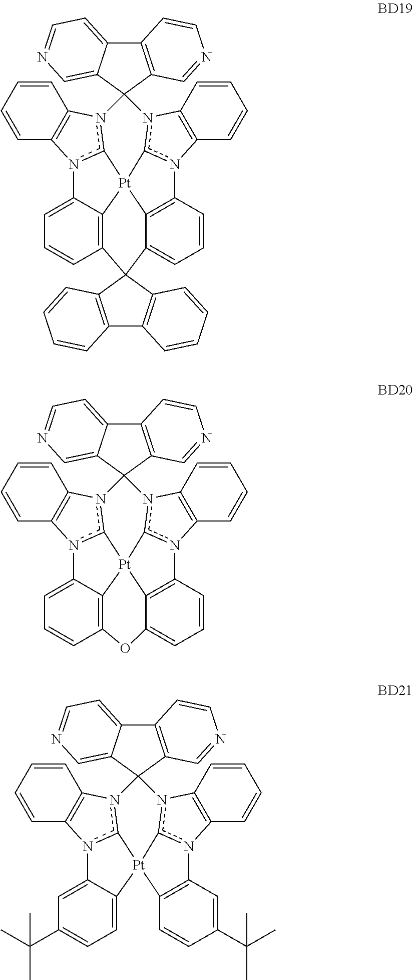

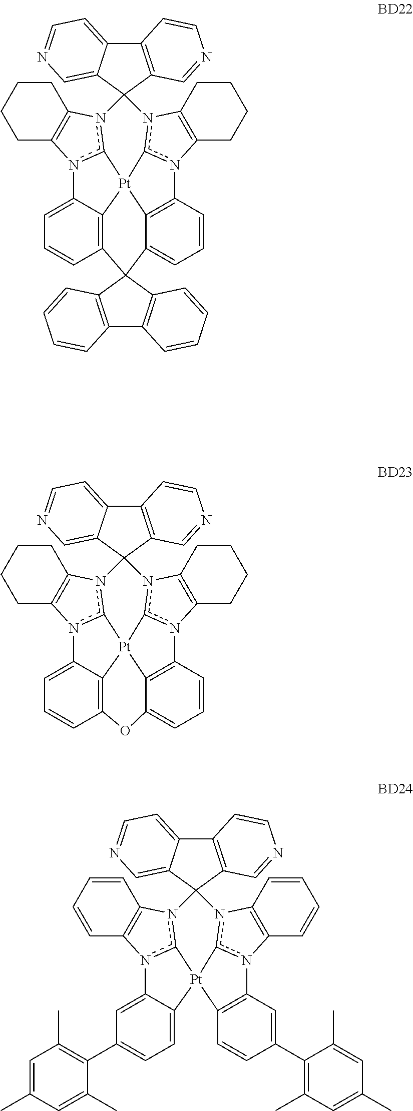

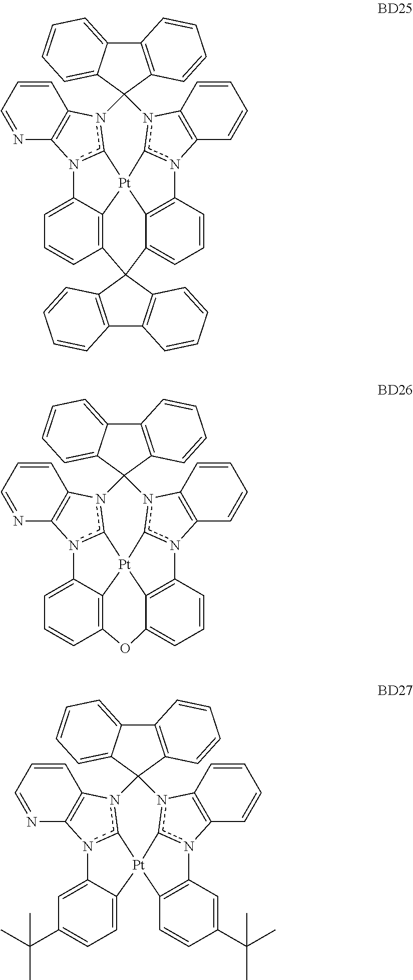

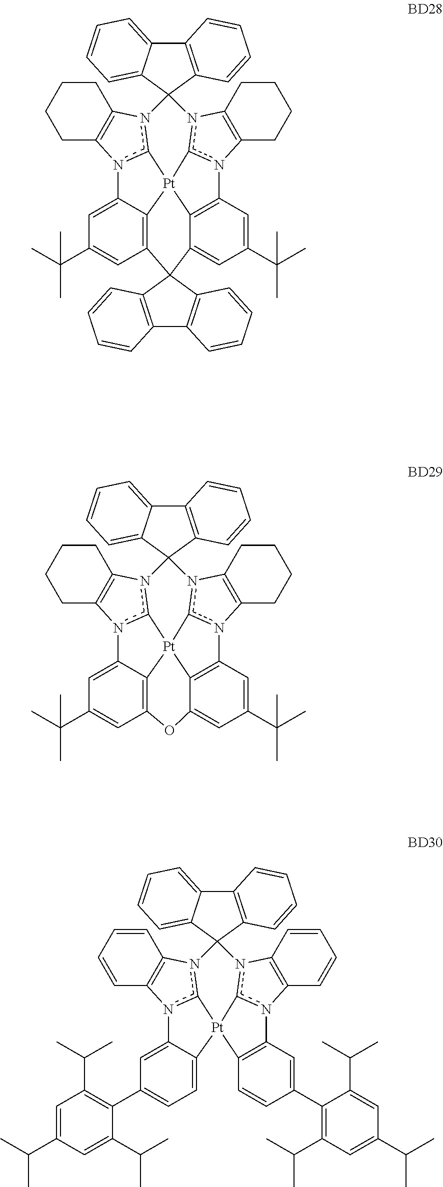

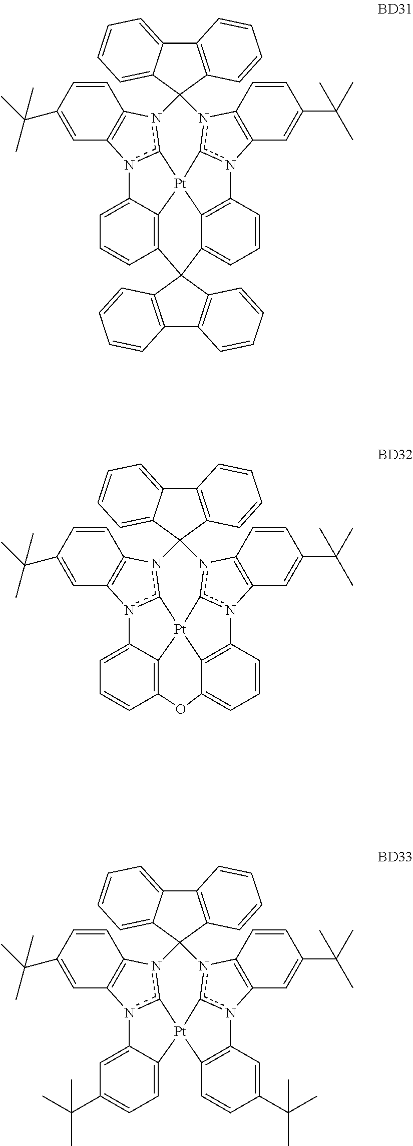

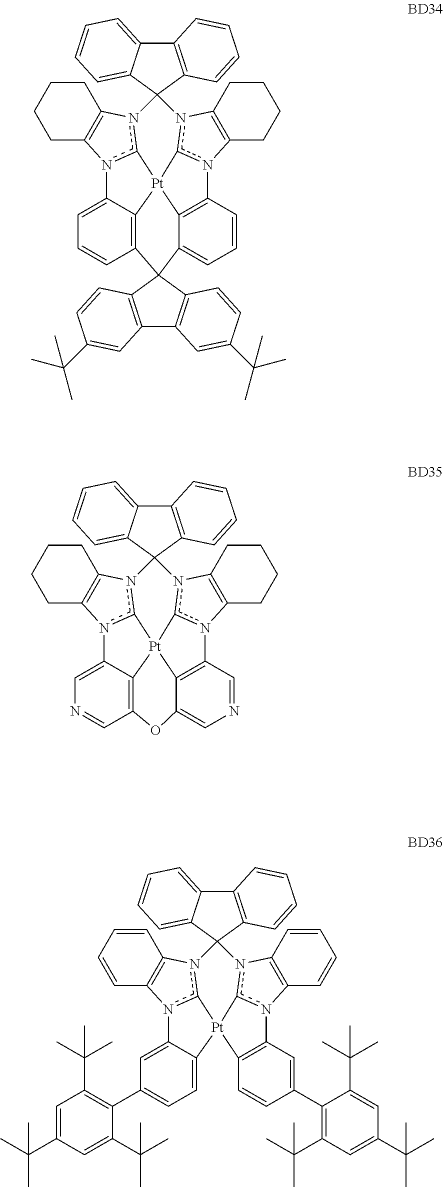

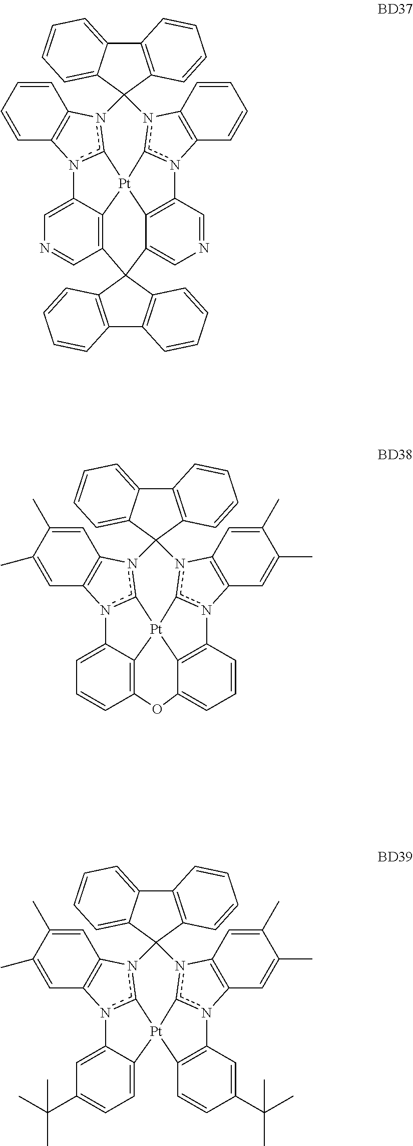

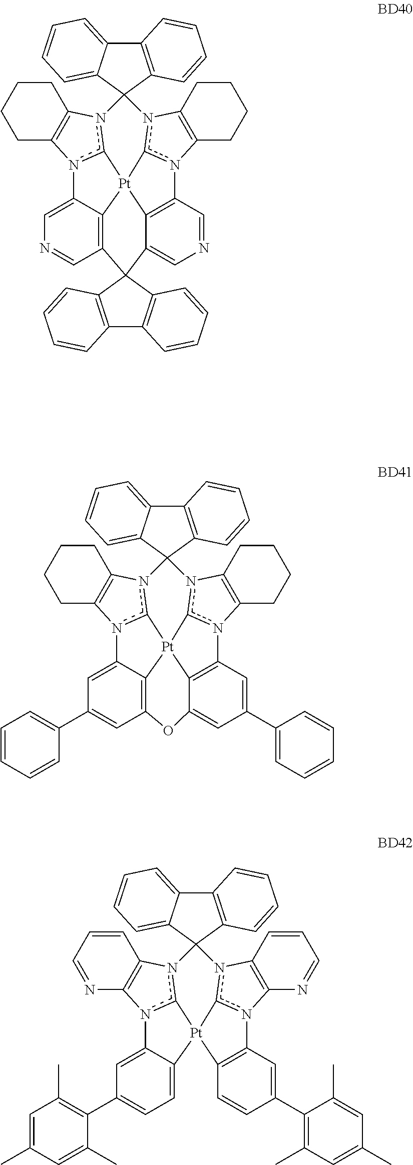

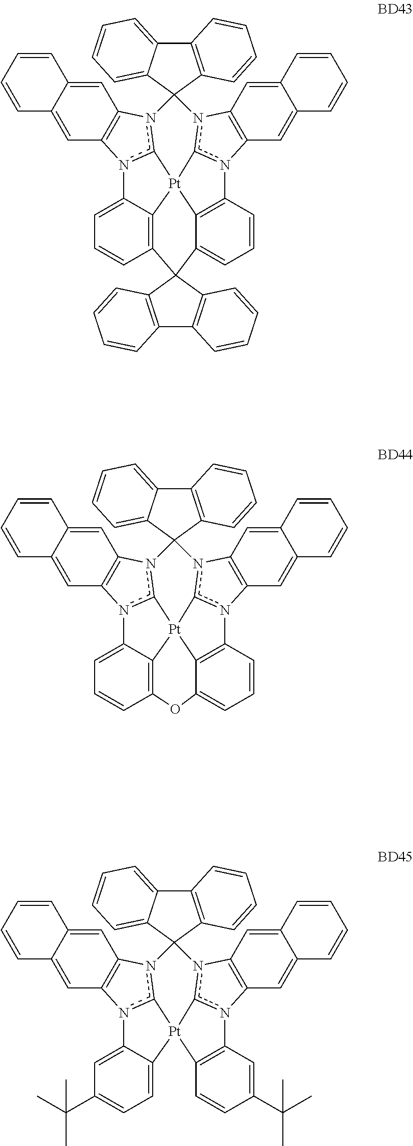

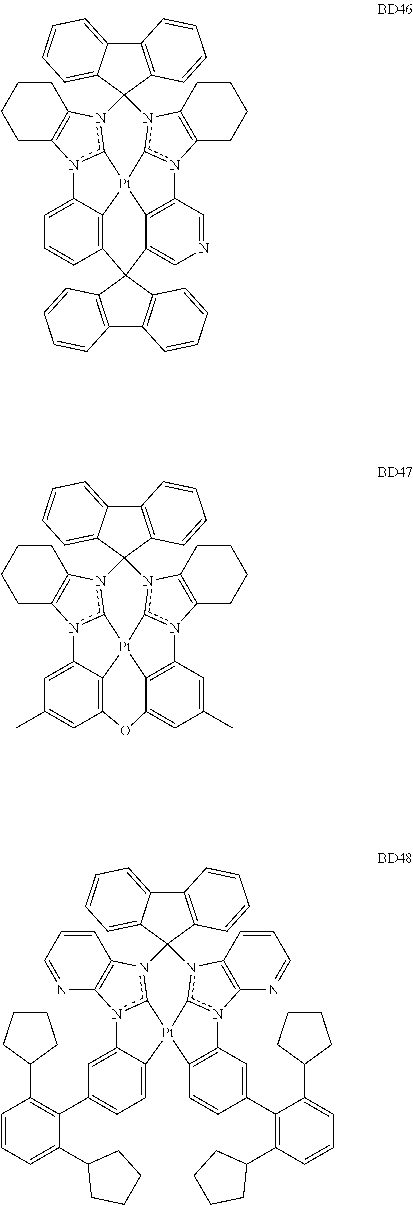

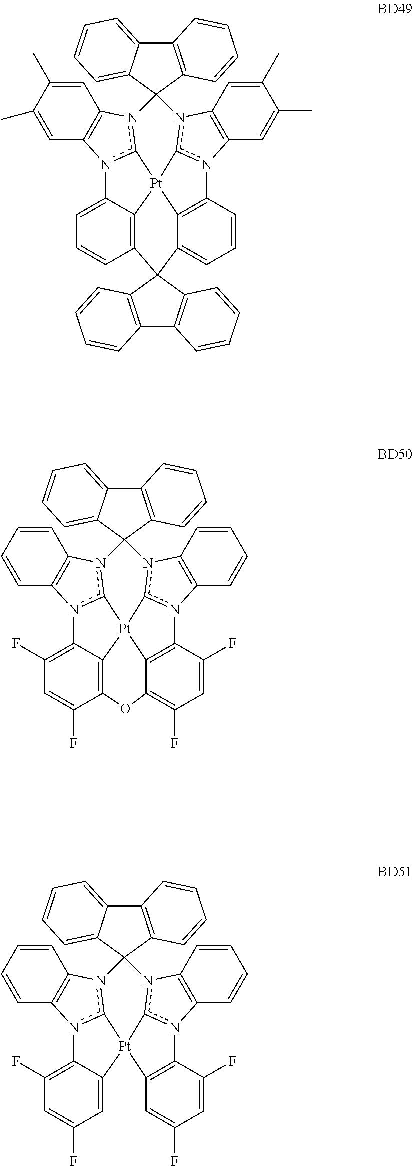

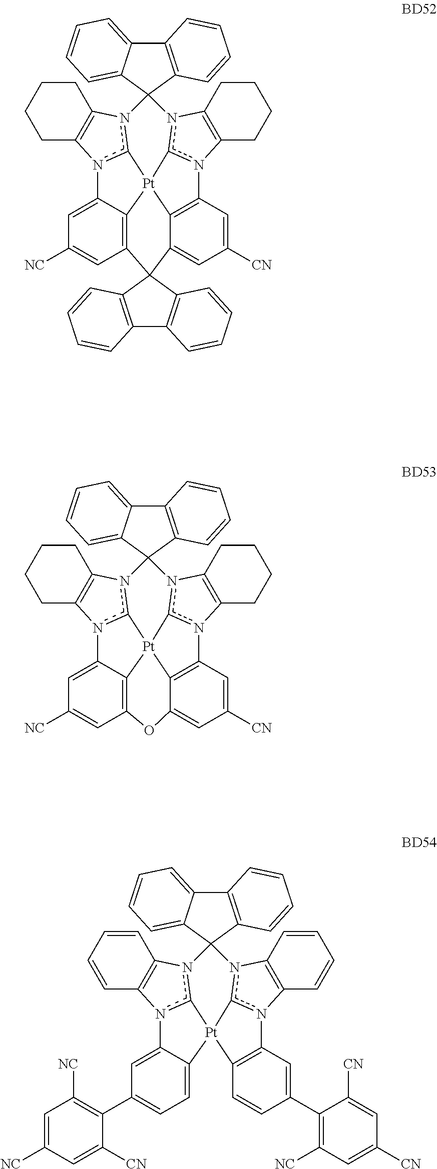

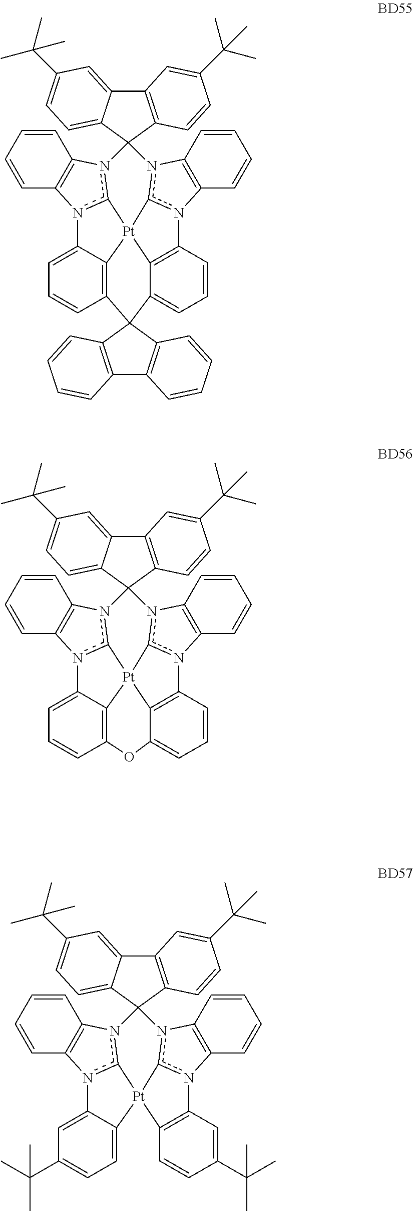

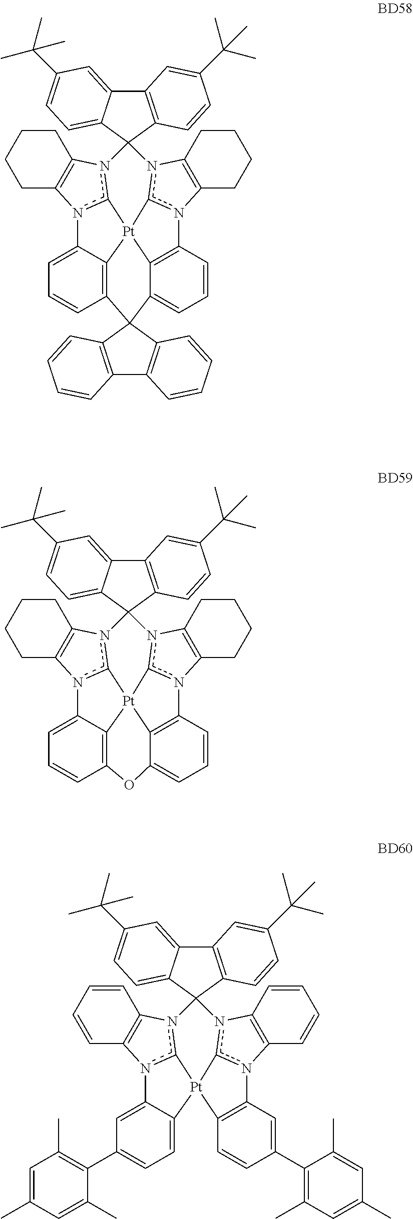

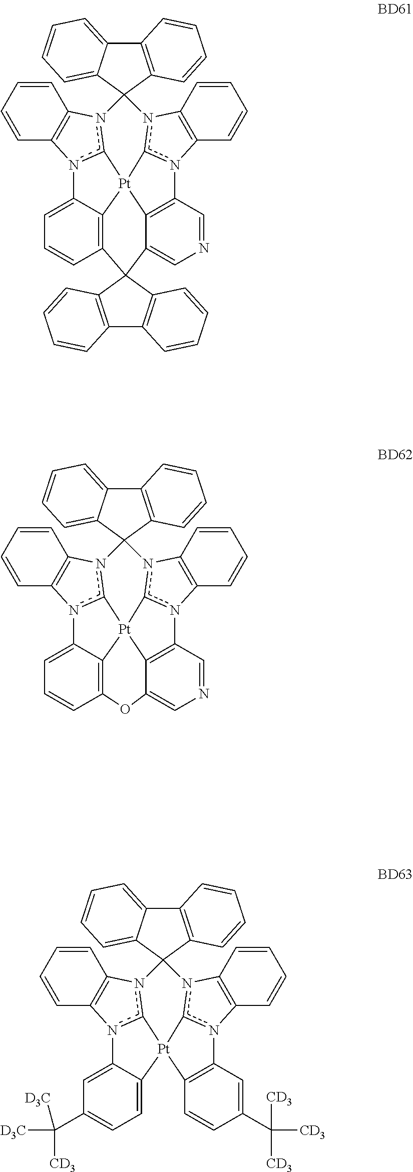

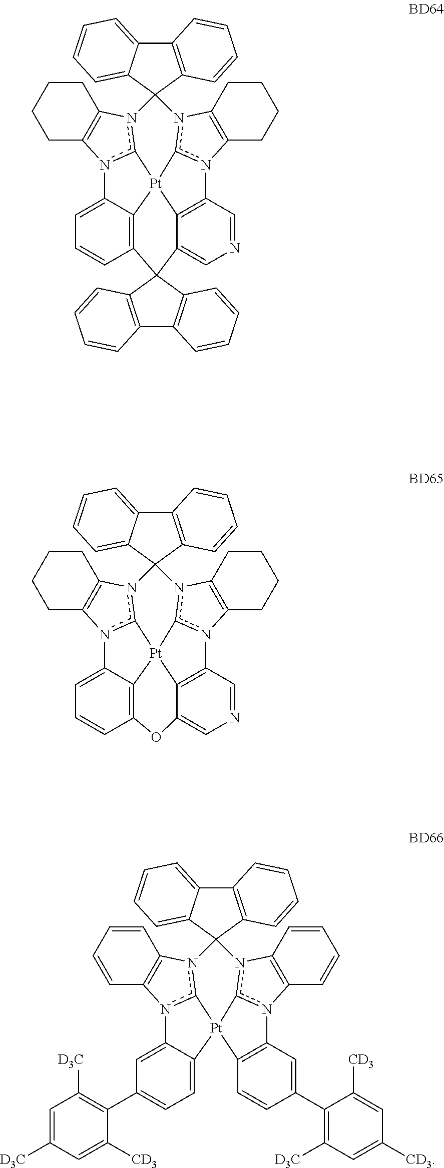

14. An organic light-emitting device comprising:
a first electrode;
a second electrode facing the first electrode;
an organic layer between the first electrode and the second electrode and comprising an emission layer; and
at least one of the organometallic compound represented by Formula 1 of claim 1.

15. The organic light-emitting device of claim 14, wherein:
the first electrode is an anode,
the second electrode is a cathode, and
the organic layer comprises a hole transport region between the first electrode and the emission layer and an electron transport region between the emission layer and the second electrode,
the hole transport region comprises at least one selected from a hole injection layer, a hole transport layer, a buffer layer, an emission auxiliary layer, and an electron blocking layer, and
the electron transport region comprises at least one selected from a buffer layer, a hole blocking layer, an electron control layer, an electron transport layer, and an electron injection layer.

16. The organic light-emitting device of claim 14, wherein the emission layer comprises the organometallic compound.

17. The organic light-emitting device of claim 16, wherein the emission layer comprises a host and a dopant, and the dopant comprises the organometallic compound.

18. The organic light-emitting device of claim 16, wherein the emission layer emits blue light having a maximum emission wavelength in a range of about 410 nanometers (nm) to 500 nm.

19. An electronic apparatus comprising the organic light-emitting device of claim 14.

20. The electronic apparatus of claim 19, the electronic apparatus further comprising a thin-film transistor, wherein the thin-film transistor comprises a source electrode and a drain electrode, and the first electrode of the organic light-emitting device is electrically coupled to the source electrode or the drain electrode.
